(19) United States
(12) Patent Application Publication
(10) Pub. No.: US 2019/0209682 A1
(43) Pub. Date: Jul. 11, 2019

(54) ISOFORM-SPECIFIC, CONTEXT-PERMISSIVE TGFB1 INHIBITORS AND USE THEREOF

(71) Applicant: Scholar Rock, Inc., Cambridge, MA (US)

(72) Inventors: Thomas Schurpf, Cambridge, MA (US); Abhishek Datta, Boston, MA (US); Gregory J. Carven, Maynard, MA (US); Constance Martin, Arlington, MA (US); Ashish Kalra, Belmont, MA (US); Kimberly Long, Boston, MA (US); Alan Buckler, Arlington, MA (US)

(21) Appl. No.: 16/361,486

(22) Filed: Mar. 22, 2019

Related U.S. Application Data
(63) Continuation of application No. 15/863,564, filed on Jan. 5, 2018.
(60) Provisional application No. 62/588,626, filed on Nov. 20, 2017.
(60) Provisional application No. 62/587,964, filed on Nov. 17, 2017.
(60) Provisional application No. 62/585,227, filed on Nov. 13, 2017.
(60) Provisional application No. 62/558,311, filed on Sep. 13, 2017.
(60) Provisional application No. 62/549,767, filed on Aug. 24, 2017.
(60) Provisional application No. 62/529,616, filed on Jul. 7, 2017.
(60) Provisional application No. 62/514,417, filed on Jun. 2, 2017.
(60) Provisional application No. 62/452,866, filed on Jan. 31, 2017.
(60) Provisional application No. 62/443,615, filed on Jan. 6, 2017.

(51) Int. Cl. A61K 39/395; C07K 16/22; G01N 33/574; A61P 35/04

(52) U.S. Cl. CPC A61K 39/39541; C07K 16/22; A61K 2039/505; A61P 35/04; C07K 2317/76; G01N 33/574

(57) ABSTRACT

Disclosed herein are therapeutic use of isoform-specific, context-permissive inhibitors of TGFβ1 in the treatment of disease that involve TGFβ1 dysregulation.

### RELATED APPLICATIONS

This application is a continuation of U.S. patent application Ser. No. 15/836,564, filed Jan. 5, 2018, which claims priority to and benefit under 35 U.S.C. § 119(e) of the following applications: U.S. Provisional Application No. 62/443,615, filed on Jan. 6, 2017; U.S. Provisional Application No. 62/452,866, filed on Jan. 31, 2017; U.S. Provisional Application No. 62/514,417, filed on Jun. 2, 2017; U.S. Provisional Application 62/529,616, filed on Jul. 7, 2017, U.S. Provisional Application No. 62/549,767, filed on Aug. 24, 2017, U.S. Provisional Application No. 62/558,311, filed on Sep. 13, 2017, U.S. Provisional Application No. 62/585,227 filed on Nov. 13, 2017, U.S. Provisional Application No. 62/587,964 filed on Nov. 17, 2017, and U.S. Provisional Application No. 62/588,626 filed on Nov. 20, 2017, the contents of each of which are expressly incorporated herein by reference in their entireties.

### SEQUENCE LISTING

The instant application contains a Sequence Listing which has been submitted electronically in ASCII format and is hereby incorporated by reference in its entirety. Said ASCII copy, created on Jan. 5, 2018, is named 2018_01_05_127036-02008_ST25.txt and is 221,825 bytes in size.

### BACKGROUND OF THE INVENTION

Transforming growth factor β (TGFβ) superfamily of growth factors are involved in a number of signaling cascades that regulate diverse biological processes including, but not limited to: inhibition of cell growth, tissue homeostasis, extracellular matrix (ECM) remodeling, endothelial to mesenchymal transition (EMT), cell migration and invasion, and immune modulation/suppression, as well as mesenchymal to epithelial transition. In relation to ECM remodeling, TGFβ signaling may increase fibroblast populations and ECM deposition (e.g., collagens). In the immune system, TGFβ ligand modulates T regulatory cell function and maintenance of immune precursor cell growth and homeostasis. In normal epithelial cells, TGFβ is a potent growth inhibitor and promoter of cellular differentiation. However, as tumors develop and progress, they frequently lose their negative growth response to TGFβ. In this setting, TGFβ may become a promoter of tumor development due to its ability to stimulate angiogenesis, alter the stromal environment, and induce local and systemic immunosuppression. For these and other reasons, TGFβ has been a therapeutic target for a number of clinical indications. Despite much effort made to date by a number of groups, successful clinical development of a TGFβ therapeutic has been challenging.

Observations from preclinical studies, including in rats and dogs, have revealed certain toxicities associated with inhibiting TGFβ in vivo. Moreover, although several TGFβ inhibitors have been developed to date, most clinical programs targeting TGFβ have been discontinued due to side effects (summarized, for example, in WO 2017/156500). Thus, despite lines of direct and indirect evidence pointing to the involvement of TGFβ signaling in the progression of diseases such as cancer and fibrosis, there is no TGFβ therapeutics available in the market which are safe and efficacious.

Among proliferative disorders, dysregulation of TGFβ has also been implicated in myelofibrosis, which is a bone marrow disorder characterized by clonal myeloproliferation, aberrant cytokine production, extramedullary hematopoiesis, and bone marrow fibrosis. Although somatic mutations in JAK2, MPL and CALR have been identified in the pathogenesis of the disease, Ruxolitinib (Jakafi), which is a JAK1/JAK2 inhibitor approved by the FDA for the treatment of myelofibrosis, has not demonstrated efficacy in ameliorating established bone marrow fibrosis in patients.

Thus, improved methods and compositions for inhibiting TGFβ signaling are needed that can be used to effectively and safely treat diseases and disorders involving TGFβ1, including, for example, proliferative disorders (e.g., cancer), fibrosis and inflammation.

### SUMMARY OF THE INVENTION

The present invention encompasses the recognition that blocking TGFβ activation at multiple sources may provide greater clinical effects in treating a number of diseases involving both an ECM aspect and an immune aspect of TGFβ dysregulation. Accordingly, provided herein are improved methods for treating such diseases with TGFβ1 inhibitors which are superior to conventional TGFβ antagonists with respect to their isoform selectivity, breadth of molecular targets within a disease niche, durability of effects and safety.

A body of evidence supports the notion that many diseases manifest complex perturbations of TGFβ signaling, which likely involve participation of heterogeneous cell types that confer different effects of TGFβ function, which are mediated by its interactions with so-called presenting molecules. At least four such presenting molecules have been identified, which can “present” TGFβ in various extracellular niches to enable its activation in response to local stimuli. In one category, TGFβ is deposited into the ECM in association with ECM-associated presenting molecules, such as LTBP1 and LTBP3, which mediate ECM-associated TGFβ activities. In another category, TGFβ is tethered onto the surface of immune cells, via presenting molecules such as GARP and LRRC33, which mediate certain immune function. These presenting molecules show differential expression, localization and/or function in various tissues and cell types, indicating that triggering events and outcome of TGFβ activation will vary, depending on the microenvironment. Based on the notion that many TGFβ effects may interact and contribute to disease progression, therapeutic agents that can antagonize multiple facets of TGFβ function may provide greater efficacy.

Previously, the inventors recognized that isoform-specific inhibition (as opposed to pan-inhibition) of TGFβ may render improved safety profiles of antagonizing TGFβ in vivo (see WO 2017/156500). Taking this into consideration, the inventors have sought to develop TGFβ1 inhibitors that are both i) isoform-specific; and, ii) capable of broadly targeting multiple TGFβ1 signaling complexes that are associated with different presenting molecules, as therapeutic agents for conditions driven by multifaceted TGFβ1 effects and dysregulation thereof.

Accordingly, the present disclosure provides isoform-specific inhibitory agents capable of targeting both ECM-associated TGFβ1 and immune cell-associated TGFβ1, thereby blocking multiple sources of TGFβ1 presented in multiple contexts. Such inhibitory agents are referred herein to as “isoform-specific, context-permissive” inhibitors of TGFβ1. The invention also provides use of these agents as a therapeutic in the treatment of conditions that are characterized by dysregulation of TGFβ1 signaling associated with multiple aspects of TGFβ1 function. Such inhibitors may function as multifunctional agents to antagonize multiple TGFβ1 activities (e.g., TGFβ1 from multiple sources or contexts) to enhance clinical effects in the context of fibrosis, myelofibrosis, cancer, and other conditions.

The rationale for the advantageous use of context-permissive (such as context-independent) inhibitors of TGFβ1 over context-specific inhibitors of TGFβ1 as a therapeutic to treat certain diseases (as described in further detail herein) include the following:

Involvement of Heterogeneous TGFβ1 Complexes in a Disease Environment:

First, various diseases involve heterogeneous populations of cells as multiple sources of TGFβ1 that collectively contribute to the pathogenesis and/or progression of the disease. More than one types of TGFβ1-containing complexes (“contexts”) likely coexist within the same disease microenvironment. In particular, such diseases may involve both an ECM component of TGFβ1 signaling and an immune component of TGFβ1 signaling. In such situations, selective targeting of a single TGFβ1 context (e.g., TGFβ1 associated with one type of presenting molecule) may offer limited relief. By contrast, context-permissive inhibitors of TGFβ1 are advantageously aimed to more broadly target inactive (pro/latent) TGFβ1 complexes and prevent activation of the growth factor at multiple sources before mature TGFβ1 can be released for receptor binding to trigger downstream signaling, while maintaining the isoform selectivity to minimize toxicities.

Common mechanisms underlining various diseases: Second, notable similarities in tissue/cellular characteristics are observed between the tumor stroma and fibrotic tissues. Indicating crosstalk between and among: i) TGFβ1-dependent pro-fibrotic phenotypes; ii) TGFβ1-dependent pro-tumor phenotypes; and, iii) TGFβ-dependent immunosuppressive phenotypes, observed in a number of pathological conditions. Thus, the use of context-permissive inhibitors that broadly act upon many of these constituents may provide optimal therapeutic effects across a diverse types of disease conditions. For example, clinical manifestations of primary myelofibrosis include abnormal proliferation of certain cell populations and fibrosis in the bone marrow.

Countering drug resistance: Third, a number of studies have reported cancer/tumors which are resistant to anti-cancer therapies, such as immuno checkpoint inhibitors. In some cases, such resistance appears intrinsic to the particular cancer/tumor-type against the patient's background (typically referred to as innate resistance, primary resistance, intrinsic resistance, or inherent resistance; these terms are used interchangeably herein). Such resistance may be represented in a subject of patients poorly responsive to cancer therapies such as immune checkpoint inhibitors and possibly reflect immune-excluded environment. This is likely mediated at least in part by a TGFβ1-dependent pathway. Thus, isoform-selective inhibitor described herein may render the resistant cancers more responsive to such therapies.

Alternatively, resistance may develop over time such that patients who show material clinical responsiveness to a treatment become poorly responsive (i.e., adaptive or acquired resistance). For example, it has been reported that PD-1 therapy can lead to adaptive resistance which is correlated with upregulation of other T cell antigens (e.g., TCR components) suggesting that cancer cells evolve to evade the PD-1 blockade via another mechanism. Subsequently, a second checkpoint inhibitor that targets a different T cell receptor component such as TIM3 can restore responsiveness to the immunotherapy. These observations suggest that blocking multiple pathways to counter adaptive responses of cancer cells may reduce the likelihood of cancer cells' ability to evade host immunity. Context-permissive inhibitors of TGFβ1 which are capable of targeting multiple TGFβ1 contexts may advantageously circumvent acquired drug resistance by providing blockade at multiple points of the TGFβ1 function.

Withstanding expression plasticity: And finally, based on the notion that expression of various presenting molecules may vary over time, for example, in response to local cues (e.g., cytokines, chemokines, ECM environment, etc.) and/or with changes in a disease microenvironment, it is reasoned that context-permissive inhibitors of TGFβ1 such as those described herein may be used to withstand such plasticity and provide broad, durable inhibitory effects even when abnormal changes in expression of the presenting molecules occur.

In any of these scenarios, the context-permissive inhibitors of TGFβ1 are advantageously aimed to target the pro/latent forms of TGFβ1 in association with various presenting molecules, all of which or different combinations of which are present in a disease microenvironment(s). More specifically, in one modality, the inhibitor targets ECM-associated TGFβ1 (LTBP1/3-TGFβ1 complexes). In another modality, the inhibitor targets immune cell-associated TGFβ1. This includes GARP-presented TGFβ1, such as GARP-TGFβ1 complexes expressed on Treg cells and LRRC33-TGFβ1 complexes expressed on macrophages and other myeloid/lymphoid cells, as well as certain cancer cells.

Such antibodies include isoform-specific inhibitors of TGFβ1 that bind and prevent activation (or release) of mature TGFβ1 growth factor from a pro/latent TGFβ1 complex in a context-permissive (or context-independent) manner, such that the antibodies can inhibit activation (or release) of TGFβ1 associated with multiple types of presenting molecules. In particular, the present invention provides antibodies capable of blocking at least one context of ECM-associated TGFβ1 (LTBP-presented and/or LTBP3-presented) and at least one context of cell-associated TGFβ1 (GARP-presented and/or LRRC33-presented).

Various disease conditions have been suggested to involve dysregulation of TGFβ signaling as a contributing factor. Indeed, the pathogenesis and/or progression of certain human conditions appear to be predominantly driven by or dependent on TGFβ1 activities. In particular, many such diseases and disorders appear to involve both an ECM component and an immune component of TGFβ1 function, suggesting that TGFβ1 activation in multiple contexts (e.g., mediated by more than one type of presenting molecules) is involved. Moreover, it is contemplated that there is crosstalk among TGFβ1-responsive cells. In some cases, interplays between multifaceted activities of the TGFβ1 axis may lead to disease progression, aggravation, and/or suppression of the host's ability to combat disease. For example, certain disease microenvironments, such as tumor microenvironment (TME), may be associated with TGFβ1 presented by multiple different presenting molecules, e.g., LTBP1-proTGFβ1, LTBP3-proTGFβ1, GARP-proTGFβ1, LRRC33-proTGFβ1, and any combinations thereof. TGFβ1 activities of one context may in turn regulate or influence TGFβ1 activities of another context, raising the possibility that when dysregulated, this may result in exacerbation of disease conditions. Therefore, it is desirable to broadly inhibit across multiple modes of TGFβ1 function (i.e., multiple contexts) while selectively limiting such inhibitory effects to the TGFβ1 isoform. The aim is not to perturb homeostatic TGFβ signaling mediated by the other isoforms, including TGFβ3, which plays an important role in would healing.

To address this, the inventors of the present disclosure sought to generate isoform-specific, context-permissive inhibitors of TGFβ1 which may be particularly advantageous for therapeutic use in the treatment of diseases that are driven by or dependent on TGFβ1 signaling or dysregulation thereof. The approach taken to meet the criteria for such inhibitors is: i) the ability to inhibit TGFβ1 signaling in an isoform-specific manner (without interfering with TGFβ2 and/or TGFβ3 activities); and, ii) the ability to inhibit both an ECM-associated and an immune cell-associated TGFβ1 signaling. The rationale for this approach is to balance the effectiveness (hence clinical efficacy) of TGFβ1 inhibition against potential toxicities. More specifically, achieving selectivity towards TGFβ1 at therapeutic dosage over the other isoforms is aimed to reduce or minimize possible toxicities (e.g., unwanted side effects and adverse events) associated with pan-inhibition of TGFβ in vivo, some of which may be required for normal biological functions (such as wound healing). On the other hand, inclusion of multiple contexts of TGFβ1 as therapeutic target is aimed at ensuring or to optimizing clinical efficacy in a disease that involves dysregulation of multiple aspects of TGFβ1 signaling. Various embodiments of clinical applications and treatment regimens are encompassed by the invention.

Accordingly, in one aspect, provided herein are isoform-specific, context-permissive inhibitors of TGFβ1, characterized in that such inhibitors have the ability to inhibit both an ECM-associated TGFβ1 signaling and an immune cell-associated TGFβ1 signaling. Specifically, such inhibitors can block TGFβ1 presented in multiple contexts, i.e., TGFβ1 activities mediated by two or more types of presenting molecules, while maintaining TGFβ2 and TGFβ3 activities intact. Thus, the TGFβ1 activities which can be inhibited by such inhibitors include two or more of the following: i) TGFβ1 signaling associated with GARP-presented TGFβ1; ii) TGFβ1 signaling associated with LRRC33-presented TGFβ1; iii) TGFβ1 signaling associated with LTBP1-presented TGFβ1; and, iv) TGFβ1 signaling associated with LTBP3-presented TGFβ1. In some embodiments, such inhibitors target at least two, or, at least three of pro-protein forms of the following complexes: i) TGFβ1-GARP; ii) TGFβ1-LRRC33; iii) TGFβ1-LTBP1; and, iv) TGFβ1-LTBP3. In some embodiments, such inhibitors are monoclonal antibodies that specifically bind and inhibit i) TGFβ1-GARP; iii) TGFβ1-LTBP1; and, iv) TGFβ1-LTBP3. In some embodiments, such monoclonal antibodies specifically bind and inhibit it ii) TGFβ1-LRRC33; iii) TGFβ1-LTBP1; and, iv) TGFβ1-LTBP3. In some embodiments, such monoclonal antibodies specifically bind and inhibit i) TGFβ1-GARP; ii) TGFβ1-LRRC33; and iii) TGFβ1-LTBP1. In some embodiments, such monoclonal antibodies specifically bind and inhibit i) TGFβ1-GARP; ii) TGFβ1-LRRC33; and iv) TGFβ1-LTBP3. In some embodiments, such monoclonal antibodies specifically inhibit all of the following complexes: i) TGFβ1-GARP; ii) TGFβ1-LRRC33; iii) TGFβ1-LTBP1; and, iv) TGFβ1-LTBP3. In some embodiments, such monoclonal antibodies do not bind mature TGFβ1 that is free TGFβ1 (e.g., growth factor that is released from or not complexed with a presenting molecule). The aspect of the invention includes compositions comprising such an inhibitor, including for example, pharmaceutical compositions which are suitable for administration in human and non-human subjects to be treated. Such pharmaceutical compositions are typically sterile. In some embodiments, such pharmaceutical compositions may also comprise at least one pharmaceutically acceptable excipient, such as a buffer and a surfactant (e.g., polysorbates). Kits comprising such a pharmaceutical composition are also encompassed by the invention.

Isoform-specific, context-permissive inhibitors described herein are suitable for use in the treatment of disease or disorder involving multiple biological functions of TGFβ1 and dysregulation thereof. In particular, such disease or disorder involves both an ECM component of TGFβ1 function and an immune component of TGFβ1 function. Administration of such an inhibitor can therefore inhibit each axis of the TGFβ1 signaling pathway in vivo, e.g., multiple TGFβ1 targets associated with the disease or disorder, enhancing therapeutic effects. Accordingly, in another aspect, the invention includes therapeutic use of such inhibitors in a method for treating a subject who suffers from a disease associated with TGFβ1 dysregulation. Isoform-specific, context-permissive or context-independent inhibitors of TGFβ1 signaling are particularly suitable for treating a disease that is driven or dependent on multiple functions (e.g., both an ECM component and an immune component) of TGFβ1. Typically, such diseases involve multiple cell types or cell status in which TGFβ1 is presented with multiple types of presenting molecules (e.g., multiple contexts).

In a related aspect, the invention provides screening, production and manufacture methods for isoform-specific, context-permissive TGFβ1 inhibitors with an improved safety profile (e.g., reduced in vivo toxicity). Such methods require that candidate agents be tested and selected for the TGFβ1 isoform specificity, e.g., candidate agents are selected for inhibitory activities against TGFβ1 signaling, and not TGFβ2 and/or TGFβ3 signaling. According to the invention, such isoform-specific inhibitors of TGFβ1 activities can inhibit multiple contexts of TGFβ1 function (see below).

In some embodiments, such agents are antibodies or antigen-binding fragments thereof that specifically bind and block activation of TGFβ1, but not TGFβ2 and/or TGFβ3. In some embodiments, such antibodies or antigen-binding fragments thereof do not bind free mature TGFβ1 growth factor that is not associated with a pro/latent complex. Thus, relevant production methods may include a screening step in which candidate agents (such as candidate antibodies or fragments thereof) are evaluated for their ability to inhibit TGFβ1 that is associated with particular presenting molecules, e.g., GARP, LRRC33, LTBP1, and/or LTBP3. In some embodiments, inactive (e.g., latent) precursor complex, such as GARP-proTGFβ1, LRRC33-proTGFβ1, LTBP1-proTGFβ1 and LTBP3-proTGFβ1, may be utilized to assay for activation of mature, active TGFβ1 growth factor. TGFβ1 activation, in the presence or absence of a test agent (i.e., candidate inhibitor) may be measured by any suitable means, including but not limited to in vitro assays and cell-based assays. Similar screening step can be utilized to test isoform specificity by the use of TGFβ2 and/or TGFβ3 counterparts. Such screening step can be carried out to identify candidate agents (such as candidate antibodies or fragments thereof) for their ability to inhibit TGFβ1 signaling in: i) an isoform-specific manner; and, ii) a context-permissive or context-independent manner.

Certain diseases are associated with dysregulation of multiple biological roles of TGFβ signaling that are not limited to a single context of TGFβ function. In such situations, it may be beneficial to modulate TGFβ effects across multiple contexts involved in the onset and/or during the course of disease progression. Thus, in some embodiments, the invention provides methods for targeting and broadly inhibiting multiple TGFβ1 contexts but in an isoform-specific manner. Such agents are herein referred to as “isoform-specific, context-permissive” TGFβ1 inhibitors. Thus, context-permissive TGFβ1 inhibitors target multiple contexts (e.g., multiple types of pro/latent-TGFβ1 complexes). Preferably, such inhibitors target at least one type (or “context”) of TGFβ1 pre-activation complex that is associated with the ECM (i.e., pro/latent TGFβ1 complex presented by an ECM-associated presenting molecule) and additionally at least one type (or “context”) of TGFβ1 pre-activation complex tethered to cell surface (i.e., pro/latent TGFβ1 complex presented by a cell or membrane-associated presenting molecule). In some embodiments, context-permissive TGFβ1 modulators target all types of pro/latent TGFβ1 complexes (e.g., GARP-associated, LRRC33-associated, LTBP-associated, etc.) so as to encompass all contexts irrespective of particular presenting molecule(s).

Whilst context-permissive TGFβ1 inhibitors are capable of targeting more than one types of pro/latent-TGFβ1 complexes (i.e., with different presenting molecules), in some embodiments, such inhibitors may favor (or show bias towards) one or more context over the other(s). Thus, in some embodiments, a context-permissive antibody that inhibits the activation of TGFβ1 may preferentially inhibit TGFβ1 activation mediated by one presenting molecule over another presenting molecule, even if such antibody is capable of binding to both types of pro/latent complexes. In some embodiments, such antibody is a monoclonal antibody that binds and inhibits activation of LTBP1/3-associated TGFβ1, GARP-associated TGFβ1, and LRRC33-associated TGFβ1, but with preferential inhibitory activities toward LTBP1/3-associated TGFβ1. In some embodiments, such antibody is a monoclonal antibody that binds and inhibits activation of LTBP1-associated TGFβ1, LTBP3-associated TGFβ1, GARP-associated TGFβ1, and LRRC33-associated TGFβ1, but with preferential inhibitory activities toward LTBP1- and LTBP-3-associated TGFβ1. In some embodiments, such antibody is a monoclonal antibody that binds and inhibits activation of LTBP1-associated TGFβ1, LTBP3-associated TGFβ1, GARP-associated TGFβ1, and LRRC33-associated TGFβ1, but with preferential inhibitory activities toward GARP-associated TGFβ1 and LRRC33-associated TGFβ1. In some embodiments, such antibody is a monoclonal antibody that binds and inhibits activation of GARP-associated TGFβ1 and LRRC33-associated TGFβ1, but with preferential inhibitory activities toward GARP-associated TGFβ1. In some embodiments, such antibody is a monoclonal antibody that binds and inhibits activation of GARP-associated TGFβ1 and LRRC33-associated TGFβ1, but with preferential inhibitory activities toward LRRC33-associated TGFβ1.

Thus, according to the invention, varying degrees of selectivity may be generated in order to target subset of TGFβ effects. Isoform-specific inhibitors of TGFβ (which target a single isoform of TGFβ) provide greater selectivity than so-called pan-TGFβ inhibitors (which target multiple or all isoforms of TGFβ).

The invention includes use of such TGFβ1 inhibitors in methods for treating a disease associated with TGFβ1 dysregulation. The use of such inhibitors is particularly advantageous in conditions where the TGFβ1 isoform plays a dominant role (over TGFβ2/3) in driving the disease, and where the disease involves both an ECM component and an immune component of TGFβ1 signaling. This approach aims to preserve normal or homeostatic TGFβ functions, while preferentially targeting disease-associated TGFβ function.

Such inhibitor is preferably a TGFβ1 activation inhibitor (i.e., inhibitor of the TGFβ1 activation step). In preferred embodiments, such inhibitor is capable of targeting the inactive forms of TGFβ1 (e.g., pro/latent-TGFβ1 complexes) prior to activation to effectuate more durable inhibition as compared to targeting a transient, already activated, soluble/free form of the growth factor that has been released from the latent complex. Determination of the source/context of disease-associated TGFβ1 may be carried out with the use of antibodies that specifically bind TGFβ1 latent complex that includes a particular presenting molecule of interest (e.g., GARP, LRRC33, LTBP1, LTBP3, etc.).

Aspects of the present disclosure relate to immunoglobulins, such as antibodies, or antigen binding portions thereof, that specifically bind at least three of the following complexes: a GARP-TGFβ1 complex, a LTBP1-TGFβ1 complex, a LTBP3-TGFβ1 complex and a LRRC33-TGFβ1 complex. According to the invention, such immunoglobulins specifically bind at least one type of ECM-associated (e.g., ECM-tethered) TGFβ1 complexes (e.g., LTBP1- and/or LTBP3-associated TGFβ1 complexes) and at least one type of cell-associated (e.g., cell surface-tethered) TGFβ1 complexes (e.g., GARP- and/or LRRC33-associated TGFβ1 complexes) to effectuate broad inhibitory action on multiple contexts. The antibodies, or antigen binding portions thereof, described herein, specifically bind to an epitope of TGFβ1 (e.g., LAP) or a component(s) of a protein complex comprising the TGFβ1 (e.g., LAP), that is available for binding by the antibodies, or antigen binding portions thereof, when the TGFβ1 is present in a GARP-TGFβ1 complex, a LTBP1-TGFβ1 complex, a LTBP3-TGFβ1 complex and/or a LRRC33-TGFβ1.

In some embodiments, the epitope is available for binding by the antibody when the TGFβ1 is present in two or more of the following protein complexes: a GARP-TGFβ1 complex, a LTBP1-TGFβ1 complex, a LTBP3-TGFβ1 complex, and a LRRC33-TGFβ1 complex; and wherein the antibody does not bind free mature TGFβ1 growth factor that is not in association with the pro/latent complex.

In some embodiments, the TGFβ1 is proTGFβ1 and/or latent TGFβ1 (e.g., pro/latent TGFβ1). In some embodiments, the TGFβ1 is latent TGFβ1. In some embodiments, the TGFβ1 is proTGFβ1.

The isoform-specific TGFβ1 inhibitors according to the invention do not bind TGFβ2. The isoform-specific TGFβ1 inhibitors according to the invention do not bind TGFβ3. In some embodiments, such inhibitors do not bind pro/latent TGFβ2. In some embodiments, such inhibitors do not bind pro/latent TGFβ3. In some embodiments, the antibody, or antigen binding portion thereof, does not prevent the ability of TGFβ1 to bind to integrin.

In some embodiments, the antibody, or antigen binding portion thereof, comprises a heavy chain variable region comprising a CDR3 having the amino acid sequence of SEQ ID NO: 87 and a light chain variable region comprising a CDR3 having the amino acid sequence of SEQ ID NO: 90. In some embodiments, the antibody, or antigen binding portion thereof, comprises a heavy chain variable region comprising a CDR2 having the amino acid sequence of SEQ ID NO: 86 and a light chain variable region comprising a CDR2 having the amino acid sequence of SEQ ID NO: 89. In some embodiments, the antibody, or antigen binding portion thereof, comprises a heavy chain variable region comprising a CDR1 having the amino acid sequence of SEQ ID NO: 85 and a light chain variable region comprising a CDR1 having the amino acid sequence of SEQ ID NO: 88.

In some embodiments, the antibody comprises a heavy chain polypeptide sequence that is at least 90% identical to the amino acid sequence set forth in SEQ ID NO: 99. In some embodiments, the antibody comprises a light chain polypeptide sequence that is at least 90% identical to the amino acid sequence set forth in SEQ ID NO: 100. In some embodiments, the antibody comprises a heavy chain polypeptide sequence that is at least 90% identical to the amino acid sequence set forth in SEQ ID NO: 99 and a light chain polypeptide sequence that is at least 90% identical to the amino acid sequence set forth in SEQ ID NO: 100. In some embodiments, such antibody comprises CDRs as set forth in SEQ ID NOs: 85-90. In some embodiments, the antibody consists of two polypeptides of SEQ ID NO: 99 and two polypeptides of SEQ ID NO:100.

In some embodiments, the antibody, or antigen binding portion thereof, comprises a heavy chain variable domain comprising an amino acid sequence having at least 70%, 75%, 80%, 85%, 90%, 95%, 96%, 97%, 98%, or 99% identity to the amino acid sequence set forth in SEQ ID NO: 95 and a light chain variable domain comprising an amino acid sequence having at least 70%, 75%, 80%, 85%, 90%, 95%, 96%, 97%, 98%, or 99% identity to the amino acid sequence set forth in SEQ ID NO: 97.

In some embodiments, the antibody, or antigen binding portion thereof, comprises a heavy chain variable domain comprising an amino acid sequence set forth in SEQ ID NO: 95 and a light chain variable domain comprising an amino acid sequence set forth in SEQ ID NO: 97.

In some embodiments, the antibody, or antigen binding portion thereof, inhibits TGFβ1 activation, but not TGFβ2 activation or TGFβ3 activation.

In some embodiments, the antibody, or antigen binding portion thereof, inhibits the release of mature TGFβ1 from the GARP-TGFβ1 complex, the LTBP1-TGFβ1 complex, the LTBP3-TGFβ1 complex, and/or the LRRC33-TGFβ1 complex.

In one aspect, provided herein is a pharmaceutical composition comprising an antibody, or antigen binding portion thereof, as described herein, and a pharmaceutically acceptable carrier. Such pharmaceutical compositions are typically sterile and are suitable for administration in human subjects. In some embodiments, such pharmaceutical compositions may be provided as kits, which are encompassed by the invention.

In another aspect, provided herein is a method for inhibiting TGFβ1 activation, the method comprising exposing a GARP-TGFβ1 complex, a LTBP1-TGFβ1 complex, a LTBP3-TGFβ1 complex, or a LRRC33-TGFβ1 complex to an antibody, an antigen binding portion thereof, or a pharmaceutical composition described herein.

In some embodiments, the antibody, or antigen binding portion thereof, inhibits the release of mature TGFβ1 from the GARP-TGFβ1 complex, the LTBP1-TGFβ1 complex, a LTBP3-TGFβ1 complex, or the LRRC33-TGFβ1 complex.

In some embodiments, the method is performed in vitro. In some embodiments, the method is performed in vivo.

Thus, the invention includes a method for treating a disease associated with dysregulation of TGFβ1 signaling in a human subject. Such method comprises a step of: administering to a human subject in need thereof a pharmaceutical composition provided herein, in an amount effective to treat the disease, wherein the amount achieves statistically significant clinical efficacy and safety when administered to a patient population having the disease.

In yet another aspect, provided herein is a TGFβ inhibitor for use in reducing adverse effects in a subject, wherein the TGFβ inhibitor is isoform-selective. In some embodiments, the TGFβ inhibitor is an antibody that specifically inhibits TGFβ1 while broadly targeting multiple contexts.

In some embodiments, the cell expressing the GARP-TGFβ1 complex or the LRRC33-TGFβ1 complex is a T-cell, a fibroblast, a myofibroblast, a macrophage, a monocyte, a dendritic cell, an antigen presenting cell, a neutrophil, a myeloid-derived suppressor cell (MDSC), a lymphocyte, a mast cell, a megakaryocyte, a natural killer (NK) cell, a microglia, or a progenitor cell of any one of such cells. In some embodiments, the cell expressing the GARP-TGFβ1 complex or the LRRC33-TGFβ1 complex is a hematopoietic stem cell. In some embodiments, the cell expressing the GARP-TGFβ1 complex or the LRRC33-TGFβ1 complex is a neural crest-derived cell. The T-cell may be a regulatory T cell (e.g., immunosuppressive T cell). The T cell may be a CD4-positive (CD4+) T cell and/or CD8-positive (CD8+) T cell. The neuprophil may be an activated neutrophil. The macrophage may be a polarized macrophage, including profibrotic and/or tumor-associated macrophages (TAM), e.g., M2c subtype and M2d subtype macrophages. The macrophage may be activated by one or more soluble factors, such as growth factors, cytokines, chemokines and/or other molecules that are present in a particular disease microenvironment (e.g., TME), which may work in an autocrine, paracrine, and/or endocrine fashion. In some embodiments, the macrophage is activated by M-CSF, such as M-CSF secreted by a solid tumor. In some embodiments, the macrophage is activated by TGFβ1.

In some embodiments, the cell expressing the GARP-TGFβ1 complex or the LRRC33-TGFβ1 complex is a cancer cell, e.g., circulating cancer cells and tumor cells. In some embodiments, the cell expressing the GARP-TGFβ1 complex or the LRRC33-TGFβ1 complex is recruited to a disease site, such as TME (e.g., tumor infiltrate). In some embodiments, the expression of the GARP-TGFβ1 complex or the LRRC33-TGFβ1 complex is induced by a disease microenvironment (e.g., TME). In some embodiments, a solid tumor comprises elevated leukocyte infiltrates, e.g., CD45+. It is contemplated that tumor-associated CD45+ cells include GARP-expressing and/or LRRC33-expressing cells.

In some embodiments, the LTBP1-TGFβ1 complex or the LTBP3-TGFβ1 complex is bound to an extracellular matrix (i.e., components of the ECM). In some embodiments, the extracellular matrix comprises fibrillin and/or fibronectin. In some embodiments, the extracellular matrix comprises a protein comprising an RGD motif. In some embodiments, cells that produce and deposit the LTBP1-TGFβ1 complex or the LTBP3-TGFβ1 complex are present in a solid tumor, such as cancer cells and stromal cells. In some embodiments, cells that produce and deposit the LTBP1-TGFβ1 complex or the LTBP3-TGFβ1 complex are present in a fibrotic tissue. In some embodiments, cells that produce and deposit the LTBP1-TGFβ1 complex or the LTBP3-TGFβ1 complex are present in a bone marrow. In some embodiments, cells that produce and deposit the LTBP1-TGFβ1 complex or the LTBP3-TGFβ1 complex are myofibroblasts or myofibroblast-like cells, including, for example, cancer-associated fibroblasts (CAFs).

In another aspect, provided herein is a method for reducing TGFβ1 activation in a subject, the method comprising administering to the subject an effective amount of an antibody, an antigen binding portion thereof, or a pharmaceutical composition, as described herein, thereby reducing TGFβ1 activation in the subject.

In some embodiments, the subject has or is at risk of having fibrotic disorder. In some embodiments, the fibrotic disorder comprises chronic inflammation of the affected tissue/organ. In some embodiments, the subject has a muscular dystrophy. In some embodiments, the subject has Duchenne muscular dystrophy (DMD). In some embodiments, the subject has or is at risk of having liver fibrosis, kidney fibrosis, lung fibrosis (e.g., idiopathic pulmonary fibrosis), endometriosis or uterine fibrosis. In some embodiments, the subject has or is at risk of having cancer (e.g., solid tumor, blood cancer, and myelofibrosis). In some embodiments, the subject has or is at risk of having dementia.

In some embodiments, the subject further receives an additional therapy. In some embodiments, the additional therapy is selected from the group consisting of a myostatin inhibitor, a VEGF agonist, an IGF1 agonist, an FXR agonist, a CCR2 inhibitor, a CCR5 inhibitor, a dual CCR2/CCR5 inhibitor, a lysyl oxidase-like-2 inhibitor, an ASK1 inhibitor, an Acetyl-CoA Carboxylase (ACC) inhibitor, a p38 kinase inhibitor, Pirfenidone, Nintedanib, a GDF11 inhibitor, JAK inhibitor (e.g., JAK2 inhibitor), or any combination thereof.

In some embodiments, the antibody, or the antigen binding portion thereof, reduces the suppressive activity of regulatory T cells (Tregs).

In some embodiments, the antibody, or the antigen binding portion thereof, does not induce organ toxicity in the subject. In some embodiments, the organ toxicity comprises cardiovascular toxicity, gastrointestinal toxicity, immunotoxicity, bone toxicity, cartilage toxicity, reproductive system toxicity, or renal toxicity.

In one aspect, provided herein is a method for treating cancer in a subject in need thereof, the method comprising administering to the subject an effective amount of an antibody, an antigen binding portion thereof, or a pharmaceutical composition, as described herein, thereby treating cancer in the subject.

In another aspect, provided herein is a method of reducing tumor growth in a subject in need thereof, the method comprising administering to the subject an effective amount of an antibody, an antigen binding portion thereof, or a pharmaceutical composition, as described herein, thereby reducing tumor growth in the subject.

In some embodiments, the antibody, or antigen binding portion thereof, is administered in combination with an additional agent or an additional therapy. In some embodiments, the additional agent is a checkpoint inhibitor. In some embodiments, the additional agent is selected from the group consisting of a PD-1 antagonist, a PDL1 antagonist, a PD-L1 or PDL2 fusion protein, a CTLA4 antagonist, etc. Such combination therapies may advantageously utilize lower dosages of the administered therapeutic agents, thus avoiding possible toxicities or complications associated with the various monotherapies or conventional combination therapies that lack the degree of selectivity/specificity achieved by the present invention.

In some embodiments, the method further comprises determining (e.g., testing or confirming) the involvement of TGFβ1 in the disease, relative to TGFβ2 and TGFβ3. In some embodiments, the method further comprises a step of: identifying a source (or context) of disease-associated TGFβ1. In some embodiments, the source/context is assessed by determining the expression of TGFβ presenting molecules, e.g., LTBP1, LTBP3, GARP and LRRC33 in a clinical sample taken from patients.

In yet another aspect, provided herein is a method for making (e.g., producing, manufacturing) a pharmaceutical composition for inhibiting TGFβ signaling, the method comprising steps of: providing one or more agents that inhibit signaling of at least one isoform of TGFβ; measuring activities of the one or more agents towards all isoforms of TGFβ; selecting an agent that is selective for TGFβ1; formulating into a pharmaceutical composition comprising an isoform-specific TGFβ1 inhibitor and a pharmaceutically acceptable excipient, such as a suitable buffer. Also provided is a pharmaceutical composition produced by such method. In some embodiments, the method further comprises a step of determining (e.g., measuring, assaying) context-dependent inhibitory activities of one or more agents.

The subject matter of the present disclosure also relates to that of PCT/US2013/068613, filed Nov. 6, 2013; PCT/US2014/036933, filed May 6, 2014; and PCT/US2017/021972, filed Mar. 10, 2017, the entire contents of each of which are incorporated herein by reference.

### BRIEF DESCRIPTION OF THE FIGURES

FIG. 1 provides a schematic depicting TGFβ1 within a latent complex in the tissue microenvironment.

FIGS. 2A-2C illustrate multiple contexts of TGFβ1 function: GARP-presented TGFβ1 is expressed on regulatory T cells, which is involved in immune regulation (FIG. 2A); LTBP1/3-presented TGFβ1 is deposited by fibroblasts and other cells into the ECM (FIG. 2B); and, LRRC33-presented TGFβ1 is expressed on myeloid cells, including macrophages (FIG. 2C).

FIG. 3 illustrates a protein expression platform for making a GARP-TGFβ1 complex and a LTBP-TGFβ1 complex. The HEK293-based expression system uses Ni-NTA affinity purification and gel filtration to obtain multimilligram quantities of purified protein. Schematics of wild-type proTGFβ1, LTPB1, sGARP, and proTGF β1 C4S are shown.

FIG. 4A depicts specific binding of Ab3 to latent TGFβ1. FIG. 4B shows binding specificity of exemplary monoclonal antibodies. FIG. 4B depicts that Ab1 and Ab2 specifically bind proTGFβ1 as measured by ELISA, but not proTGFβ2, proTGFβ3, or mature TGFβ1. FIG. 4C depicts an example of an antibody which binds (as measured by ELISA) specifically to the LTBP1-proTGFβ1 complex.

FIG. 5 provides a panel of prior art antibodies made against mature TGFβ growth factor, and their respective binding profiles for all three isoforms.

FIGS. 6A-6B provide binding profiles, as measured by Octet, of Ab1, Ab2 and Ab3, which are isoform-specific, context-permissive/independent TGFβ1 inhibitors.

FIGS. 7A-7H provide cell-based inhibition assays.

FIG. 8 shows inhibitory effects of Ab3 on Kallikrein-induced activation of TGFβ1 in vitro.

FIGS. 9A-9B show inhibitory effects of Ab1 and Ab3 on regulatory T cell-dependent suppression of effector T cell proliferation.

FIGS. 10A-10C show upregulation of cell surface LRRC33 expression in polarized macrophages.

FIG. 11 provides results from a T cell co-transfer colitis model.

FIGS. 12A-12K show inhibitory effects of Ab2 on TGFb1-dependent mechanistic disease model of UUO.

FIGS. 13A-13C show inhibitory effects of Ab3 on TGFb1-dependent mechanistic disease model of UUO.

FIG. 14 provides inhibitory effects of Ab3 on carbon tetrachloride-induced fibrosis model.

FIG. 15 provides inhibitory effects of Ab3 on a translational model of fibrosis in Alport mice.

FIG. 16 shows inhibitory effects of Ab2 on tumor growth in MC38 carcinoma.

FIG. 17 provides effects of Ab3 in combination with a PD-1 antagonist on survival in EMT-6 tumor model.

FIGS. 18A-18F provide toxicology/tolerability data showing improved safety profiles of Ab2 in rats.

FIGS. 19A-19B provide toxicology/tolerability data showing improved safety profiles of Ab3 in rats.

FIG. 20 provides data showing in vivo isoform-selectivity of Ab3 in homeostatic rat BAL cells.

FIGS. 21A-21D provide relative expression of TGFβ isoforms. FIG. 21A shows TGFβ isoform expression vs. normal comparator (by cancer type). FIG. 21B shows frequency of TGFβ Isoform Expression by Human Cancer Type. FIG. 21C shows TGFβ isoform expression in individual tumor samples, by cancer type. FIG. 21D shows TGFβ isoform expression in mouse syngeneic cancer cell model lines.

FIG. 22 depicts microscopic heart findings from a pan-TGFβ antibody from a 1-week study.

### DETAILED DESCRIPTION OF CERTAIN EMBODIMENTS

In mammals, the transforming growth factor-beta (TGFβ) superfamily is comprised of at least 33 gene products. These include the bone morphogenic proteins (BMPs), activins, growth and differentiation factors (GDFs), and the three isoforms of the TGFβ family: TGFβ1, TGFβ2, and TGFβ3. The TGFβs are thought to play key roles in diverse processes, such as inhibition of cell proliferation, extracellular matrix (ECM) remodeling, and immune homeostasis. The importance of TGFβ1 for T cell homeostasis is demonstrated by the observation that TGFβ1−/− mice survive only 3-4 weeks, succumbing to multiorgan failure due to massive immune activation (Kulkarni, A. B., et al., Proc Natl Acad Sci USA, 1993. 90(2): p. 770-4; Shull, M. M., et al., Nature, 1992. 359(6397): p. 693-9). The roles of TGFβ2 and TGFβ3 are less clear. Whilst the three TGFβ isoforms have distinct temporal and spatial expression patterns, they signal through the same receptors, TGFβRI and TGFβRII, although in some cases, for example for TGFβ2 signaling, type III receptors such as betaglycan are also required (Feng, X. H. and R. Derynck, Annu Rev Cell Dev Biol, 2005. 21: p. 659-93; Massague, J., Annu Rev Biochem, 1998. 67: p. 753-91). Ligand-induced oligomerization of TGFβRI/II triggers the phosphorylation of SMAD transcription factors, resulting in the transcription of target genes, such as Col1a1, Col3a1, ACTA2, and SERPINE1 (Massague, J., J. Seoane, and D. Wotton, Genes Dev, 2005. 19(23): p. 2783-810). SMAD-independent TGFβ signaling pathways have also been described, for example in cancer or in the aortic lesions of Marfan mice (Derynck, R. and Y. E. Zhang, Nature, 2003. 425(6958): p. 577-84; Holm, T. M., et al., Science, 2011. 332(6027): p. 358-61).

The biological importance of the TGFβ pathway in humans has been validated by genetic diseases. Camurati-Engelman disease results in bone dysplasia due to an autosomal dominant mutation in the TGFB1 gene, leading to constitutive activation of TGFβ1 signaling (Janssens, K., et al., J Med Genet, 2006. 43(1): p. 1-11). Patients with Loeys/Dietz syndrome carry autosomal dominant mutations in components of the TGFβ signaling pathway, which cause aortic aneurism, hypertelorism, and bifid uvula (Van Laer, L., H. Dietz, and B. Loeys, Adv Exp Med Biol, 2014. 802: p. 95-105). As TGFβ pathway dysregulation has been implicated in multiple diseases, several drugs that target the TGFβ pathway have been developed and tested in patients, but with limited success.

Dysregulation of the TGFβ signaling has been associated with a wide range of human diseases. Indeed, in a number of disease conditions, such dysregulation may involve multiple facets of TGFβ function. Diseased tissue, such as fibrotic and/or inflamed tissues and tumors, may create a local environment in which TGFβ activation can cause exacerbation or progression of the disease, which may be at least in part mediated by interactions between multiple TGFβ-responsive cells, which are activated in an autocrine and/or paracrine fashion, together with a number of other cytokines, chemokines and growth factors that play a role in a particular disease setting. For example, a tumor microenvironment (TME) contains multiple cell types expressing TGFβ1, such as activated myofibroblast-like fibroblasts, stromal cells, infiltrating macrophages, MDSCs and other immune cells, in addition to cancer (i.e., malignant) cells. Thus, the TME represents a heterogeneous population of cells expressing and/or responsive to TGFβ1 but in association with more than one types of presenting molecules, e.g., LTBP1, LTBP3, LRRC33 and GARP, within the niche.

To effectively inhibit dysregulated or disease-driving TGFβ1 activities involving multiple cell types and signaling “contexts,” the inventors of the present disclosure sought to develop a class of agents that has the ability to inhibit multiple TGFβ1 functions but in an isoform-specific manner. Such agents are referred to as “isoform-specific, context-permissive” inhibitors of TGFβ1, as defined herein. In some embodiments, such inhibitors are isoform-specific, context-independent inhibitors of TGFβ1. It is contemplated that use of an isoform-specific, context-permissive or context-independent inhibitor of TGFβ1 can exert its inhibitory effects upon multiple modes of TGFβ1 function in a disease that involve an interplay of various cell types that express and/or respond to TGFβ1 signaling, thereby enhancing therapeutic effects by targeting multiple types of TGFβ1 precursor complexes. Accordingly, the therapeutic targets of such an inhibitor include at least three of the following complexes: i) proTGFβ1 presented by GARP; ii) proTGFβ1 presented by LRRC33; iii) proTGFβ1 presented by LTBP1; and iv) proTGFβ1 presented by LTBP3. Typically, complexes (i) and (ii) above are present on cell surface because both GARP and LRRC33 are transmembrane proteins capable of presenting TGFβ1 on the extracellular face, whilst complexes (iii) and (iv) are components of the extracellular matrix. A number of studies have shed light on the mechanisms of TGFβ1 activation. Three integrins, αVβ6, αVβ8, and αVβ1 have been demonstrated to be key activators of latent TGFβ1 (Reed, N. I., et al., Sci Transl Med, 2015. 7(288): p. 288ra79; Travis, M. A. and D. Sheppard, Annu Rev Immunol, 2014. 32: p. 51-82; Munger, J. S., et al., Cell, 1999. 96(3): p. 319-28). αV integrins bind the RGD sequence present in TGFβ1 and TGFβ1 LAPs with high affinity (Dong, X., et al., Nat Struct Mol Biol, 2014. 21(12): p. 1091-6). Transgenic mice with a mutation in the TGFβ1 RGD site that prevents integrin binding, but not secretion, phenocopy the TGFβ1−/− mouse (Yang, Z., et al., J Cell Biol, 2007. 176(6): p. 787-93). Mice that lack both 136 and 138 integrins recapitulate all essential phenotypes of TGFβ1 and TGFβ3 knockout mice, including multiorgan inflammation and cleft palate, confirming the essential role of these two integrins for TGFβ1 activation in development and homeostasis (Aluwihare, P., et al., J Cell Sci, 2009. 122(Pt 2): p. 227-32). Key for integrin-dependent activation of latent TGFβ1 is the covalent tether to presenting molecules; disruption of the disulfide bonds between GARP and TGFβ1 LAP by mutagenesis does not impair complex formation, but completely abolishes TGFβ1 activation by αVβ6 (Wang, R., et al., Mol Biol Cell, 2012. 23(6): p. 1129-39). The recent structure of latent TGFβ1 illuminates how integrins enable release of active TGFβ1 from the latent complex: the covalent link of latent TGFβ1 to its presenting molecule anchors latent TGFβ1, either to the ECM through LTBPs, or to the cytoskeleton through GARP or LRRC33. Integrin binding to the RGD sequence results in a force-dependent change in the structure of LAP, allowing active TGFβ1 to be released and bind nearby receptors (Shi, M., et al., Nature, 2011. 474(7351): p. 343-9). The importance of integrin-dependent TGFβ1 activation in disease has also been well validated. A small molecular inhibitor of αVβ1 protects against bleomycin-induced lung fibrosis and carbon tetrachloride-induced liver fibrosis (Reed, N. I., et al., Sci Transl Med, 2015. 7(288): p. 288ra79), and αVβ6 blockade with an antibody or loss of integrin 36 expression suppresses bleomycin-induced lung fibrosis and radiation-induced fibrosis (Munger, J. S., et al., Cell, 1999. 96(3): p. 319-28); Horan, G. S., et al., Am J Respir Crit Care Med, 2008. 177(1): p. 56-65). In addition to integrins, other mechanisms of TGFβ1 activation have been implicated, including thrombospondin-1 and activation by proteases such as matrix metalloproteinases (MMPs), cathepsin D or kallikrein. However, the majority of these studies were performed in vitro using purified proteins; there is less evidence for the role of these molecules from in vivo studies. Knockout of thrombospondin-1 recapitulates some aspects of the TGFβ1−/− phenotype in some tissues, but is not protective in bleomycin-induced lung fibrosis, known to be TGFβ-dependent (Ezzie, M. E., et al., Am J Respir Cell Mol Biol, 2011. 44(4): p. 556-61). Additionally, knockout of candidate proteases did not result in a TGFβ1 phenotype (Worthington, J. J., J. E. Klementowicz, and M. A. Travis, Trends Biochem Sci, 2011. 36(1): p. 47-54). This could be explained by redundancies or by these mechanisms being critical in specific diseases rather than development and homeostasis.

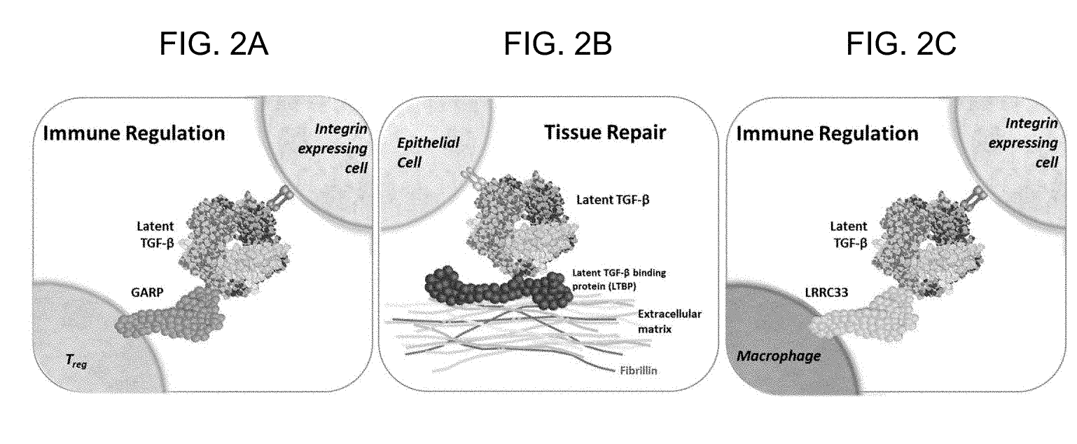

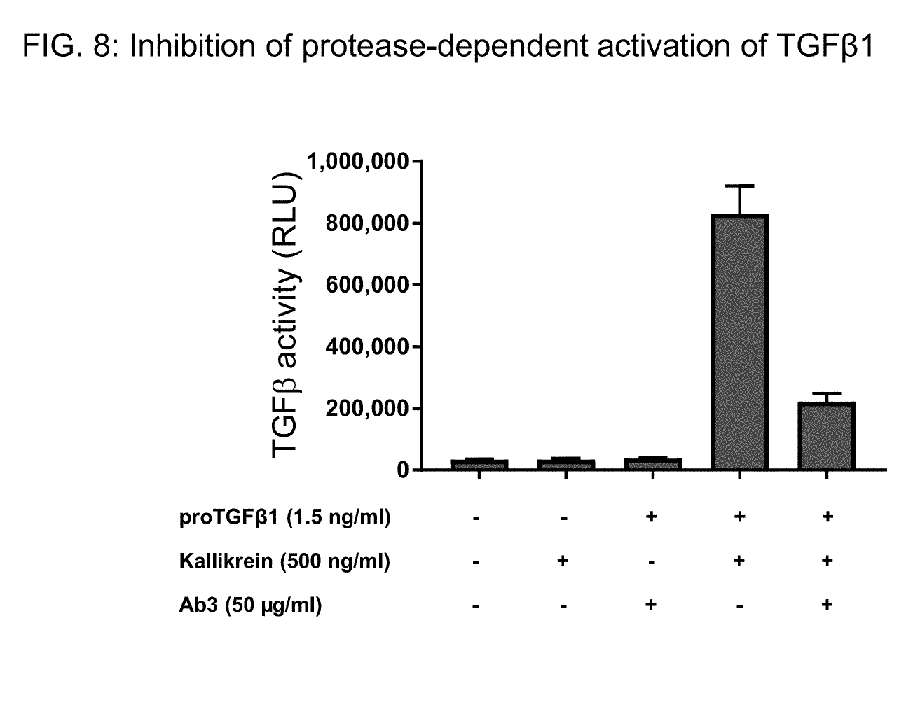

Thus, the isoform-specific, context permissive inhibitors of TGFβ1 described herein include inhibitors that work by preventing the step of TGFβ1 activation. In some embodiments, such inhibitors can inhibit integrin-dependent (e.g., mechanical or force-driven) activation of TGFβ1 (see FIG. 2). In some embodiments, such inhibitors can inhibit protease-dependent or protease-induced activation of TGFβ1. The latter includes inhibitors that inhibit the TGFβ1 activation step in an integrin-independent manner. In some embodiments, such inhibitors can inhibit TGFβ1 activation irrespective of the mode of activation, e.g., inhibit both integrin-dependent activation and protease-dependent activation of TGFβ1. Non-limiting examples of proteases which may activate TGFβ1 include serine proteases, such as Kallikreins, Chemotrypsin, Trypsin, Elastases, Plasmin, as well as zinc metalloproteases (MMP family) such as MMP-2, MMP-9 and MMP-13. Kallikreins include plasma-Kallikreins and tissue Kallikreins, such as KLK1, KLK2, KLK3, KLK4, KLK5, KLK6, KLK7, KLK8, KLK9, KLK10, KLK11, KLK12, KLK13, KLK14 and KLK15. FIG. 8 provides one example of an isoform-specific, context-independent inhibitor of TGFβ1, which can inhibit Kallikrein-dependent activation of TGFβ1 in vitro. In some embodiments, inhibitors of the present invention prevent release or dissociation of active (mature) TGFβ1 growth factor from the latent complex. In some embodiment, such inhibitors may work by stabilizing the inactive (e.g., latent) conformation of the complex.

TGFβ has been implicated in a number of biological processes, including fibrosis, immune-modulation and cancer progression. TGFβ1 was the first identified member of the TGFβ superfamily of proteins. Like other members of the TGFβ superfamily, TGFβ1 and the isoforms TGFβ2 and TGFβ3, are initially expressed as inactive precursor pro-protein forms (termed proTGFβ). TGFβ proteins (e.g., TGFβ1, TGFβ2 and TGFβ3) are proteolytically cleaved by proprotein convertases (e.g., furin) to yield the latent form (termed latent TGFβ). In some embodiments, a pro-protein form or latent form of a TGFβ protein (e.g., TGFβ1, TGFβ2 and TGFβ3) may be referred to as “pro/latent TGFβ protein”. TGFβ1 may be presented to other molecules in complex with multiple molecules including, for example, GARP (to form a GARP-TGFβ1 complex), LRRC33 (to form a LRRC33-TGFβ1 complex), LTBP1 (to form a LTBP1-TGFβ1 complex), and/or LTBP3 (to form a LTBP3-TGFβ1 complex). The TGFβ1 present in these complexes may be in either latent form (latent TGFβ1) or in precursor form (proTGFβ1).

The invention is particularly useful for therapeutic use for certain diseases that are associated with multiple biological roles of TGFβ1 signaling that are not limited to a single context of TGFβ1 function. In such situations, it may be beneficial to inhibit TGFβ1 effects across multiple contexts. Thus, in some embodiments, the invention provides methods for targeting and inhibiting TGFβ1 in an isoform-specific manner, rather than in a context-specific manner. Such agents may be referred to as “isoform-specific, context-permissive” TGFβ1 modulators. In some embodiments, context-permissive TGFβ1 modulators target multiple contexts (e.g., multiple types of pro/latent-TGFβ1 complexes). In some embodiments, context-permissive TGFβ1 modulators target all types of pro/latent TGFβ1 complexes (e.g., GARP-associated, LRRC33-associated, LTBP-associated, etc.) so as to encompass all contexts.

Whilst context-permissive TGFβ1 inhibitors are capable of targeting more than one types of pro/latent-TGFβ1 complexes (i.e., with different presenting molecules), in some embodiments, such inhibitors may favor one or more context over the other. Thus, in some embodiments, a context-permissive antibody that inhibits the activation of TGFβ1 may preferentially inhibit TGFβ1 activation mediated by one presenting molecule over another presenting molecule, even if such antibody is capable of binding to both types of pro/latent complexes. In some embodiments, such antibody is a monoclonal antibody that binds and inhibits activation of LTBP-associated TGFβ1, GARP-associated TGFβ1, and LRRC33-associated TGFβ1, but with preferential inhibitory activities toward LTBP-associated TGFβ1. In some embodiments, such antibody is a monoclonal antibody that binds and inhibits activation of LTBP1-associated TGFβ1, LTBP3-associated TGFβ1, GARP-associated TGFβ1, and LRRC33-associated TGFβ1, but with preferential inhibitory activities toward LTBP1- and LTBP-3-associated TGFβ1. In some embodiments, such antibody is a monoclonal antibody that binds and inhibits activation of LTBP1-associated TGFβ1, LTBP3-associated TGFβ1, GARP-associated TGFβ1, and LRRC33-associated TGFβ1, but with preferential inhibitory activities toward GARP-associated TGFβ1 and LRRC33-associated TGFβ1. In some embodiments, such antibody is a monoclonal antibody that binds and inhibits activation of GARP-associated TGFβ1 and LRRC33-associated TGFβ1, but with preferential inhibitory activities toward GARP-associated TGFβ1. In some embodiments, such antibody is a monoclonal antibody that binds and inhibits activation of GARP-associated TGFβ1 and LRRC33-associated TGFβ1, but with preferential inhibitory activities toward LRRC33-associated TGFβ1.

Thus, according to the invention, varying degrees of selectivity may be generated in order to target subset of TGFβ effects. Isoform-specific inhibitors of TGFβ1 (which target a single isoform of TGFβ, e.g., TGFβ1) provide greater selectivity than pan-TGFβ inhibitors (which target multiple or all isoforms of TGFβ). Isoform-specific, context-permissive inhibitors of TGFβ1 (which target multiple contexts of a single isoform of TGFβ1) provide greater selectivity than isoform-specific inhibitors. Isoform-specific, context-independent inhibitors of TGFβ1 (which target and inhibit TGFβ1 functions regardless of which presenting molecule is associated with) provides isoform specificity while allowing broader coverage of inhibitory effects across multiple activities of TGFβ1.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

Antibody: The term “antibody” encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen binding portions thereof, can be produced in hybridomas, by recombinant DNA techniques, or by enzymatic or chemical cleavage of intact antibodies. The term antibodies, as used herein, includes monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), respectively. In some embodiments, the term also encompasses peptibodies.

Antigen: The term “antigen” refers to a molecular structure that provides an epitope, e.g., a molecule or a portion of a molecule, or a complex of molecules or portions of molecules, capable of being bound by a selective binding agent, such as an antigen binding protein (including, e.g., an antibody). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen binding proteins, e.g., antibodies. Antigen-binding portion/fragment: The terms “antigen-binding portion” or “antigen-binding fragment” of an antibody, as used herein, refers to one or more fragments of an antibody that retain the ability to specifically bind to an antigen (e.g., TGFβ1). Antigen binding portions include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments, a monovalent fragment consisting of the VL, VH, CL and CH1 domains; (ii) F(ab′)2 fragments, a bivalent fragment comprising two Fab fragments linked by a disulfide bridge at the hinge region; (iii) Fd fragments consisting of the VH and CH1 domains; (iv) Fv fragments consisting of the VL and VH domains of a single arm of an antibody; (v) single-chain Fv (scFv) molecules (see, e.g., Bird et al. (1988) SCIENCE 242:423-426; and Huston et al. (1988) PROC. NAT'L. ACAD. SCI. USA 85:5879-5883); (vi) dAb fragments (see, e.g., Ward et al. (1989) NATURE 341: 544-546); and (vii) minimal recognition units consisting of the amino acid residues that mimic the hypervariable region of an antibody (e.g., an isolated complementarity determining region (CDR)). Other forms of single chain antibodies, such as diabodies are also encompassed. The term antigen binding portion of an antibody includes a “single chain Fab fragment” otherwise known as an “scFab,” comprising an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids.

Cancer: The term “cancer” as used herein refers to the physiological condition in multicellular eukaryotes that is typically characterized by unregulated cell proliferation and malignancy. Thus, the term broadly encompasses, solid tumors, blood cancers (e.g., leukemias), as well as myelofibrosis and multiple myeloma.

Cell-associated TGFβ1: The term refers to TGFβ1 or its signaling complex (e.g., pro/latent TGFβ1) that is membrane-bound (e.g., tethered to cell surface). Typically, such cell is an immune cell. TGFβ1 that is presented by GARP or LRRC33 is a cell-associated TGFβ1.

Checkpoint inhibitor: In the context of this disclosure, checkpoint inhibitors refer to immune checkpoint inhibitors and carries the meaning as understood in the art. Typically, target is a receptor molecule on T cells or NK cells, or corresponding cell surface ligand on antigen-presenting cells (APCs) or tumor cells. Immune checkpoints are activated in immune cells to prevent inflammatory immunity developing against the “self”. Therefore, changing the balance of the immune system via checkpoint inhibition should allow it to be fully activated to detect and eliminate the cancer. The best known inhibitory receptors implicated in control of the immune response are cytotoxic T-lymphocyte antigen-4 (CTLA-4), programmed cell death protein 1 (PD-1), T-cell immunoglobulin domain and mucin domain-3 (TIM3), lymphocyte-activation gene 3 (LAG3), killer cell immunoglobulin-like receptor (KIR), glucocorticoid-induced tumor necrosis factor receptor (GITR) and V-domain immunoglobulin (Ig)-containing suppressor of T-cell activation (VISTA). Non-limiting examples of checkpoint inhibitors include: Nivolumab, Pembrolizumab, BMS-936559, Atezolizumab, Avelumab, Durvalumab, Ipilimumab, Tremelimumab, IMP-321, BMS-986016, and Lirilumab.

Clinical benefit: As used herein, the term “clinical benefits” is intended to include both efficacy and safety of a therapy. Thus, therapeutic treatment that achieves a desirable clinical benefit is both efficacious and safe (e.g., with tolerable or acceptable toxicities or adverse events).

Combination therapy: “Combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

Combinatory or combinatorial epitope: In some embodiments, inhibitory antibodies of the invention may bind an epitope formed by two or more components (e.g., portions or segments) of a pro/latent TGFβ1 complex. Such an epitope is referred to as a combinatory or combinatorial epitope. Thus, a combinatory epitope may comprise amino acid residue(s) from a first component of the complex, and amino acid residue(s) from a second component of the complex, and so on. Each component may be of a single protein or of two or more proteins of an antigenic complex. Binding of an antibody to a combinatory epitope does not merely depend on a primary amino acid sequence of the antigen. Rather, a combinatory epitope is formed with structural contributions from two or more components (e.g., portions or segments, such as amino acid residues) of an antigen or antigen complex.

Compete or cross-compete: The term “compete” when used in the context of antigen binding proteins (e.g., an antibody or antigen binding portion thereof) that compete for the same epitope means competition between antigen binding proteins as determined by an assay in which the antigen binding protein being tested prevents or inhibits (e.g., reduces) specific binding of a reference antigen binding protein to a common antigen (e.g., TGFβ1 or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay; solid phase direct biotin-avidin EIA; solid phase direct labeled assay, and solid phase direct labeled sandwich assay. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more. In some embodiments, a first antibody or antigen-binding portion thereof and a second antibody or antigen-binding portion thereof cross-block with each other with respect to the same antigen, for example, as assayed by Biacor or Octet, using standard test conditions, e.g., according to the manufacturer's instructions (e.g., binding assayed at room temperature, ˜20-25° C.). In some embodiments, the first antibody or fragment thereof and the second antibody or fragment thereof may have the same epitope. In other embodiments, the first antibody or fragment thereof and the second antibody or fragment thereof may have non-identical but overlapping epitopes. In yet further embodiments, the first antibody or fragment thereof and the second antibody or fragment thereof may have separate (different) epitopes which are in close proximity in a three-dimensional space, such that antibody binding is cross-blocked via steric hinderance. “Cross-block” means that binding of the first antibody to an antigen prevents binding of the second antibody to the same antigen, and similarly, binding of the second antibody to an antigen prevents binding of the first antibody to the same antigen.

Complementary determining region: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region that can bind the antigen. The exact boundaries of these CDRs have been defined differently according to different systems. The system described by Kabat (Kabat et al. (1987; 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk (1987) J. Mol. Biol. 196: 901-917; and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2 and L3 or H1, H2 and H3, where the “L” and the “H” designate the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

Conformational epitope: In some embodiments, inhibitory antibodies of the invention may bind an epitope which is conformation-specific. Such an epitope is referred to as a conformational epitope, conformation-specific epitope, conformation-dependent epitope, or conformation-sensitive epitope. A corresponding antibody or fragment thereof that specifically binds such an epitope may be referred to as conformation-specific antibody, conformation-selective antibody, or conformation-dependent antibody. Binding of an antigen to a conformational epitope depends on the three-dimensional structure (conformation) of the antigen or antigen complex.

Constant region: An immunoglobulin constant domain refers to a heavy or light chain constant domain. Human IgG heavy chain and light chain constant domain amino acid sequences are known in the art.

Context-permissive; context-independent: “Context-permissive” and “context-independent” TGFβ inhibitors are broad-context inhibitors which can act upon more than one modes of TGFβ function. A “context-permissive inhibitor” of TGFβ is an agent capable of inhibiting multiple contexts of TGFβ function, e.g., TGFβ activities associated with at least two of the following: GARP (also referred to as LRRC32), LRRC33, LTBP1, and LTBP3. Among context-permissive inhibitors, where an agent is capable of inhibiting TGFβ activities irrespective of specific presenting molecules, such an inhibitor is referred to as a “context-independent” inhibitor. Thus, a context-independent inhibitor of TGFβ can inhibit TGFβ activities associated with all of the following: GARP, LRRC33, LTBP1, and LTBP3. In some embodiments, context-permissive and context-independent inhibitors may exert preferential or biased inhibitory activities towards one or more contexts over others.

ECM-associated TGF1: The term refers to TGFβ1 or its signaling complex (e.g., pro/latent TGFβ1) that is a component of (e.g., deposited into) the extracellular matrix. TGFβ1 that is presented by LTBP1 or LTBP3 is an ECM-associated TGFβ1.

Effective amount: An “effective amount” (or therapeutically effective amount) is a dosage or dosing regimen that achieves statistically significant clinical benefits in a patient population.

Epitope: The term “epitope” includes any molecular determinant (e.g., polypeptide determinant) that can specifically bind to a binding agent, immunoglobulin or T-cell receptor. In certain embodiments, epitope determinants include chemically active surface groupings of molecules, such as amino acids, sugar side chains, phosphoryl, or sulfonyl, and, in certain embodiments, may have specific three-dimensional structural characteristics, and/or specific charge characteristics. An epitope is a region of an antigen that is bound by a binding protein. An epitope thus consists of the amino acid residues of a region of an antigen (or fragment thereof) known to bind to the complementary site on the specific binding partner. An antigenic fragment can contain more than one epitope. In certain embodiments, an antibody is the to specifically bind an antigen when it recognizes its target antigen in a complex mixture of proteins and/or macromolecules. For example, antibodies are said to “bind to the same epitope” if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

Fibrosis: The term “fibrosis” or “fibrotic condition/disorder” refers to the process or manifestation characterized by the pathological accumulation of extracellular matrix (ECM) components, such as collagens, within a tissue or organ.

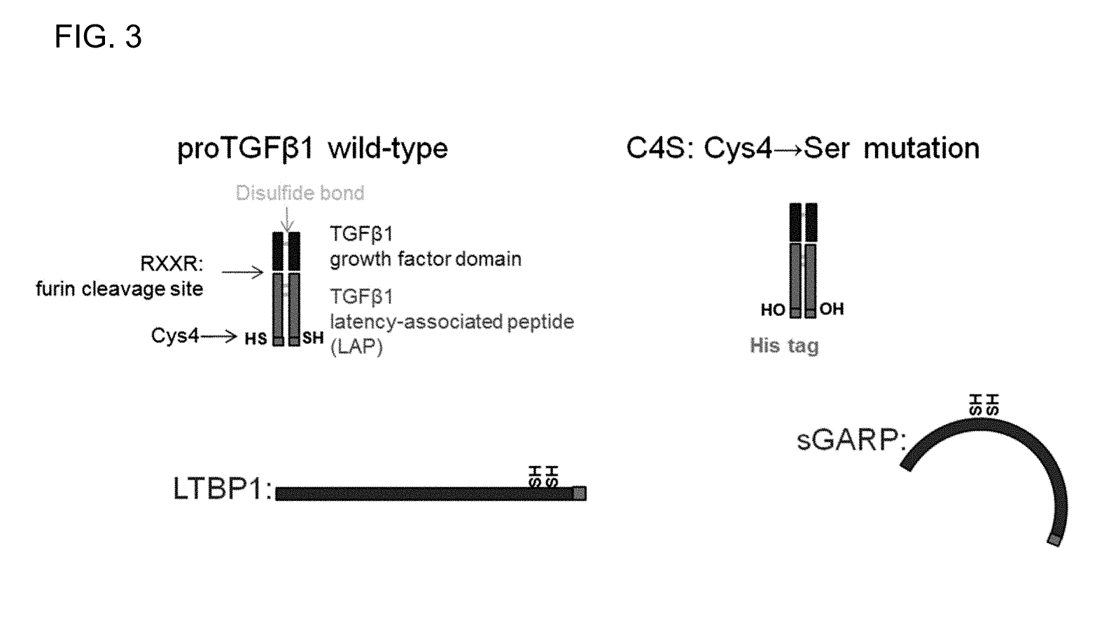

GARP-TGFβ1 complex: As used herein, the term “GARP-TGFβ1 complex” refers to a protein complex comprising a pro-protein form or latent form of a transforming growth factor-β1 (TGFβ1) protein and a glycoprotein-A repetitions predominant protein (GARP) or fragment or variant thereof. In some embodiments, a pro-protein form or latent form of TGFβ1 protein may be referred to as “pro/latent TGFβ1 protein”. In some embodiments, a GARP-TGFβ1 complex comprises GARP covalently linked with pro/latent TGFβ1 via one or more disulfide bonds. In other embodiments, a GARP-TGFβ1 complex comprises GARP non-covalently linked with pro/latent TGFβ1. In some embodiments, a GARP-TGFβ1 complex is a naturally-occurring complex, for example a GARP-TGFβ1 complex in a cell. An exemplary GARP-TGFβ1 complex is shown in FIG. 3.

Human antibody: The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Humanized antibody: The term “humanized antibody” refers to antibodies, which comprise heavy and light chain variable region sequences from a non-human species (e.g., a mouse) but in which at least a portion of the VH and/or VL sequence has been altered to be more “human-like,” i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody, or a variant, derivative, analog or fragment thereof, which immunospecifically binds to an antigen of interest and which comprises an FR region having substantially the amino acid sequence of a human antibody and a CDR region having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the FR regions are those of a human immunoglobulin consensus sequence. In an embodiment a humanized antibody also comprises at least a portion of an immunoglobulin Fc region, typically that of a human immunoglobulin. In some embodiments a humanized antibody contains the light chain as well as at least the variable domain of a heavy chain. The antibody also may include the CH1, hinge, CH2, CH3, and CH4 regions of the heavy chain. In some embodiments a humanized antibody only contains a humanized light chain. In some embodiments a humanized antibody only contains a humanized heavy chain. In specific embodiments a humanized antibody only contains a humanized variable domain of a light chain and/or humanized heavy chain.

Isoform-specific: The term “isoform specificity” refers to an agent's ability to discriminate one isoform over other structurally related isoforms (i.e., selectivity). An isoform-specific TGFβ inhibitor exerts its inhibitory activity towards one isoform of TGFβ but not the other isoforms of TGFβ at a given concentration. For example, an isoform-specific TGFβ1 antibody selectively binds TGFβ1. A TGFβ1-specific inhibitor (antibody) preferentially targets (binds thereby inhibits) the TGFβ1 isoform over TGFβ2 or TGFβ3 with substantially greater affinity. For example, the selectivity in this context may refer to at least a 500-1000-fold difference in respective affinities as measured by an in vitro binding assay such as Octet and Biacor. In some embodiments, the selectivity is such that the inhibitor when used at a dosage effective to inhibit TGFβ1 in vivo does not inhibit TGFβ2 and TGFβ3. For instance, an antibody may preferentially bind TGFβ1 at affinity of ˜1 pM, while the same antibody may bind TGFβ2 and/or TGFβ3 at ˜0.5-50 nM. For such an inhibitor to be useful as a therapeutic, dosage to achieve desirable effects (e.g., therapeutically effective amounts) must fall within the window within which the inhibitor can effectively inhibit the TGFβ1 isoform without inhibiting TGFβ2 or TGFβ3.

Isolated: An “isolated” antibody as used herein, refers to an antibody that is substantially free of other antibodies having different antigenic specificities. In some embodiments, an isolated antibody is substantially free of other unintended cellular material and/or chemicals.

Localized: In the context of the present disclosure, the term “localized” (as in “localized tumor”) refers to anatomically isolated or isolatable abnormalities, such as solid malignancies, as opposed to systemic disease. Certain leukemia, for example, may have both a localized component (for instance the bone marrow) and a systemic component (for instance circulating blood cells) to the disease.

LRRC33-TGFβ1 complex: As used herein, the term “LRRC33-TGFβ1 complex” refers to a complex between a pro-protein form or latent form of transforming growth factor-β1 (TGFβ1) protein and a Leucine-Rich Repeat-Containing Protein 33 (LRRC33; also known as Negative Regulator Of Reactive Oxygen Species or NRROS) or fragment or variant thereof. In some embodiments, a LRRC33-TGFβ1 complex comprises LRRC33 covalently linked with pro/latent TGFβ1 via one or more disulfide bonds. In other embodiments, a LRRC33-TGFβ1 complex comprises LRRC33 non-covalently linked with pro/latent TGFβ1. In some embodiments, a LRRC33-TGFβ1 complex is a naturally-occurring complex, for example a LRRC33-TGFβ1 complex in a cell.

LTBP1-TGFβ1 complex: As used herein, the term “LTBP1-TGFβ1 complex” refers to a protein complex comprising a pro-protein form or latent form of transforming growth factor-β1 (TGFβ1) protein and a latent TGF-beta binding protein 1 (LTBP1) or fragment or variant thereof. In some embodiments, a LTBP1-TGFβ1 complex comprises LTBP1 covalently linked with pro/latent TGFβ1 via one or more disulfide bonds. In other embodiments, a LTBP1-TGFβ1 complex comprises LTBP1 non-covalently linked with pro/latent TGFβ1. In some embodiments, a LTBP1-TGFβ1 complex is a naturally-occurring complex, for example a LTBP1-TGFβ1 complex in a cell. An exemplary LTBP1-TGFβ1 complex is shown in FIG. 3.

LTBP3-TGFβ1 complex: As used herein, the term “LTBP3-TGFβ1 complex” refers to a protein complex comprising a pro-protein form or latent form of transforming growth factor-β1 (TGFβ1) protein and a latent TGF-beta binding protein 3 (LTBP3) or fragment or variant thereof. In some embodiments, a LTBP3-TGFβ1 complex comprises LTBP3 covalently linked with pro/latent TGFβ1 via one or more disulfide bonds. In other embodiments, a LTBP3-TGFβ1 complex comprises LTBP1 non-covalently linked with pro/latent TGFβ1. In some embodiments, a LTBP3-TGFβ1 complex is a naturally-occurring complex, for example a LTBP3-TGFβ1 complex in a cell. An exemplary LTBP3-TGFβ1 complex is shown in FIG. 3.

Myelofibrosis: “Myelofibrosis,” also known as osteomyelofibrosis, is a relatively rare bone marrow proliferative disorder (e.g., cancer), which belongs to a group of diseases called myeloproliferative disorders. Myelofibrosis is classified into the Philadelphia chromosome-negative (−) branch of myeloproliferative neoplasms. Myelofibrosis is characterized by the proliferation of an abnormal clone of hematopoietic stem cells in the bone marrow and other sites results in fibrosis, or the replacement of the marrow with scar tissue. The term myelofibrosis, unless otherwise specified, refers to primary myelofibrosis (PMF). This may also be referred to as chronic idiopathic myelofibrosis (cIMF) (the terms idiopathic and primary mean that in these cases the disease is of unknown or spontaneous origin). This is in contrast with myelofibrosis that develops secondary to polycythemia vera or essential thrombocythaemia. Myelofibrosis is a form of myeloid metaplasia, which refers to a change in cell type in the blood-forming tissue of the bone marrow, and often the two terms are used synonymously. The terms agnogenic myeloid metaplasia and myelofibrosis with myeloid metaplasia (MMM) are also used to refer to primary myelofibrosis.

Pan-TGFβ inhibitor: The term “pan-TGFβ inhibitor” refers to any agent that is capable of inhibiting or antagonizing multiple isoforms of TGFβ. Such an inhibitor may be a small molecule inhibitor of TGFβ isoforms. The term includes pan-TGFβ antibody which refers to any agent that is capable of binding to more than one isoform of TGFβ, for example, at least two of TGFβ1, TGFβ2, and TGFβ3. In some embodiments, a pan-TGFβ antibody binds all three isoforms, i.e., TGFβ1, TGFβ2, and TGFβ3. In some embodiments, a pan-TGFβ antibody binds and neutralizes all three isoforms, i.e., TGFβ1, TGFβ2, and TGFβ3.

Presenting molecule: The term “presenting molecule” or “presentation molecule” of TGFβ is a protein entity that is capable of binding/linking to inactive form(s) of TGFβ thereby “presenting” the pro-protein in an extracellular domain. Four TGFβ presenting molecules have been identified to date: Latent TGFβ Binding Protein-1 (LTBP1) and LTBP3 are deposited into the extracellular matrix (i.e., components of the ECM), while Glycoprotein-A Repetitions Predominant (GARP/LRRC32) and Leucine-Rich Repeat-Containing Protein 33 (LRRC33) contain a transmembrane domain and present latent TGFβ1 on the surface of certain cells, such as immune cells. The TGFβ1 isoform alone has been implicated in a number of biological processes in both normal and disease conditions. These include, but are not limited to, maintenance of tissue homeostasis, inflammation response, ECM reorganization such as wound healing, and regulation of immune responses, as well as organ fibrosis, cancer, and autoimmunity.

ProTGFβ1: The term “proTGFβ1” as used herein is intended to encompass precursor forms of inactive TGFβ1 complex that comprises a prodomain sequence of TGFβ1 within the complex. Thus, the term can include the pro-, as well as the latent-forms of TGFβ1. The expression “pro/latent TGFβ1” may be used interchangeably. The “pro” form of TGFβ1 exists prior to proteolytic cleavage at the furin site. Once cleaved, the resulting form is said to be the “latent” form of TGFβ1. The “latent” complex remains associated until further activation trigger, such as integrin-driven activation event. As illustrated in FIG. 3, the proTGFβ1 complex is comprised of dimeric TGFβ1 pro-protein polypeptides, linked with disulfide bonds. It should be noted that the adjective “latent” may be used generally to describe the “inactive” state of TGFβ1, prior to integrin-mediated or other activation events.

Regulatory T cells: “Regulatory T cells,” or Tregs, are characterized by the expression of the biomarkers CD4, FOXP3, and CD25. Tregs are sometimes referred to as suppressor T cells and represent a subpopulation of T cells that modulate the immune system, maintain tolerance to self-antigens, and prevent autoimmune disease. Tregs are immunosuppressive and generally suppress or downregulate induction and proliferation of effector T (Teff) cells. Tregs can develop in the thymus (so-called CD4+ Foxp3+“natural” Tregs) or differentiate from naïve CD4+ T cells in the periphery, for example, following exposure to TGFβ or retinoic acid.

Solid tumor: The term “solid tumor” refers to proliferative disorders resulting in an abnormal growth or mass of tissue that usually does not contain cysts or liquid areas. Solid tumors may be benign (non-cancerous), or malignant (cancerous). Solid tumors may be comprised of cancerous (malignant) cells, stromal cells including CAFs, and infiltrating leukocytes, such as macrophages and lymphocytes.

Specific binding: As used herein, the term “specific binding” or “specifically binds” means that the interaction of the antibody, or antigen binding portion thereof, with an antigen is dependent upon the presence of a particular structure (e.g., an antigenic determinant or epitope). For example, the antibody, or antigen binding portion thereof, binds to a specific protein rather than to proteins generally. In some embodiments, an antibody, or antigen binding portion thereof, specifically binds to a target, e.g., TGFβ1, if the antibody has a KD for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the term “specific binding to an epitope of TGFβ1”, “specifically binds to an epitope of TGFβ1”, “specific binding to TGFβ1”, or “specifically binds to TGFβ1” as used herein, refers to an antibody, or antigen binding portion thereof, that binds to TGFβ1 and has a dissociation constant (KD) of 1.0×10⁻⁷ M or less, as determined by surface plasmon resonance. In one embodiment, an antibody, or antigen binding portion thereof, can specifically bind to both human and a non-human (e.g., mouse) orthologues of TGFβ1.

Subject: The term “subject” in the context of therapeutic applications refers to an individual who receives clinical care or intervention, such as treatment, diagnosis, etc. Suitable subjects include vertebrates, including but not limited to mammals (e.g., human and non-human mammals). Where the subject is a human subject, the term “patient” may be used interchangeably. In a clinical context, the term “a patient population” or “patient subpopulation” is used to refer to a group of individuals that falls within a set of criteria, such as clinical criteria (e.g., disease presentations, disease stages, susceptibility to certain conditions, responsiveness to therapy, etc.), medical history, health status, gender, age group, genetic criteria (e.g., carrier of certain mutation, polymorphism, gene duplications, DNA sequence repeats, etc.) and lifestyle factors (e.g., smoking, alcohol consumption, exercise, etc.).

TGFβ1-associated disorder: A “TGFβ1-associated disorder” means any disease or disorder, in which at least part of the pathogenesis and/or progression is attributable to TGFβ1 signaling or dysregulation thereof.

TGFβ inhibitor: The term “TGFβ inhibitor” refers to any agent capable of antagonizing biological activities or function of TGFβ growth factor (e.g., TGFβ1, TGFβ2 and/or TGFβ3). The term is not intended to limit its mechanism of action and includes, for example, neutralizing inhibitors, receptor antagonists, soluble ligand traps, and activation inhibitors of TGFβ.

The “TGFβ family” is a class within the TGFβ superfamily and contains three isoforms: TGFβ1, TGFβ2, and TGFβ3, which are structurally similar.

Toxicity: As used herein, the term “toxicity” or “toxicities” refers to unwanted in vivo effects in patients associated with a therapy administered to the patients, such as undesirable side effects and adverse events. “Tolerability” refers to a level of toxicities associated with a therapy or therapeutic regimen, which can be reasonably tolerated by patients, without discontinuing the therapy due to the toxicities.

Treat/treatment: The term “treat” or “treatment” includes therapeutic treatments, prophylactic treatments, and applications in which one reduces the risk that a subject will develop a disorder or other risk factor. Thus the term is intended to broadly mean: causing therapeutic benefits in a patient by, for example, enhancing or boosting the body's immunity; reducing or reversing immune suppression; reducing, removing or eradicating harmful cells or substances from the body; reducing disease burden (e.g., tumor burden); preventing recurrence or relapse; prolonging a refractory period, and/or otherwise improving survival. The term includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In the context of combination therapy, the term may also refer to: i) the ability of a second therapeutic to reduce the effective dosage of a first therapeutic so as to reduce side effects and increase tolerability; ii) the ability of a second therapy to render the patient more responsive to a first therapy; and/or iii) the ability to effectuate additive or synergistic clinical benefits.

Tumor-associated macrophage: “Tumor-associated macrophages (TAMs)” are polarized/activated macrophages with pro-tumor phenotypes. TAMs can be either marrow-originated monocytes/macrophages recruited to the tumor site or tissue-resident macrophages which are derived from erythro-myeloid progenitors. Differentiation of monocytes/macrophages into TAMs is influenced by a number of factors, including local chemical signals such as cytokines, chemokines, growth factors and other molecules that act as ligands, as well as cell-cell interactions between the monocytes/macrophages that are present in the niche (tumor microenvironment). Generally, monocytes/macrophages can be polarized into so-called “M1” or “M2” subtypes, the latter being associated with more pro-tumor phenotype. In a solid tumor, up to 50% of the tumor mass may correspond to macrophages, which are preferentially M2-polarized.

Tumor microenvironment: The term “tumor microenvironment (TME)” refers to a local disease niche, in which a tumor (e.g., solid tumor) resides in vivo.

Variable region: The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

Other than in the operating examples, or where otherwise indicated, all numbers expressing quantities of ingredients or reaction conditions used herein should be understood as modified in all instances by the term “about.” The term “about” when used in connection with percentages can mean ±1%.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, e.g., 10-20, 1-10, 30-40, etc.

### Isoform-Selective, Context-Permissive/Context-Independent Antibodies of TGFβ1

The present invention provides antibodies, and antigen binding portions thereof, that bind two or more of the following complexes comprising pro/latent-TGFβ1: a GARP-TGFβ1 complex, a LTBP1-TGFβ1 complex, a LTBP3-TGFβ1 complex, and a LRRC33-TGFβ1 complex. Accordingly, some aspects of the invention relate to antibodies, or antigen binding portions thereof, that specifically bind to an epitope within such TGFβ1 complex, wherein the epitope is available for binding by the antibody, or antigen-binding portions thereof, when the TGFβ1 is present in a GARP-TGFβ1 complex, a LTBP1-TGFβ1 complex, a LTBP3-TGFβ1 complex, and/or a LRRC33-TGFβ1 complex. In some embodiments, the epitope is available due to a conformational change in TGFβ1 when in complex with GARP, LTBP1, LTBP3, and/or LRRC33. In some embodiments, the epitope in TGFβ1 to which the antibodies, or antigen binding portions thereof, bind is not available when TGFβ1 is not in complex with GARP, LTBP1, LTBP3, and/or LRRC33. In some embodiments, the antibodies, or antigen binding portions thereof, do not specifically bind to TGFβ2. In some embodiments, the antibodies, or antigen binding portions thereof, do not specifically bind to TGFβ3. In some embodiments, the antibodies, or antigen binding portions thereof, do not prevent TGFβ1 from binding to integrin. For example, in some embodiments, the antibodies, or antigen binding portions thereof, do not mask the integrin-binding site of TGFβ1. In some embodiments, the antibodies, or antigen binding portions thereof, inhibit the activation of TGFβ1. In some embodiments, the antibodies, or antigen binding portions thereof, inhibit the release of mature TGFβ1 from a GARP-TGFβ1 complex, a LTBP1-TGFβ1 complex, a LTBP3-TGFβ1 complex, and/or a LRRC33-TGFβ1 complex.

Antibodies, or antigen binding portions thereof, provided herein specifically bind to an epitope of multiple (i.e., two or more) TGFβ1 complexes, wherein the epitope is available for binding by the antibody, or antigen binding portions thereof, when the TGFβ1 is present in a GARP-TGFβ1 complex, a LTBP1-TGFβ1 complex, a LTBP2-TGFβ1 complex, LTBP3-TGFβ1 complex, LTBP4-TGFβ1 complex and/or a LRRC33-TGFβ1 complex. In some embodiments, the TGFβ1 comprises a naturally occurring mammalian amino acid sequence. In some embodiment, the TGFβ1 comprises a naturally occurring human amino acid sequence. In some embodiments, the TGFβ1 comprises a human, a monkey, a rat or a mouse amino acid sequence. In some embodiments, an antibody, or antigen binding portion thereof, described herein does not specifically bind to TGFβ2. In some embodiments, an antibody, or antigen binding portion thereof, described herein does not specifically bind to TGFβ3. In some embodiments, an antibody, or antigen binding portion thereof, described herein does not specifically bind to TGFβ2 or TGFβ3. In some embodiments, an antibody, or antigen binding portion thereof, described herein specifically binds to a TGFβ1 comprising the amino acid sequence set forth in SEQ ID NO: 21. The amino acid sequences of TGFβ2, and TGFβ3 amino acid sequence are set forth in SEQ ID NOs: 22 and 23, respectively. In some embodiments, an antibody, or antigen binding portion thereof, described herein specifically binds to a TGFβ1 comprising a non-naturally-occurring amino acid sequence (otherwise referred to herein as a non-naturally-occurring TGFβ1). For example, a non-naturally-occurring TGFβ1 may comprise one or more recombinantly generated mutations relative to a naturally-occurring TGFβ1 amino acid sequence. In some embodiments, a TGFβ1, TGFβ2, or TGFβ3 amino acid sequence comprises the amino acid sequence as set forth in SEQ ID NOs: 24-35, as shown in Table 1. In some embodiments, a TGFβ1, TGFβ2, or TGFβ3 amino acid sequence comprises the amino acid sequence as set forth in SEQ ID NOs: 36-43, as shown in Table 2.

TGFβ1
(SEQ ID NO: 21)
LSTCKTIDMELVKRKRIEAIRGQILSKLRLASPPSQGEVPPGPLPEAVLA
LYNSTRDRVAGESAEPEPEPEADYYAKEVTRVLMVETHNEIYDKFKQSTH
SIYMFFNTSELREAVPEPVLLSRAELRLLRLKLKVEQHVELYQKYSNNSW
RYLSNRLLAPSDSPEWLSFDVTGVVRQWLSRGGEIEGFRLSAHCSCDSRD
NTLQVDINGFTTGRRGDLATIHGMNRPFLLLMATPLERAQHLQSSRHRRA
LDTNYCFSSTEKNCCVRQLYIDFRKDLGWKWIHEPKGYHANFCLGPCPYI
WSLDTQYSKVLALYNQHNPGASAAPCCVPQALEPLPIVYYVGRKPKVEQL
SNMIVRSCKCS
TGFβ2
(SEQ ID NO: 22)
SLSTCSTLDMDQFMRKRIEAIRGQILSKLKLTSPPEDYPEPEEVPPEVIS
IYNSTRDLLQEKASRRAAACERERSDEEYYAKEVYKIDMPPFFPSENAIP
PTFYRPYFRIVRFDVSAMEKNASNLVKAEFRVFRLQNPKARVPEQRIELY
QILKSKDLTSPTQRYIDSKVVKTRAEGEWLSFDVTDAVHEWLHHKDRNLG
FKISLHCPCCTFVPSNNYIIPNKSEELEARFAGIDGTSTYTSGDQKTIKS
TRKKNSGKTPHLLLMLLPSYRLESQQTNRRKKRALDAAYCFRNVQDNCCL
RPLYIDFKRDLGWKWIHEPKGYNANFCAGACPYLWSSDTQHSRVLSLYNT
INPEASASPCCVSQDLEPLTILYYIGKTPKIEQLSNMIVKSCKCS
TGFβ3
(SEQ ID NO: 23)
SLSLSTCTTLDFGHIKKKRVEAIRGQILSKLRLTSPPEPTVMTHVPYQVL
ALYNSTRELLEEMHGEREEGCTQENTESEYYAKEIHKFDMIQGLAEHNEL
AVCPKGITSKVFRFNVSSVEKNRTNLFRAEFRVLRVPNPSSKRNEQRIEL
FQILRPDEHIAKQRYIGGKNLPTRGTAEWLSFDVTDTVREWLLRRESNLG
LEISIHCPCHTFQPNGDILENIHEVMEIKFKGVDNEDDHGRGDLGRLKKQ
KDHHNPHLILMMIPPHRLDNPGQGGQRKKRALDTNYCFRNLEENCCVRPL
YIDFRQDLGWKWVHEPKGYYANFCSGPCPYLRSADTTHSTVLGLYNTLNP
EASASPCCVPQDLEPLTILYYVGRTPKVEQLSNMVVKSCKCS

TABLE 1
Exemplary TGFβ1, TGFβ2, and TGFβ3 amino acid sequences

| | | SEQ ID |
|---|---|---|
| Protein | Sequence | NO |
| proTGFβ1 | LSTCKTIDMELVKRKRIEAIRGQILSKLRLASPPSQGEVPPGPLPE | 24 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHRRALDTNYCFSSTEKNC | |
| | CVRQLYIDFRKDLGWKWIHEPKGYHANFCLGPCPYIWSLDTQY | |
| | SKVLALYNQHNPGASAAPCCVPQALEPLPIVYYVGRKPKVEQLS | |
| | NMIVRSCKCS | |
| proTGFβ1 C4S | LSTSKTIDMELVKRKRIEAIRGQILSKLRLASPPSQGEVPPGPLPE | 25 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHRRALDTNYCFSSTEKNC | |
| | CVRQLYIDFRKDLGWKWIHEPKGYHANFCLGPCPYIWSLDTQY | |
| | SKVLALYNQHNPGASAAPCCVPQALEPLPIVYYVGRKPKVEQLS | |
| | NMIVRSCKCS | |
| proTGFβ1 D2G | LSTCKTIDMELVKRKRIEAIRGQILSKLRLASPPSQGEVPPGPLPE | 26 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHGALDTNYCFSSTEKNCC | |
| | VRQLYIDFRKDLGWKWIHEPKGYHANFCLGPCPYIWSLDTQYSK | |
| | VLALYNQHNPGASAAPCCVPQALEPLPIVYYVGRKPKVEQLSNM | |
| | IVRSCKCS | |
| proTGFβ1 C4S D2G | LSTSKTIDMELVKRKRIEAIRGQILSKLRLASPPSQGEVPPGPLPE | 27 |
| | AVLALYNSTRDRVAGESAEPEPEPEADYYAKEVTRVLMVETHN | |
| | EIYDKFKQSTHSIYMFFNTSELREAVPEPVLLSRAELRLLRLKLKV | |
| | EQHVELYQKYSNNSWRYLSNRLLAPSDSPEWLSFDVTGVVRQ | |
| | WLSRGGEIEGFRLSAHCSCDSRDNTLQVDINGFTTGRRGDLATI | |
| | HGMNRPFLLLMATPLERAQHLQSSRHGALDTNYCFSSTEKNCC | |
| | VRQLYIDFRKDLGWKWIHEPKGYHANFCLGPCPYIWSLDTQYSK | |
| | VLALYNQHNPGASAAPCCVPQALEPLPIVYYVGRKPKVEQLSNM | |
| | IVRSCKCS | |
| proTGFβ2 | SLSTCSTLDMDQFMRKRIEAIRGQILSKLKLTSPPEDYPEPEEVP | 28 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKKRALDAAYCFRNVQDNCCLRPLYIDFKRDLGW | |
| | KWIHEPKGYNANFCAGACPYLWSSDTQHSRVLSLYNTINPEASA | |
| | SPCCVSQDLEPLTILYYIGKTPKIEQLSNMIVKSCKCS | |
| proTGFβ2 C5S | SLSTSSTLDMDQFMRKRIEAIRGQILSKLKLTSPPEDYPEPEEVP | 29 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKKRALDAAYCFRNVQDNCCLRPLYIDFKRDLGW | |
| | KWIHEPKGYNANFCAGACPYLWSSDTQHSRVLSLYNTINPEASA | |
| | SPCCVSQDLEPLTILYYIGKTPKIEQLSNMIVKSCKCS | |
| proTGFβ2 C5S D2G | SLSTSSTLDMDQFMRKRIEAIRGQILSKLKLTSPPEDYPEPEEVP | 30 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKGALDAAYCFRNVQDNCCLRPLYIDFKRDLGWK | |
| | WIHEPKGYNANFCAGACPYLWSSDTQHSRVLSLYNTINPEASAS | |
| | PCCVSQDLEPLTILYYIGKTPKIEQLSNMIVKSCKCS | |
| proTGFβ2 D2G | SLSTCSTLDMDQFMRKRIEAIRGQILSKLKLTSPPEDYPEPEEVP | 31 |
| | PEVISIYNSTRDLLQEKASRRAAACERERSDEEYYAKEVYKIDMP | |
| | PFFPSENAIPPTFYRPYFRIVRFDVSAMEKNASNLVKAEFRVFRL | |
| | QNPKARVPEQRIELYQILKSKDLTSPTQRYIDSKVVKTRAEGEWL | |
| | SFDVTDAVHEWLHHKDRNLGFKISLHCPCCTFVPSNNYIIPNKSE | |
| | ELEARFAGIDGTSTYTSGDQKTIKSTRKKNSGKTPHLLLMLLPSY | |
| | RLESQQTNRRKGALDAAYCFRNVQDNCCLRPLYIDFKRDLGWK | |
| | WIHEPKGYNANFCAGACPYLWSSDTQHSRVLSLYNTINPEASAS | |
| | PCCVSQDLEPLTILYYIGKTPKIEQLSNMIVKSCKCS | |
| proTGFβ3 | SLSLSTCTTLDFGHIKKKRVEAIRGQILSKLRLTSPPEPTVMTHVP | 32 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKKRALDTNYCFRNLEENCCVRPLYIDFRQDLGWK | |
| | WVHEPKGYYANFCSGPCPYLRSADTTHSTVLGLYNTLNPEASA | |
| | SPCCVPQDLEPLTILYYVGRTPKVEQLSNMVVKSCKCS | |
| proTGFβ3 C7S | SLSLSTSTTLDFGHIKKKRVEAIRGQILSKLRLTSPPEPTVMTHVP | 33 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKKRALDTNYCFRNLEENCCVRPLYIDFRQDLGWK | |
| | WVHEPKGYYANFCSGPCPYLRSADTTHSTVLGLYNTLNPEASA | |
| | SPCCVPQDLEPLTILYYVGRTPKVEQLSNMVVKSCKCS | |
| proTGFβ3 C7S D2G | SLSLSTSTTLDFGHIKKKRVEAIRGQILSKLRLTSPPEPTVMTHVP | 34 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKGALDTNYCFRNLEENCCVRPLYIDFRQDLGWKW | |
| | VHEPKGYYANFCSGPCPYLRSADTTHSTVLGLYNTLNPEASASP | |
| | CCVPQDLEPLTILYYVGRTPKVEQLSNMVVKSCKCS | |
| proTGFβ3 D2G | SLSLSTCTTLDFGHIKKKRVEAIRGQILSKLRLTSPPEPTVMTHVP | 35 |
| | YQVLALYNSTRELLEEMHGEREEGCTQENTESEYYAKEIHKFDM | |
| | IQGLAEHNELAVCPKGITSKVFRFNVSSVEKNRTNLFRAEFRVLR | |
| | VPNPSSKRNEQRIELFQILRPDEHIAKQRYIGGKNLPTRGTAEWL | |
| | SFDVTDTVREWLLRRESNLGLEISIHCPCHTFQPNGDILENIHEV | |
| | MEIKFKGVDNEDDHGRGDLGRLKKQKDHHNPHLILMMIPPHRLD | |
| | NPGQGGQRKGALDTNYCFRNLEENCCVRPLYIDFRQDLGWKW | |
| | VHEPKGYYANFCSGPCPYLRSADTTHSTVLGLYNTLNPEASASP | |
| | CCVPQDLEPLTILYYVGRTPKVEQLSNMVVKSCKCS | |

TABLE 2
Exemplary non-human amino acid sequences

| | | | SEQ ID |
|---|---|---|---|
| Protein | Species | Sequence | NO |
| proTGFβ1 | Mouse | LSTCKTIDMELVKRKRIEAIRGQILSKLRLASPPSQGEVPPGPL | 36 |
| | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRRALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFβ1 | Cyno | LSTCKTIDMELVKRKRIEAIRGQILSKLRLASPPSQGEVPPGPL | 37 |
| | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRRAL | |
| | | DTNYCFSSTEKNCCVRQLYIDFRKDLGWKWIHEPKGYHANF | |
| | | CLGPCPYIWSLDTQYSKVLALYNQHNPGASAAPCCVPQALE | |
| | | PLPIVYYVGRKPKVEQLSNMIVRSCKCS | |
| TGFβ1 LAP | Mouse | LSTSKTIDMELVKRKRIEAIRGQILSKLRLASPPSQGEVPPGPL | 38 |
| C4S | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRR | |
| TGFβ1 LAP | Cyno | LSTSKTIDMELVKRKRIEAIRGQILSKLRLASPPSQGEVPPGPL | 39 |
| C4S | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRR | |
| proTGFβ1 | Mouse | LSTSKTIDMELVKRKRIEAIRGQILSKLRLASPPSQGEVPPGPL | 40 |
| C4S D2G | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHGALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFβ1 | Mouse | LSTSKTIDMELVKRKRIEAIRGQILSKLRLASPPSQGEVPPGPL | 41 |
| C4S | | PEAVLALYNSTRDRVAGESADPEPEPEADYYAKEVTRVLMV | |
| | | DRNNAIYEKTKDISHSIYMFFNTSDIREAVPEPPLLSRAELRLQ | |
| | | RLKSSVEQHVELYQKYSNNSWRYLGNRLLTPTDTPEWLSFD | |
| | | VTGVVRQWLNQGDGIQGFRFSAHCSCDSKDNKLHVEINGIS | |
| | | PKRRGDLGTIHDMNRPFLLLMATPLERAQHLHSSRHRRALDT | |
| | | NYCFSSTEKNCCVRQLYIDFRKDLGWKWIHEPKGYHANFCL | |
| | | GPCPYIWSLDTQYSKVLALYNQHNPGASASPCCVPQALEPL | |
| | | PIVYYVGRKPKVEQLSNMIVRSCKCS | |
| proTGFβ1 | Cyno | LSTSKTIDMELVKRKRIEAIRGQILSKLRLASPPSQGEVPPGPL | 42 |
| C4S | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHRRAL | |
| | | DTNYCFSSTEKNCCVRQLYIDFRKDLGWKWIHEPKGYHANF | |
| | | CLGPCPYIWSLDTQYSKVLALYNQHNPGASAAPCCVPQALE | |
| | | PLPIVYYVGRKPKVEQLSNMIVRSCKCS | |
| proTGFβ31 | Cyno | LSTSKTIDMELVKRKRIEAIRGQILSKLRLASPPSQGEVPPGPL | 43 |
| C4S D2G | | PEAVLALYNSTRDRVAGESAEPEPEPEADYYAKEVTRVLMV | |
| | | ETHNEIYDKFKQSTHSIYMFFNTSELREAVPEPVLLSRAELRL | |
| | | LRLKLKVEQHVELYQKYSNNSWRYLSNRLLAPSDSPEWLSF | |
| | | DVTGVVRQWLSRGGEIEGFRLSAHCSCDSKDNTLQVDINGF | |
| | | TTGRRGDLATIHGMNRPFLLLMATPLERAQHLQSSRHGALDT | |
| | | NYCFSSTEKNCCVRQLYIDFRKDLGWKWIHEPKGYHANFCL | |
| | | GPCPYIWSLDTQYSKVLALYNQHNPGASAAPCCVPQALEPL | |
| | | PIVYYVGRKPKVEQLSNMIVRSCKCS | |
| LTBP3 | CYNO | GPAGERGAGGGGALARERFKVVFAPVICKRTCLKGQCRDSC | 44 |
| | | QQGSNMTLIGENGHSTDTLTGSGFRVVVCPLPCMNGGQCS | |
| | | SRNQCLCPPDFTGRFCQVPAGGAGGGTGGSGPGLSRAGAL | |
| | | STGALPPLAPEGDSVASKHAIYAVQVIADPPGPGEGPPAQHA | |
| | | AFLVPLGPGQISAEVQAPPPVVNVRVHHPPEASVQVHRIESS | |
| | | NAEGAAPSQHLLPHPKPSHPRPPTQKPLGRCFQDTLPKQPC | |
| | | GSNPLPGLTKQEDCCGSIGTAWGQSKCHKCPQLQYTGVQK | |
| | | PGPVRGEVGADCPQGYKRLNSTHCQDINECAMPGVCRHGD | |
| | | CLNNPGSYRCVCPPGHSLGPSRTQCIADKPEEKSLCFRLVS | |
| | | PEHQCQHPLTTRLTRQLCCCSVGKAWGARCQRCPADGTAA | |
| | | FKEICPAGKGYHILTSHQTLTIQGESDFSLFLHPDGPPKPQQL | |
| | | PESPSQAPPPEDTEEERGVTTDSPVSEERSVQQSHPTATTS | |
| | | PARPYPELISRPSPPTMRWFLPDLPPSRSAVEIAPTQVTETD | |
| | | ECRLNQNICGHGECVPGPPDYSCHCNPGYRSHPQHRYCVD | |
| | | VNECEAEPCGPGRGICMNTGGSYNCHCNRGYRLHVGAGGR | |
| | | SCVDLNECAKPHLCGDGGFCINFPGHYKCNCYPGYRLKASR | |
| | | PPVCEDIDECRDPSSCPDGKCENKPGSFKCIACQPGYRSQG | |
| | | GGACRDVNECAEGSPCSPGWCENLPGSFRCTCAQGYAPAP | |
| | | DGRSCVDVDECEAGDVCDNGICTNTPGSFQCQCLSGYHLS | |
| | | RDRSHCEDIDECDFPAACIGGDCINTNGSYRCLCPQGHRLV | |
| | | GGRKCQDIDECTQDPGLCLPHGACKNLQGSYVCVCDEGFT | |
| | | PTQDQHGCEEVEQPHHKKECYLNFDDTVFCDSVLATNVTQQ | |
| | | ECCCSLGAGWGDHCEIYPCPVYSSAEFHSLCPDGKGYTQD | |
| | | NNIVNYGIPAHRDIDECMLFGAEICKEGKCVNTQPGYECYCK | |
| | | QGFYYDGNLLECVDVDECLDESNCRNGVCENTRGGYRCAC | |
| | | TPPAEYSPAQRQCLSPEEMDVDECQDPAACRPGRCVNLPG | |
| | | SYRCECRPPWVPGPSGRDCQLPESPAERAPERRDVCWSQ | |
| | | RGEDGMCAGPQAGPALTFDDCCCRQGRGWGAQCRPCPPR | |
| | | GAGSQCPTSQSESNSFWDTSPLLLGKPRRDEDSSEEDSDE | |
| | | CRCVSGRCVPRPGGAVCECPGGFQLDASRARCVDIDECRE | |
| | | LNQRGLLCKSERCVNTSGSFRCVCKAGFARSRPHGACVPQ | |
| | | RRR | |
| LTBP3 | Mouse | GPAGERGTGGGGALARERFKVVFAPVICKRTCLKGQCRDSC | 45 |
| | | QQGSNMTLIGENGHSTDTLTGSAFRVVVCPLPCMNGGQCS | |
| | | SRNQCLCPPDFTGRFCQVPAAGTGAGTGSSGPGLARTGAM | |
| | | STGPLPPLAPEGESVASKHAIYAVQVIADPPGPGEGPPAQHA | |
| | | AFLVPLGPGQISAEVQAPPPVVNVRVHHPPEASVQVHRIEGP | |
| | | NAEGPASSQHLLPHPKPPHPRPPTQKPLGRCFQDTLPKQPC | |
| | | GSNPLPGLTKQEDCCGSIGTAWGQSKCHKCPQLQYTGVQK | |
| | | PVPVRGEVGADCPQGYKRLNSTHCQDINECAMPGNVCHGD | |
| | | CLNNPGSYRCVCPPGHSLGPLAAQCIADKPEEKSLCFRLVST | |
| | | EHQCQHPLTTRLTRQLCCCSVGKAWGARCQRCPADGTAAF | |
| | | KEICPGKGYHILTSHQTLTIQGESDFSLFLHPDGPPKPQQLPE | |
| | | SPSRAPPLEDTEEERGVTMDPPVSEERSVQQSHPTTTTSPP | |
| | | RPYPELISRPSPPTFHRFLPDLPPSRSAVEIAPTQVTETDECR | |
| | | LNQNICGHGQCVPGPSDYSCHCNAGYRSHPQHRYCVDVNE | |
| | | CEAEPCGPGKGICMNTGGSYNCHCNRGYRLHVGAGGRSCV | |
| | | DLNECAKPHLCGDGGFCINFPGHYKCNCYPGYRLKASRPPI | |
| | | CEDIDECRDPSTCPDGKCENKPGSFKCIACQPGYRSQGGGA | |
| | | CRDVNECSEGTPCSPGWCENLPGSYRCTCAQYEPAQDGLS | |
| | | CIDVDECEAGKVCQDGICTNTPGSFQCQCLSGYHLSRDRSR | |
| | | CEDIDECDFPAACIGGDCINTNGSYRCLCPLGHRLVGGRKCK | |
| | | KDIDECSQDPGLCLPHACENLQGSYVCVCDEGFTLTQDQHG | |
| | | CEEVEQPHHKKECYLNFDDTVFCDSVLATNVTQQECCCSLG | |
| | | AGWGDHCEIYPCPVYSSAEFHSLVPDGKRLHSGQQHCELCI | |
| | | PAHRDIDECILFGAEICKEGKCVNTQPGYECYCKQGFYYDGN | |
| | | LLECVDVDECLDESNCRNGVCENTRGGYRCACTPPAEYSPA | |
| | | QAQCLIPERWSTPQRDVKCAGASEERTACVWGPWAGPALT | |
| | | FDDCCCRQPRLGTQCRPCPPRGTGSQCPTSQSESNSFWDT | |
| | | SPLLLGKSPRDEDSSEEDSDECRCVSGRCVPRPGGAVCEC | |
| | | PGGFQLDASRARCVDIDECRELNQRGLLCKSERCVNTSGSF | |
| | | RCVCKAGFTRSRPHGPACLSAAADDAAIAHTSVIDHRGYFH | |
| LTBP1S | Cyno | NHTGRIKVVFTPSICKVTCTKGSCQNSCEKGNTTTLISENGHA | 46 |
| | | ADTLTATNFRVVLCHLPCMNGGQCSSRDKCQCPPNFTGKLC | |
| | | QIPVHGASVPKLYQHSQQPGKALGTHVIHSTHTLPLTVTSQQ | |
| | | GVKVKFPPNIVNIHVKHPPEASVQIHQVSRIDGPTGQKTKEAQ | |
| | | PGQSQVSYQGLPVQKTQTIHSTYSHQQVIPHVYPVAAKTQL | |
| | | GRCFQETIGSQCGKALPGLSKQEDCCGTVGTSWGFNKCQK | |
| | | CPKKPSYHGYNQMMECLPGYKRVNNTFCQDINECQLQGVC | |
| | | PNGECLNTMGSYRCTCKIGFGPDPTFSSCVPDPPVISEEKGP | |
| | | CYRLVSSGRQCMHPLSVHLTKQLCCCSVGKAWGPHCEKCP | |
| | | LPGTAAFKEICPGGMGYTVSGVHRRRPIHHHVGKGPVFVKP | |
| | | KNTQPVAKSTHPPPLPAKEEPVEALTFSREHGPGVAEPEVAT | |
| | | APPEKEIPSLDQEKTKLEPGQPQLSPGISTIHLHPQFPVVIEKT | |
| | | SPPVPVEVAPEASTSSASQVIAPTQVTEINECTVNPDICGAGH | |
| | | CINLPVRYTCICYEGYKFSEQQRKCVDIDECTQVQHLCSQGR | |
| | | CENTEGSFLCICPAGFMASEEGTNCIDVDECLRPDVCGEGH | |
| | | CVNTVGAFRCEYCDSGYRMTQRGRCEDIDECLNPSTCPDE | |
| | | QCVNSPGSYQCVPCTEGFRGWNGQCLDVDECLEPNVCTN | |
| | | GDCSNLEGSYMCSCHKGYTRTPDHKHCKDIDECQQGNLCV | |
| | | NGQCKNTEGSFRCTCGQGYQLSAAKDQCEDIDECQHHHLC | |
| | | AHGQCRNTEGSFQCVCDQGYRASGLGDHCEDINECLEDKS | |
| | | VCQRGDCINTAGSYDCTCPDGFQLDDNKTCQDINECEHPGL | |
| | | CGPQGECLNTEGSFHCVCQQGFSISADGRTCEDIDECVNNT | |
| | | VCDSHGFCDNTAGSFRCLCYQGFQAPQDGQGCVDVNECEL | |
| | | LSGVCGEAFCENVEGSFLCVCADENQEYSPMTGQCRSRTS | |
| | | TDLDVEQPKEEKKECYYNLNDASLCDNVLAPNVTKQECCCT | |
| | | SGAGWGDNCEIFPCPVLGTAEFTEMCPKGKGFVPAGESSSE | |
| | | AGGENYKDADECLLFGQEICKNGFCLNTRPGYECYCKQGTY | |
| | | YDPVKLQCFDMDECQDPSSCIDGQCVNTEGSYNCFCTHPM | |
| | | VLDASEKRCIRPAESNEQIEETDVYQDLCWEHLSDEYVCSRP | |
| | | LVGKQTTYTECCCLYGEAWGMQCALCPMKDSDDYAQLCNI | |
| | | PVTGRRQPYGRDALVDFSEQYAPEADPYFIQDRFLNSFEEL | |
| | | QAEECGILNGCENGRCVRVQEGYTCDCFDGYHLDTAKMTC | |
| | | VDVNECDELNNRMSLCKNAKCINTEGSYKCLCLPGYVPSDK | |
| | | PNYCTPLNTALNLEKDSDLE | |
| LTBP1S | mouse | NHTGRIKVVFTPSICKVTCTKGNCQNSCQKGNTTTLISENGH | 47 |
| | | AADTLTATNFRVVICHLPCMNGGQCSSRDKCQCPPNFTGKL | |
| | | CQIPVLGASMPKLYQHAQQQGKALGSHVIHSTHTLPLTMTSQ | |
| | | QGVKVKFPPNIVNIHVKHPPEASVQIHQVSRIDSPGGQKVKE | |
| | | AQPGQSQVSYQGLPVQKTQTVHSTYSHQQLIPHVYPVAAKT | |
| | | QLGRCFQETIGSQCGKALPGLSKQEDCCGTVGTSWGFNKC | |
| | | QKCPKKQSYHGYTQMMECLQGYKRVNNTFCQDINECQLQG | |
| | | VCPNGECLNTMGSYRCSCKMGFGPDPTFSSCVPDPPVISEE | |
| | | KGPCYRLVSPGRHCMHPLSVHLTKQICCCSVGKAWGPHCE | |
| | | KCPLPGTAAFKEICPGGMGYTVSGVHRRRPIHQHIGKEAVYV | |
| | | KPKNTQPVAKSTHPPPLPAKEEPVEALTSSWEHGPRGAEPE | |
| | | VVTAPPEKEIPSLDQEKTRLEPGQPQLSPGVSTIHLHPQFPVV | |
| | | VEKTSPPVPVEVAPEASTSSASQVIAPTQVTEINECTVNPDIC | |
| | | GAGHCINLPVRYTCICYEGYKFSEQLRKCVDIDECAQVRHLC | |
| | | SQGRCENTEGSFLCVCPAGFMASEEGTNCIDVDECLRPDMC | |
| | | RDGRCINTAGAFRCEYCDSGYRMSRRGYCEDIDECLKPSTC | |
| | | PEEQCVNTPGSYQCVPCTEGFRGWNGQCLDVDECLQPKVC | |
| | | TNGSCTNLEGSYMCSCHRGYSPTPDHRHCQDIDECQQGNL | |
| | | CMNGQCRNTDGSFRCTCGQGYQLSAAKDQCEDIDECEHHH | |
| | | LCSHGQCRNTEGSFQCVCNQGYRASVLGDHCEDINECLED | |
| | | SSVCQGGDCINTAGSYDCTCPDGFQLNDNKGCQDINECAQP | |
| | | GLCGSHGECLNTQGSFHCVCEQGFSISADGRTCEDIDECVN | |
| | | NTVCDSHGFCDNTAGSFRCLCYQGFQAPQDGQGCVDVNEC | |
| | | ELLSGVCGEAFCENVEGSFLCVCADENQEYSPMTGQCRSR | |
| | | VTEDSGVDRQPREEKKECYYNLNDASLCDNVLAPNVTKQEC | |
| | | CCTSGAGWGDNCEIFPCPVQGTAEFTEMCPRGKGLVPAGE | |
| | | SSYDTGGENYKDADECLLFGEEICKNGYCLNTQPGYECYCK | |
| | | QGTYYDPVKLQCFDMDECQDPNSCIDGQCVNTEGSYNCFC | |
| | | THPMVLDASEKRCVQPTESNEQIEETDVYQDLCWEHLSEEY | |
| | | VCSRPLVGKQTTYTECCCLYGEAWGMQCALCPMKDSDDYA | |
| | | QLCNIPVTGRRRPYGRDALVDFSEQYGPETDPYFIQDRFLNS | |
| | | FEELQAEECGILNGCENGRCVRVQEGYTCDCFDGYHLDMAK | |
| | | MTCVDVNECSELNNRMSLCKNAKCINTEGSYKCLCLPGYIPS | |
| | | DKPNYCTPLNSALNLDKESDLE | |
| GARP | mouse | ISQRREQVPCRTVNKEALCHGLGLLQVPSVLSLDIQALYLSG | 48 |
| | | NQLQSILVSPLGFYTALRHLDLSDNQISFLQAGVFQALPYLEH | |
| | | LNLAHNRLATGMALNSGGLGRLPLLVSLDLSGNSLHGNLVER | |
| | | LLGETPRLRTLSLAENSLTRLARHTFWGMPAVEQLDLHSNVL | |
| | | MDIEDGAFEALPHLTHLNLSRNSLTCISDFSLQQLQVLDLSCN | |
| | | SIEAFQTAPEPQAQFQLAWLDLRENKLLHFPDLAVFPRLIYLN | |
| | | VSNNLIQLPAGLPRGSEDLHAPSEGWSASPLSNPSRNASTH | |
| | | PLSQLLNLDLSYNEIELVPASFLEHLTSLRFLNLSRNCLRSFEA | |
| | | RQVDSLPCLVLLDLSHNVLEALELGTKVLGSLQTLLLQDNALQ | |
| | | ELPPYTFASLASLQRLNLQGNQVSPCGGPAEPGPPGCVDFS | |
| | | GIPTLHVLNMAGNSMGMLRAGSFLHTPLTELDLSTNPGLDVA | |
| | | TGALVGLEASLEVLELQGNGLTVLRVDLPCFLRLKRLNLAEN | |
| | | QLSHLPAWTRAVSLEVLDLRNNSFSLLPGNAMGGLETSLRRL | |
| | | YLQGNPLSCCGNGWLAAQLHQGRVDVDATQDLICRFGSQE | |
| | | ELSLSLVRPEDCEKGGLKNVNLILLLSFTLVSAIVLTTLATICFL | |
| | | RRQKLSQQYKA | |
| sGARP | mouse | ISQRREQVPCRTVNKEALCHGLGLLQVPSVLSLDIQALYLSG | 49 |
| | | NQLQSILVSPLGFYTALRHLDLSDNQISFLQAGVFQALPYLEH | |
| | | LNLAHNRLATGMALNSGGLGRLPLLVSLDLSGNSLHGNLVER | |
| | | LLGETPRLRTLSLAENSLTRLARHTFWGMPAVEQLDLHSNVL | |
| | | MDIEDGAFEALPHLTHLNLSRNSLTCISDFSLQQLQVLDLSCN | |
| | | SIEAFQTAPEPQAQFQLAWLDLRENKLLHFPDLAVFPRLIYLN | |
| | | VSNNLIQLPAGLPRGSEDLHAPSEGWSASPLSNPSRNASTH | |
| | | PLSQLLNLDLSYNEIELVPASFLEHLTSLRFLNLSRNCLRSFEA | |
| | | RQVDSLPCLVLLDLSHNVLEALELGTKVLGSLQTLLLQDNALQ | |
| | | ELPPYTFASLASLQRLNLQGNQVSPCGGPAEPGPPGCVDFS | |
| | | GIPTLHVLNMAGNSMGMLRAGSFLHTPLTELDLSTNPGLDVA | |
| | | TGALVGLEASLEVLELQGNGLTVLRVDLPCFLRLKRLNLAEN | |
| | | QLSHLPAWTRAVSLEVLDLRNNSFSLLPGNAMGGLETSLRRL | |
| | | YLQGNPLSCCGNGWLAAQLHQGRVDVDATQDLICRFGSQE | |
| | | ELSLSLVRPEDCEKGGLKNVN | |

In some embodiments, antigenic protein complexes (e.g., a LTBP-TGFβ1 complex) may comprise one or more LTBP proteins (e.g., LTBP1, LTBP2, LTBP3, and LTBP4) or fragment(s) thereof. In some embodiments, an antibody, or antigen binding portion thereof, as described herein, is capable of binding to a LTBP1-TGFβ1 complex. In some embodiments, the LTBP1 protein is a naturally-occurring protein or fragment thereof. In some embodiments, the LTBP1 protein is a non-naturally occurring protein or fragment thereof. In some embodiments, the LTBP1 protein is a recombinant protein. Such recombinant LTBP1 protein may comprise LTBP1, alternatively spliced variants thereof and/or fragments thereof. Recombinant LTBP1 proteins may also be modified to comprise one or more detectable labels. In some embodiments, the LTBP1 protein comprises a leader sequence (e.g., a native or non-native leader sequence). In some embodiments, the LTBP1 protein does not comprise a leader sequence (i.e., the leader sequence has been processed or cleaved). Such detectable labels may include, but are not limited to biotin labels, polyhistidine tags, myc tags, HA tags and/or fluorescent tags. In some embodiments, the LTBP1 protein is a mammalian LTBP1 protein. In some embodiments, the LTBP1 protein is a human, a monkey, a mouse, or a rat LTBP1 protein. In some embodiments, the LTBP1 protein comprises an amino acid sequence as set forth in SEQ ID NOs: 46 and 47 in Table 2. In some embodiments, the LTBP1 protein comprises an amino acid sequence as set forth in SEQ ID NO: 50 in Table 3.

In some embodiments, an antibody, or antigen binding portion thereof, as described herein, is capable of binding to a LTBP3-TGFβ1 complex. In some embodiments, the LTBP3 protein is a naturally-occurring protein or fragment thereof. In some embodiments, the LTBP3 protein is a non-naturally occurring protein or fragment thereof. In some embodiments, the LTBP3 protein is a recombinant protein. Such recombinant LTBP3 protein may comprise LTBP3, alternatively spliced variants thereof and/or fragments thereof. In some embodiments, the LTBP3 protein comprises a leader sequence (e.g., a native or non-native leader sequence). In some embodiments, the LTBP3 protein does not comprise a leader sequence (i.e., the leader sequence has been processed or cleaved). Recombinant LTBP3 proteins may also be modified to comprise one or more detectable labels. Such detectable labels may include, but are not limited to biotin labels, polyhistidine tags, myc tags, HA tags and/or fluorescent tags. In some embodiments, the LTBP3 protein is a mammalian LTBP3 protein. In some embodiments, the LTBP3 protein is a human, a monkey, a mouse, or a rat LTBP3 protein. In some embodiments, the LTBP3 protein comprises an amino acid sequence as set forth in SEQ ID NOs: 44 and 45 in Table 2. In some embodiments, the LTBP1 protein comprises an amino acid sequence as set forth in SEQ ID NO: 51 in Table 3.

In some embodiments, an antibody, or antigen binding portion thereof, as described herein, is capable of binding to a GARP-TGFβ1 complex. In some embodiments, the GARP protein is a naturally-occurring protein or fragment thereof. In some embodiments, the GARP protein is a non-naturally occurring protein or fragment thereof. In some embodiments, the GARP protein is a recombinant protein. Such a GARP may be recombinant, referred to herein as recombinant GARP. Some recombinant GARPs may comprise one or more modifications, truncations and/or mutations as compared to wild type GARP. Recombinant GARPs may be modified to be soluble. In some embodiments, the GARP protein comprises a leader sequence (e.g., a native or non-native leader sequence). In some embodiments, the GARP protein does not comprise a leader sequence (i.e., the leader sequence has been processed or cleaved). In other embodiments, recombinant GARPs are modified to comprise one or more detectable labels. In further embodiments, such detectable labels may include, but are not limited to biotin labels, polyhistidine tags, flag tags, myc tags, HA tags and/or fluorescent tags. In some embodiments, the GARP protein is a mammalian GARP protein. In some embodiments, the GARP protein is a human, a monkey, a mouse, or a rat GARP protein. In some embodiments, the GARP protein comprises an amino acid sequence as set forth in SEQ ID NOs: 48-49 in Table 2. In some embodiments, the GARP protein comprises an amino acid sequence as set forth in SEQ ID NOs: 52 and 53 in Table 4. In some embodiments, the antibodies, or antigen binding portions thereof, described herein do not bind to TGFβ1 in a context-dependent manner, for example binding to TGFβ1 would only occur when the TGFβ1 molecule was complexed with a specific presenting molecule, such as GARP. Instead, the antibodies, and antigen-binding portions thereof, bind to TGFβ1 in a context-independent manner. In other words, the antibodies, or antigen-binding portions thereof, bind to TGFβ1 when bound to any presenting molecule: GARP, LTBP1, LTBP3, and/or LRCC33.

In some embodiments, an antibody, or antigen binding portion thereof, as described herein, is capable of binding to a LRRC33-TGFβ1 complex. In some embodiments, the LRRC33 protein is a naturally-occurring protein or fragment thereof. In some embodiments, the LRRC33 protein is a non-naturally occurring protein or fragment thereof. In some embodiments, the LRRC33 protein is a recombinant protein. Such a LRRC33 may be recombinant, referred to herein as recombinant LRRC33. Some recombinant LRRC33 proteins may comprise one or more modifications, truncations and/or mutations as compared to wild type LRRC33. Recombinant LRRC33 proteins may be modified to be soluble. For example, in some embodiments, the ectodomain of LRRC33 may be expressed with a C-terminal His-tag in order to express soluble LRRC33 protein (sLRRC33; see, e.g., SEQ ID NO: 84). In some embodiments, the LRRC33 protein comprises a leader sequence (e.g., a native or non-native leader sequence). In some embodiments, the LRRC33 protein does not comprise a leader sequence (i.e., the leader sequence has been processed or cleaved). In other embodiments, recombinant LRRC33 proteins are modified to comprise one or more detectable labels. In further embodiments, such detectable labels may include, but are not limited to biotin labels, polyhistidine tags, flag tags, myc tags, HA tags and/or fluorescent tags. In some embodiments, the LRRC33 protein is a mammalian LRRC33 protein. In some embodiments, the LRRC33 protein is a human, a monkey, a mouse, or a rat LRRC33 protein. In some embodiments, the LRRC33 protein comprises an amino acid sequence as set forth in SEQ ID NOs: 83, 84, and 101 in Table 4.

TABLE 3
Exemplary LTBP amino acid sequences

| | | SEQ |
|---|---|---|
| | | ID |
| Protein | Sequence | NO |
| LTBP1S | NHTGRIKVVFTPSICKVTCTKGSCQNSCEKGNTTTLISENGHA | 50 |
| | ADTLTATNFRVVICHLPCMNGGQCSSRDKCQCPPNFTGKLCQ | |
| | IPVHGASVPKLYQHSQQPGKALGTHVIHSTHTLPLTVTSQQGV | |
| | KVKFPPNIVNIHVKHPPEASVQIHQVSRIDGPTGQKTKEAQPG | |
| | QSQVSYQGLPVQKTQTIHSTYSHQQVIPHVYPVAAKTQLGRC | |
| | FQETIGSQCGKALPGLSKQEDCCGTVGTSWGFNKCQKCPKK | |
| | PSYHGYNQMMECLPGYKRVNNTFCQDINECQLQGVCPNGEC | |
| | LNTMGSYRCTCKIGFGPDPTFSSCVPDPPVISEEKGPCYRLVS | |
| | SGRQCMHPLSVHLTKQLCCCSVGKAWGPHCEKCPLPGTAAF | |
| | KEICPGGMGYTVSGVHRRRPIHHHVGKGPVFVKPKNTQPVAK | |
| | STHPPPLPAKEEPVEALTFSREHGPGVAEPEVATAPPEKEIPS | |
| | LDQEKTKLEPGQPQLSPGISTIHLHPQFPVVIEKTSPPVPVEVA | |
| | PEASTSSASQVIAPTQVTEINECTVNPDICGAGHCINLPVRYTC | |
| | ICYEGYRFSEQQRKCVDIDECTQVQHLCSQGRCENTEGSFLC | |
| | ICPAGFMASEEGTNCIDVDECLRPDVCGEGHCVNTVGAFRCE | |
| | YCDSGYRMTQRGRCEDIDECLNPSTCPDEQCVNSPGSYQCV | |
| | PCTEGFRGWNGQCLDVDECLEPNVCANGDCSNLEGSYMCS | |
| | CHKGYTRTPDHKHCRDIDECQQGNLCVNGQCKNTEGSFRCT | |
| | CGQGYQLSAAKDQCEDIDECQHRHLCAHGQCRNTEGSFQCV | |
| | CDQGYRASGLGDHCEDINECLEDKSVCQRGDCINTAGSYDCT | |
| | CPDGFQLDDNKTCQDINECEHPGLCGPQGECLNTEGSFHCV | |
| | CQQGFSISADGRTCEDIDECVNNTVCDSHGFCDNTAGSFRCL | |
| | CYQGFQAPQDGQGCVDVNECELLSGVCGEAFCENVEGSFLC | |
| | VCADENQEYSPMTGQCRSRTSTDLDVDVDQPKEEKKECYYN | |
| | LNDASLCDNVLAPNVTKQECCCTSGVGWGDNCEIFPCPVLGT | |
| | AEFTEMCPKGKGFVPAGESSSEAGGENYKDADECLLFGQEIC | |
| | KNGFCLNTRPGYECYCKQGTYYDPVKLQCFDMDECQDPSSC | |
| | IDGQCVNTEGSYNCFCTHPMVLDASEKRCIRPAESNEQIEETD | |
| | VYQDLCWEHLSDEYVCSRPLVGKQTTYTECCCLYGEAWGMQ | |
| | CALCPLKDSDDYAQLCNIPVTGRRQPYGRDALVDFSEQYTPE | |
| | ADPYFIQDRFLNSFEELQAEECGILNGCENGRCVRVQEGYTC | |
| | DCFDGYHLDTAKMTCVDVNECDELNNRMSLCKNAKCINTDGS | |
| | YKCLCLPGYVPSDKPNYCTPLNTALNLEKDSDLE | |
| LTBP3 | GPAGERGAGGGGALARERFKVVFAPVICKRTCLKGQCRDSC | 51 |
| | QQGSNMTLIGENGHSTDTLTGSGFRVVVCPLPCMNGGQCSS | |
| | RNQCLCPPDFTGRFCQVPAGGAGGGTGGSGPGLSRTGALST | |
| | GALPPLAPEGDSVASKHAIYAVQVIADPPGPGEGPPAQHAAFL | |
| | VPLGPGQISAEVQAPPPVVNVRVHHPPEASVQVHRIESSNAE | |
| | SAAPSQHLLPHPKPSHPRPPTQKPLGRCFQDTLPKQPCGSNP | |
| | LPGLTKQEDCCGSIGTAWGQSKCHKCPQLQYTGVQKPGPVR | |
| | GEVGADCPQGYKRLNSTHCQDINECAMPGVCRHGDCLNNPG | |
| | SYRCVCPPGHSLGPSRTQCIADKPEEKSLCFRLVSPEHQCQH | |
| | PLTTRLTRQLCCCSVGKAWGARCQRCPTDGTAAFKEICPAGK | |
| | GYHILTSHQTLTIQGESDFSLFLHPDGPPKPQQLPESPSQAPP | |
| | PEDTEEERGVTTDSPVSEERSVQQSHPTATTTPARPYPELISR | |
| | PSPPTMRWFLPDLPPSRSAVEIAPTQVTETDECRLNQNICGH | |
| | GECVPGPPDYSCHCNPGYRSHPQHRYCVDVNECEAEPCGP | |
| | GRGICMNTGGSYNCHCNRGYRLHVGAGGRSCVDLNECAKP | |
| | HLCGDGGFCINFPGHYKCNCYPGYRLKASRPPVCEDIDECRD | |
| | PSSCPDGKCENKPGSFKCIACQPGYRSQGGGACRDVNECAE | |
| | GSPCSPGWCENLPGSFRCTCAQGYAPAPDGRSCLDVDECEA | |
| | GDVCDNGICSNTPGSFQCQCLSGYHLSRDRSHCEDIDECDFP | |
| | AACIGGDCINTNGSYRCLCPQGHRLVGGRKCQDIDECSQDPS | |
| | LCLPHGACKNLQGSYVCVCDEGFTPTQDQHGCEEVEQPHHK | |
| | KECYLNFDDTVFCDSVLATNVTQQECCCSLGAGWGDHCEIYP | |
| | CPVYSSAEFHSLCPDGKGYTQDNNIVNYGIPAHRDIDECMLFG | |
| | SEICKEGKCVNTQPGYECYCKQGFYYDGNLLECVDVDECLDE | |
| | SNCRNGVCENTRGGYRCACTPPAEYSPAQRQCLSPEEMDVD | |
| | ECQDPAACRPGRCVNLPGSYRCECRPPWVPGPSGRDCQLP | |
| | ESPAERAPERRDVCWSQRGEDGMCAGPLAGPALTFDDCCC | |
| | RQGRGWGAQCRPCPPRGAGSHCPTSQSESNSFWDTSPLLL | |
| | GKPPRDEDSSEEDSDECRCVSGRCVPRPGGAVCECPGGFQ | |
| | LDASRARCVDIDECRELNQRGLLCKSERCVNTSGSFRCVCKA | |
| | GFARSRPHGACVPQRRR | |

TABLE 4
Exemplary GARP and LRRC33 amino acid sequences

| | | SEQ |
|---|---|---|
| | | ID |
| Protein | Sequence | NO |
| GARP | AQHQDKVPCKMVDKKVSCQVLGLLQVPSVLPPDTETLDLSGNQ | 52 |
| | LRSILASPLGFYTALRHLDLSTNEISFLQPGAFQALTHLEHLSLAH | |
| | NRLAMATALSAGGLGPLPRVTSLDLSGNSLYSGLLERLLGEAPS | |
| | LHTLSLAENSLTRLTRHTFRDMPALEQLDLHSNVLMDIEDGAFE | |
| | GLPRLTHLNLSRNSLTCISDFSLQQLRVLDLSCNSIEAFQTASQP | |
| | QAEFQLTWLDLRENKLLHFPDLAALPRLIYLNLSNNLIRLPTGPP | |
| | QDSKGIHAPSEGWSALPLSAPSGNASGRPLSQLLNLDLSYNEIE | |
| | LIPDSFLEHLTSLCFLNLSRNCLRTFEARRLGSLPCLMLLDLSHN | |
| | ALETLELGARALGSLRTLLLQGNALRDLPPYTFANLASLQRLNLQ | |
| | GNRVSPCGGPDEPGPSGCVAFSGITSLRSLSLVDNEIELLRAGA | |
| | FLHTPLTELDLSSNPGLEVATGALGGLEASLEVLALQGNGLMVL | |
| | QVDLPCFICLKRLNLAENRLSHLPAWTQAVSLEVLDLRNNSFSLL | |
| | PGSAMGGLETSLRRLYLQGNPLSCCGNGWLAAQLHQGRVDVD | |
| | ATQDLICRFSSQEEVSLSHVRPEDCEKGGLKNINLIIILTFILVSAIL | |
| | LTTLAACCCVRRQKFNQQYKA | |
| sGARP | AQHQDKVPCKMVDKKVSCQVLGLLQVPSVLPPDTETLDLSGNQ | 53 |
| | LRSILASPLGFYTALRHLDLSTNEISFLQPGAFQALTHLEHLSLAH | |
| | NRLAMATALSAGGLGPLPRVTSLDLSGNSLYSGLLERLLGEAPS | |
| | LHTLSLAENSLTRLTRHTFRDMPALEQLDLHSNVLMDIEDGAFE | |
| | GLPRLTHLNLSRNSLTCISDFSLQQLRVLDLSCNSIEAFQTASQP | |
| | QAEFQLTWLDLRENKLLHFPDLAALPRLIYLNLSNNLIRLPTGPP | |
| | QDSKGIHAPSEGWSALPLSAPSGNASGRPLSQLLNLDLSYNEIE | |
| | LIPDSFLEHLTSLCFLNLSRNCLRTFEARRLGSLPCLMLLDLSHN | |
| | ALETLELGARALGSLRTLLLQGNALRDLPPYTFANLASLQRLNLQ | |
| | GNRVSPCGGPDEPGPSGCVAFSGITSLRSLSLVDNEIELLRAGA | |
| | FLHTPLTELDLSSNPGLEVATGALGGLEASLEVLALQGNGLMVL | |
| | QVDLPCFICLKRLNLAENRLSHLPAWTQAVSLEVLDLRNNSFSLL | |
| | PGSAMGGLETSLRRLYLQGNPLSCCGNGWLAAQLHQGRVDVD | |
| | ATQDLICRFSSQEEVSLSHVRPEDCEKGGLKNIN | |
| LRRC33 (also known | MELLPLWLCLGFHFLTVGWRNRSGTATAASQGVCKLVGGAAD | 83 |
| as NRROS; Uniprot | CRGQSLASVPSSLPPHARMLTLDANPLKTLWNHSLQPYPLLESL | |
| Accession No. | SLHSCHLERISRGAFQEQGHLRSLVLGDNCLSENYEETAAALHA | |
| Q86YC3) | LPGLRRLDLSGNALTEDMAALMLQNLSSLRSVSLAGNTIMRLDD | |
| | SVFEGLERLRELDLQRNYIFEIEGGAFDGLAELRHLNLAFNNLPCI | |
| | VDFGLTRLRVLNVSYNVLEWFLATGGEAAFELETLDLSHNQLLF | |
| | FPLLPQYSKLRTLLLRDNNMGFYRDLYNTSSPREMVAQFLLVDG | |
| | NVTNITTVSLWEEFSSSDLADLRFLDMSQNQFQYLPDGFLRKMP | |
| | SLSHLNLHQNCLMTLHIREHEPPGALTELDLSHNQLSELHLAPGL | |
| | ASCLGSLRLFNLSSNQLLGVPPGLFANARNITTLDMSHNQISLCP | |
| | LPAASDRVGPPSCVDFRNMASLRSLSLEGCGLGALPDCPFQGT | |
| | SLTYLDLSSNWGVLNGSLAPLQDVAPMLQVLSLRNMGLHSSFM | |
| | ALDFSGFGNLRDLDLSGNCLTTFPRFGGSLALETLDLRRNSLTA | |
| | LPQKAVSEQLSRGLRTIYLSQNPYDCCGVDGWGALQHGQTVAD | |
| | WAMVTCNLSSKIIRVTELPGGVPRDCKWERLDLGLLYLVLILPSC | |
| | LTLLVACTVIVLTFKKPLLQVIKSRCHWSSVY | |
| | * Native signal peptide is depicted in bold font. | |
| soluble LRRC33 | MDMRVPAQLLGLLLLWFSGVLGWRNRSGTATAASQGVCKLVG | 84 |
| (sLRRC33) | GAADCRGQSLASVPSSLPPHARMLTLDANPLKTLWNHSLQPYP | |
| | LLESLSLHSCHLERISRGAFQEQGHLRSLVLGDNCLSENYEETA | |
| | AALHALPGLRRLDLSGNALTEDMAALMLQNLSSLRSVSLAGNTI | |
| | MRLDDSVFEGLERLRELDLQRNYIFEIEGGAFDGLAELRHLNLAF | |
| | NNLPCIVDFGLTRLRVLNVSYNVLEWFLATGGEAAFELETLDLSH | |
| | NQLLFFPLLPQYSKLRTLLLRDNNMGFYRDLYNTSSPREMVAQF | |
| | LLVDGNVTNITTVSLWEEFSSSDLADLRFLDMSQNQFQYLPDG | |
| | LRKMPSLSHLNLHQNCLMTLHIREHEPPGALTELDLSHNQLSEL | |
| | HLAPGLASCLGSLRLFNLSSNQLLGVPPGLFANARNITTLDMSH | |
| | NQISLCPLPAASDRVGPPSCVDFRNMASLRSLSLEGCGLGALPD | |
| | CPFQGTSLTYLDLSSNWGVLNGSLAPLQDVAPMLQVLSLRNMG | |
| | LHSSFMALDFSGFGNLRDLDLSGNCLTTFPRFGGSLALETLDLR | |
| | RNSLTALPQKAVSEQLSRGLRTIYLSQNPYDCCGVDGWGALQH | |
| | GQTVADWAMVTCNLSSKIIRVTELPGGVPRDCKWERLDLGLHH | |
| | HHHH | |
| | * Modified human kappa light chain signal peptide | |
| | is depicted in bold font. | |
| | ** Histidine tag is underlined. | |
| Human LRRC33- | MDMRVPAQLLGLLLLWFSGVLGWRNRSGTATAASQGVCKLVG | 101 |
| GARP chimera | GAADCRGQSLASVPSSLPPHARMLTLDANPLKTLWNHSLQPYP | |
| | LLESLSLHSCHLERISRGAFQEQGHLRSLVLGDNCLSENYEETA | |
| | AALHALPGLRRLDLSGNALTEDMAALMLQNLSSLRSVSLAGNTI | |
| | MRLDDSVFEGLERLRELDLQRNYIFEIEGGAFDGLAELRHLNLAF | |
| | NNLPCIVDFGLTRLRVLNVSYNVLEWFLATGGEAAFELETLDLSH | |
| | NQLLFFPLLPQYSKLRTLLLRDNNMGFYRDLYNTSSPREMVAQF | |
| | LLVDGNVTNITTVSLWEEFSSSDLADLRFLDMSQNQFQYLPDGF | |
| | LRKMPSLSHLNLHQNCLMTLHIREHEPPGALTELDLSHNQLSEL | |
| | HLAPGLASCLGSLRLFNLSSNQLLGVPPGLFANARNITTLDMSH | |
| | NQISLCPLPAASDRVGPPSCVDFRNMASLRSLSLEGCGLGALPD | |
| | CPFQGTSLTYLDLSSNWGVLNGSLAPLQDVAPMLQVLSLRNMG | |
| | LHSSFMALDFSGFGNLRDLDLSGNCLTTFPRFGGSLALETLDLR | |
| | RNSLTALPQKAVSEQLSRGLRTIYLSQNPYDCCGVDGWGALQH | |
| | GQTVADWAMVTCNLSSKIIRVTELPGGVPRDCKWERLDLGLLIII | |
| | LTFILVSAILLTTLAACCCVRRQKFNQQYKA | |
| | * Modified human kappa light chain signal peptide | |
| | is depicted in bold font. | |
| | ** LRRC33 ectodomain is underlined. | |
| | # GARP transmembrane domain is italicized. | |
| | ## GARP intracellular tail is double underlined. | |

### TGFβ1 Antagonists

To carry out the methods of the present invention, any suitable inhibitory agents of TGFβ1 may be employed, provided that the such agents inhibit or antagonize TGFβ1 across multiple biological effects (e.g., TGFβ1 from multiple cellular sources) with sufficient selectivity for the TGFβ1 isoform. Preferably, such inhibitory agents of TGFβ1 have no measurable inhibitory activities towards TGFβ2 and TGFβ3 at dosage that provides clinical benefits (e.g., therapeutic efficacy and acceptable toxicity profiles) when administered to human subjects. Suitable inhibitory agents include small molecules, nucleic acid-based agents, biologics (e.g., polypeptide-based agents such as antibodies and other finding-agents), and any combinations thereof. In some embodiments, such agents are antibodies or fragments thereof, as further described below. These include neutralizing antibodies that bind TGFβ1 growth factor thereby neutralizing its action.

Functional Antibodies that Selectively Inhibit TGFβ1

The present invention in one aspect encompasses the use of functional antibodies. As used herein, “a functional antibody” confers one or more biological activities by virtue of its ability to bind an antigen. Functional antibodies therefore include those capable of modulating the activity/function of target molecules (i.e., antigen). Such modulating antibodies include inhibiting antibodies (or inhibitory antibodies) and activating antibodies. The present disclosure includes TGFβ antibodies which can inhibit a biological process mediated by TGFβ1 signaling associated with multiple contexts of TGFβ1. Inhibitory agents used to carry out the present invention, such as the antibodies described herein, are intended to be TGFβ1-selective and not to target or interfere with TGFβ2 and TGFβ3 when administered at a therapeutically effective dose (dose at which sufficient efficacy is achieved within acceptable toxicity levels).

Building upon the earlier recognition by the applicant of the present disclosure (see PCT/US2017/021972) that lack of isoform-specificity of conventional TGFβ antagonists may underlie the source of toxicities associated with TGFβ inhibition, the present inventors sought to further achieve broad-spectrum TGFβ1 inhibition for treating various diseases that manifest multifaceted TGFβ1 dysregulation, while maintaining the safety/tolerability aspect of isoform-selective inhibitors.

In a broad sense, the term “inhibiting antibody” refers to an antibody that antagonizes or neutralizes the target function, e.g., growth factor activity. Advantageously, preferred inhibitory antibodies of the present disclosure are capable of inhibiting mature growth factor release from a latent complex, thereby reducing growth factor signaling. Inhibiting antibodies include antibodies targeting any epitope that reduces growth factor release or activity when associated with such antibodies. Such epitopes may lie on prodomains of TGFβ proteins (e.g. TGFβ1), growth factors or other epitopes that lead to reduced growth factor activity when bound by antibody. Inhibiting antibodies of the present invention include, but are not limited to, TGFβ1-inhibiting antibodies. In some embodiments, inhibitory antibodies of the present disclosure specifically bind a combinatory epitope, i.e., an epitope formed by two or more components/portions of an antigen or antigen complex. For example, a combinatorial epitope may be formed by contributions from multiple portions of a single protein, i.e., amino acid residues from more than one non-contiguous segments of the same protein. Alternatively, a combinatorial epitope may be formed by contributions from multiple protein components of an antigen complex. In some embodiments, inhibitory antibodies of the present disclosure specifically bind a conformational epitope (or conformation-specific epitope), e.g., an epitope that is sensitive to the three-dimensional structure (i.e., conformation) of an antigen or antigen complex.

Traditional approaches to antagonizing TGFβ signaling have been to i) directly neutralize the mature growth factor after it has already become active so as to deplete free ligands (e.g., released from its latent precursor complex) that are available for receptor binding; ii) employ soluble receptor fragments capable of sequestering free ligands (e.g., so-called ligand traps); or, iii) target its cell-surface receptor(s) to block ligand-receptor interactions. Each of these conventional approaches requires the antagonist to compete against endogenous counterparts. Moreover, the first two approaches (i and ii) above target the active ligand, which is a transient species. Therefore, such antagonist must be capable of kinetically outcompeting the endogenous receptor during the brief temporal window. The third approach may provide a more durable effect in comparison but inadvertently results in unwanted inhibitory effects (hence possible toxicities) because many growth factors (e.g., up to ˜20) signal via the same receptor(s).

To provide solutions to these drawbacks, and to further enable greater selectivity and localized action, the preferred mechanism of action underlining the inhibitory antibodies such as those described herein acts upstream of TGFβ1 activation and ligand-receptor interaction. Thus, it is contemplated that isoform-specific, context-permissive inhibitors of TGFβ1 suitable for carrying out the present invention should preferably target the inactive (e.g., latent) precursor TGFβ1 complex (e.g., a complex comprising pro/latent TGFβ1) prior to its activation, in order to block the activation step at its source (such as in a disease microenvironment). According to preferred embodiments of the invention, such inhibitors target ECM-associated and/or cell surface-tethered pro/latent TGFβ1 complexes, rather than free ligands that are transiently available for receptor binding.

Accordingly, some embodiments of the present invention employ agents that specifically bind to an TGFβ1-containing complexes, thereby inhibiting the function of TGFβ1 in an isoform-selective manner. Such agents are preferably antibodies that bind an epitope within a protein complex comprising pro/latent TGFβ1 (e.g., inactive TGFβ1 precursor). In some embodiments, the epitope is available for binding by the antibody when the TGFβ1 is present in two or more of the following: a GARP-TGFβ1 complex, a LTBP1-TGFβ1 complex, a LTBP3-TGFβ1 complex, and a LRRC33-TGFβ1 complex. In some embodiments, such antibodies bind two or more of the TGFβ1-containing complexes provided above (e.g., “context-permissive”), while in other embodiments, such antibodies bind all four of the TGFβ1-containing complexes provided above (e.g., “context-independent”). In some embodiments, any of such antibodies may show differential species selectivity. The epitope may be within the pro-domain of the TGFβ1 complex. The epitope may be a combinatory epitope, such that the epitope is formed by two or more portions/segments (e.g., amino acid residues) of one or more component(s) of the complex. The epitope may be a conformational epitope, such that the epitope is sensitive to a particular three-dimensional structure of an antigen (e.g., the TGFβ1 complex). An antibody or a fragment thereof that specifically binds to a conformational epitope is referred as a conformational antibody or conformation-specific antibody.

Embodiments of the present disclosure include methods of using inhibiting antibodies in solution, in cell culture and/or in subjects to modify growth factor signaling, including for purposes of conferring clinical benefits to patients.

Exemplary antibodies and corresponding nucleic acid sequences that encode the antibodies useful for carrying out the present invention include one or more of the CDR amino acid sequences shown in Table 5.

TABLE 5
Complementary determining regions of the heavy chain (CDRHs) and the light chain
(CDRLs) as determined using the Kabat numbering scheme are shown for antibodies
Ab1, Ab2 and Ab3

| Antibody | Ab1 | Ab2 | Ab3 |
|---|---|---|---|
| CDRH1 | SYGMH | SDWIG | NYAMS |
| | (SEQ ID NO: 1) | (SEQ ID NO: 2) | (SEQ ID NO: 85) |
| CDRH2 | VISYDGSNKYYADSVKG | VIYPGDSDTRYSASFQG | SISGSGGATYYADSVKG |
| | (SEQ ID NO: 3) | (SEQ ID NO: 4) | (SEQ ID NO: 86) |
| CDRH3 | DIRPYGDYSAAFDI | AAGIAAAGHVTAFDI | ARVSSGHWDFDY |
| | (SEQ ID NO: 5) | (SEQ ID NO: 6) | (SEQ ID NO: 87) |
| CDRL1 | TGSSGSIASNYVQ | KSSQSVLYSSNNKNYLA | RASQSISSYLN |
| | (SEQ ID NO: 7) | (SEQ ID NO: 8) | (SEQ ID NO: 88) |
| CDRL2 | EDNQRPS | WASTRES | SSLQS |
| | (SEQ ID NO: 9) | (SEQ ID NO: 10) | (SEQ ID NO: 89) |
| CDRL3 | QSYDSSNHGGV | QQYYSTPVT | QQSYSAPFT |
| | (SEQ ID NO: 11) | (SEQ ID NO: 12) | (SEQ ID NO: 90) |

In some embodiments, antibodies of the present invention that specifically bind to GARP-TGFβ1 complex, a LTBP1-TGFβ1 complex, a LTBP3-TGFβ1 complex, and/or a LRRC33-TGFβ1 complex include any antibody, or antigen binding portion thereof, comprising a CDRH1, CDRH2, CDRH3, CDRL1, CDRL2, or CDRL3, or combinations thereof, as provided for any one of the antibodies shown in Table 5. In some embodiments, antibodies that specifically bind to GARP-TGFβ1 complex, a LTBP1-TGFβ1 complex, a LTBP3-TGFβ1 complex, and/or a LRRC33-TGFβ1 complex include the CDRH1, CDRH2, CDRH3, CDRL1, CDRL2, and CDRL3 of any one of the antibodies shown in Table 5. The present invention also provides any nucleic acid sequence that encodes a molecule comprising a CDRH1, CDRH2, CDRH3, CDRL1, CDRL2, or CDRL3 as provided for any one of the antibodies shown in Table 5. Antibody heavy and light chain CDR3 domains may play a particularly important role in the binding specificity/affinity of an antibody for an antigen. Accordingly, the antibodies that specifically bind to GARP-TGFβ1 complex, a LTBP1-TGFβ1 complex, a LTBP3-TGFβ1 complex, and/or a LRRC33-TGFβ1 complex of the disclosure, or the nucleic acid molecules encoding these antibodies, or antigen binding portions thereof, may include at least the heavy and/or light chain CDR3s of the antibodies as shown in Table 5.

Aspects of the invention relate to a monoclonal antibody, or antigen binding portion thereof, that binds specifically to a GARP-TGFβ1 complex, a LTBP1-TGFβ1 complex, a LTBP3-TGFβ1 complex, and/or a LRRC33-TGFβ1 complex, and that comprises six complementarity determining regions (CDRs): CDRH1, CDRH2, CDRH3, CDRL1, CDRL2, and CDRL3.

In some embodiments, CDRH1 comprises a sequence as set forth in any one of SEQ ID NOs: 1, 2 and 85. In some embodiments, CDRH2 comprises a sequence as set forth in any one of SEQ ID NOs: 3, 4 and 86. In some embodiments, CDRH3 comprises a sequence as set forth in any one of SEQ ID NOs: 5, 6 and 87. CDRL1 comprises a sequence as set forth in any one of SEQ ID NOs: 7, 8 and 88. In some embodiments, CDRL2 comprises a sequence as set forth in any one of SEQ ID NOs: 9, 10 and 89. In some embodiments, CDRL3 comprises a sequence as set forth in any one of SEQ ID NOs: 11, 12 and 90.

In some embodiments (e.g., as for antibody Ab1, shown in Table 5), the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises: a CDRH1 comprising an amino acid sequence as set forth in SEQ ID NO: 1, a CDRH2 comprising an amino acid sequence as set forth in SEQ ID NO: 3, a CDRH3 comprising an amino acid sequence as set forth in SEQ ID NO: 5, a CDRL1 comprising an amino acid sequence as set forth in SEQ ID NO: 7, a CDRL2 comprising an amino acid sequence as set forth in SEQ ID NO: 9, and a CDRL3 comprising an amino acid sequence as set forth in SEQ ID NO: 11.

In some embodiments, the antibody, or antigen binding portion thereof, comprises a heavy chain variable region comprising a complementarity determining region 3 (CDR3) having the amino acid sequence of SEQ ID NO: 5 and a light chain variable region comprising a CDR3 having the amino acid sequence of SEQ ID NO: 11. In some embodiments, the antibody, or antigen binding portion thereof, comprises a heavy chain variable region comprising a complementarity determining region 2 (CDR2) having the amino acid sequence of SEQ ID NO: 3 and a light chain variable region comprising a CDR2 having the amino acid sequence of SEQ ID NO: 9. In some embodiments, the antibody, or antigen binding portion thereof, comprises a heavy chain variable region comprising a complementarity determining region 1 (CDR1) having the amino acid sequence of SEQ ID NO: 1 and a light chain variable region comprising a CDR1 having the amino acid sequence of SEQ ID NO: 7.

In some embodiments, the antibody, or antigen binding portion thereof, comprises a heavy chain variable domain comprising an amino acid sequence having at least 70%, 75%, 80%, 85%, 90%, 95%, 96%, 97%, 98%, or 99% identity to the amino acid sequence set forth in SEQ ID NO: 13 and a light chain variable domain comprising an amino acid sequence having at least 70%, 75%, 80%, 85%, 90%, 95%, 96%, 97%, 98%, or 99% identity to the amino acid sequence set forth in SEQ ID NO: 14. In some embodiments, the antibody, or antigen binding portion thereof, comprises a heavy chain variable domain comprising an amino acid sequence set forth in SEQ ID NO: 13 and a light chain variable domain comprising an amino acid sequence set forth in SEQ ID NO: 14.

In some embodiments, the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a heavy chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 91, and a light chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 92. In some embodiments, the antibody or antigen binding portion thereof, comprises a heavy chain variable domain amino acid sequence encoded by the nucleic acid sequence set forth in SEQ ID NO: 91, and a light chain variable domain amino acid sequence encoded by the nucleic acid sequence set forth in SEQ ID NO: 92.

In some embodiments (e.g., as for antibody Ab2, shown in Table 5), the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a CDRH1 comprising an amino acid sequence as set forth in SEQ ID NO: 2, a CDRH2 comprising an amino acid sequence as set forth in SEQ ID NO: 3, a CDRH3 comprising an amino acid sequence as set forth in SEQ ID NO: 6, a CDRL1 comprising an amino acid sequence as set forth in SEQ ID NO: 8, a CDRL2 comprising an amino acid sequence as set forth in SEQ ID NO: 10, and a CDRL3 comprising an amino acid sequence as set forth in SEQ ID NO: 12.

In some embodiments, the antibody, or antigen binding portion thereof, comprises a heavy chain variable region comprising a CDR3 having the amino acid sequence of SEQ ID NO: 6 and a light chain variable region comprising a CDR3 having the amino acid sequence of SEQ ID NO: 12. In some embodiments, the antibody, or antigen binding portion thereof, comprises a heavy chain variable region comprising a CDR2 having the amino acid sequence of SEQ ID NO: 4 and a light chain variable region comprising a CDR2 having the amino acid sequence of SEQ ID NO: 10. In some embodiments, the antibody, or antigen binding portion thereof, comprises a heavy chain variable region comprising a CDR1 having the amino acid sequence of SEQ ID NO: 2 and a light chain variable region comprising a CDR1 having the amino acid sequence of SEQ ID NO: 8.

In some embodiments, the antibody, or antigen binding portion thereof, comprises a heavy chain variable domain comprising an amino acid sequence having at least 70%, 75%, 80%, 85%, 90%, 95%, 96%, 97%, 98%, or 99% identity to the amino acid sequence set forth in SEQ ID NO: 15 and a light chain variable domain comprising an amino acid sequence having at least 70%, 75%, 80%, 85%, 90%, 95%, 96%, 97%, 98%, or 99% identity to the amino acid sequence set forth in SEQ ID NO: 16. In some embodiments, the antibody, or antigen binding portion thereof, comprises a heavy chain variable domain comprising an amino acid sequence set forth in SEQ ID NO: 15 and a light chain variable domain comprising an amino acid sequence set forth in SEQ ID NO: 16.

In some embodiments, the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a heavy chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 93, and a light chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 94. In some embodiments, the antibody or antigen binding portion thereof, comprises a heavy chain variable domain amino acid sequence encoded by the nucleic acid sequence set forth in SEQ ID NO: 93, and a light chain variable domain amino acid sequence encoded by the nucleic acid sequence set forth in SEQ ID NO: 94.

In some embodiments (e.g., as for antibody Ab3, shown in Table 5), the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a CDRH1 comprising an amino acid sequence as set forth in SEQ ID NO: 85, a CDRH2 comprising an amino acid sequence as set forth in SEQ ID NO: 86, a CDRH3 comprising an amino acid sequence as set forth in SEQ ID NO: 87, a CDRL1 comprising an amino acid sequence as set forth in SEQ ID NO: 88, a CDRL2 comprising an amino acid sequence as set forth in SEQ ID NO: 89, and a CDRL3 comprising an amino acid sequence as set forth in SEQ ID NO: 90.

In some embodiments, the antibody, or antigen binding portion thereof, comprises a heavy chain variable region comprising a CDR3 having the amino acid sequence of SEQ ID NO: 87 and a light chain variable region comprising a CDR3 having the amino acid sequence of SEQ ID NO: 90. In some embodiments, the antibody, or antigen binding portion thereof, comprises a heavy chain variable region comprising a CDR2 having the amino acid sequence of SEQ ID NO: 86 and a light chain variable region comprising a CDR2 having the amino acid sequence of SEQ ID NO: 89. In some embodiments, the antibody, or antigen binding portion thereof, comprises a heavy chain variable region comprising a CDR1 having the amino acid sequence of SEQ ID NO: 85 and a light chain variable region comprising a CDR1 having the amino acid sequence of SEQ ID NO: 88.

In some embodiments, the antibody, or antigen binding portion thereof, comprises a heavy chain variable domain comprising an amino acid sequence having at least 70%, 75%, 80%, 85%, 90%, 95%, 96%, 97%, 98%, or 99% identity to the amino acid sequence set forth in SEQ ID NO: 95 and a light chain variable domain comprising an amino acid sequence having at least 70%, 75%, 80%, 85%, 90%, 95%, 96%, 97%, 98%, or 99% identity to the amino acid sequence set forth in SEQ ID NO: 97. In some embodiments, the antibody, or antigen binding portion thereof, comprises a heavy chain variable domain comprising an amino acid sequence set forth in SEQ ID NO: 95 and a light chain variable domain comprising an amino acid sequence set forth in SEQ ID NO: 97.

In some embodiments, the antibody or antigen binding portion thereof, that specifically binds to a GARP-TGFβ1 complex, a LTBP1-TGFβ1 complex, a LTBP3-TGFβ1 complex, and/or a LRRC33-TGFβ1 complex comprises a heavy chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 96, and a light chain variable domain amino acid sequence encoded by a nucleic acid sequence having at least 70%, 75%, 80%, 85%, 90%, 95%, 96%, 97%, 98%, or 99% identity to the nucleic acid sequence set forth in SEQ ID NO: 98. In some embodiments, the antibody or antigen binding portion thereof, comprises a heavy chain variable domain amino acid sequence encoded by the nucleic acid sequence set forth in SEQ ID NO: 96, and a light chain variable domain amino acid sequence encoded by the nucleic acid sequence set forth in SEQ ID NO: 98.

In some examples, any of the antibodies of the disclosure that specifically bind to a GARP-TGFβ1 complex, a LTBP1-TGFβ1 complex, a LTBP3-TGFβ1 complex, and/or a LRRC33-TGFβ1 complex include any antibody (including antigen binding portions thereof) having one or more CDR (e.g., CDRH or CDRL) sequences substantially similar to CDRH1, CDRH2, CDRH3, CDRL1, CDRL2, and/or CDRL3. For example, the antibodies may include one or more CDR sequences as shown in Table 5 (SEQ ID NOs: 1-12 and 85-90) containing up to 5, 4, 3, 2, or 1 amino acid residue variations as compared to the corresponding CDR region in any one of SEQ ID NOs: 1-12 and 85-90. The complete amino acid sequences for the heavy chain variable region and light chain variable region of the antibodies listed in Table 5 (e.g., Ab1, Ab2 and Ab3), as well as nucleic acid sequences encoding the heavy chain variable region and light chain variable region of the antibodies are provided below:

Ab1 - Heavy chain variable region amino acid
sequence
(SEQ ID NO: 13)
EVQLVESGGGLVQPGRSLRLSCAASGFTFSSYGMHWVRQAPGKGLEWVAV
ISYDGSNKYYADSVKGRFTISRDNSKNTLYLQMNSLRAEDTAVYYCARDI
RPYGDYSAAFDIWGQGTLVTVSS
Ab1 - Heavy chain variable region nucleic acid
sequence
(SEQ ID NO: 91)
GAGGTGCAACTCGTGGAGTCAGGCGGTGGACTTGTTCAGCCTGGGCGAAG
TCTGAGACTCTCATGTGCAGCAAGTGGATTCACTTTCTCCAGTTACGGCA
TGCACTGGGTGAGACAGGCGCCTGGAAAGGGTTTGGAATGGGTCGCTGTG
ATCTCTTACGACGGGTCAAACAAATATTACGCGGATTCAGTGAAAGGGCG
GTTCACTATTTCACGGGATAACTCCAAGAACACCCTGTATCTGCAGATGA
ATAGCCTGAGGGCAGAGGACACCGCTGTGTACTATTGTGCCCGGGACATA
AGGCCTTACGGCGATTACAGCGCCGCATTTGATATTTGGGGACAAGGCAC
CCTTGTGACAGTATCTTCT
Ab1 - Light chain variable region amino acid
sequence
(SEQ ID NO: 14)
NFMLTQPHSVSESPGKTVTISCTGSSGSIASNYVQWYQQRPGSAPSIVIF
EDNQRPSGAPDRFSGSIDSSSNSASLTISGLKTEDEADYYCQSYDSSNHG
GVFGGGTQLTVL
Ab1 - Light chain variable region nucleic acid
sequence
(SEQ ID NO: 92)
AATTTTATGCTTACCCAACCACATAGTGTGAGTGAGTCTCCCGGCAAGAC
TGTAACAATTTCATGTACCGGCAGCAGTGGCTCCATCGCTAGCAATTATG
TGCAATGGTACCAACAGCGCCCCGGGAGCGCACCTTCAATAGTGATATTC
GAGGATAACCAACGGCCTAGTGGGGCTCCCGATAGATTTAGTGGGAGTAT
AGATAGCTCCTCCAACTCTGCCTCTCTCACCATTAGCGGGCTGAAAACAG
AGGATGAAGCCGACTATTACTGCCAAAGCTATGATTCTAGCAACCACGGC
GGAGTGTTTGGCGGAGGAACACAGCTGACAGTCCTAGG
Ab1 - Heavy chain amino acid sequence
(SEQ ID NO: 15)
EVQLVESGGGLVQPGRSLRLSCAASGFTFSSYGMHWVRQAPGKGLEWVAV
ISYDGSNKYYADSVKGRFTISRDNSKNTLYLQMNSLRAEDTAVYYCARDI
RPYGDYSAAFDIWGQGTLVTVSSASTKGPSVFPLAPCSRSTSESTAALGC
LVKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLG
Ab1 - Light chain amino acid sequence
(SEQ ID NO: 16)
NFMLTQPHSVSESPGKTVTISCTGSSGSIASNYVQWYQQRPGSAPSIVIF
EDNQRPSGAPDRFSGSIDSSSNSASLTISGLKTEDEADYYCQSYDSSNHG
GVFGGGTQLTVLGQPKAAPSVTLFPPSSEELQANKATLVCLISDFYPGAV
TVAWKADSSPVKAGVETTTPSKQSNNKYAASSYLSLTPEQWKSHRSYSCQ
VTHEGSTVEKTVAPTECS
Ab2 - Heavy chain variable region amino acid
sequence
(SEQ ID NO: 17)
EVQLVQSGAEMKKPGESLKISCKGSGYNFASDWIGWVRQTPGKGLEWMGV
IYPGDSDTRYSASFQGQVTISADKSINTAYLQWSSLKASDTAMYYCASAA
GIAAAGHVTAFDIWGQGTMVTVSS
Ab2 - Heavy chain variable region nucleic acid
sequence
(SEQ ID NO: 93)
GAGGTGCAACTGGTGCAATCCGGAGCCGAGATGAAAAAGCCAGGGGAGAG
CCTGAAGATCTCTTGTAAGGGCTCTGGCTATAACTTCGCTAGTGATTGGA
TCGGATGGGTGAGGCAAACCCCCGGAAAGGGCCTCGAGTGGATGGGCGTG
ATCTACCCCGGCGACTCCGACACACGCTATAGCGCCTCATTCCAGGGCCA
GGTCACCATAAGTGCTGATAAATCAATAAATACAGCCTACTTGCAATGGT
CAAGTCTGAAAGCCTCAGATACTGCCATGTACTATTGTGCCTCTGCCGCC
GGCATTGCCGCGGCCGGTCACGTCACCGCCTTCGACATTTGGGGTCAGGG
CACTATGGTCACTGTAAGCTCC
Ab2 - Light chain variable region amino acid
sequence
(SEQ ID NO: 18)
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYST
PVTFGQGTKLEIK
Ab2 - Light chain variable region nucleic acid
sequence
(SEQ ID NO: 94)
GACATAGTCATGACCCAGTCACCTGACTCTTTGGCCGTGTCTCTGGGGGA
GAGAGCCACAATAAATTGCAAGTCATCACAGAGCGTCCTGTACTCCTCCA
ATAATAAAAATTACCTGGCCTGGTACCAGCAAAAGCCCGGGCAACCCCCC
AAATTGTTGATTTACTGGGCTAGTACAAGGGAATCTGGAGTGCCAGACCG
GTTTTCTGGTTCTGGATCTGGTACTGACTTCACCCTGACAATCAGCTCCC
TGCAGGCCGAAGACGTGGCTGTGTACTATTGTCAGCAGTACTATAGTACA
CCAGTTACTTTCGGCCAAGGCACTAAACTCGAAATCAAG
Ab2 - Heavy chain amino acid sequence
(SEQ ID NO: 19)
EVQLVQSGAEMKKPGESLKISCKGSGYNFASDWIGWVRQTPGKGLEWMGV
IYPGDSDTRYSASFQGQVTISADKSINTAYLQWSSLKASDTAMYYCASAA
GIAAAGHVTAFDIWGQGTMVTVSSASTKGPSVFPLAPCSRSTSESTAALG
CLVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLG
Ab2 - Light chain amino acid sequence
(SEQ ID NO: 20)
DIVMTQSPDSLAVSLGERATINCKSSQSVLYSSNNKNYLAWYQQKPGQPP
KLLIYWASTRESGVPDRFSGSGSGTDFTLTISSLQAEDVAVYYCQQYYST
PVTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
Ab3 - Heavy chain variable region amino acid
sequence
(SEQ ID NO: 95)
EVQLLESGGGLVQPGGSLRLSCAASGFTFRNYAMSWVRQAPGKGLEWVSS
ISGSGGATYYADSVKGRFTISRDNSKNTLYLQMNSLRAEDTAVYYCARVS
SGHWDFDYWGQGTLVTVSS
Ab3 - Heavy chain variable region nucleic acid
sequence
(SEQ ID NO: 96)
GAGGTTCAGCTTCTGGAGAGCGGCGGTGGTCTTGTACAACCTGGAGGATC
ACTCAGGTTGTCATGTGCCGCAAGCGGGTTTACATTCAGGAACTATGCAA
TGAGCTGGGTCAGACAGGCTCCCGGCAAGGGACTTGAGTGGGTATCTTCC
ATCAGCGGATCTGGAGGAGCAACATATTATGCAGATAGTGTCAAAGGCAG
GTTCACAATAAGCCGCGACAATTCTAAAAATACTCTTTATCTTCAAATGA
ATAGCCTTAGGGCTGAGGATACGGCGGTGTATTATTGTGCCCGCGTCTCA
AGCGGGCATTGGGACTTCGATTATTGGGGGCAGGGTACTCTGGTTACTGT
TTCCTCC
Ab3 - Light chain variable region amino acid
sequence
(SEQ ID NO: 97)
DIQMTQSPSSLSASVGDRVTITCRASQSISSYLNWYQQKPGKAPKLLIYD
ASSLQSGVPSRFSGSGSGTDFTLTISSLQPEDFATYYCQQSYSAPFTFGQ
GTKVEIK
Ab3 - Light chain variable region nucleic acid
sequence
(SEQ ID NO: 98)
GACATCCAAATGACACAGAGCCCGTCTTCCCTCTCAGCTTCAGTCGGTGA
TCGAGTGACGATTACGTGCCGCGCCAGCCAAAGCATCTCCTCCTATCTTA
ACTGGTATCAGCAGAAACCCGGAAAGGCCCCAAAGTTGCTTATTTACGAC
GCATCCTCCCTTCAATCTGGTGTGCCCAGCAGGTTCTCAGGCAGCGGTTC
AGGAACGGATTTTACTCTTACCATTTCTAGTCTTCAACCTGAGGATTTTG
CGACGTATTACTGTCAACAGAGCTACAGTGCGCCGTTCACCTTTGGGCAG
GGTACTAAGGTTGAGATAAAGC
Ab3 - Heavy chain amino acid sequence
(SEQ ID NO: 99)
EVQLLESGGGLVQPGGSLRLSCAASGFTFRNYAMSWVRQAPGKGLEWVSS
ISGSGGATYYADSVKGRFTISRDNSKNTLYLQMNSLRAEDTAVYYCARVS
SGHWDFDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
Ab3 - Light chain amino acid sequence
(SEQ ID NO: 100)
DIQMTQSPSSLSASVGDRVTITCRASQSISSYLNWYQQKPGKAPKLLIYD
ASSLQSGVPSRFSGSGSGTDFTLTISSLQPEDFATYYCQQSYSAPFTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, antibodies of the disclosure that specifically bind to a GARP-TGFβ1 complex, a LTBP1-TGFβ1 complex, a LTBP3-TGFβ1 complex, and a LRRC33-TGFβ1 complex include any antibody that includes a heavy chain variable domain of SEQ ID NO: 13, 17 or 95, or a light chain variable domain of SEQ ID NO: 14, 18 or 97. In some embodiments, antibodies of the disclosure that specifically bind to a GARP-TGFβ1 complex, a LTBP1-TGFβ1 complex, a LTBP3-TGFβ1 complex, and a LRRC33-TGFβ1 complex include any antibody that includes the heavy chain variable and light chain variable pairs of SEQ ID NOs: 13 and 14; 17 and 18; and 95 and 97.

Aspects of the disclosure provide antibodies that specifically bind to two or more of the following complexes: a GARP-TGFβ1 complex, a LTBP1-TGFβ1 complex, a LTBP3-TGFβ1 complex, and a LRRC33-TGFβ1 complex, having a heavy chain variable and/or a light chain variable amino acid sequence homologous to any of those described herein. In some embodiments, the antibody that that specifically binds to a GARP-TGFβ1 complex, a LTBP1-TGFβ1 complex, a LTBP3-TGFβ1 complex, and a LRRC33-TGFβ1 complex comprises a heavy chain variable sequence or a light chain variable sequence that is at least 75% (e.g., 80%, 81%, 82%, 83%, 84%, 85%, 86%, 87%, 88%, 89%, 90%, 91%, 92%, 93%, 94%, 95%, 96%, 97%, 98%, or 99%) identical to the heavy chain variable amino acid sequence of SEQ ID NO: 13, 17 or 95, or a light chain variable sequence of SEQ ID NO: 14, 18 or 97. In some embodiments, the homologous heavy chain variable and/or a light chain variable amino acid sequences do not vary within any of the CDR sequences provided herein. For example, in some embodiments, the degree of sequence variation (e.g., 80%, 81%, 82%, 83%, 84%, 85%, 86%, 87%, 88%, 89%, 90%, 91%, 92%, 93%, 94%, 95%, 96%, 97%, 98%, or 99%) may occur within a heavy chain variable and/or a light chain variable amino acid sequence excluding any of the CDR sequences provided herein.

In some embodiments, antibodies of the disclosure that specifically bind to two or more of: a GARP-TGFβ1 complex, a LTBP1-TGFβ1 complex, a LTBP3-TGFβ1 complex, and a LRRC33-TGFβ1 complex include any antibody, or antigen binding portion thereof, that includes a heavy chain of SEQ ID NO: 15 or 19, or a light chain of SEQ ID NO: 16 or 20. In some embodiments, antibodies of the disclosure that specifically bind to a GARP-TGFβ1 complex, a LTBP1-TGFβ1 complex, a LTBP3-TGFβ1 complex, and/or a LRRC33-TGFβ1 complex include any antibody that includes the heavy chain and light chain pairs of SEQ ID NOs: 15 and 16; or 19 and 20.

Aspects of the disclosure provide antibodies that specifically bind to two or more of: a GARP-TGFβ1 complex, a LTBP1-TGFβ1 complex, a LTBP3-TGFβ1 complex, and a LRRC33-TGFβ1 complex having a heavy chain and/or a light chain amino acid sequence homologous to any of those described herein. In some embodiments, the antibody that specifically binds to a GARP-TGFβ1 complex, a LTBP1-TGFβ1 complex, a LTBP3-TGFβ1 complex, and/or a LRRC33-TGFβ1 complex comprises a heavy chain sequence or a light chain sequence that is at least 75% (e.g., 80%, 81%, 82%, 83%, 84%, 85%, 86%, 87%, 88%, 89%, 90%, 91%, 92%, 93%, 94%, 95%, 96%, 97%, 98%, or 99%) identical to the heavy chain sequence of SEQ ID NO: 15, or 19, or a light chain amino acid sequence of SEQ ID NO: 16, or 20. In some embodiments, the homologous heavy chain and/or a light chain amino acid sequences do not vary within any of the CDR sequences provided herein. For example, in some embodiments, the degree of sequence variation (e.g., 80%, 81%, 82%, 83%, 84%, 85%, 86%, 87%, 88%, 89%, 90%, 91%, 92%, 93%, 94%, 95%, 96%, 97%, 98%, or 99%) may occur within a heavy chain and/or a light chain amino acid sequence excluding any of the CDR sequences provided herein.

In some embodiments, antibodies of the disclosure that specifically bind to two or more of: a GARP-TGFβ1 complex, a LTBP1-TGFβ1 complex, a LTBP3-TGFβ1 complex, and/or a LRRC33-TGFβ1 complex include any antibody, or antigen binding portion thereof, that includes a heavy chain of SEQ ID NO: 15 or 19, or a light chain of SEQ ID NO: 16 or 20. In some embodiments, antibodies of the disclosure that specifically bind to a GARP-TGFβ1 complex, a LTBP1-TGFβ1 complex, a LTBP3-TGFβ1 complex, and/or a LRRC33-TGFβ1 complex include any antibody that includes the heavy chain and light chain pairs of SEQ ID NOs: 15 and 16; or 19 and 20.

Aspects of the disclosure provide antibodies that specifically bind to two or more of: a GARP-TGFβ1 complex, a LTBP1-TGFβ1 complex, a LTBP3-TGFβ1 complex, and/or a LRRC33-TGFβ1 complex having a heavy chain and/or a light chain amino acid sequence homologous to any of those described herein. In some embodiments, the antibody that that specifically binds to a GARP-TGFβ1 complex, a LTBP1-TGFβ1 complex, a LTBP3-TGFβ1 complex, and/or a LRRC33-TGFβ1 complex comprises a heavy chain sequence or a light chain sequence that is at least 75% (e.g., 80%, 81%, 82%, 83%, 84%, 85%, 86%, 87%, 88%, 89%, 90%, 91%, 92%, 93%, 94%, 95%, 96%, 97%, 98%, or 99%) identical to the heavy chain sequence of SEQ ID NO: 15 or 19, or a light chain amino acid sequence of SEQ ID NO: 16 or 20. In some embodiments, the homologous heavy chain and/or a light chain amino acid sequences do not vary within any of the CDR sequences provided herein. For example, in some embodiments, the degree of sequence variation (e.g., 80%, 81%, 82%, 83%, 84%, 85%, 86%, 87%, 88%, 89%, 90%, 91%, 92%, 93%, 94%, 95%, 96%, 97%, 98%, or 99%) may occur within a heavy chain and/or a light chain amino acid sequence excluding any of the CDR sequences provided herein.

In some embodiments, the “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In any of the antibodies or antigen-binding fragments described herein, one or more conservative mutations can be introduced into the CDRs or framework sequences at positions where the residues are not likely to be involved in an antibody-antigen interaction. In some embodiments, such conservative mutation(s) can be introduced into the CDRs or framework sequences at position(s) where the residues are not likely to be involved in interacting with a GARP-TGFβ1 complex, a LTBP1-TGFβ1 complex, a LTBP3-TGFβ1 complex, and a LRRC33-TGFβ1 complex as determined based on the crystal structure. In some embodiments, likely interface (e.g., residues involved in an antigen-antibody interaction) may be deduced from known structural information on another antigen sharing structural similarities.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like (CPPCP (SEQ ID NO: 54)) hinge sequence. Accordingly, any of the antibodies may include a stabilizing ‘Adair’ mutation or the amino acid sequence CPPCP (SEQ ID NO: 54).

Isoform-specific, context-permissive inhibitors (which encompass context-independent inhibitors) of TGFβ1 of the present disclosure, e.g., antibodies that specifically bind to two or more of: a GARP-TGFβ1 complex, a LTBP1-TGFβ1 complex, a LTBP3-TGFβ1 complex, and a LRRC33-TGFβ1 complex, may optionally comprise antibody constant regions or parts thereof. For example, a VL domain may be attached at its C-terminal end to a light chain constant domain like CK or CA. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

Additionally or alternatively, such antibodies may or may not include the framework region of the antibodies of SEQ ID NOs: 13-20. In some embodiments, antibodies that specifically bind to a GARP-TGFβ1 complex, a LTBP1-TGFβ1 complex, a LTBP3-TGFβ1 complex, and/or a LRRC33-TGFβ1 complex are murine antibodies and include murine framework region sequences.

In some embodiments, such antibodies bind to a GARP-TGFβ1 complex, a LTBP1-TGFβ1 complex, a LTBP3-TGFβ1 complex, and/or a LRRC33-TGFβ1 complex with relatively high affinity, e.g., with a KD less than 10⁻⁶ M, 10⁻⁷ M, 10⁻⁸ M, 10⁻⁹ M, 10⁻¹⁰ M, 10⁻¹¹ M or lower. For example, such antibodies may bind a GARP-TGFβ1 complex, a LTBP1-TGFβ1 complex, a LTBP3-TGFβ1 complex, and/or a LRRC33-TGFβ1 complex with an affinity between 5 pM and 500 nM, e.g., between 50 pM and 100 nM, e.g., between 500 pM and 50 nM. The disclosure also includes antibodies or antigen binding fragments that compete with any of the antibodies described herein for binding to a GARP-TGFβ1 complex, a LTBP1-TGFβ1 complex, a LTBP3-TGFβ1 complex, and/or a LRRC33-TGFβ1 complex and that have an affinity of 50 nM or lower (e.g., 20 nM or lower, 10 nM or lower, 500 pM or lower, 50 pM or lower, or 5 pM or lower). The affinity and binding kinetics of the antibodies that specifically bind to a GARP-TGFβ1 complex, a LTBP1-TGFβ1 complex, a LTBP3-TGFβ1 complex, and/or a LRRC33-TGFβ1 complex can be tested using any suitable method including but not limited to biosensor technology (e.g., OCTET or BIACORE).

In some embodiments, inhibitors of cell-associated TGFβ1 (e.g., GARP-presented TGFβ1 and LRRC33-presented TGFβ1) according to the invention include antibodies or fragments thereof that specifically bind such complex (e.g., GARP-pro/latent TGFβ1 and LRRC33-pro/latent TGFβ1) and trigger internalization of the complex. This mode of action causes removal or depletion of the inactive TGFβ1 complexes from the cell surface (e.g., Treg, macropahges, etc.), hence reducing TGFβ1 available for activation. In some embodiments, such antibodies or fragments thereof bind the target complex in a pH-dependent manner such that binding occurs at a neutral or physiological pH, but the antibody dissociates from its antigen at an acidic pH. Such antibodies or fragments thereof may function as recycling antibodies.

### Polypeptides

Some aspects of the disclosure relate to a polypeptide having a sequence selected from the group consisting of SEQ ID NO: 13, SEQ ID NO: 17, SEQ ID NO: 95, SEQ ID NO: 15, and SEQ ID NO: 19. In some embodiments, the polypeptide is a variable heavy chain domain or a heavy chain domain. In some embodiments, the polypeptide is at least 75% (e.g., 80%, 81%, 82%, 83%, 84%, 85%, 86%, 87%, 88%, 89%, 90%, 91%, 92%, 93%, 94%, 95%, 96%, 97%, 98%, or 99%) identical to any one of the amino acid sequences set forth in SEQ ID NO: 13, SEQ ID NO: 17, SEQ ID NO: 95, SEQ ID NO: 15, and SEQ ID NO: 19.

Some aspects of the disclosure relate to a polypeptide having a sequence selected from the group consisting of SEQ ID NO: 14, SEQ ID NO: 18, SEQ ID NO: 97, SEQ ID NO: 16, and SEQ ID NO: 20. In some embodiments, the polypeptide is a variable light chain domain or a light chain domain. In some embodiments, the polypeptide is at least 75% (e.g., 80%, 81%, 82%, 83%, 84%, 85%, 86%, 87%, 88%, 89%, 90%, 91%, 92%, 93%, 94%, 95%, 96%, 97%, 98%, or 99%) identical to any one of the amino acid sequences set forth in SEQ ID NO: 14, SEQ ID NO: 18, SEQ ID NO: 97, SEQ ID NO: 16, and SEQ ID NO: 20.

Antibodies Competing with Isoform-Specific, Context-Permissive Inhibitory Antibodies of TGFβ1

Aspects of the disclosure relate to antibodies that compete or cross-compete with any of the antibodies provided herein. The term “compete”, as used herein with regard to an antibody, means that a first antibody binds to an epitope (e.g., an epitope of a GARP-TGFβ1 complex, a LTBP1-TGFβ1 complex, a LTBP3-TGFβ1 complex, and/or a LRRC33-TGFβ1 complex) in a manner sufficiently similar to the binding of a second antibody, such that the result of binding of the first antibody with its epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its epitope or ligand, whether to the same, greater, or lesser extent, the antibodies are said to “cross-compete” with each other for binding of their respective epitope(s). Both competing and cross-competing antibodies are within the scope of this disclosure. Regardless of the mechanism by which such competition or cross-competition occurs (e.g., steric hindrance, conformational change, or binding to a common epitope, or portion thereof), the skilled artisan would appreciate that such competing and/or cross-competing antibodies are encompassed and can be useful for the methods and/or compositions provided herein.

Aspects of the disclosure relate to antibodies that compete or cross-compete with any of the specific antibodies, or antigen binding portions thereof, as provided herein. In some embodiments, an antibody, or antigen binding portion thereof, binds at or near the same epitope as any of the antibodies provided herein. In some embodiments, an antibody, or antigen binding portion thereof, binds near an epitope if it binds within 15 or fewer amino acid residues of the epitope. In some embodiments, any of the antibody, or antigen binding portion thereof, as provided herein, binds within 1, 2, 3, 4, 5, 6, 7, 8, 9, 10, 11, 12, 13, 14 or 15 amino acid residues of an epitope that is bound by any of the antibodies provided herein.

In another embodiment, provided herein is an antibody, or antigen binding portion thereof, competes or cross-competes for binding to any of the antigens provided herein (e.g., a GARP-TGFβ1 complex, a LTBP1-TGFβ1 complex, a LTBP3-TGFβ1 complex, and/or a LRRC33-TGFβ1 complex) with an equilibrium dissociation constant, KD, between the antibody and the protein of less than 10⁻⁶M. In other embodiments, an antibody competes or cross-competes for binding to any of the antigens provided herein with a KD in a range from 10⁻¹¹ M to 10⁻⁶ M. In some embodiments, provided herein is an anti-TGFβ1 antibody, or antigen binding portion thereof, that competes for binding with an antibody, or antigen binding portion thereof, described herein. In some embodiments, provided herein is an anti-TGFβ1 antibody, or antigen binding portion thereof, that binds to the same epitope as an antibody, or antigen binding portion thereof, described herein.

Any of the antibodies provided herein can be characterized using any suitable methods. For example, one method is to identify the epitope to which the antigen binds, or “epitope mapping.” There are many suitable methods for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). In some embodiments, the epitope is a TGFβ1 epitope that is only available for binding by the antibody, or antigen binding portion thereof, described herein, when the TGFβ1 is in a GARP-TGFβ1 complex, a LTBP1-TGFβ1 complex, a LTBP3-TGFβ1 complex, and/or a LRRC33-TGFβ1 complex. Peptides of varying lengths (e.g., at least 4-6 amino acids long) can be isolated or synthesized (e.g., recombinantly) and used for binding assays with an antibody. In another example, the epitope to which the antibody binds can be determined in a systematic screen by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the GARP-TGFβ1 complex, a LTBP1-TGFβ1 complex, a LTBP3-TGFβ1 complex, and/or a LRRC33-TGFβ1 complex have been replaced (swapped) with sequences from a closely related, but antigenically distinct protein, such as another member of the TGFβ protein family (e.g., GDF11). By assessing binding of the antibody to the mutant of the a GARP-TGFβ1 complex, a LTBP1-TGFβ1 complex, a LTBP3-TGFβ1 complex, and/or a LRRC33-TGFβ1 complex, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

Further, the interaction of the any of the antibodies provided herein with one or more residues in a GARP-TGFβ1 complex, a LTBP1-TGFβ1 complex, a LTBP3-TGFβ1 complex, and/or a LRRC33-TGFβ1 complex can be determined by routine technology. For example, a crystal structure can be determined, and the distances between the residues in a GARP-TGFβ1 complex, a LTBP1-TGFβ1 complex, a LTBP3-TGFβ1 complex, and/or a LRRC33-TGFβ1 complex and one or more residues in the antibody can be determined accordingly. Based on such distance, whether a specific residue in a GARP-TGFβ1 complex, a LTBP1-TGFβ1 complex, a LTBP3-TGFβ1 complex, and/or a LRRC33-TGFβ1 complex interacts with one or more residues in the antibody can be determined. Further, suitable methods, such as competition assays and target mutagenesis assays can be applied to determine the preferential binding of a candidate antibody.

### Various Modifications and Variations of Antibodies

Non-limiting variations, modifications, and features of any of the antibodies or antigen-binding fragments thereof encompassed by the present disclosure are briefly discussed below. Embodiments of related analytical methods are also provided.

Naturally-occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human antibody light chains are typically classified as kappa and lambda light chains. Heavy chains are typically classified as mu, delta, gamma, alpha, or epsilon, and define the isotype of the antibody. An antibody can be of any type (e.g., IgM, IgD, IgG, IgA, IgY, and IgE) and class (e.g., IgG₁, IgG₂, IgG₃, IgG₄, IgM₁, IgM₂, IgA₁, and IgA₂). Within full-length light and heavy chains, typically, the variable and constant regions are joined by a “J” region of about 12 or more amino acids, with the heavy chain also including a “D” region of about 10 more amino acids (see, e.g., Fundamental Immunology, Ch. 7 (Paul, W., ed., 2nd ed. Raven Press, N.Y. (1989)) (incorporated by reference in its entirety)). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk (1987) J. Mol. Biol. 196: 901-917; Chothia et al. (1989) Nature 342: 878-883. The CDRs of a light chain can also be referred to as CDR-L1, CDR-L2, and CDR-L3, and the CDRs of a heavy chain can also be referred to as CDR-H1, CDR-H2, and CDR-H3. In some embodiments, an antibody can comprise a small number of amino acid deletions from the carboxy end of the heavy chain(s). In some embodiments, an antibody comprises a heavy chain having 1-5 amino acid deletions in the carboxy end of the heavy chain. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition, and the contact definition.

An “affinity matured” antibody is an antibody with one or more alterations in one or more CDRs thereof, which result an improvement in the affinity of the antibody for antigen compared to a parent antibody, which does not possess those alteration(s). Exemplary affinity matured antibodies will have nanomolar or even picomolar affinities for the target antigen. Affinity matured antibodies are produced by procedures known in the art. Marks et al. (1992) Bio/Technology 10: 779-783 describes affinity maturation by VH and VL domain shuffling. Random mutagenesis of CDR and/or framework residues is described by Barbas, et al. (1994) Proc Nat. Acad. Sci. USA 91: 3809-3813; Schier et al. (1995) Gene 169: 147-155; Yelton et al., (1995) J. Immunol. 155: 1994-2004; Jackson et al. (1995) J. Immunol. 154(7): 3310-9; and Hawkins et al. (1992) J. Mol. Biol. 226: 889-896; and selective mutation at selective mutagenesis positions, contact or hypermutation positions with an activity enhancing amino acid residue is described in U.S. Pat. No. 6,914,128.

The term “CDR-grafted antibody” refers to antibodies, which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “chimeric antibody” refers to antibodies, which comprise heavy and light chain variable region sequences from one species and constant region sequences from another species, such as antibodies having murine heavy and light chain variable regions linked to human constant regions.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

In some embodiments, the antibody, or antigen binding portion thereof, comprises a heavy chain immunoglobulin constant domain of a human IgM constant domain, a human IgG constant domain, a human IgG1 constant domain, a human IgG2 constant domain, a human IgG2A constant domain, a human IgG2B constant domain, a human IgG2 constant domain, a human IgG3 constant domain, a human IgG3 constant domain, a human IgG4 constant domain, a human IgA constant domain, a human IgA1 constant domain, a human IgA2 constant domain, a human IgD constant domain, or a human IgE constant domain. In some embodiments, the antibody, or antigen binding portion thereof, comprises a heavy chain immunoglobulin constant domain of a human IgG1 constant domain or a human IgG4 constant domain. In some embodiments, the antibody, or antigen binding portion thereof, comprises a heavy chain immunoglobulin constant domain of a human IgG4 constant domain. In some embodiments, the antibody, or antigen binding portion thereof, comprises a heavy chain immunoglobulin constant domain of a human IgG4 constant domain having a backbone substitution of Ser to Pro that produces an IgG1-like hinge and permits formation of inter-chain disulfide bonds.

In some embodiments, the antibody or antigen binding portion thereof, further comprises a light chain immunoglobulin constant domain comprising a human Ig lambda constant domain or a human Ig kappa constant domain.

In some embodiments, the antibody is an IgG having four polypeptide chains which are two heavy chains and two light chains.

In some embodiments, wherein the antibody is a humanized antibody, a diabody, or a chimeric antibody. In some embodiments, the antibody is a humanized antibody. In some embodiments, the antibody is a human antibody. In some embodiments, the antibody comprises a framework having a human germline amino acid sequence.

In some embodiments, the antigen binding portion is a Fab fragment, a F(ab′)2 fragment, a scFab fragment, or an scFv fragment.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin (see, e.g., Shapiro et al. (2002) Crit. Rev. Immunol. 22(3): 183-200; Marchalonis et al. (2001) Adv. Exp. Med. Biol. 484: 13-30). One of the advantages provided by various embodiments of the present disclosure stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “neutralizing” refers to counteracting the biological activity of an antigen when a binding protein specifically binds to the antigen. In an embodiment, the neutralizing binding protein binds to the antigen/target, e.g., cytokine, kinase, growth factor, cell surface protein, soluble protein, phosphatase, or receptor ligand, and reduces its biologically activity by at least about 20%, 40%, 60%, 80%, 85%, 90%, 95%. 96%, 97%. 98%, 99% or more.

The term “binding protein” as used herein includes any polypeptide that specifically binds to an antigen (e.g., TGFβ1), including, but not limited to, an antibody, or antigen binding portions thereof, a DVD-Ig™, a TVD-Ig, a RAb-Ig, a bispecific antibody and a dual specific antibody.

The term “monoclonal antibody” or “mAb” when used in a context of a composition comprising the same may refer to an antibody preparation obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “recombinant human antibody,” as used herein, is intended to include all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. R. (1997) TIB Tech. 15: 62-70; Azzazy, H. and Highsmith, W. E. (2002) Clin. Biochem. 35: 425-445; Gavilondo, J. V. and Larrick, J. W. (2002) BioTechniques 29: 128-145; Hoogenboom, H. and Chames, P. (2000) Immunol. Today 21: 371-378, incorporated herein by reference), antibodies isolated from an animal (e.g., a mouse) that is transgenic for human immunoglobulin genes (see, Taylor, L. D. et al. (1992) Nucl. Acids Res. 20: 6287-6295; Kellermann, S-A. and Green, L. L. (2002) Cur. Opin. in Biotechnol. 13: 593-597; Little, M. et al. (2000) Immunol. Today 21: 364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “Dual Variable Domain Immunoglobulin” or “DVD-Ig™” and the like include binding proteins comprising a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms bound to each other at least in part by dimerization of the CH3 domains, with each arm of the DVD being bispecific, providing an immunoglobulin with four binding sites. DVD-Ig™ are provided in US Patent Publication Nos. 2010/0260668 and 2009/0304693, each of which are incorporated herein by reference including sequence listings.

As used herein, “Triple Variable Domain Immunoglobulin” or “TVD-Ig” and the like are binding proteins comprising a paired heavy chain TVD binding protein polypeptide and a light chain TVD binding protein polypeptide with each paired heavy and light chain providing three antigen binding sites. Each binding site includes a total of 6 CDRs involved in antigen binding per antigen binding site. A TVD binding protein may have two arms bound to each other at least in part by dimerization of the CH3 domains, with each arm of the TVD binding protein being trispecific, providing a binding protein with six binding sites.

As used herein, “Receptor-Antibody Immunoglobulin” or “RAb-Ig” and the like are binding proteins comprising a heavy chain RAb polypeptide, and a light chain RAb polypeptide, which together form three antigen binding sites in total. One antigen binding site is formed by the pairing of the heavy and light antibody variable domains present in each of the heavy chain RAb polypeptide and the light chain RAb polypeptide to form a single binding site with a total of 6 CDRs providing a first antigen binding site. Each the heavy chain RAb polypeptide and the light chain RAb polypeptide include a receptor sequence that independently binds a ligand providing the second and third “antigen” binding sites. A RAb-Ig is typically has two arms bound to each other at least in part by dimerization of the CH3 domains, with each arm of the RAb-Ig being trispecific, providing an immunoglobulin with six binding sites. RAb-Igs are described in US Patent Application Publication No. 2002/0127231, the entire contents of which including sequence listings are incorporated herein by reference).

The term “bispecific antibody,” as used herein, and as differentiated from a “bispecific half-Ig binding protein” or “bispecific (half-Ig) binding protein”, refers to full-length antibodies that are generated by quadroma technology (see Milstein, C. and Cuello, A. C. (1983) Nature 305(5934): p. 537-540), by chemical conjugation of two different monoclonal antibodies (see Staerz, U. D. et al. (1985) Nature 314(6012): 628-631), or by knob-into-hole or similar approaches, which introduce mutations in the Fc region that do not inhibit CH3-CH3 dimerization (see Holliger, P. et al. (1993) Proc. Natl. Acad. Sci USA 90(14): 6444-6448), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen it binds to.

The term “dual-specific antibody,” as used herein, and as differentiated from a bispecific half-Ig binding protein or bispecific binding protein, refers to full-length antibodies that can bind two different antigens (or epitopes) in each of its two binding arms (a pair of HC/LC) (see PCT Publication No. WO 02/02773). Accordingly, a dual-specific binding protein has two identical antigen binding arms, with identical specificity and identical CDR sequences, and is bivalent for each antigen to which it binds.

The term “Kon,” as used herein, is intended to refer to the on rate constant for association of a binding protein (e.g., an antibody) to the antigen to form the, e.g., antibody/antigen complex as is known in the art. The “Kon” also is known by the terms “association rate constant,” or “ka,” as used interchangeably herein. This value indicating the binding rate of an antibody to its target antigen or the rate of complex formation between an antibody and antigen also is shown by the equation: Antibody (“Ab”)+Antigen (“Ag”)→Ab-Ag.

The term “Koff,” as used herein, is intended to refer to the off rate constant for dissociation of a binding protein (e.g., an antibody) from the, e.g., antibody/antigen complex as is known in the art. The “Koff” also is known by the terms “dissociation rate constant” or “kd” as used interchangeably herein. This value indicates the dissociation rate of an antibody from its target antigen or separation of Ab-Ag complex over time into free antibody and antigen as shown by the equation: Ab+Ag←Ab-Ag.

The terms “equilibrium dissociation constant” or “KD,” as used interchangeably herein, refer to the value obtained in a titration measurement at equilibrium, or by dividing the dissociation rate constant (koff) by the association rate constant (kon). The association rate constant, the dissociation rate constant, and the equilibrium dissociation constant are used to represent the binding affinity of a binding protein, e.g., antibody, to an antigen. Methods for determining association and dissociation rate constants are well known in the art. Using fluorescence-based techniques offers high sensitivity and the ability to examine samples in physiological buffers at equilibrium. Other experimental approaches and instruments, such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The terms “crystal” and “crystallized” as used herein, refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 201-16, Oxford University Press, New York, N.Y., (1999). The term “linker” is used to denote polypeptides comprising two or more amino acid residues joined by peptide bonds and are used to link one or more antigen binding portions. Such linker polypeptides are well known in the art (see, e.g., Holliger, P. et al. (1993) Proc. Natl. Acad. Sci. USA 90: 6444-6448; Poljak, R. J. et al. (1994) Structure 2:1121-1123). Exemplary linkers include, but are not limited to, ASTKGPSVFPLAP (SEQ ID NO: 55), ASTKGP (SEQ ID NO: 56); TVAAPSVFIFPP (SEQ ID NO: 57); TVAAP (SEQ ID NO: 58); AKTTPKLEEGEFSEAR (SEQ ID NO: 59); AKTTPKLEEGEFSEARV (SEQ ID NO: 60); AKTTPKLGG (SEQ ID NO: 61); SAKTTPKLGG (SEQ ID NO: 62); SAKTTP (SEQ ID NO: 63); RADAAP (SEQ ID NO: 64); RADAAPTVS (SEQ ID NO: 65); RADAAAAGGPGS (SEQ ID NO: 66); RADAAAA(G4S)4 (SEQ ID NO: 67); SAKTTPKLEEGEFSEARV (SEQ ID NO: 68); ADAAP (SEQ ID NO: 69); ADAAPTVSIFPP (SEQ ID NO: 70); QPKAAP (SEQ ID NO: 71); QPKAAPSVTLFPP (SEQ ID NO: 72); AKTTPP (SEQ ID NO: 73); AKTTPPSVTPLAP (SEQ ID NO: 74); AKTTAP (SEQ ID NO: 75); AKTTAPSVYPLAP (SEQ ID NO: 76); GGGGSGGGGSGGGGS (SEQ ID NO: 77); GENKVEYAPALMALS (SEQ ID NO: 78); GPAKELTPLKEAKVS (SEQ ID NO: 79); GHEAAAVMQVQYPAS (SEQ ID NO: 80); TVAAPSVFIFPPTVAAPSVFIFPP (SEQ ID NO: 81); and ASTKGPSVFPLAPASTKGPSVFPLAP (SEQ ID NO: 82).

“Label” and “detectable label” or “detectable moiety” mean a moiety attached to a specific binding partner, such as an antibody or an analyte, e.g., to render the reaction between members of a specific binding pair, such as an antibody and an analyte, detectable, and the specific binding partner, e.g., antibody or analyte, so labeled is referred to as “detectably labeled.” Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., 3H, 14C, 35S, 90Y, 99Tc, 111In, 125I, 131I, 177Lu, 166Ho, and 153Sm); chromogens; fluorescent labels (e.g., FITC, rhodamine, and lanthanide phosphors); enzymatic labels (e.g., horseradish peroxidase, luciferase, and alkaline phosphatase); chemiluminescent markers; biotinyl groups; predetermined polypeptide epitopes recognized by a secondary reporter (e.g., leucine zipper pair sequences, binding sites for secondary antibodies, metal binding domains, and epitope tags); and magnetic agents, such as gadolinium chelates. Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of “detectably labeled” is intended to encompass the latter type of detectable labeling.

In some embodiments, the binding affinity of an antibody, or antigen binding portion thereof, to an antigen (e.g., protein complex), such as a GARP-TGFβ1 complex, a LTBP1-TGFβ1 complex, a LTBP3-TGFβ1 complex, and/or a LRRC33-TGFβ1 complex is determined using an Octet assay. In some embodiments, an Octet assay is an assay that determines one or more a kinetic parameters indicative of binding between an antibody and antigen. In some embodiments, an Octet® system (ForteBio, Menlo Park, Calif.) is used to determine the binding affinity of an antibody, or antigen binding portion thereof, to a GARP-TGFβ1 complex, a LTBP1-TGFβ1 complex, a LTBP3-TGFβ1 complex, and/or a LRRC33-TGFβ1 complex. For example, binding affinities of antibodies may be determined using the fortéBio Octet QKe dip and read label free assay system utilizing bio-layer interferometry. In some embodiments, antigens are immobilized to biosensors (e.g., streptavidin-coated biosensors) and the antibodies and complexes (e.g., biotinylated GARP-TGFβ1 complexes and biotinylated LTBP-TGFβ1 complexes) are presented in solution at high concentration (50 μg/mL) to measure binding interactions. In some embodiments, the binding affinity of an antibody, or antigen binding portion thereof, to a GARP-TGFβ1 complex, a LTBP1-TGFβ1 complex, a LTBP3-TGFβ1 complex, and/or a LRRC33-TGFβ1 complex is determined using the protocol outlined in Table 6. The term “surface plasmon resonance,” as used herein, refers to an optical phenomenon that allows for the analysis of real-time bispecific interactions by detection of alterations in protein concentrations within a biosensor matrix, for example, using the BIAcore® system (BIAcore International AB, a GE Healthcare company, Uppsala, Sweden and Piscataway, N.J.). For further descriptions, see Jonsson, U. et al. (1993) Ann. Biol. Clin. 51: 19-26; Jinsson, U. et al. (1991) Biotechniques 11: 620-627; Johnsson, B. et al. (1995) J. Mol. Recognit. 8: 125-131; and Johnnson, B. et al. (1991) Anal. Biochem. 198: 268-277.

### Identification and Production/Manufacture of Isoform-Specific, Context-Permissive Inhibitors of TGFβ1

The invention encompasses screening methods, production methods and manufacture processes of antibodies or fragments thereof which bind two or more of: a GARP-TGFβ1 complex, a LTBP1-TGFβ1 complex, a LTBP3-TGFβ1 complex, and/or a LRRC33-TGFβ1 complex, and pharmaceutical compositions and related kits comprising the same.

Numerous methods may be used for obtaining antibodies, or antigen binding fragments thereof, of the disclosure. For example, antibodies can be produced using recombinant DNA methods. Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody that specifically binds to a specified antigen. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597; WO 92/18619; WO 91/17271; WO 92/20791; WO 92/15679; WO 93/01288; WO 92/01047; WO 92/09690; and WO 90/02809.

In addition to the use of display libraries, the specified antigen (e.g., a GARP-TGFβ1 complex, a LTBP1-TGFβ1 complex, a LTBP3-TGFβ1 complex, and/or a LRRC33-TGFβ1 complex) can be used to immunize a non-human host, e.g., rabbit, guinea pig, rat, mouse, hamster, sheep, goat, chicken, camelid, as well as non-mammalian hosts such as shark. In one embodiment, the non-human animal is a mouse.

In another embodiment, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

Some aspects of the present disclosure relate to host cells transformed with a polynucleotide or vector. Host cells may be a prokaryotic or eukaryotic cell. The polynucleotide or vector which is present in the host cell may either be integrated into the genome of the host cell or it may be maintained extrachromosomally. The host cell can be any prokaryotic or eukaryotic cell, such as a bacterial, insect, fungal, plant, animal or human cell. In some embodiments, fungal cells are, for example, those of the genus Saccharomyces, in particular those of the species S. cerevisiae. The term “prokaryotic” includes all bacteria which can be transformed or transfected with a DNA or RNA molecules for the expression of an antibody or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” includes yeast, higher plants, insects and vertebrate cells, e.g., mammalian cells, such as NSO and CHO cells. Depending upon the host employed in a recombinant production procedure, the antibodies or immunoglobulin chains encoded by the polynucleotide may be glycosylated or may be non-glycosylated. Antibodies or the corresponding immunoglobulin chains may also include an initial methionine amino acid residue.

In some embodiments, once a vector has been incorporated into an appropriate host, the host may be maintained under conditions suitable for high level expression of the nucleotide sequences, and, as desired, the collection and purification of the immunoglobulin light chains, heavy chains, light/heavy chain dimers or intact antibodies, antigen binding fragments or other immunoglobulin forms may follow; see, Beychok, Cells of Immunoglobulin Synthesis, Academic Press, N.Y., (1979). Thus, polynucleotides or vectors are introduced into the cells which in turn produce the antibody or antigen binding fragments. Furthermore, transgenic animals, preferably mammals, comprising the aforementioned host cells may be used for the large scale production of the antibody or antibody fragments.

The transformed host cells can be grown in fermenters and cultured using any suitable techniques to achieve optimal cell growth. Once expressed, the whole antibodies, their dimers, individual light and heavy chains, other immunoglobulin forms, or antigen binding fragments, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or antigen binding fragments can then be isolated from the growth medium, cellular lysates, or cellular membrane fractions. The isolation and purification of the, e.g., microbially expressed antibodies or antigen binding fragments may be by any conventional means such as, for example, preparative chromatographic separations and immunological separations such as those involving the use of monoclonal or polyclonal antibodies directed, e.g., against the constant region of the antibody.

Aspects of the disclosure relate to a hybridoma, which provides an indefinitely prolonged source of monoclonal antibodies. As an alternative to obtaining immunoglobulins directly from the culture of hybridomas, immortalized hybridoma cells can be used as a source of rearranged heavy chain and light chain loci for subsequent expression and/or genetic manipulation. Rearranged antibody genes can be reverse transcribed from appropriate mRNAs to produce cDNA. In some embodiments, heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Any appropriate method may be used for cloning of antibody variable regions and generation of recombinant antibodies.

In some embodiments, an appropriate nucleic acid that encodes variable regions of a heavy and/or light chain is obtained and inserted into an expression vectors which can be transfected into standard recombinant host cells. A variety of such host cells may be used. In some embodiments, mammalian host cells may be advantageous for efficient processing and production. Typical mammalian cell lines useful for this purpose include CHO cells, 293 cells, or NSO cells. The production of the antibody or antigen binding fragment may be undertaken by culturing a modified recombinant host under culture conditions appropriate for the growth of the host cells and the expression of the coding sequences. The antibodies or antigen binding fragments may be recovered by isolating them from the culture. The expression systems may be designed to include signal peptides so that the resulting antibodies are secreted into the medium; however, intracellular production is also possible.

The disclosure also includes a polynucleotide encoding at least a variable region of an immunoglobulin chain of the antibodies described herein. In some embodiments, the variable region encoded by the polynucleotide comprises at least one complementarity determining region (CDR) of the VH and/or VL of the variable region of the antibody produced by any one of the above described hybridomas.

Polynucleotides encoding antibody or antigen binding fragments may be, e.g., DNA, cDNA, RNA or synthetically produced DNA or RNA or a recombinantly produced chimeric nucleic acid molecule comprising any of those polynucleotides either alone or in combination. In some embodiments, a polynucleotide is part of a vector. Such vectors may comprise further genes such as marker genes which allow for the selection of the vector in a suitable host cell and under suitable conditions.

In some embodiments, a polynucleotide is operatively linked to expression control sequences allowing expression in prokaryotic or eukaryotic cells. Expression of the polynucleotide comprises transcription of the polynucleotide into a translatable mRNA. Regulatory elements ensuring expression in eukaryotic cells, preferably mammalian cells, are well known to those skilled in the art. They may include regulatory sequences that facilitate initiation of transcription and optionally poly-A signals that facilitate termination of transcription and stabilization of the transcript. Additional regulatory elements may include transcriptional as well as translational enhancers, and/or naturally associated or heterologous promoter regions. Possible regulatory elements permitting expression in prokaryotic host cells include, e.g., the PL, Lac, Trp or Tac promoter in E. coli, and examples of regulatory elements permitting expression in eukaryotic host cells are the AOX1 or GAL1 promoter in yeast or the CMV-promoter, SV40-promoter, RSV-promoter (Rous sarcoma virus), CMV-enhancer, SV40-enhancer or a globin intron in mammalian and other animal cells.

Beside elements which are responsible for the initiation of transcription such regulatory elements may also include transcription termination signals, such as the SV40-poly-A site or the tk-poly-A site, downstream of the polynucleotide. Furthermore, depending on the expression system employed, leader sequences capable of directing the polypeptide to a cellular compartment or secreting it into the medium may be added to the coding sequence of the polynucleotide and have been described previously. The leader sequence(s) is (are) assembled in appropriate phase with translation, initiation and termination sequences, and preferably, a leader sequence capable of directing secretion of translated protein, or a portion thereof, into, for example, the extracellular medium. Optionally, a heterologous polynucleotide sequence can be used that encode a fusion protein including a C- or N-terminal identification peptide imparting desired characteristics, e.g., stabilization or simplified purification of expressed recombinant product.

In some embodiments, polynucleotides encoding at least the variable domain of the light and/or heavy chain may encode the variable domains of both immunoglobulin chains or only one. Likewise, polynucleotides may be under the control of the same promoter or may be separately controlled for expression. Furthermore, some aspects relate to vectors, particularly plasmids, cosmids, viruses and bacteriophages used conventionally in genetic engineering that comprise a polynucleotide encoding a variable domain of an immunoglobulin chain of an antibody or antigen binding fragment; optionally in combination with a polynucleotide that encodes the variable domain of the other immunoglobulin chain of the antibody.

In some embodiments, expression control sequences are provided as eukaryotic promoter systems in vectors capable of transforming or transfecting eukaryotic host cells, but control sequences for prokaryotic hosts may also be used. Expression vectors derived from viruses such as retroviruses, vaccinia virus, adeno-associated virus, herpes viruses, or bovine papilloma virus, may be used for delivery of the polynucleotides or vector into targeted cell population (e.g., to engineer a cell to express an antibody or antigen binding fragment). A variety of appropriate methods can be used to construct recombinant viral vectors. In some embodiments, polynucleotides and vectors can be reconstituted into liposomes for delivery to target cells. The vectors containing the polynucleotides (e.g., the heavy and/or light variable domain(s) of the immunoglobulin chains encoding sequences and expression control sequences) can be transferred into the host cell by suitable methods, which vary depending on the type of cellular host.

The screening methods may include a step of evaluating or confirming desired activities of the antibody or fragment thereof. In some embodiments, the step comprises selecting for the ability to inhibit target function, e.g., inhibition of release of mature TGFβ1 from a latent complex. In some embodiments, the step comprises selecting for antibodies or fragments thereof that promote internalization and subsequent removal of antibody-antigen complexes from the cell surface. In some embodiments, the step comprises selecting for antibodies or fragments thereof that induce ADCC. In some embodiments, the step comprises selecting for antibodies or fragments thereof that accumulate to a desired site(s) in vivo (e.g., cell type, tissue or organ). In some embodiments, the step comprises selecting for antibodies or fragments thereof with the ability to cross the blood brain barrier. The methods may optionally include a step of optimizing one or more antibodies or fragments thereof to provide variant counterparts that possess desirable profiles, as determined by criteria such as stability, binding affinity, functionality (e.g., inhibitory activities, Fc function, etc.), immunogenicity, pH sensitivity and developability (e.g., high solubility, low self-association, etc.). Such step may include affinity maturation of an antibody or fragment thereof. The resulting optimized antibody is preferably a fully human antibody or humanized antibody suitable for human administration. Manufacture process for a pharmaceutical composition comprising such an antibody or fragment thereof may comprise the steps of purification, formulation, sterile filtration, packaging, etc. Certain steps such as sterile filtration, for example, are performed in accordance with the guidelines set forth by relevant regulatory agencies, such as the FDA. Such compositions may be made available in a form of single-use containers, such as pre-filled syringes, or multi-dosage containers, such as vials.

### Modifications

Antibodies, or antigen binding portions thereof, of the disclosure may be modified with a detectable label or detectable moiety, including, but not limited to, an enzyme, prosthetic group, fluorescent material, luminescent material, bioluminescent material, radioactive material, positron emitting metal, nonradioactive paramagnetic metal ion, and affinity label for detection and isolation of a GARP-TGFβ1 complex, a LTBP1-TGFβ1 complex, a LTBP3-TGFβ1 complex, and/or a LRRC33-TGFβ1 complex. The detectable substance or moiety may be coupled or conjugated either directly to the polypeptides of the disclosure or indirectly, through an intermediate (such as, for example, a linker (e.g., a cleavable linker)) using suitable techniques. Non-limiting examples of suitable enzymes include horseradish peroxidase, alkaline phosphatase, D-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., alpha-emitters or other radioisotopes such as, for example, iodine (131I, 125I, 123I, 121I), carbon (14C), sulfur (35S), tritium (3H), indium (115mIn, 113mIn, 1121n, 1111n), and technetium (99Tc, 99mTc), thallium (201Ti), gallium (68Ga, 67Ga), palladium (103Pd), molybdenum (99Mo), xenon (133Xe), fluorine (18F), 153Sm, Lu, 159Gd, 149Pm, 140La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, and tin (113Sn, 117Sn). The detectable substance may be coupled or conjugated either directly to the antibodies of the disclosure that bind specifically to a GARP-TGFβ1 complex, a LTBP1-TGFβ1 complex, a LTBP3-TGFβ1 complex, and/or a LRRC33-TGFβ1 complex, or indirectly, through an intermediate (such as, for example, a linker) using suitable techniques. Any of the antibodies provided herein that are conjugated to a detectable substance may be used for any suitable diagnostic assays, such as those described herein.

In addition, antibodies, or antigen binding portions thereof, of the disclosure may also be modified with a drug. The drug may be coupled or conjugated either directly to the polypeptides of the disclosure, or indirectly, through an intermediate (such as, for example, a linker (e.g., a cleavable linker)) using suitable techniques.

### Targeting Agents

In some embodiments methods of the present disclosure comprise the use of one or more targeting agents to target an antibody, or antigen binding portion thereof, as disclosed herein, to a particular site in a subject for purposes of modulating mature TGFβ release from a GARP-TGFβ1 complex, a LTBP1-TGFβ1 complex, a LTBP3-TGFβ1 complex, and/or a LRRC33-TGFβ1 complex. For example, LTBP1-TGFβ1 and LTBP3-TGFβ1 complexes are typically localized to extracellular matrix. Thus, in some embodiments, antibodies disclosed herein can be conjugated to extracellular matrix targeting agents for purposes of localizing the antibodies to sites where LTBP1-TGFβ1 and LTBP3-TGFβ1 complexes reside. In such embodiments, selective targeting of antibodies leads to selective modulation of LTBP1-TGFβ1 and/or LTBP3-TGFβ1 complexes. In some embodiments, selective targeting of antibodies leads to selective inhibition of LTBP1-TGFβ1 and/or LTBP3-TGFβ1 complexes (e.g., for purposes of treating fibrosis). In some embodiments, extracellular matrix targeting agents include heparin binding agents, matrix metalloproteinase binding agents, lysyl oxidase binding domains, fibrillin-binding agents, hyaluronic acid binding agents, and others.

Similarly, GARP-TGFβ1 complexes are typically localized to the surface of cells, e.g., activated FOXP3+ regulatory T cells (Tregs). Thus, in some embodiments, antibodies disclosed herein can be conjugated to immune cell (e.g., Treg cell) binding agents for purposes of localizing antibodies to sites where GARP-TGFβ1 complexes reside. In such embodiments, selective targeting of antibodies leads to selective modulation of GARP-TGFβ1 complexes. In some embodiments, selective targeting of antibodies leads to selective inhibition of GARP-TGFβ1 complexes (e.g., selective inhibition of the release of mature TGFβ1 for purposes of immune modulation, e.g., in the treatment of cancer). In such embodiments, Treg cell targeting agents may include, for example, CCL22 and CXCL12 proteins or fragments thereof.

In some embodiments, bispecific antibodies may be used having a first portion that selectively binds GARP-TGFβ1 complex and a LTBP-TGFβ1 complex and a second portion that selectively binds a component of a target site, e.g., a component of the ECM (e.g., fibrillin) or a component of a Treg cell (e.g., CTLA-4).

### Pharmaceutical Compositions

The invention further provides pharmaceutical compositions used as a medicament suitable for administration in human and non-human subjects. One or more antibodies that specifically binds a GARP-TGFβ1 complex, a LTBP1-TGFβ1 complex, a LTBP3-TGFβ1 complex, and/or a LRRC33-TGFβ1 complex can be formulated or admixed with a pharmaceutically acceptable carrier (excipient), including, for example, a buffer, to form a pharmaceutical composition. Such formulations may be used for the treatment of a disease or disorder that involves TGFβ signaling. In some embodiments, such disease or disorder associated with TGFβ signaling involves one or more contexts, i.e., the TGFβ is associated with a particular type or types of presenting molecules. In some embodiments, such context occurs in a cell type-specific and/or tissue-specific manner. In some embodiments, for example, such context-dependent action of TGFβ signaling is mediated in part via GARP, LRRC33, LTBP1 and/or LTBP3.

In some embodiments, the antibody of the present invention binds specifically to two or more contexts of TGFβ, such that the antibody binds TGFβ in a complex with presenting molecules selected from two or more of: GARP, LRRC33, LTBP1 and LTBP3. Thus, such pharmaceutical compositions may be administered to patients for alleviating a TGFβ-related indication (e.g., fibrosis, immune disorders, and/or cancer). “Acceptable” means that the carrier is compatible with the active ingredient of the composition (and preferably, capable of stabilizing the active ingredient) and not deleterious to the subject to be treated. Examples of pharmaceutically acceptable excipients (carriers), including buffers, would be apparent to the skilled artisan and have been described previously. See, e.g., Remington: The Science and Practice of Pharmacy 20th Ed. (2000) Lippincott Williams and Wilkins, Ed. K. E. Hoover. In one example, a pharmaceutical composition described herein contains more than one antibody that specifically binds a GARP-TGFβ1 complex, a LTBP1-TGFβ1 complex, a LTBP3-TGFβ1 complex, and/or a LRRC33-TGFβ1 complex where the antibodies recognize different epitopes/residues of the a GARP-TGFβ1 complex, a LTBP1-TGFβ1 complex, a LTBP3-TGFβ1 complex, and/or a LRRC33-TGFβ1 complex.

The pharmaceutical compositions to be used in the present methods can comprise pharmaceutically acceptable carriers, excipients, or stabilizers in the form of lyophilized formulations or aqueous solutions (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In some examples, the pharmaceutical composition described herein comprises liposomes containing an antibody that specifically binds a GARP-TGFβ1 complex, a LTBP1-TGFβ1 complex, a LTBP3-TGFβ1 complex, and/or a LRRC33-TGFβ1 complex, which can be prepared by any suitable method, such as described in Epstein et al., Proc. Natl. Acad. Sci. USA 82:3688 (1985); Hwang et al. Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, pharmaceutical compositions of the invention may comprise or may be used in conjunction with an adjuvant. It is contemplated that certain adjuvant can boost the subject's immune responses to, for example, tumor antigens, and facilitate Teffector function, DC differentiation from monocytes, enhanced antigen uptake and presentation by APCs, etc. Suitable adjuvants include but are not limited to retinoic acid-based adjuvants and derivatives thereof, oil-in-water emulsion-based adjuvants, such as MF59 and other squalene-containing adjuvants, Toll-like receptor (TRL) ligands, α-tocopherol (vitamin E) and derivatives thereof.

The antibodies that specifically bind a GARP-TGFβ1 complex, a LTBP1-TGFβ1 complex, a LTBP3-TGFβ1 complex, and/or a LRRC33-TGFβ1 complex may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Exemplary techniques have been described previously, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 mg to about 500 mg of the active ingredient of the present disclosure. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an antibody that specifically binds a GARP-TGFβ1 complex, a LTBP1-TGFβ1 complex, a LTBP3-TGFβ1 complex, and/or a LRRC33-TGFβ1 complex with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Selection of Therapeutic Indications and/or Subjects Likely to Respond to a Therapy Comprising a TGFβ1-Selective, Broadly-Inhibiting Agent

Two inquiries may be made as to the identification/selection of suitable indications and/or patient populations for which isoform-specific context-permissive inhibitors of TGFβ1, such as those described herein, are likely to have advantageous effects: i) whether the disease is driven by or dependent on predominantly the TGFβ1 isoform over the other isoforms in human; and, ii) whether the disease involves dysregulation of multiple aspects of TGFβ1 function.

Differential expression of the three known TGFβ isoforms, namely, TGFβ1, TGFβ2, and TGFβ3, has been observed under normal (healthy; homeostatic) as well as disease conditions in various tissues. Nevertheless, the concept of isoform selectivity has neither been fully exploited nor achieved with conventional approaches that favor pan-inhibition of TGFβ across multiple isoforms. Moreover, expression patterns of the isoforms may be differentially regulated, not only in normal (homeostatic) vs, abnormal (pathologic) conditions, but also in different subpopulations of patients. Because most preclinical studies are conducted in a limited number of animal models, data obtained with the use of such models may be biased, resulting in misinterpretations of data or misleading conclusions as to the applicability to human conditions.

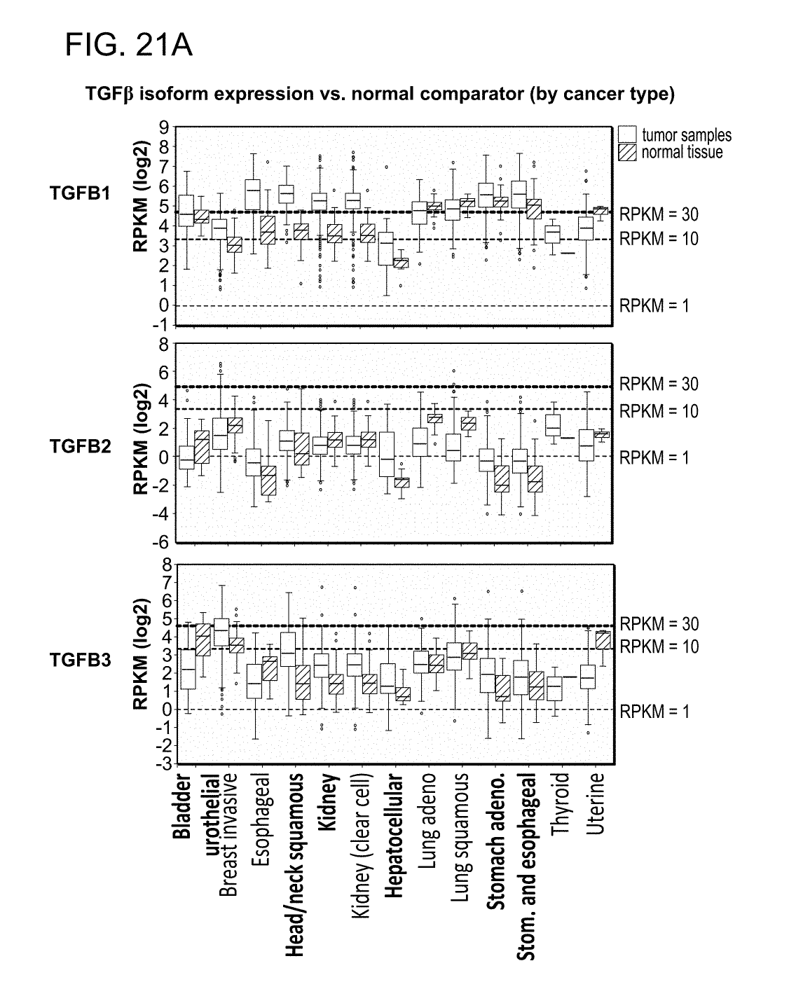

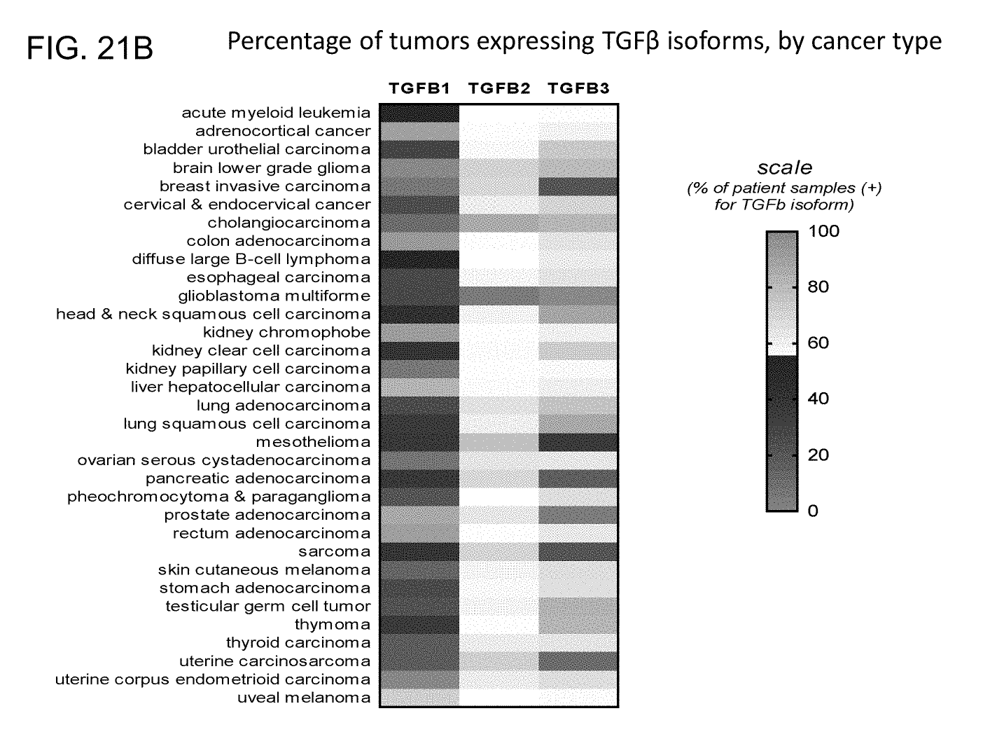

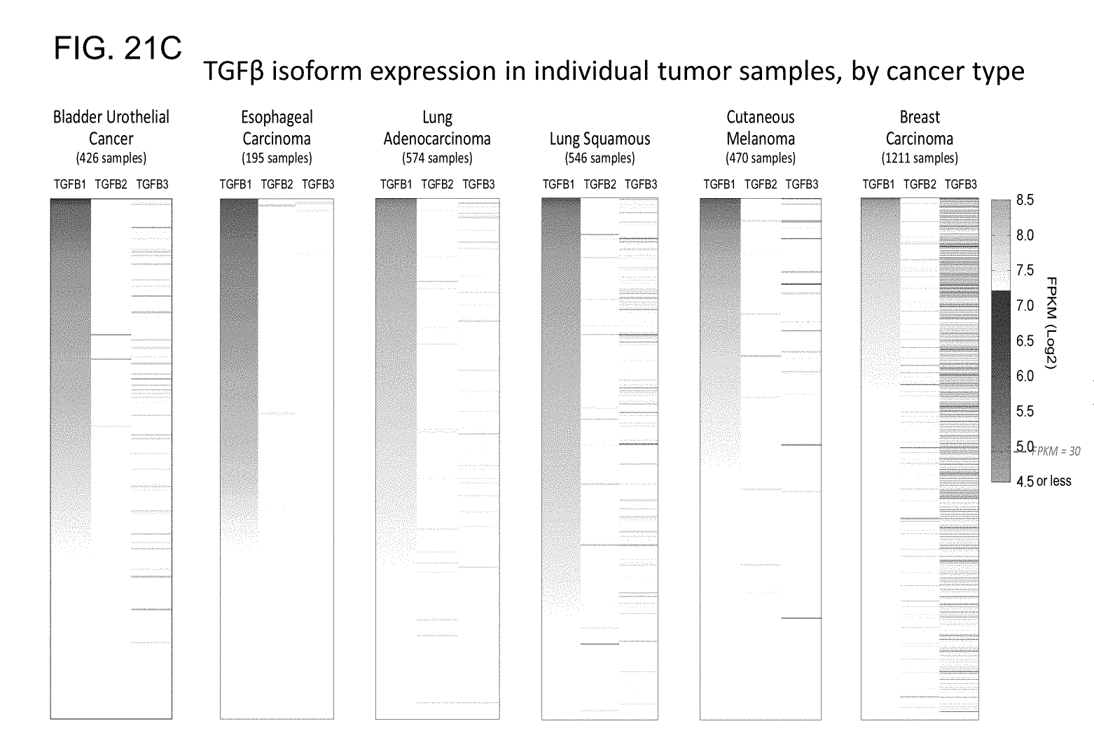

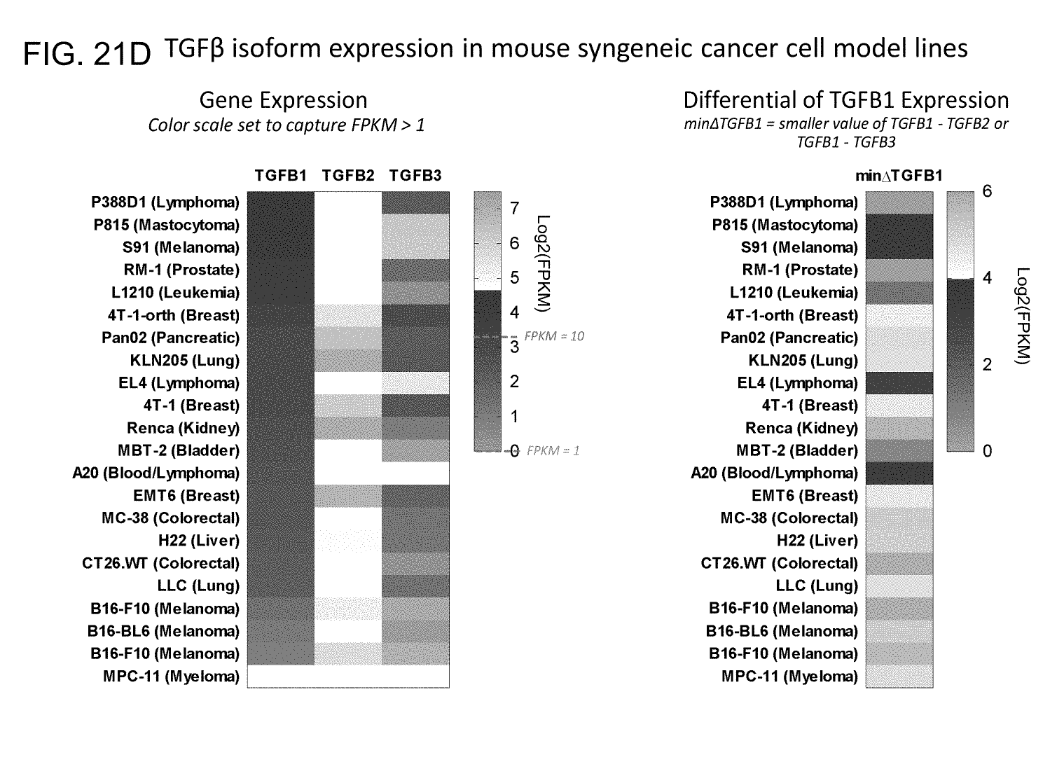

Accordingly, the present invention includes the recognition that differential expression of TGFβ isoforms must be taken into account in predicting effectiveness of particular inhibitors, as well as in interpretating preclinical data as to the translationability into human conditions. As exemplified in FIG. 21, TGFβ1 and TGFβ3 are co-dominant in certain murine syngeneic cancer models (e.g., EMT-6 and 4T1) that are widely used in preclinical studies. By contrast, numerous other cancer models (e.g., S91, B16 and MBT-2) express almost exclusively TGFβ1, similar to that observed in many human tumors, in which TGFβ1 appears to be more frequently the dominant isoform over TGFβ2/3. Furthermore, the TGFβ isoform(s) predominantly expressed under homeostatic conditions may not be the disease-associated isoform(s). For example, in normal lung tissues in healthy rats, tonic TGFβ signaling appears to be mediated mainly by TGFβ3. However, TGFβ1 appears to become markedly upregulated in disease conditions, such as lung fibrosis. Taken together, it is beneficial to test or confirm relative expression of TGFβ isoforms in clinical samples so as to select suitable therapeutics to which the patient is likely to respond.

As described herein, the isoform-selective TGFβ1 inhibitors are particularly advantageous for the treatment of diseases in which the TGFβ1 isoform is predominantly expressed relative to the other isoforms. As an example, FIG. 21D provides a non-limiting list of human cancer clinical samples with relative expression levels of TGF1 (left), TGFβ2 (center) and TGFβ3 (right). Each horizontal lime across the three isoforms represents a single patient. As can be seen, overall TGFβ1 expression is significantly higher in most of these human tumors than the other two isoforms across many tumor types, suggesting that TGFβ1-selective inhibition may be beneficial. Certain exceptions should be noted, however. First, such trend is not always applicable in certain individual patients. That is, even in a type of cancer that shows almost uniformly TGFβ1-dominance over TGFβ2/3, there are a few individuals that do not follow this general rule. Patients that fall within the minority subpopulation therefore may not respond to an isoform-specific inhibitor therapy in the way that works for a majority of patients. Second, there are a few cancer types in which TGFβ1 is co-dominant with another isoform or in which TGFβ2 and/or TGFβ3 expression is significantly greater than TGFβ1. In these situations, TGFβ1-selective inhibitors such as those described herein are not likely to be efficatious. Therefore, it is beneficial to test or confirm relative expression levels of the three TGFβ isoforms (i.e., TGFβ1, TGFβ2 and TGFβ3) in clinical samples collected from individual patients. Such information may provide better prediction as to the effectiveness of a particular therapy in individual patients, which can help ensure selection of appropriate treatment (e.g., individualized treatment) in order to increase the likelihood of a clinical response.

Accordingly, the invention includes a method for selecting a patient population or a subject who is likely to respond to a therapy comprising an isoform-specific, context-permissive TGFβ1 inhibitor. Such method comprises the steps of: providing a biological sample (e.g., clinical sample) collected from a subject, determining (e.g., measuring or assaying) relative levels of TGFβ1, TGFβ2 and TGFβ3 in the sample, and, administering to the subject a composition comprising an isoform-specific, context-permissive TGFβ1 inhibitor, if TGFβ1 is the dominant isoform over TGFβ2 and TGFβ3; and/or, if TGFβ1 is significantly overexpressed or upregulated as compared to control. Relative levels of the isoforms may be determined by RNA-based assays and/or protein-based assays, which are well-known in the art. In some embodiments, the step of administration may also include another therapy, such as immune checkpoint inhibitors, or other agents provided elsewhere herein. Such methods may optionally include a step of evaluating a therapeutic response by monitoring changes in relative levels of TGFβ1, TGFβ2 and TGFβ3 at two or more time points. In some embodiments, clinical samples (such as biopsies) are collected both prior to and following administration. In some embodiments, clinical samples (such as biopsies) are collected multiple times following treatment to assess in vivo effects over time.

In addition to the first inquiry drawn to the aspect of isoform sepectivity, the second inquiry interrogates the breadth of TGFβ1 function involved in a particular disease. This may be represented by the number of TGFβ1 contexts, namely, which presenting molecule(s) mediate disease-associated TGFβ1 function. TGFβ1-specific, broad-context inhibitors, such as context-permissive and context-independent inhibitors, are advantageous for the treatment of diseases that involve both an ECM component and an immune component of TGFβ1 function. Such disease may be associated with dysregulation in the ECM as well as perturbation in immune cell function or immune response. Thus, the TGFβ1 inhibitors described herein are capable of targeting ECM-associated TGFβ1 (e.g., presented by LTBP1 or LTBP3) as well as immune cell-associated TGFβ1 (e.g., presented by GARP or LRRC33). In some embodiments, such inhibitors target at least three of the following therapeutic targets (e.g., “context-permissive” inhibitors): GARP-associated pro/latent TGFβ1; LRRC33-associated pro/latent TGFβ1; LTBP1-associated pro/latent TGFβ1; and, LTBP3-associated pro/latent TGFβ1. In some embodiments, such inhibitors inhibit all four of the therapeutic targets (e.g., “context-independent” inhibitors): GARP-associated pro/latent TGFβ1; LRRC33-associated pro/latent TGFβ1; LTBP1-associated pro/latent TGFβ1; and, LTBP3-associated pro/latent TGFβ1, so as to broadly inhibit TGFβ1 function in these contexts.

Whether or not a particular condition of a patient involves or is driven by multiple aspects of TGFβ1 function may be assessed by evaluating expression profiles of the presenting molecules, in a clinical sample collected from the patient. Various assays are known in the art, including RNA-based assays and protein-based assays, which may be performed to obtain expression profiles. Relative expression levels (and/or changes/alterations thereof) of LTBP1, LTBP3, GARP, and LRRC33 in the sample(s) may indicate the source and/or context of TGFβ1 activities associated with the condition. For instance, a biopsy sample taken from a solid tumor may exhibit high expression of all four presenting molecules. For example, LTBP1 and LTBP3 may be highly expressed in CAFs within the tumor stroma, while GARP and LRRC33 may be highly expressed by tumor-associated immune cells, such as Tregs and leukocyte infiltrate, respectively.

Accordingly, the invention includes a method for determining (e.g., testing or confirming) the involvement of TGFβ1 in the disease, relative to TGFβ2 and TGFβ3. In some embodiments, the method further comprises a step of: identifying a source (or context) of disease-associated TGFβ1. In some embodiments, the source/context is assessed by determining the expression of TGFβ presenting molecules, e.g., LTBP1, LTBP3, GARP and LRRC33 in a clinical sample taken from patients.

Isoform-selective TGFβ1 inhibitors, such as those described herein, may be used to treat a wide variety of diseases, disorders and/or conditions that are associated with TGFβ1 dysregulation (i.e., TGFβ1-related indications) in human subjects, As used herein, “disease (disorder or condition) associated with TGFβ1 dysregulation” or “TGFβ1-related indication” means any disease, disorder and/or condition related to expression, activity and/or metabolism of a TGFβ1 or any disease, disorder and/or condition that may benefit from inhibition of the activity and/or levels TGFβ1.

Accordingly, the present invention includes the use of an isoform-specific, context-permissive TGFβ1 inhibitor in a method for treating a disease associated with TGFβ1 dysregulation in a human subject. Such inhibitor is typically formulated into a pharmaceutical composition that further comprises a pharmaceutically acceptable excipient. Advantageously, the inhibitor targets both ECM-associated TGFβ1 and immune cell-associated TGFβ1 but does not target TGFβ2 or TGFβ3 in vivo. In some embodiments, the inhibitor inhibits the activation step of TGFβ1. The disease is characterized by dysregulation or impairment in at least two of the following attributes: a) regulatory T cells (Treg); b) effector T cell (Teff) proliferation or function; c) myeloid cell proliferation or differentiation; d) monocyte recruitment or differentiation; e) macrophage function; f) epithelial-to-mesencymal transition (EMT) and/or endothelial-to-mesenchymal transition (EndMT); g) gene expression in one or more of marker genes selected from the group consisting of: PAI-1, ACTA2, CCL2, Col1a1, Col3a1, FN-1, CTGF, and TGFβ1; h) ECM components or function; i) fibroblast differentiation. A therapeutically effective amount of such inhibitor is administered to the subject suffering from or diagnosed with the disease.

In some embodiments, the disease involves dysregulation or impairment of ECM components or function comprises that show increased collagen I deposition.

In some embodiments, the dysregulation or impairment of fibroblast differentiation comprises increased myofibroblasts or myofibroblast-like cells. In some embodiments, the myofibroblasts or myofibroblast-like cells are cancer-associated fibroblasts (CAFs). In some embodiments, the CAFs are associated with a tumor stroma and may produce CCL2/MCP-1 and/or CXCL12/SDF-1.

In some embodiments, the dysregulation or impairment of regulatory T cells comprises increased Treg activity.

In some embodiments, the dysregulation or impairment of effector T cell (Teff) proliferation or function comprises suppressed CD4+/CD8+ cell proliferation.

In some embodiments, the dysregulation or impairment of myeloid cell proliferation or differentiation comprises increased proliferation of myeloid progenitor cells. The increased proliferation of myeloid cells may occur in a bone marrow,

In some embodiments, the dysregulation or impairment of monocyte differentiation comprises increased differentiation of bone marrow-derived and/or tissue resident monocytes into macrophages at a disesase site, such as a fibrotic tissue and/or a solid tumor.

In some embodiments, the dysregulation or impairment of monocyte recruitment comprises increased bone marrow-derived monocyte recruitment into a disease site such as TME, leading to increased macrophage differentiation and M2 polarization, followed by increased TAMs.

In some embodiments, the dysregulation or impairment of macrophage function comprises increased polarization of the macrophages into M2 phenotypes.

In some embodiments, the dysregulation or impairment of myeloid cell proliferation or differentiation comprises an increased number of Tregs, MDSCs and/or TANs.

TGFβ-related indications may include conditions comprising an immune-excluded disease microenvironment, such as tumor or cancerous tissue that suppresses the body's normal defense mechanism/immunity in part by excluding effector immune cells (e.g., CD4+ and/or CD8+ T cells). In some embodiments, such immune-excluding conditions are associated with poor responsiveness to treatment. Without intending to be bound by particular theory, it is contemplated that TGFβ inhibitors, such as those described herein, may help counter the tumor's ability to exclude anti-cancer immunity by restoring T cell access.

Non-limiting examples of TGFβ-related indications include: fibrosis, including organ fibrosis (e.g., kidney fibrosis, liver fibrosis, cardiac/cardiovascular fibrosis and lung fibrosis), scleroderma, Alport syndrome, cancer (including, but not limited to: blood cancers such as leukemia, myelofibrosis, multiple myeloma, colon cancer, renal cancer, breast cancer, malignant melanoma, glioblastoma), fibrosis associated with solid tumors (e.g., cancer desmoplasia, such as desmoplastic melanoma, pancreatic cancer-associated desmoplasia and breast carcinoma desmoplasia), stromal fibrosis (e.g., stromal fibrosis of the breast), radiation-induced fibrosis (e.g., radiation fibrosis syndrome), facilitation of rapid hematopoiesis following chemotherapy, bone healing, wound healing, dementia, myelofibrosis, myelodysplasia (e.g., myelodysplasic syndromes or MDS), a renal disease (e.g., end-stage renal disease or ESRD), unilateral ureteral obstruction (UUO), tooth loss and/or degeneration, endothelial proliferation syndromes, asthma and allergy, gastrointestinal disorders, anemia of the aging, aortic aneurysm, orphan indications (such as Marfan's syndrome and Camurati-Engelmann disease), obesity, diabetes, arthritis, multiple sclerosis, muscular dystrophy, amyotrophic lateral sclerosis (ALS), Parkinson's disease, osteoporosis, osteoarthritis, osteopenia, metabolic syndromes, nutritional disorders, organ atrophy, chronic obstructive pulmonary disease (COPD), and anorexia. Additional indications may include any of those disclosed in US Pub. No. 2013/0122007, U.S. Pat. No. 8,415,459 or International Pub. No. WO 2011/151432, the contents of each of which are herein incorporated by reference in their entirety.

In preferred embodiments, antibodies, antigen binding portions thereof, and compositions of the disclosure may be used to treat a wide variety of diseases, disorders and/or conditions associated with TGFβ1 signaling. In some embodiment, target tissues/cells preferentially express the TGFβ1 isoform over the other isoforms. Thus, the invention includes methods for treating such a condition associated with TGFβ1 expression (e.g., dysregulation of TGFβ1 signaling and/or upregulation of TGFβ1 expression) using a pharmaceutical composition that comprises an antibody or antigen-binding portion thereof described herein.

In some embodiments, the disease involves TGFβ1 associated with (e.g., presented on or deposited from) multiple cellular sources. In some embodiments, such disease involves both an immune component and an ECM component of TGFβ1 function. In some embodiments, such disease involves: i) dysregulation of the ECM (e.g., overproduction/deposition of ECM components such as collagens and proteases; altered stiffness of the ECM substrate; abnormal or pathological activation or differentiation of fibroblasts, such as myofibroblasts and CAFs); ii) immune suppression due to increased Tregs and/or suppressed effector T cells (Teff), e.g., elevated ratios of Treg/Teff; increased leukocyte infiltrate (e.g., macrophage and MDSCs) that causes suppression of CD4 and/or CD8 T cells; and/or iii) abnormal or pathological activation, differentiation, and/or recruitment of myeloid cells, such as macrophages (e.g., bone marrow-derived monocytic/macrophages and tissue resident macropahges), monocytes, myeloid-derived suppresser cells (MDSCs), neutrophils, dendritic cells, and NK cells.

In some embodiments, the condition involves TGFβ1 presented by more than one types of presenting molecules (e.g., two or more of: GAPR, LRRC33, LTBP1 and/or LTBP3). In some embodiments, an affected tissues/organs/cells that include TGFβ1 from multiple cellular sources. To give but one example, a solid tumor (which may also include a proliferative disease involving the bone marrow, e.g., myelofibrosis and multiple myeloma) may include TGFβ1 from multiple sources, such as the cancer cells, stromal cells, surrounding healthy cells, and/or infiltrating immune cells (e.g., CD45+ leukocytes), involving different types of presenting molecules. Relevant immune cells include but are not limited to myeloid cells and lymphoid cells, for example, neutrophils, eosinophils, basophils, lymphocytes (e.g., B cells, T cells, and NK cells), and monocytes. Context-independent or context-permissive inhibitors of TGFβ1 may be useful for treating such conditions.

Non-limiting examples of conditions or disorders that may be treated with isoform-specific context-permissive inhibitors of TGFβ1, such as antibodies or fragments thereof described herein, are provided below.

Diseases with Aberrant Gene Expression:

It has been observed that abnormal activation of the TGFβ1 signal transduction pathway in various disease conditions is associated with altered gene expression of a number of markers. These gene expression markers (e.g., as measured by mRNA) include, but are not limited to: Serpine 1 (encoding PAI-1), MCP-1 (also known as CCL2), Col1a1, Col3a1, FN1, TGFβ1, CTGF, and ACTA2 (encoding α-SMA). Interestingly, many of these genes are implicated to play a role in a diverse set of disease conditions, including various types of organ fibrosis, as well as in many cancers, which include myelofibrosis. Indeed, pathophysiological link between fibrotic conditions and abnormal cell proliferation, tumorigenesis and metastasis has been suggested. See for example, Cox and Erler (2014) Clinical Cancer Research 20(14): 3637-43 “Molecular pathways: connecting fibrosis and solid tumor metastasis”; Shiga et al. (2015) Cancers 7:2443-2458 “Cancer-associated fibroblasts: their characteristics and their roles in tumor growth”; Wynn and Barron (2010) Semin. Liver Dis. 30(3): 245-257 “Macrophages: master regulators of inflammation and fibrosis”, contents of which are incorporated herein by reference. Without wishing to be bound by a particular theory, the inventors of the present disclosure contemplate that the TGFβ1 signaling pathway may in fact be a key link between these broad pathologies.

For example, MCP-1/CCL2 is thought to play a role in both fibrosis and cancer. MCP-1/CCL2 is characterized as a profibrotic chemokine and is a monocyte chemoattractant, and evidence suggests that it may be involved in both initiation and progression of cancer. In fibrosis, MCP-1/CCL2 has been shown to play an important role in the inflammatory phase of fibrosis. For example, neutralization of MCP-1 resulted in a dramatic decrease in glomerular crescent formation and deposition of type I collagen.

The ability of MCP-1/CCL2 to recruit monocytes/macrophages has crucial consequences in cancer progression. Tumor-derived MCP-1/CCL2 can promote “pro-cancer” phenotypes in macrophages. For example, in lung cancer, MCP-1/CCL2 has been shown to be produced by stromal cells and promote metastasis. In human pancreatic cancer, tumors secrete CCL2, and immunosuppressive CCR2-positive macrophages infiltrate these tumors. Patients with tumors that exhibit high CCL2 expression/low CD8 T-cell infiltrate have significantly decreased survival.

Similarly, involvement of PAI-1/Serpine1 has been implicated in a variety of cancers, angiogenesis, inflammation, neurodegenerative diseases (e.g., Alzheimer's Disease). Elevated expression of PAI-1 in tumor and/or serum is correlated with poor prognosis (e.g., shorter survival, increased metastasis) in various cancers, such as breast cancer and bladder cancer (e.g., transitional cell carcinoma) as well as myelofibrosis. In the context of fibrotic conditions, PAI-1 has been recognized as an important downstream effector of TGFβ1-induced fibrosis, and increased PAI-1 expression has been observed in various forms of tissue fibrosis, including lung fibrosis (such as IPF), kidney fibrosis, liver fibrosis and scleroderma.

In some embodiments, in vivo effects of the TGFβ1 inhibitor therapy may be assessed by measuring changes in gene markers. Suitable markers include TGFβ (e.g., TGFβ1, TGFβ2, and TGFβ3). In some embodiments, suitable markers include mesenchymal transition genes (e.g., AXL, ROR2, WNT5A, LOXL2, TWIST2, TAGLN, and/or FAP), immunosuppressive genes (e.g., IL10, VEGFA, VEGFC), monocyte and macrophage chemotactic genes (e.g., CCL2, CCL7, CCL8 and CCL13), and/or various fibrotic markers discussed herein. Preferred markers are plasma markers.

As shown in the Example herein, isoform-specific, context-independent inhibitors of TGFβ1 described herein can reduce expression levels of many of these markers in a mechanistic animal model, such as UUO, which has been shown to be TGFβ1-dependent. Therefore, such inhibitors may be used to treat a disease or disorder characterized by abnormal expression (e.g., overexpression/upregulation or underexpression/downregulation) of one or more of the gene expression markers.

Thus, in some embodiments, an isoform-specific, context-permissive or context-independent inhibitor of TGFβ1 is used in the treatment of a disease associated with overexpression of one or more of the following: PAI-1 (also known as Serpine1), MCP-1 (also known as CCL2), Col1a1, Col3a1, FN1, TGFβ1, CTGF, α-SMA, ITGA11, and ACTA2, wherein the treatment comprises administration of the inhibitor to a subject suffering from the disease in an amount effective to treat the disease. In some embodiments, the inhibitor is used to treat a disease associated with overexpression of PAI-1, MCP-1/CCL2, CTGF, and/or α-SMA. In some embodiments, the disease is myelofibrosis. In some embodiments, the disease is cancer, for example, cancer comprising a solid tumor. In some embodiments, the disease is organ fibrosis, e.g., fibrosis of the liver, the kidney, the lung and/or the cardiac or cardiovascular tissue.

### Diseases Involving Proteases:

Activation of TGFβ from its latent complex may be triggered by integrin in a force-dependent manner, and/or by proteases. Evidence suggests that certain classes of proteases may be involved in the process, including but are not limited to Ser/Thr proteases such as Kallikreins, chemotrypsin, elastases, plasmin, as well as zinc metalloproteases of MMP family, such as MMP-2, MMP-9 and MMP-13. MMP-2 degrades the most abundant component of the basement membrane, Collagen IV, raising the possibility that it may play a role in ECM-associated TGFβ1 regulation. MMP-9 has been implicated to play a central role in tumor progression, angiogenesis, stromal remodeling and metastasis. Thus, protease-dependent activation of TGFβ1 in the ECM may be important for treating cancer.

Kallikreins (KLKs) are trypsin- or chymotrypsin-like serine proteases that include plasma Kallikreins and tissue Kallikreins. The ECM plays a role in tissue homeostasis acting as a structural and signaling scaffold and barrier to suppress malignant outgrowth. KLKs may play a role in degrading ECM proteins and other components which may facilitate tumor expansion and invasion. For example, KLK1 is highly upregulated in certain breast cancers and can activate pro-MMP-2 and pro-MMP-9. KLK2 activates latent TGFβ1, rendering prostate cancer adjacent to fibroblasts permissive to cancer growth. KLK3 has been widely studied as a diagnostic marker for prostate cancer (PSA). KLK3 may directly activate TGFβ1 by processing plasminogen into plasmin, which proteolytically cleaves LAP. KLK6 may be a potential marker for Alzheimer's disease.

Moreover, data provided in Example 8 indicate that such proteases may be a Kallikrein. Thus, the invention encompasses the use of an isoform-specific, context-independent or permissive inhibitor of TGFβ in a method for treating a disease associated with Kallikrein or a Kallikrein-like protease.

Known activators of TGFβ1, such as plasmin, TSP-1 and αVβ6 integrin, all interact directly with LAP. It is postulated that proteolytic cleavage of LAP may destabilize the LAP-TGFβ interaction, thereby releasing active TGFβ1. It has been suggested that the region containing 54-LSKLRL-59 is important for maintaining TGFβ1 latency. Thus, agents (e.g., antibodies) that stabilize the interaction, or block the proteolytic cleavage of LAP may prevent TGFβ activation.

### Diseases Involving Epithelial-to-Mesenchymal Transition (EMT):

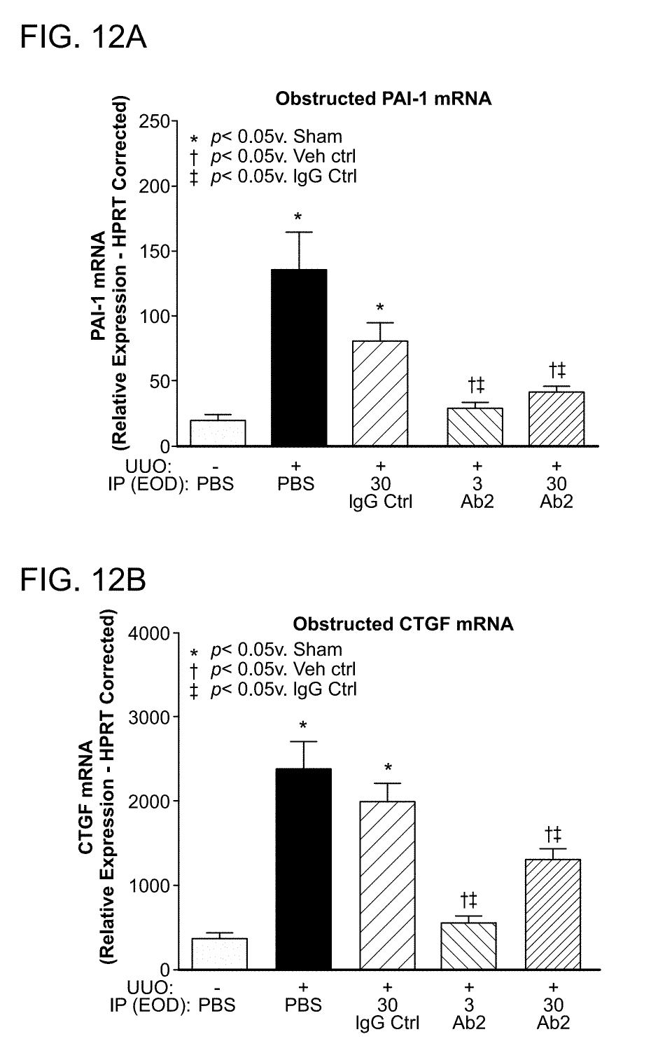

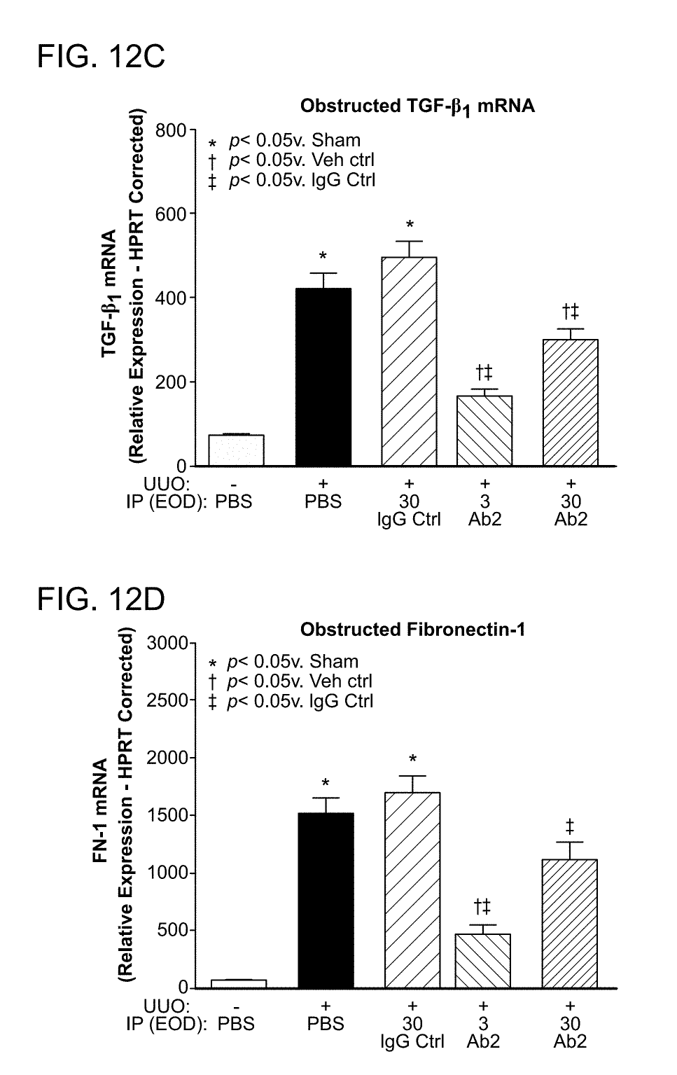

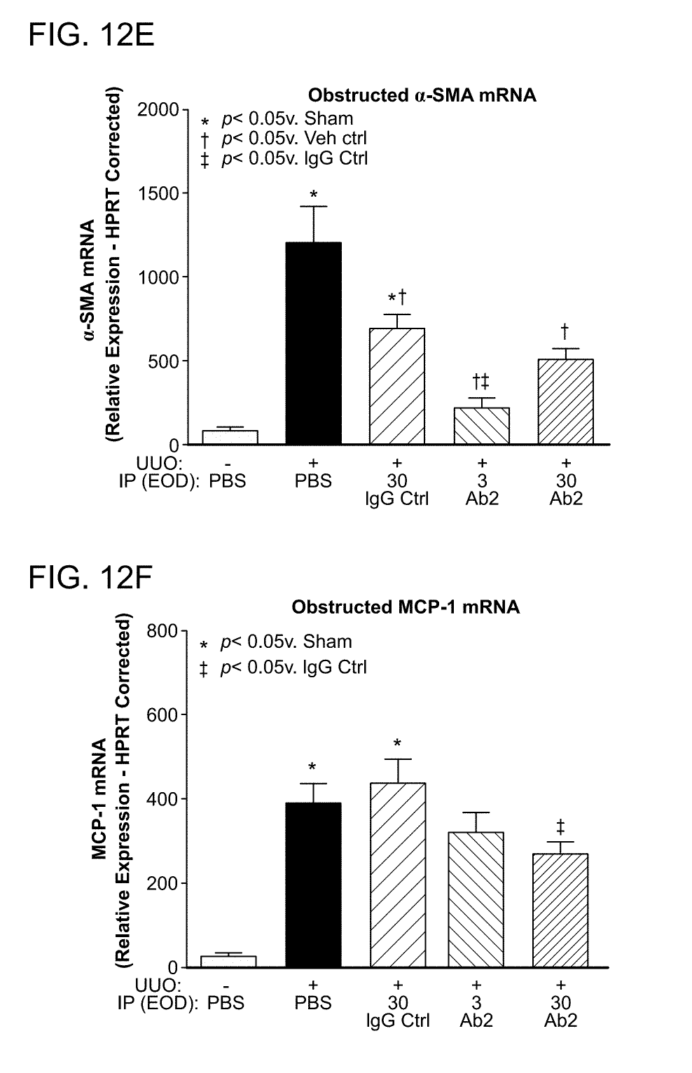

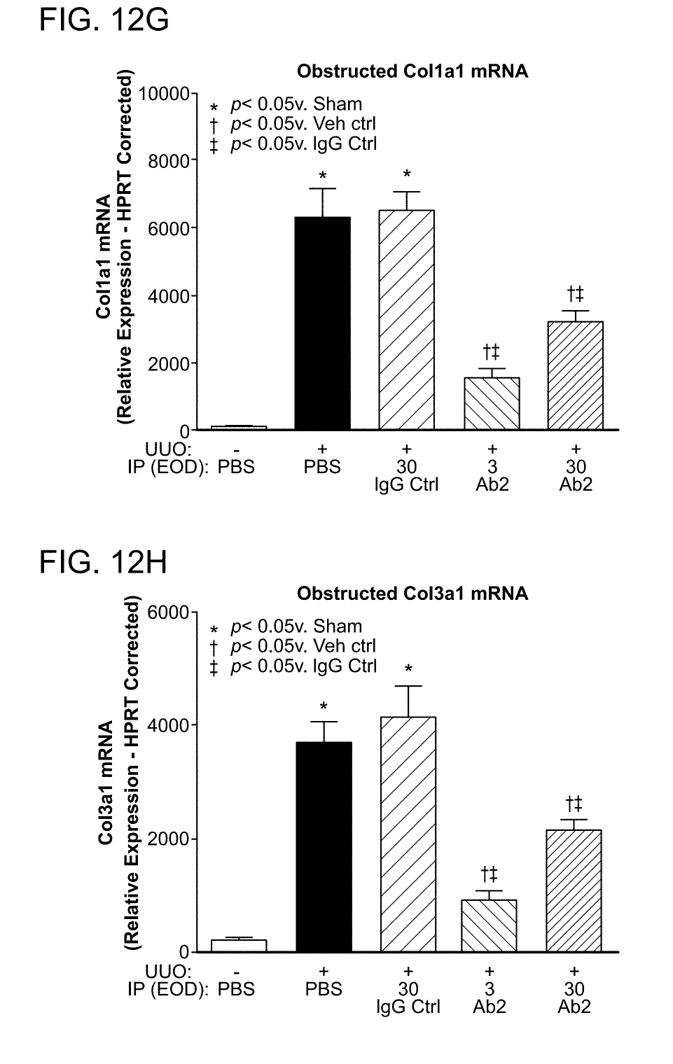

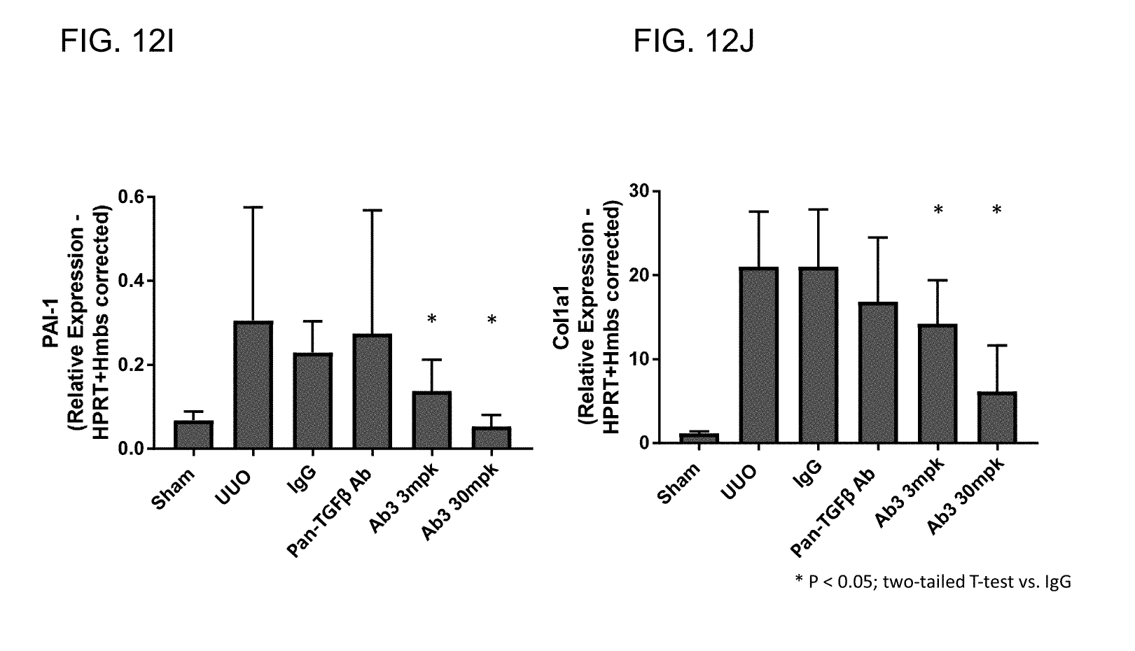

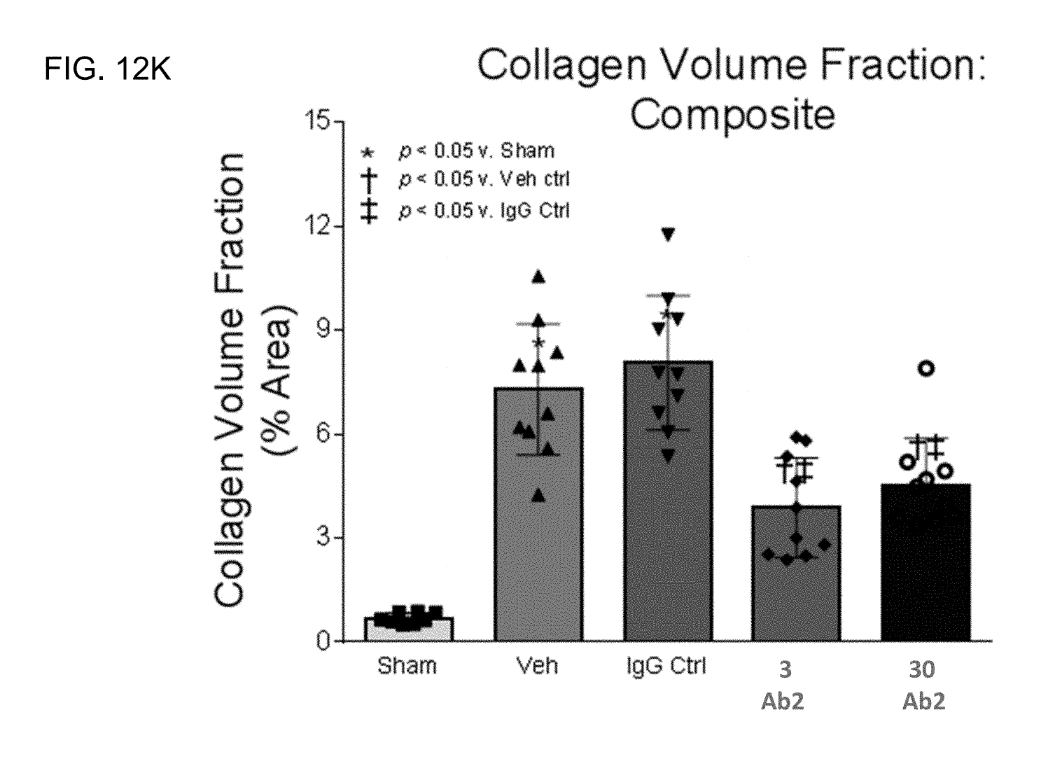

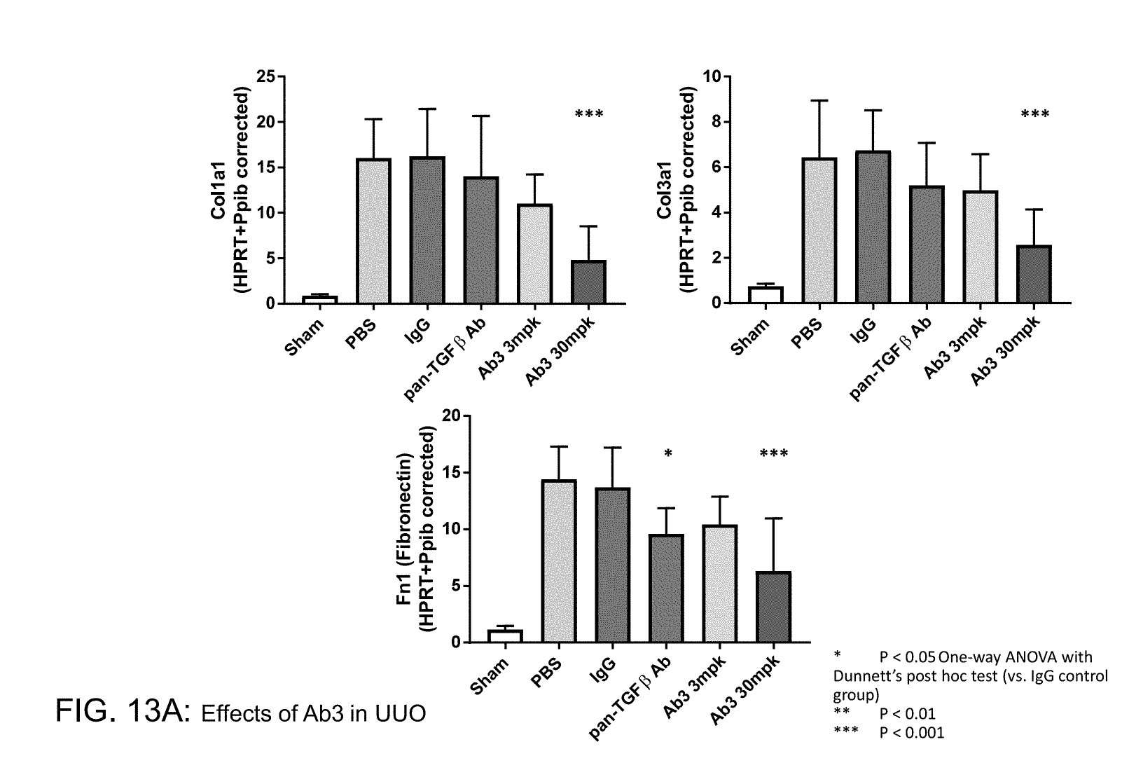

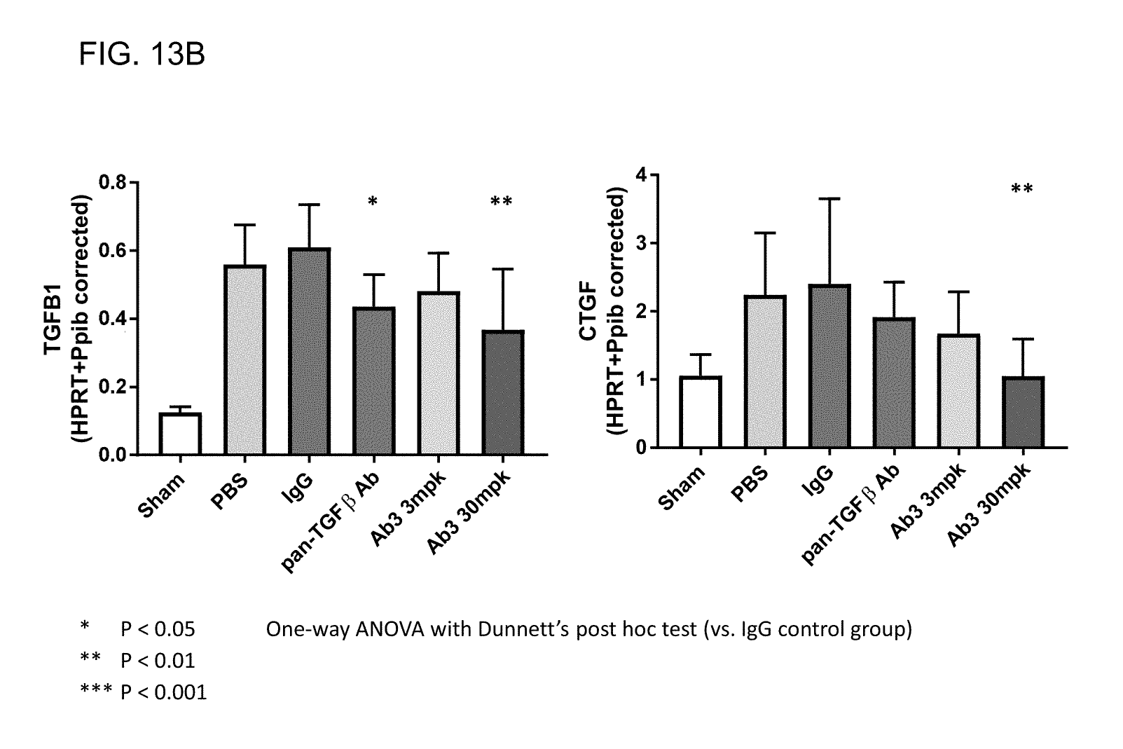

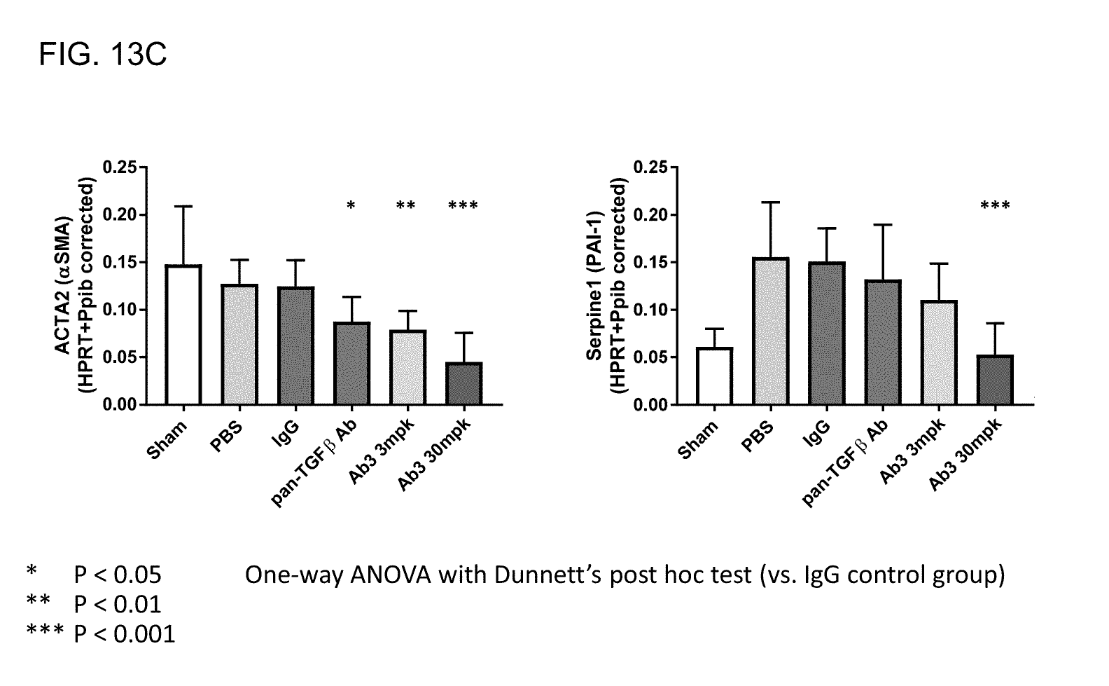

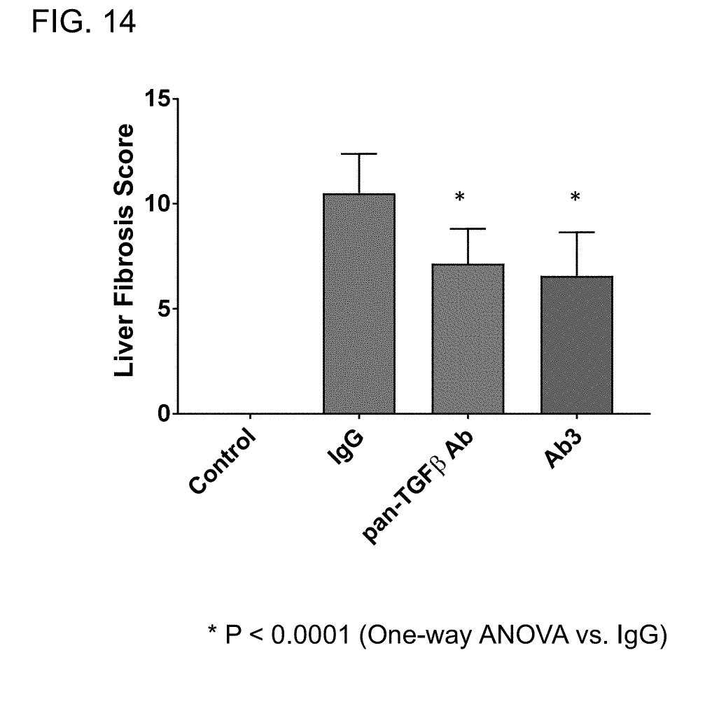

EMT (epithelial mesenchymal transition) is the process by which epithelial cells with tight junctions switch to mesenchymal properties (phenotypes) such as loose cell-cell contacts. The process is observed in a number of normal biological processes as well as pathological situations, including embryogenesis, wound healing, cancer matastasis and fibrosis (reviewed in, for example, Shiga et al. (2015) “Cancer-Associated Fibroblasts: Their Characteristics and Their Roles in Tumor Growth.” Cancers, 7: 2443-2458). Generally, it is believed that EMT signals are induced mainly by TGFβ. Many types of cancer, for example, appear to involve transdifferentiation of cells towards mesenchymal phenotype (such as CAFs) which correlate with poorer prognosis. Thus, isoform-specific, context-permissive inhibitors of TGFβ1, such as those described herein, may be used to treat a disease that is initiated or driven by EMT. Indeed, data exemplified herein (e.g., FIGS. 12 and 13) show that such inhibitors have the ability to suppress expression of CAF markers in vivo, such as α-SMA, Col1 (Type I collagen), and FN (fibronectin).

### Diseases Involving Endothelial-to-Mesenchymal Transition (EndMT):

Similarly, TGFβ is also a key regulator of the endothelial-mesenchymal transition (EndMT) observed in normal development, such as heart formation. However, the same or similar phenomenon is also seen in many diseases, such as cancer stroma. In some disease processes, endothelial markers such as CD31 become downregulated upon TGFβ1 exposure and instead the expression of mesenchymal markers such as FSP-1, α-SMA and fibronectin becomes induced. Indeed, stromal CAFs may be derived from vascular endothelial cells. Thus, isoform-specific, context-permissive inhibitors of TGFβ1, such as those described herein, may be used to treat a disease that is initiated or driven by EndMT.

### Diseases Involving Matrix Stiffening and Remodeling:

Progression of fibrotic conditions involves increased levels of matrix components deposited into the ECM and/or maintenance/remodeling of the ECM. TGFβ1 at least in part contributes to this process. This is supported, for example, by the observation that increased deposition of ECM components such as collagens can alter the mechanophysical properties of the ECM (e.g., the stiffness of the matrix/substrate) and this phenomenon is associated with TGFβ1 signaling. To confirm this notion, the present inventors have evaluated the role of matrix stiffness in affecting integrin-dependent activation of TGFβ in primary fibroblasts transfected with proTGFβ and LTBP1, and grown on silicon-based substrates with defined stiffness (e.g., 5 kPa, 15 kPa or 100 kPa). As summarized in the Example section below, matrices with greater stiffness enhance TGFβ1 activation, and this can be suppressed by isoform-specific, context-permissive inhibitors of TGFβ1, such as those described herein. These observations suggest that TGFβ1 influences ECM properties (such as stiffness), which in turn can further induce TGFβ1 activation, reflective of disease progression. Thus, isoform-specific, context-permissive inhibitors of TGFβ1, such as those described herein may be used to block this process to counter disease progression involving ECM alterations, such as fibrosis, tumor growth, invasion, metastasis and desmoplasia. The LTBP-arm of such inhibitors can directly block ECM-associated pro/latent TGFβ complexes which are presented by LTBP1 and/or LTBP3, thereby preventing activation/release of the growth factor from the complex in the disease niche. In some embodiments, the isoform-specific, context-permissive TGFβ1 inhibitors such as those described herein may normalize ECM stiffness to treat a disease that involves integrin-dependent signaling. In some embodiments, the integrin comprises an al 1 chain, 31 chain, or both.

### Fibrosis:

According to the invention, isoform-specific, context-permissive inhibitors TGFβ1 such as those described herein are used in the treatment of fibrosis (e.g., fibrotic indications, fibrotic conditions) in a subject. Suitable inhibitors to carry out the present invention include antibodies and/or compositions according to the present disclosure which may be useful for altering or ameliorating fibrosis. More specifically, such antibodies and/or compositions are selective antagonists of TGFβ1 that are capable of targeting TGFβ1 presented by various types of presenting molecules. TGFβ1 is recognized as the central orchestrator of the fibrotic response. Antibodies targeting TGFβ decrease fibrosis in numerous preclinical models. Such antibodies and/or antibody-based compounds include LY2382770 (Eli Lilly, Indianapolis, Ind.). Also included are those described in U.S. Pat. Nos. 6,492,497, 7,151,169, 7,723,486 and U.S. Appl. Publ. No. 2011/0008364, the contents of each of which are herein incorporated by reference in their entirety. Prior art TGFβ antagonists include, for example, agents that target and block integrin-dependent activation of TGFβ.

However, evidence suggests that such prior art agents may not mediate isoform-specific inhibition and may cause unwanted effects by inadvertently blocking normal function of TGFβ2 and/or TGFβ3. Indeed, data presented herein support this notion. Normal (undiseased) lung tissues contain relatively low but measurable levels of TGFβ2 and TGFβ3, but notably less TGFβ1. In comparison, in certain disease conditions such as fibrosis, TGFβ1 becomes preferentially upregulated relative to the other isoforms. Preferably, TGFβ antagonists for use in the treatment of such conditions exert their inhibitory activities only towards the disease-induced or disease-associated isoform, while preserving the function of the other isoforms that are normally expressed to mediate tonic signaling in the tissue. Advantageously, as demonstrated in Example 20 below, an isoform-specific, context-permissive TGFβ1 inhibitor encompassed by the present disclosure shows little effect in bronchoalveolar lavage (BAL) of healthy rats, supporting the notion that tonic TGFβ signaling (e.g., TGFβ2 and/or TGFβ3) is unperturbed. By contrast, prior art inhibitors (LY2109761, a small molecule TGFβ receptor antagonist, and a monoclonal antibody that targets αVβ6 integrin) both are shown to inhibit TGFβ downstream tonic signaling in non-diseased rat BAL, raising the possibility that these inhibitors may cause unwanted side effects. Alternatively or additionally, agents that target and block integrin-dependent activation of TGFβ may be capable of blocking only a subset of integrins responsible for disease-associated TGFβ1 activation, among numerous integrin types that are expressed by various cell types and play a role in the pathogenesis. Furthermore, even where such antagonists may selectively block integrin-mediated activation of the TGFβ1 isoform, it may be ineffective in blocking TGFβ1 activation triggered by other modes, such as protease-dependent activation. By contrast, the isoform-specific, context-permissive inhibitors of TGFβ1 such as those described herein are aimed to prevent the activation step of TGFβ1 regardless of the particular mode of activation, while maintaining isoform selectivity.

Fibrotic indications for which antibodies and/or compositions of the present disclosure may be used therapeutically include, but are not limited to lung indications (e.g. idiopathic pulmonary fibrosis (IPF), chronic obstructive pulmonary disorder (COPD), allergic asthma, acute lung injury, eosinophilic esophagitis, pulmonary arterial hypertension and chemical gas-injury), kidney indications (e.g., diabetic glomerulosclerosis, focal segmental glomeruloclerosis (FSGS), chronic kidney disease (CKD), fibrosis associated with kidney transplantation and chronic rejection, IgA nephropathy, and hemolytic uremic syndrome), liver fibrosis (e.g., non-alcoholic steatohepatitis (NASH), chronic viral hepatitis, parasitemia, inborn errors of metabolism, toxin-mediated fibrosis, such as alcohol fibrosis, non-alcoholic steatohepatitis-hepatocellular carcinoma (NASH-HCC), primary biliary cirrhosis, and sclerosing cholangitis), cardiovascular fibrosis (e.g., cardiomyopathy, hypertrophic cardiomyopathy, atherosclerosis and restenosis) systemic sclerosis, skin fibrosis (e.g. skin fibrosis in systemic sclerosis, diffuse cutaneous systemic sclerosis, scleroderma, pathological skin scarring, keloid, post-surgical scarring, scar revision surgery, radiation-induced scarring and chronic wounds) and cancers or secondary fibrosis (e.g. myelofibrosis, head and neck cancer, M7 acute megakaryoblastic leukemia and mucositis). Other diseases, disorders or conditions related to fibrosis (including degenerative disorders) that may be treated using compounds and/or compositions of the present disclosure, include, but are not limited to adenomyosis, endometriosis, Marfan's syndrome, stiff skin syndrome, scleroderma, rheumatoid arthritis, bone marrow fibrosis, Crohn's disease, ulcerative colitis, systemic lupus erythematosus, muscular dystrophy (such as DMD), Parkinson's disease, ALS, Dupuytren's contracture, Camurati-Engelmann disease, neural scarring, dementia, proliferative vitreoretinopathy, corneal injury, complications after glaucoma drainage surgery, and multiple sclerosis. Many such fibrotic indications are also associated with inflammation of the affected tissue(s), indicating involvement of an immune component.

In some embodiments, fibrotic indications that may be treated with the compositions and/or methods described herein include organ fibrosis, such as fibrosis of the lung (e.g., IPF), fibrosis of the kidney (e.g., fibrosis associated with CKD), fibrosis of the liver, fibrosis of the heart or cardiac tissues, fibrosis of the skin (e.g., scleroderma), fibrosis of the uterus (e.g., endometrium, myometrium), and fibrosis of the bone marrow. In some embodiments, such therapy may reduce or delay the need for organ transplantation in patients. In some embodiments, such therapy may prolong the survival of the patients.

To treat IPF, patients who may benefit from the treatment include those with familial IPF and those with sporadic IPF. Administration of a therapeutically effective amount of an isoform-specific, context-permissive inhibitor of TGFβ1 may reduce myofibroblast accumulation in the lung tissues, reduce collagen deposits, reduce IPF symptoms, improve or maintain lung function, and prolong survival. In some embodiments, the inhibitor blocks activation of ECM-associated TGFβ1 (e.g., pro/latent TGFβ1 presented by LTBP1/3) within the fibrotic environment of IPF. The inhibitor may optionally further block activation of macrophage-associated TGFβ1 (e.g., pro/latent TGFβ1 presented by LRRC33), for example, alveolar macrophages. As a result, the inhibitor may suppress fibronectin release and other fibrosis-associated factors.

The isoform-specific, context-permissive TGFβ1 inhibitors such as those provided herein may be used to treat fibrotic conditions of the liver, such as fatty liver (e.g., NASH). The fatty liver may or may not be inflamed. Inflammation of the liver due to fatty liver (i.e., steatohepatitis) may develop into scarring (fibrosis), which then often progresses to cirrhosis (scarring that distorts the structure of the liver and impairs its function). The inhibitor may therefore be used to treat such conditions. In some embodiments, the inhibitor blocks activation of ECM-associated TGFβ1 (e.g., pro/latent TGFβ1 presented by LTBP1/3) within the fibrotic environment of the liver. The inhibitor may optionally further block activation of macrophage-associated TGFβ1 (e.g., pro/latent TGFβ1 presented by LRRC33), for example, Kupffer cells (also known as stellate macrophages) as well as infiltrating monocyte-derived macrophages and MDSCs. As a result, the inhibitor may suppress fibrosis-associated factors. Administration of the inhibitor in a subject with such conditions may reduce one or more symptoms, prevent or retard progression of the disease, reduce or stabilize fat accumulations in the liver, reduce disease-associated biomarkers (such as serum collagen fragments), reduce liver scarring, reduce liver stiffness, and/or otherwise produce clinically meaningful outcome in a patient population treated with the inhibitor, as compared to a control population not treated with the inhibitor. In some embodiments, an effective amount of the inhibitor may achieve both reduced liver fat and reduced fibrosis (e.g., scarring) in NASH patients. In some embodiment, an effective amount of the inhibitor may achieve improvement in fibrosis by at least one stage with no worsening steatohepatitis in NASH patients. In some embodiments, an effective amount of the inhibitor may reduce the rate of occurrence of liver failure and/or liver cancer in NASH patients. In some embodiments, an effective amount of the inhibitor may normalize, as compared to control, the levels of multiple inflammatory or fibrotic serum biomarkers as assessed following the start of the therapy, at, for example, 12-36 weeks. In some embodiments in NASH patients, the isoform-specific, context-permissive TGFβ1 inhibitors may be administered in patients who receive one or more additional therapies, including, but are not limited to myostatin inhibitors, which may generally enhance metabolic regulation in patients with clinical manifestation of metabolic syndrome, including NASH.

The isoform-specific, context-permissive TGFβ1 inhibitors such as those provided herein may be used to treat fibrotic conditions of the kidney, e.g., diseases characterized by extracellular matrix accumulation (IgA nephropathy, focal and segmental glomerulosclerosis, crescentic glomerulonephritis, lupus nephritis and diabetic nephropathy) in which significantly increased expression of TGFβ in glomeruli and the tubulointerstitium has been observed. While glomerular and tubulointerstitial deposition of two matrix components induced by TGFβ, fibronectin EDA+ and PAI-1, was significantly elevated in all diseases with matrix accumulation, correlation analysis has revealed a close relationship primarily with the TGFβ1 isoform. Accordingly, the isoform-specific, context-permissive TGFβ1 inhibitors are useful as therapeutic for a spectrum of human glomerular disorders, in which TGFβ is associated with pathological accumulation of extracellular matrix.

In some embodiments, the fibrotic condition of the kidney is associated with chronic kidney disease (CKD). CKD is caused primarily by high blood pressure or diabetes and claims more than one million lives each year. CKD patients require lifetime medical care that ranges from strict diets and medications to dialysis and transplants. In some embodiments, the TGFβ1 inhibitor therapy described herein may reduce or delay the need for dialysis and/or transplantation. In some embodiments, such therapy may reduce the need (e.g., dosage, frequency) for other treatments. In some embodiments, the isoform-specific, context-permissive TGFβ1 inhibitors may be administered in patients who receive one or more additional therapies, including, but are not limited to myostatin inhibitors, which may generally enhance metabolic regulation in patients with CKD.

The organ fibrosis which may be treated with the methods provided herein includes cardiac (e.g., cardiovascular) fibrosis. In some embodiments, the cardiac fibrosis is associated with heart failure, e.g., chronic heart failure (CHF). In some embodiments, the heart failure may be associated with myocardial diseases and/or metabolic diseases. In some embodiments, the isoform-specific, context-permissive TGFβ1 inhibitors may be administered in patients who receive one or more additional therapies, including, but are not limited to myostatin inhibitors in patients with cardiac dysfunction that involves heart fibrosis and metabolic disorder.

In some embodiments, fibrotic conditions that may be treated with the compositions and/or methods described herein include desmoplasia. Desmoplasia may occur around a neoplasm, causing dense fibrosis around the tumor (e.g., desmoplastic stroma), or scar tissue within the abdomen after abdominal surgery. In some embodiments, desmoplasia is associated with malignant tumor. Due to its dense formation surrounding the malignancy, conventional anti-cancer therapeutics (e.g., chemotherapy) may not effectively penetrate to reach cancerous cells for clinical effects. Isoform-specific, context-permissive inhibitors of TGFβ1 such as those described herein may be used to disrupt the desmoplasia, such that the fibrotic formation can be loosened to aid effects of anti-cancer therapy. In some embodiments, the isoform-specific, context-permissive inhibitors of TGFβ1 can be used as monotherapy (more below).

To treat patients with fibrotic conditions, TGFβ1 isoform-specific, context-permissive inhibitors are administered to a subject in an amount effective to treat the fibrosis. The effective amount of such an antibody is an amount effective to achieve both therapeutic efficacy and clinical safety in the subject. In some embodiments, the inhibitor is a context-permissive antibody that can block activation of an LTBP-mediated TGFβ1 localized (e.g., tethered) in the ECM and GARP-mediated TGFβ1 localized in (e.g., tethered on) immune cells. In some embodiments, antibody is a context-permissive antibody that can block activation of an LTBP-mediated TGFβ1 localized in the ECM and LRRC33-mediated TGFβ1 localized in (e.g., tethered on) monocytes/macrophages. In some embodiments, the LTBP is LTBP1 and/or LTBP3. In some embodiments, targeting and inhibiting TGFβ1 presented by LRRC33 on profibrotic, M2-like macrophages in the fibrotic microenvironment may be beneficial.

Assays useful in determining the efficacy of the antibodies and/or compositions of the present disclosure for the alteration of fibrosis include, but are not limited to, histological assays for counting fibroblasts and basic immunohistochemical analyses known in the art.

### Myelofibrosis:

Myelofibrosis, also known as osteomyelofibrosis, is a relatively rare bone marrow proliferative disorder (cancer), which belongs to a group of diseases called myeloproliferative disorders. Myelofibrosis is classified into the Philadelphia chromosome-negative (−) branch of myeloproliferative neoplasms. Myelofibrosis is characterized by clonal myeloproliferation, aberrant cytokine production, extramedullary hematopoiesis, and bone marrow fibrosis. The proliferation of an abnormal clone of hematopoietic stem cells in the bone marrow and other sites results in fibrosis, or the replacement of the marrow with scar tissue. The term myelofibrosis, unless otherwise specified, refers to primary myelofibrosis (PMF). This may also be referred to as chronic idiopathic myelofibrosis (cIMF) (the terms idiopathic and primary mean that in these cases the disease is of unknown or spontaneous origin). This is in contrast with myelofibrosis that develops secondary to polycythemia vera or essential thrombocythaemia. Myelofibrosis is a form of myeloid metaplasia, which refers to a change in cell type in the blood-forming tissue of the bone marrow, and often the two terms are used synonymously. The terms agnogenic myeloid metaplasia and myelofibrosis with myeloid metaplasia (MMM) are also used to refer to primary myelofibrosis. In some embodiments, the hematologic proliferative disorders which may be treated in accordance with the present invention include myeloproliferative disorders, such as myelofibrosis. So-called “classical” group of BCR-ABL (Ph) negative chronic myeloproliferative disorders includes essential thrombocythemia (ET), polycythemia vera (PV) and primary myelofibrosis (PMF).

Myelofibrosis disrupts the body's normal production of blood cells. The result is extensive scarring in the bone marrow, leading to severe anemia, weakness, fatigue and often an enlarged spleen. Production of cytokines such as fibroblast growth factor by the abnormal hematopoietic cell clone (particularly by megakaryocytes) leads to replacement of the hematopoietic tissue of the bone marrow by connective tissue via collagen fibrosis. The decrease in hematopoietic tissue impairs the patient's ability to generate new blood cells, resulting in progressive pancytopenia, a shortage of all blood cell types. However, the proliferation of fibroblasts and deposition of collagen is thought to be a secondary phenomenon, and the fibroblasts themselves may not be part of the abnormal cell clone.

Myelofibrosis may be caused by abnormal blood stem cells in the bone marrow. The abnormal stem cells produce mature and poorly differentiated cells that grow quickly and take over the bone marrow, causing both fibrosis (scar tissue formation) and chronic inflammation.

Primary myelofibrosis is associated with mutations in Janus kinase 2 (JAK2), thrombopoietin receptor (MPL) and calreticulin (CALR), which can lead to constitutive activation of the JAK-STAT pathway, progressive scarring, or fibrosis, of the bone marrow occurs. Patients may develop extramedullary hematopoiesis, i.e., blood cell formation occurring in sites other than the bone marrow, as the haemopoetic cells are forced to migrate to other areas, particularly the liver and spleen. This causes an enlargement of these organs. In the liver, the abnormal size is called hepatomegaly. Enlargement of the spleen is called splenomegaly, which also contributes to causing pancytopenia, particularly thrombocytopenia and anemia. Another complication of extramedullary hematopoiesis is poikilocytosis, or the presence of abnormally shaped red blood cells.

The principal site of extramedullary hematopoiesis in myelofibrosis is the spleen, which is usually markedly enlarged in patients suffering from myelofibrosis. As a result of massive enlargement of the spleen, multiple subcapsular infarcts often occur in the spleen, meaning that due to interrupted oxygen supply to the spleen partial or complete tissue death happens. On the cellular level, the spleen contains red blood cell precursors, granulocyte precursors and megakaryocytes, with the megakaryocytes prominent in their number and in their abnormal shapes. Megakaryocytes may be involved in causing the secondary fibrosis seen in this condition.

It has been suggested that TGFβ may be involved in the fibrotic aspect of the pathogenesis of myelofibrosis (see, for example, Agarwal et al., “Bone marrow fibrosis in primary myelofibrosis: pathogenic mechanisms and the role of TGFβ” (2016) Stem Cell Investig 3:5). Bone marrow pathology in primary myelofibrosis is characterized by fibrosis, neoangeogenesis and osteosclerosis, and the fibrosis is associated with an increase in production of collagens deposited in the ECM.

A number of biomarkers have been described, alternations of which are indicative of or correlate with the disease. In some embodiments, the biomarkers are cellular markers. Such disease-associated biomarkers are useful for the diagnosis and/or monitoring of the disease progression as well as effectiveness of therapy (e.g., patients' responsiveness to the therapy). These biomarkers include a number of fibrotic markers, as well as cellular markers. In lung cancer, for example, TGFβ1 concentrations in the bronchoalveolar lavages (BAL) fluid are reported to be significantly higher in patients with lung cancer compared with patients with benign diseases (2+ fold increase), which may also serve as a biomarker for diagnosing and/or monitoring the progression or treatment effects of lung cancer.

Because primary myelofibrosis is associated with abnormal megakaryocyte development, certain cellular markers of megakaryocytes as well as their progenitors of the stem cell lineage may serve as markers to diagnose and/or monitor the disease progression as well as effectiveness of therapy. In some embodiments, useful markers include, but are not limited to: cellular markers of differentiated megakaryocytes (e.g., CD41, CD42 and Tpo R), cellular markers of megakaryocyte-erythroid progenitor cells (e.g., CD34, CD38, and CD45RA−), cellular markers of common myeloid progenitor cells (e.g., IL-3a/CD127, CD34, SCF R/c-kit and Flt-3/Flk-2), and cellular markers of hematopoietic stem cells (e.g., CD34, CD38-, Flt-3/Flk-2). In some embodiments, useful biomarkers include fibrotic markers. These include, without limitation: TGFβ1, PAI-1 (also known as Serpine1), MCP-1 (also known as CCL2), Col1a1, Col3a1, FN1, CTGF, α-SMA, ACTA2, Timp1, Mmp8, and Mmp9. In some embodiments, useful biomarkers are serum markers (e.g., proteins or fragments found and detected in serum samples).

Based on the finding that TGFβ is a component of the leukemic bone marrow niche, it is contemplated that targeting the bone marrow microenvironment with TGFβ inhibitors may be a promising approach to reduce leukemic cells expressing presenting molecules that regulate local TGFβ availability in the effected tissue.

Indeed, due to the multifaceted nature of the pathology which manifests TGFβ-dependent dysregulation in both myelo-proliferative and fibrotic aspects (as the term “myelofibrosis” itself suggests), isoform-specific, context-permissive inhibitors of TGFβ1, such as those described herein, may provide particularly advantageous therapeutic effects for patients suffering from myelofibrosis. It is contemplated that the LTBP-arm of such inhibitor can target ECM-associated TGFβ1 complex in the bone marrow, whilst the LRRC33-arm of the inhibitor can block myeloid cell-associated TGFβ1. In addition, abnormal megakaryocyte biology associated with myelofibrosis may involve both GARP- and LTBP-mediated TGFβ1 activities. The isoform-specific, context-permissive inhibitor of TGFβ1 is capable of targeting such complexes thereby inhibiting release of active TGFβ1 in the niche.

Thus, such TGFβ1 inhibitors are useful for treatment of patients with polycythemia vera who have had an inadequate response to or are intolerant of other (or standard-of-care) treatments, such as hydroxyurea and JAK inhibitors. Such inhibitors are also useful for treatment of patients with intermediate or high-risk myelofibrosis (MF), including primary MF, post-polycythemia vera MF and post-essential thrombocythemia MF.

Accordingly, one aspect of the invention relates to methods for treating primary myelofibrosis. The method comprises administering to a patient suffering from primary myelofibrosis a therapeutically effective amount of a composition comprising a TGFβ inhibitor that causes reduced TGFβ availability. In some embodiments, an isoform-specific, context-permissive monoclonal antibody inhibitor of TGFβ1 activation is administered to patients with myelofibrosis. Such antibody may be administered at dosages ranging between 0.1 and 100 mg/kg, such as between 1 and 30 mg, e.g., 1 mg/kg, 3 mg/kg, 5 mg/kg, 10 mg/kg, 15 mg/kg, 20 mg/kg, etc. Preferred routes of administration of a pharmaceutical composition comprising the antibody is intravenous or subcutaneous administration. When the composition is administered intravenously, the patient may be given the therapeutic over a suitable duration of time, e.g., approximately 60 minutes, per treatment, and then repeated every several weeks, e.g., 3 weeks, 4 weeks, 6 weeks, etc., for a total of several cycles, e.g., 4 cycles, 6, cycles, 8 cycles, 10 cycles, 12 cycles, etc. In some embodiments, patients are treated with a composition comprising the inhibitory antibody at dose level of 1-10 mg/kg (e.g., 1, 2, 3, 4, 5, 6, 7, 8, 9, or 10 mg/kg) via intravenous administration every 28 days (4 weeks) for 6 cycles or 12 cycles. In some embodiments, such treatment is administered as a chronic (long-term) therapy (e.g., to be continued indefinitely, as long as deemed beneficial) in lieu of discontinuing following a set number of cycles of administration.

In some embodiments, the TGFβ inhibitor is an antibody or antigen-binding portion thereof that binds an inactive (e.g., latent) proTGFβ complex, thereby preventing the release of active or mature TGFβ from the complex, effectively inhibiting the activation step. In some embodiments, such an antibody or antigen-binding portion specifically binds a proTGFβ complex that is associated with LRRC33, GARP, LTBP1, LTBP3 or any combination thereof. In some embodiments, such an antibody or antigen-binding portion specifically binds a cell-tethered proTGFβ complex. In some embodiments, the antibody or portion thereof selectively binds a proTGFβ complex that is associated with either LRRC33 and/or GARP (but not with LTBP1 or LTBP3). In some embodiments, the antibody or portion thereof specifically binds a proTGFβ complex that is associated with LRRC33. In some embodiments, the antibody or portion thereof specifically binds a proTGFβ complex that is associated with GARP. In some embodiments, the antibody or portion thereof specifically binds a proTGFβ complex that is associated with LRRC33 as well as a proTGFβ complex that is associated with GARP.

Alternatively or additionally to the embodiments discussed above, the TGFβ inhibitor is an antibody or antigen-binding portion thereof that binds LRRC33 and/or GARP and comprises a domain for additional effector functions. In some embodiments, the domain for additional effector function may be an Fc or Fc-like domain to mediate ADCC in target cells. Preferably, ADCC-inducing antibody does not trigger or facilitate internalization so as to sufficiently allow ADCC-mediated target cell killing.

Alternatively or additionally to the embodiments discussed above, the antibody or antigen-binding portion thereof may include an additional moiety for carrying “a payload” of interest (e.g., antibody-drug conjugates, or ADC). Examples of suitable payload include, but are not limited to: therapeutics/drugs, toxins, markers and detection/imaging labels, etc. Such payload may be chemical entities, small molecules, polypeptides, nucleic acids, radio-isotopes, etc. Preferably, antibodies that are suitable for ADC-mediated mechanism of action can upon binding to cell-surface target, trigger effective internalization of the antigen-antibody complex so as to deliver the payload into the cell.

Because myelofibrosis is a progressive disease that manifests many facets of pathology in multiple affected tissues or organs, therapeutic approach may vary depending on the disease progression. For example, at the primary site of the disease (the bone marrow), it is contemplated that suitable therapy includes an LRRC33 inhibitor described herein, which can target hematopoietic cells expressing LRRC33. This may be achieved by administration of a composition comprising an antibody that binds an LRRC33-presented proTGFβ complex and inhibits activation of TGFβ in the patient. It can also be achieved by administration of a composition comprising an antibody that binds an LRRC33 and inducing killing of target cells in the patient. Alternatively, these approaches may be combined to use an antibody that is a TGFβ activation inhibitor and also contains an additional moiety to mediate cellular cytotoxicity. For example, the additional moiety may be an Fc or Fc-like domain to induce ADCC or a toxin conjugated to the antibody as a payload (e.g., antibody-drug conjugates, or ADC).

While myelofibrosis may be considered a type of leukemia, it is characterized by the manifestation of fibrosis. Because TGFβ is known to regulate aspects of ECM homeostasis, the dysregulation of which can lead to tissue fibrosis, it is contemplated that in some embodiments, it is desirable to inhibit TGFβ activities associated with the ECM. Accordingly, antibodies or fragments thereof that bind and inhibit proTGFβ presented by LTBPs (such as LTBP1 and LTBP3) are encompassed by this invention. In some embodiments, antibodies or fragments thereof suitable for treating myelofibrosis are “context-permissive” in that they can bind multiple contexts of proTGFβ complex, such as those associated with LRRC33, GARP, LTBP1, LTBP3, or any combination thereof. In some embodiments, such antibody is a context-independent inhibitor of TGFβ activation, characterized in that the antibody can bind and inhibit any of the following latent complexes: LTBP1-proTGFβ, LTBP3-proTGFβ, GARP-proTGFβ and LRRC33-proTGFβ. In some embodiments, such an antibody is an isoform-specific antibody that binds and inhibits such latent complexes that comprise one but not the other isoforms of TGFβ. These include, for example, LTBP1-proTGFβ1, LTBP3-proTGFβ1, GARP-proTGFβ1 and LRRC33-proTGFβ1. In some embodiments, such antibody is an isoform-selective antibody that p referentially binds and inhibits one or more isoforms of TGFβ. It is contemplated that antibodies that can inhibit TGFβ1 activation in a context-permissive or context-independent manner are advantageous for use in the treatment of myelofibrosis.

Suitable patient populations of myeloproliferative neoplasms who may be treated with the compositions and methods described herein may include, but are not limited to: a) a patient population that is Philadelphia (+); b) a patient population that is Philadelphia (−); c) a patient population that is categorized “classical” (PV, ET and PMF); d) a patient population carrying the mutation JAK2V617F(+); e) a patient population carrying JAK2V617F(−); f) a patient population with JAK2 exon 12(+); g) a patient population with MPL(+); and h) a patient population with CALR(+).

In some embodiments, the patient population includes patients with intermediate-2 or high-risk myelofibrosis. In some embodiments, the patient population comprises subjects with myelofibrosis who are refractory to or not candidates for available therapy. In some embodiments, the subject has platelet counts between 100-200×10⁹/L. In some embodiments, the subject has platelet counts >200×10⁹/L prior to receiving the treatment.

In some embodiments, a subject to receive (and who may benefit from receiving) an isoform-specific, context-permissive TGFβ1 inhibitor therapy is diagnosed with intermediate-1 or higher primary myelofibrosis (PMF), or post-polycythemia vera/essential thrombocythemia myelofibrosis (post-PV/ET MF). In some embodiments, the subject has documented bone marrow fibrosis prior to the treatment. In some embodiments, the subject has MF-2 or higher as assessed by the European consensus grading score and grade 3 or higher by modified Bauermeister scale prior to the treatment. In some embodiments, the subject has the ECOG performance status of 1 prior to the treatment. In some embodiments, the subject has white blood cell count (10⁹/L) ranging between 5 and 120 prior to the treatment. In some embodiments, the subject has the JAK2V617F allele burden that ranges between 10-100%.

In some embodiments, a subject to receive (and who may benefit from receiving) an isoform-specific, context-permissive TGFβ1 inhibitor therapy is transfusion-dependent (prior to the treatment) characterized in that the subject has a history of at least two units of red blood cell transfusions in the last month for a hemoglobin level of less than 8.5 g/dL that is not associated with clinically overt bleeding.

In some embodiments, a subject to receive (and who may benefit from receiving) an isoform-specific, context-permissive TGFβ1 inhibitor therapy previously received a therapy to treat myelofibrosis. In some embodiments, the subject has been treated with one or more of therapies, including but are not limited to: AZD1480, panobinostat, EPO, IFNα, hydroxyurea, pegylated interferon, thalidomide, prednisone, and JAK2 inhibitor (e.g., Lestaurtinib, CEP-701).

In some embodiments, the patient has extramedullary hematopoiesis. In some embodiments, the extramedullary hematopoiesis is in the liver, lung, spleen, and/or lymph nodes. In some embodiments, the pharmaceutical composition of the present invention is administered locally to one or more of the localized sites of disease manifestation.

The isoform-specific, context-permissive TGFβ1 inhibitor is administered to patients in an amount effective to treat myelofibrosis. The therapeutically effective amount is an amount sufficient to relieve one or more symptoms and/or complications of myelofibrosis in patients, including but are not limited to: excessive deposition of ECM in bone marrow stroma, neoangiogenesis, osteosclerosis, splenomegaly, hematomegaly, anemia, bleeding, bone pain and other bone-related morbidity, extramedullary hematopoiesis, thrombocytosis, leukopenia, cachexia, infections, thrombosis and death.

In some embodiments, the amount is effective to reduce TGFβ1 expression and/or secretion (such as of megakaryocytic cells) in patients. Such inhibitor may therefore reduce TGFβ1 mRNA levels in treated patients. In some embodiments, such inhibitor reduces TGFβ1 mRNA levels in bone marrow, such as in mononuclear cells. PMF patients typically show elevated plasma TGFβ1 levels of above ˜2,500 pg/mL, e.g., above 3,000, 3,500, 4,000, 4,500, 5,000, 6,000, 7,000, 8,000, 9,000, and 10,000 pg/mL (contrast to normal ranges of ˜600-2,000 pg/mL as measured by ELISA) (see, for example, Mascaremhas et al. (Leukemia & Lymphoma, 2014, 55(2): 450-452)). Zingariello (Blood, 2013, 121(17): 3345-3363) quantified bioactive and total TGFβ1 contents in the plasma of PMF patients and control individuals. According to this reference, the median bioactive TGFβ1 in PMF patients was 43 ng/mL (ranging between 4-218 ng/mL) and total TGFβ1 was 153 ng/mL (32-1000 ng/mL), while in control counterparts, the values were 18 (0.05-144) and 52 (8-860), respectively. Thus, based on these reports, plasma TGFβ1 contents in PMF patients are elevated by several fold, e.g., 2-fold, 3-fold, 4-fold, 5-fold, etc., as compared to control or healthy plasma samples. Treatment with the inhibitor, e.g., following 4-12 cycles of administration (e.g., 2, 4, 6, 8, 10, 12 cycles) or chronic or long-term treatment, for example every 4 weeks, at dosage of 0.1-100 mg/kg, for example, 1-30 mg/kg monoclonal antibody) described herein may reduce the plasma TGFβ1 levels by at least 10% relative to the corresponding baseline (pre-treatment), e.g., at least 15%, 20%, 25%, 30%, 35%, 40%, 45%, and 50%.

Some of the therapeutic effects may be observed relatively rapidly following the commencement of the treatment, for example, after 1 week, 2 weeks, 3 weeks, 4 weeks, 5 weeks or 6 weeks. For example, the inhibitor may effectively increase the number of stem cells and/or precursor cells within the bone marrow of patients treated with the inhibitor within 1-8 weeks. These include hematopoietic stem cells and blood precursor cells. A bone marrow biopsy may be performed to assess changes in the frequencies/number of marrow cells. Correspondingly, the patient may show improved symptoms such as bone pain and fatigue.

One of the morphological hallmarks of myelofibrosis is fibrosis in the bone marrow (e.g., marrow stroma), characterized in part by aberrant ECM. In some embodiments, the amount is effective to reduce excessive collagen deposition, e.g., by mesenchymal stromal cells. In some embodiments, the inhibitor is effective to reduce the number of CD41-positive cells, e.g., megakaryocytes, in treated subjects, as compared to control subjects that do not receive the treatment. In some embodiments, baseline frequencies of megakaryocytes in PMF bone marrow may range between 200-700 cells per square millimeters (mm²), and between 40-300 megakaryocites per square-millimeters (mm²) in PMF spleen, as determined with randomly chosen sections. In contrast, megakaryocyte frequencies in bone marrow and spleen of normal donors are fewer than 140 and fewer than 10, respectively. Treatment with the inhibitor may reduce the number (e.g., frequencies) of megakaryocytes in bone marrow and/or spleen. In some embodiments, treatments with the inhibitor can cause reduced levels of downstream effector signaling, such as phosphorylation of SMAD2/3.

Patients with myelofibrosis may suffer from enlarged spleen. Thus, clinical effects of a therapeutic may be evaluated by monitoring changes in spleen size. Spleen size may be examined by known techniques, such as assessment of the spleen length by palpation and/or assessment of the spleen volume by ultrasound. In some embodiments, the subject to be treated with an isoform-specific, context-permissive inhibitor of TGFβ1 has a baseline spleen length (prior to the treatment) of 5 cm or greater, e.g., ranging between 5 and 30 cm as assessed by palpation. In some embodiments, the subject to be treated with an isoform-specific, context-permissive inhibitor of TGFβ1 has a baseline spleen volume (prior to the treatment) of 300 mL or greater, e.g., ranging between 300-1500 mL, as assessed by ultrasound. Treatment with the inhibitor, e.g., following 4-12 cycles of administration (e.g., 2, 4, 6, 8, 10, 12 cycles), for example every 4 weeks, at dosage of 0.1-30 mg/kg monoclonal antibody) described herein may reduce spleen size in the subject. In some embodiments, the effective amount of the inhibitor is sufficient to reduce spleen size in a patient population that receives the inhibitor treatment by at least 10%, 20%, 30%, 35%, 40%, 50%, and 60%, relative to corresponding baseline values. For example, the treatment is effective to achieve a ≥35% reduction in spleen volume from baseline in 12-24 weeks as measured by MRI or CT scan, as compare to placebo control. In some embodiments, the treatment is effective to achieve a ≥35% reduction in spleen volume from baseline in 24-48 weeks as measured by MRI or CT scan, as compare to best available therapy control. Best available therapy may include hydroxyurea, glucocorticoids, as well as no medication, anagrelide, epoetin alfa, thalidomide, lenalidomide, mercaptopurine, thioguanine, danazol, peginterferon alfa-2a, interferon-α, melphalan, acetylsalicylic acid, cytarabine, and colchicine.

In some embodiments, a patient population treated with an isoform-specific, context-permissive TGFβ1 inhibitor such as those described herein, shows a statistically improved treatment response as assessed by, for example, International Working Group for Myelofibrosis Research and Treatment (IWG-MRT) criteria, degree of change in bone marrow fibrosis grade measured by the modified Bauermeister scale and European consensus grading system after treatment (e.g., 4, 6, 8, or 12 cycles), symptom response using the Myeloproliferative Neoplasm Symptom Assessment Form (MPN-SAF).

In some embodiments, the treatment with an isoform-specific, context-permissive TGFβ1 inhibitor such as those described herein, achieves a statistically improved treatment response as assessed by, for example, modified Myelofibrosis Symptom Assessment Form (MFSAF), in which symptoms are measured by the MFSAF tool (such as v2.0), a daukt diary capturing the debilitating symptoms of myelofibrosis (abdominal discomfort, early satiety, pain under left ribs, pruritus, night sweats, and bone/muscle pain) using a scale of 0 to 10, where 0 is absent and 10 is the worst imaginable. In some embodiments, the treatment is effective to achieve a 50%≥reduction in total MFSAF score from the baseline in, for example, 12-24 weeks. In some embodiments, a significant fraction of patients who receive the therapy achieves a ≥50% improvement in Total Symptom Score, as compared to patients taking placebo. For example, the fraction of the patient pool to achieve ≥50% improvement may be over 40%, 50%, 55%, 60%, 65%, 70%, 75% or 80%.

In some embodiments, the therapeutically effective amount of the inhibitor is an amount sufficient to attain clinical improvement as assessed by an anemia response. For example, an improved anemia response may include longer durations of transfusion-independence, e.g., 8 weeks or longer, following the treatment of 4-12 cycles, e.g., 6 cycles.

In some embodiments, the therapeutically effective amount of the inhibitor is an amount sufficient to maintain stable disease for a duration of time, e.g., 6 weeks, 8 weeks, 12 weeks, six months, etc. In some embodiments, progression of the disease may be evaluated by changes in overall bone marrow cellularity, the degree of reticulin or collagen fibrosis, and/or a change in JAK2V617F allele burden.

In some embodiments, a patient population treated with an isoform-specific, context-permissive TGFβ1 inhibitor such as those described herein, shows statistically improved survival, as compared to a control population that does not receive the treatment. For example, in control groups, median survival of PMF patients is approximately six years (approximately 16 months in high-risk patients), and fewer than 20% of the patients are expected to survive 10 years or longer post-diagnosis. Treatment with the isoform-specific, context-permissive TGFβ1 inhibitor such as those described herein, may prolong the survival time by, at least 6 months, 12 months, 18 months, 24 months, 30 months, 36 months, or 48 months. In some embodiments, the treatment is effective to achieve improved overall survival at 26 weeks, 52 weeks, 78 weeks, 104 weeks, 130 weeks, 144 weeks, or 156 weeks, as compared to patients who receive placebo.

Clinical benefits of the therapy, such as those exemplified above, may be seen in patients with or without new onset anemia.

One of the advantageous features of the isoform-specific, context-permissive TGFβ1 inhibitors is that they maintain improved safety profiles enabled by isoform selectivity, as compared to conventional TGFβ antagonists that lack the selectivity. Therefore, it is anticipated that treatment with an isoform-specific, context-permissive inhibitor, such as those described herein, may reduce adverse events in a patient population, in comparison to equivalent patient populations treated with conventional TGFβ antagonists, with respect to the frequency and/or severity of such events. Thus, the isoform-specific, context-permissive TGFβ1 inhibitors may provide a greater therapeutic window as to dosage and/or duration of treatment.

Adverse events may be graded by art-recognized suitable methods, such as Common Terminology Criteria for Adverse Events (CTCAE) version 4. Previously reported adverse events in human patients who received TGFβ antagonists, such as GC1008, include: leukocytosis (grade 3), fatigue (grade 3), hypoxia (grade 3), asystole (grade 5), leukopenia (grade 1), recurrent, transient, tender erythematous, nodular skin lesions, suppurative dermatitis, and herpes zoster.

The isoform-specific, context-permissive TGFβ1 inhibitor therapy may cause less frequent and/or less severe adverse events (side effects) as compared to JAK inhibitor therapy in myelofibrosis patients, with respect to, for example, anemia, thrombocytopenia, neutropenia, hypercholesterolemia, elevated alanine transaminase (ALT), elevated aspartate transaminase (AST), bruising, dizziness, and headache, thus offering a safer treatment option.

It is contemplated that inhibitors of TGFβ1 signaling may be used in conjunction with one or more therapeutics for the treatment of myelofibrosis as a combination therapy. In some embodiments, an inhibitor of TGFβ1 activation described herein is administered to patients suffering from myelofibrosis, who have received a JAK1 inhibitor, JAK2 inhibitor or JAK1/JAK2 inhibitor. In some embodiments, such patients are responsive to the JAK1 inhibitor, JAK2 inhibitor or JAK1/JAK2 inhibitor therapy, while in other embodiments such patients are poorly responsive or not responsive to the JAK1 inhibitor, JAK2 inhibitor or JAK1/JAK2 inhibitor therapy. In some embodiments, use of an isoform-specific inhibitor of TGFβ1 described herein may render those who are poorly responsive or not responsive to the JAK1 inhibitor, JAK2 inhibitor or JAK1/JAK2 inhibitor therapy more responsive. In some embodiments, use of an isoform-specific inhibitor of TGFβ1 described herein may allow reduced dosage of the JAK1 inhibitor, JAK2 inhibitor or JAK1/JAK2 inhibitor which still produces equivalent clinical efficacy in patients but fewer or lesser degrees of drug-related toxicities or adverse events (such as those listed above). In some embodiments, treatment with the inhibitor of TGFβ1 activation described herein used in conjunction with JAK1 inhibitor, JAK2 inhibitor or JAK1/JAK2 inhibitor therapy may produce synergistic or additive therapeutic effects in patients. In some embodiments, treatment with the inhibitor of TGFβ1 activation described herein may boost the benefits of JAK1 inhibitor, JAK2 inhibitor or JAK1/JAK2 inhibitor or other therapy given to treat myelofibrosis. In some embodiments, patients may additionally receive a therapeutic to address anemia associated with myelofibrosis.

### Cancer:

Various cancers involve TGFβ1 activities and may be treated with antibodies and/or compositions of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be localized (e.g., solid tumors) or systemic. In the context of the present disclosure, the term “localized” (as in “localized tumor”) refers to anatomically isolated or isolatable abnormalities, such as solid malignancies, as opposed to systemic disease. Certain cancers, such as certain leukemia (e.g., myelofibrosis) and multiple myeloma, for example, may have both a localized component (for instance the bone marrow) and a systemic component (for instance circulating blood cells) to the disease. In some embodiments, cancers may be systemic, such as hematological malignancies. Cancers that may be treated according to the present disclosure include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. In cancer, TGFβ (e.g., TGFβ1) may be either growth promoting or growth inhibitory. As an example, in pancreatic cancers, SMAD4 wild type tumors may experience inhibited growth in response to TGFβ, but as the disease progresses, constitutively activated type II receptor is typically present. Additionally, there are SMAD4-null pancreatic cancers. In some embodiments, antibodies, antigen binding portions thereof, and/or compositions of the present disclosure are designed to selectively target components of TGFβ signaling pathways that function uniquely in one or more forms of cancer. Leukemias, or cancers of the blood or bone marrow that are characterized by an abnormal proliferation of white blood cells, i.e., leukocytes, can be divided into four major classifications including acute lymphoblastic leukemia (ALL), chronic lymphocytic leukemia (CLL), acute myelogenous leukemia or acute myeloid leukemia (AML) (AML with translocations between chromosome 10 and 11 [t(10, 11)], chromosome 8 and 21 [t(8;21)], chromosome 15 and 17 [t(15;17)], and inversions in chromosome 16 [inv(16)]; AML with multilineage dysplasia, which includes patients who have had a prior myelodysplastic syndrome (MDS) or myeloproliferative disease that transforms into AML; AML and myelodysplastic syndrome (MDS), therapy-related, which category includes patients who have had prior chemotherapy and/or radiation and subsequently develop AML or MDS; d) AML not otherwise categorized, which includes subtypes of AML that do not fall into the above categories; and e) acute leukemias of ambiguous lineage, which occur when the leukemic cells cannot be classified as either myeloid or lymphoid cells, or where both types of cells are present); and chronic myelogenous leukemia (CML).

Isoform-specific, context-permissive inhibitors of TGFβ1, such as those described herein, may be used to treat multiple myeloma. Multiple myeloma is a cancer of B lymphocytes (e.g., plasma cells, plasmablasts, memory B cells) that develops and expands in the bone marrow, causing destructive bone lesions (i.e., osteolytic lesion). Typically, the disease manifests enhanced osteoclastic bone resorption, suppressed osteoblast differentiation (e.g., differentiation arrest) and impaired bone formation, characterized in part, by osteolytic lesions, osteopenia, osteoporosis, hypercalcemia, as well as plasmacytoma, thrombocytopenia, neutropenia and neuropathy. The TGFβ1-selective, context-permissive inhibitor therapy described herein may be effective to ameliorate one or more such clinical manifestations or symptoms in patients. The TGFβ1 inhibitor may be administered to patients who receive additional therapy or therapies to treat multiple myeloma, including those listed elsewhere herein. In some embodiments, multiple myeloma may be treated with a TGFβ1 inhibitor (such as an isoform-specific context-permissive inhibitor) in combination with a myostatin inhibitor or an IL-6 inhibitor. In some embodiments, the TGFβ1 inhibitor may be used in conjunction with traditional multiple myeloma therapies, such as bortezomib, lenalidomide, carfilzomib, pomalidomide, thalidomide, doxorubicin, corticosteroids (e.g., dexamethasone and prednisone), chemotherapy (e.g., melphalan), radiation therapy, stem cell transplantation, plitidepsin, Elotuzumab, Ixazomib, Masitinib, and/or Panobinostat.

The types of carcinomas which may be treated by the methods of the present invention include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

Isoform-selective, context-permissive/independent inhibitors of TGFβ1 activation, such as those described herein, may be suited for treating malignancies involving cells of neural crest origin. Cancers of the neural crest lineage (i.e., neural crest-derived tumors) include, but are not limited to: melanoma (cancer of melanocytes), neuroblastoma (cancer of sympathoadrenal precursors), ganglioneuroma (cancer of peripheral nervous system ganglia), medullary thyroid carcinoma (cancer of thyroid C cells), pheochromocytoma (cancer of chromaffin cells of the adrenal medulla), and MPNST (cancer of Schwann cells). In some embodiments, antibodies and methods of the disclosure may be used to treat one or more types of cancer or cancer-related conditions that may include, but are not limited to colon cancer, renal cancer, breast cancer, malignant melanoma and glioblastomas (Schlingensiepen et al., 2008; Ouhtit et al., 2013).

Increasing lines of evidence suggest the role of macrophages in tumor/cancer progression. The present invention encompasses the notion that this is in part mediated by TGFβ1 activation in the disease environment, such as TME. Bone marrow-derived monocytes (e.g., CD1 b+) are recruited to tumor sites in response to tumor-derived cytokines/chemokines, where monocytes undergo differentiation and polarization to acquire pro-cancer phenotype (e.g., M2-biased, TAMs or TAM-like cells). As demonstrated in the Examples provided in the present disclosure, monocytes isolated from human PBMCs can be induced to polarize into different subtypes of macrophages, e.g., M1 (pro-fibrotic, anti-cancer) and M2 (pro-cancer). A majority of TAMs in many tumors are M2-biased. Among the M2-like macrophages, M2c and M2d subtypes, but not M1, are found to express elevated LRRC33 on the cell surface. Moreover, macrophages can be further skewed or activated by an M-CSF exposure, resulting in a marked increase in LRRC33 expression, which coincides with TGFβ1 expression. Increased circulating M-CSF (i.e., serum M-CSF concentrations) in patients with myeloproliferative disease (e.g., myelofibrosis) has also been observed. Generally, tumors with high macrophage (TAM) and/or MDSC infiltrate are associated with poor prognosis. Similarly, elevated levels of M-CSF are also indicative of poor prognosis.

As mentioned above, context-permissive/independent inhibitors of TGFβ1 activation may be used in the treatment of Melanoma. The types of melanoma that may be treated with such inhibitors include, but are not limited to: Lentigo maligna; Lentigo maligna melanoma; Superficial spreading melanoma; Acral lentiginous melanoma; Mucosal melanoma; Nodular melanoma; Polypoid melanoma and Desmoplastic melanoma. In some embodiments, the melanoma is a metastatic melanoma.

More recently, immune checkpoint inhibitors have been used to effectively treat advanced melanoma patients. In particular, anti-programmed death (PD)-1 antibodies (e.g., nivolumab and pembrolizumab) have now become the standard of care for certain types of cancer such as advanced melanoma, which have demonstrated significant activity and durable response with a manageable toxicity profile. However, effective clinical application of PD-1 antagonists is encumbered by a high rate of innate resistance (˜60-70%) (see Hugo et al. (2016) Cell 165: 35-44), illustrating that ongoing challenges continue to include the questions of patient selection and predictors of response and resistance as well as optimizing combination strategies (Perrot et al. (2013) Ann Dermatol 25(2): 135-144). Moreover, studies have suggested that approximately 25% of melanoma patients who initially responded to an anti-PD-1 therapy eventually developed acquired resistance (Ribas et al. (2016) JAMA 315: 1600-9).

The number of tumor-infiltrating CD8+ T cells expressing PD-1 and/or CTLA-4 appears to be a key indicator of success with checkpoint inhibition, and both PD-1 and CTLA-4 blockade may increase the infiltrating T cells. In patients with higher macrophage infiltration, however, anti-cancer effects of the CD8 cells may be suppressed.

It is contemplated that LRRC33-expressing cells, such as myeloid cells, including myeloid precursors, MDSCs and TAMs, may create or support an immunosuppressive environment (such as TME and myelofibrotic bone marrow) by inhibiting T cells (e.g., T cell depletion), such as CD4 and/or CD8 T cells, which may at least in part underline the observed anti-PD-1 resistance in certain patient populations. Indeed, evidence suggests that resistance to anti-PD-1 monotherapy was marked by failure to accumulate CD8+ cytotoxic T cells and rescued Teff/Treg ratio. Notably, the present inventors have recognized that there is a bifurcation among certain cancer patients, such as a melanoma patient population, with respect to LRRC33 expression levels: one group exhibits high LRRC33 expression (LRRC33^{high}), while the other group exhibits relatively low LRRC33 expression (LRRC33^{low}). Thus, the invention includes the notion that the LRRC33^{high} patient population may represent those who are poorly responsive to or resistant to immuno checkpoint inhibitor therapy. Accordingly, agents that inhibit LRRC33, such as those described herein, may be particularly beneficial for the treatment of cancer, such as melanoma, lymphoma, and myeloproliferative disorders, that is resistant to checkpoint inhibitor therapy (e.g., anti-PD-1).

In some embodiments, cancer/tumor is intrinsically resistant to or unresponsive to an immune checkpoint inhibitor. To give but one example, certain lymphomas appear poorly responsive to immune checkpoint inhibition such as anti-PD-1 therapy. Similarly, a subset of melanoma patient population is known to show resistance to immune checkpoint inhibitors. Without intending to be bound by particular theory, the inventors of the present disclosure contemplate that this may be at least partly due to upregulation of TGFβ1 signaling pathways, which may create an immunosuppressive microenvironment where checkpoint inhibitors fail to exert their effects. TGFβ1 inhibition may render such cancer more responsive to checkpoint inhibitor therapy. Non-limiting examples of cancer types which may benefit from a combination of an immune checkpoint inhibitor and a TGFβ1 inhibitor include: myelofibrosis, melanoma, renal cell carcinoma, bladder cancer, colon cancer, hematologic malignancies, non-small cell carcinoma, non-small cell lung cancer (NSCLC), lymphoma (classical Hodgkin's and non-Hodgkin's), head and neck cancer, urothelial cancer, cancer with high microsatellite instability, cancer with mismatch repair deficiency, gastric cancer, renal cancer, and hepatocellular cancer. However, any cancer (e.g., patients with such cancer) in which TGFβ1 is overexpressed or is the dominant isoform over TGFβ2/3, as determined by, for example biopsy, may be treated with an isoform-selective inhibitor of TGFβ1 in accordance with the present disclosure.

In some embodiments, a cancer/tumor becomes resistant over time. This phenomenon is referred to as acquired resistance or adaptive resistance. Like intrinsic resistance, in some embodiments, acquired resistance is at least in part mediated by TGFβ1-dependent pathways, Isoform-specific TGFβ1 inhibitors described herein may be effective in restoring anti-cancer immunity in these cases.

In some embodiments, combination therapy comprising an immuno checkpoint inhibitor and an LRRC33 inhibitor (such as those described herein) may be effective to treat such cancer. In addition, high LRRC33-positive cell infiltrate in tumors, or otherwise sites/tissues with abnormal cell proliferation, may serve as a biomarker for host immunosuppression and immuno checkpoint resistance. Similarly, effector T cells may be precluded from the immunosuppressive niche which limits the body's ability to combat cancer. Moreover, as demonstrated in the Example section below, Tregs that express GARP-presented TGFβ1 suppress effector T cell proliferation. Together, TGFβ1 is likely a key driver in the generation and maintenance of an immune inhibitory disease microenvironment (such as TME), and multiple TGFβ1 presentation contexts are relevant for tumors. In some embodiments, the combination therapy may achieve more favorable Teff/Treg ratios.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating cancer in a subject in need thereof, said method comprising administering the antibody, or antigen binding portion thereof, to the subject such that the cancer is treated. In certain embodiments, the cancer is colon cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for treating solid tumors. In some embodiments, solid tumors may be desmoplastic tumors, which are typically dense and hard for therapeutic molecules to penetrate. By targeting the ECM component of such tumors, such antibodies may “loosen” the dense tumor tissue to disintegrate, facilitating therapeutic access to exert its anti-cancer effects. Thus, additional therapeutics, such as any known anti-tumor drugs, may be used in combination.

Additionally or alternatively, isoform-specific, context-permissive antibodies for fragments thereof that are capable of inhibiting TGFβ1 activation, such as those disclosed herein, may be used in conjunction with the chimeric antigen receptor T-cell (“CAR-T”) technology as cell-based immunotherapy, such as cancer immunotherapy for combating cancer.

In some embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, may be used in methods for inhibiting or decreasing solid tumor growth in a subject having a solid tumor, said method comprising administering the antibody, or antigen binding portion thereof, to the subject such that the solid tumor growth is inhibited or decreased. In certain embodiments, the solid tumor is a colon carcinoma tumor. In some embodiments, the antibodies, or antigen binding portions thereof useful for treating a cancer is an isoform-specific, context-permissive inhibitor of TGFβ1 activation. In some embodiments, such antibodies target a GARP-TGFβ1 complex, a LTBP1-TGFβ1 complex, a LTBP3-TGFβ1 complex, and a LRRC33-TGFβ1 complex. In some embodiments, such antibodies target a GARP-TGFβ1 complex, a LTBP1-TGFβ1 complex, and a LTBP3-TGFβ1 complex. In some embodiments, such antibodies target a LTBP1-TGFβ1 complex, a LTBP3-TGFβ1 complex, and a LRRC33-TGFβ1 complex. In some embodiments, such antibodies target a GARP-TGFβ1 complex and a LRRC33-TGFβ1 complex.

The invention includes the use of context-permissive (context-independent), isoform-specific inhibitors of TGFβ1 in the treatment of cancer comprising a solid tumor in a subject. In some embodiments, such context permissive (context-independent), isoform-specific inhibitor may inhibit the activation of TGFβ1. In preferred embodiments, such activation inhibitor is an antibody or antigen-binding portion thereof that binds a proTGFβ1 complex. The binding can occur when the complex is associated with any one of the presenting molecules, e.g., LTBP1, LTBP3, GARP or LRRC33, thereby inhibiting release of mature TGFβ1 growth factor from the complex. In some embodiments, the solid tumor is characterized by having stroma enriched with CD8+ T cells making direct contact with CAFs and collagen fibers. Such a tumor may create an immuno-suppressive environment that prevents anti-tumor immune cells (e.g., effector T cells) from effectively infiltrating the tumor, limiting the body's ability to fight cancer. Instead, such cells may accumulate within or near the tumor stroma. These features may render such tumors poorly responsive to an immune checkpoint inhibitor therapy. As discussed in more detail below, TGFβ1 inhibitors disclosed herein may unblock the suppression so as to allow effector cells to reach and kill cancer cells, for example, used in conjunction with an immune checkpoint inhibitor.

TGFβ1 is contemplated to play multifaceted roles in a tumor microenvironment, including tumor growth, host immune suppression, malignant cell proliferation, vascularity, angiogenesis, migration, invasion, metastatis, and chemo-resistance. Each “context” of TGFβ1 presentation in the environment may therefore participate in the regulation (or dysregyulation) of disease progression. For example, the GARP axis is particularly important in Treg response that regulates effector T cell response for mediating host immune response to combat cancer cells. The LTBP1/3 axis may regulate the ECM, including the stroma, where cancer-associated fibroblasts (CAFs) play a role in the pathogenesis and progression of cancer. The LRRC33 axis may play a crucial role in recruitment of circulating monocytes to the tumor microenvironment, subsequent differentiation into tumor-associated macrophages (TAMs), infiltration into the tumor tissue and exacerbation of the disease.

In some embodiments, TGFβ1-expressing cells infiltrate the tumor, creating an immunosuppressive local environment. The degree by which such infiltration is observed may correlate with worse prognosis. In some embodiments, higher infiltration is indicative of poorer treatment response to another cancer therapy, such as immune checkpoint inhibitors. In some embodiments, TGFβ1-expressing cells in the tumor microenvironment comprise Tregs and/or myeloid cells. In some embodiments, the myeloid cells include, but are not limited to: macrophages, monocytes (tissue resident or bone marrow-derived), and MDSCs.

In some embodiments, LRRC33-expressing cells in the TME are myeloid-derived suppressor cells (MDSCs). MDSC infiltration (e.g., solid tumor infiltrate) may underline at least one mechanism of immune escape, by creating an immunosuppressive niche from which host's anti-tumor immune cells become excluded. Evidence suggest that MDSCs are mobilized by inflammation-associated signals, such as tumor-associated inflammatory factors, Opon mobilization, MDSCs can influence immunosuppressive effects by impairing disease-combating cells, such as CD8+ T cells and NK cells. In addition, MDSCs may induce differentiation of Tregs by secreting TGFβ and IL-10. Thus, an isoform-specific, context-permissive TGFβ1 inhibitor, such as those described herein, may be administered to patients with immune evasion (e.g., compromised immune surveillance) to restore or boost the body's ability to fight the disease (such as tumor). As described in more detail herein, this may further enhance (e.g., restore or potentiate) the body's responsiveness or sensitivity to another therapy, such as cancer therapy.

In some embodiments, elevated frequencies (e.g., number) of circulating MDSCs in patients are predictive of poor responsiveness to checkpoint blockade therapies, such as PD-1 antagonists and PD-L1 antagonists. For example, biomarker studies showed that circulating pre-treatment HLA-DR lo/CD14+/CD11b+ myeloid-derived suppressor cells (MDSC) were associated with progression and worse OS (p=0.0001 and 0.0009). In addition, resistance to PD-1 checkpoint blockade in inflamed head and neck carcinoma (HNC) associates with expression of GM-CSF and Myeloid Derived Suppressor Cell (MDSC) markers. This observation suggested that strategies to deplete MDSCs, such as chemotherapy, should be considered in combination or sequentially with anti-PD-1. LRRC33 or LRRC33-TGFβ complexes represent a novel target for cancer immunotherapy due to selective expression on immunosuppressive myeloid cells. Therefore, without intending to be bound by particular theory, targeting this complex may enhance the effectiveness of standard-of-care checkpoint inhibitor therapies in the patient population.

The invention therefore provides the use of an isoform-specific, context-permissive or context-independent TGFβ1 inhibitor described herein for the treatment of cancer that comprises a solid tumor. Such treatment comprises administration of the isoform-specific, context-permissive or context-independent TGFβ1 inhibitor to a subject diagnosed with cancer that includes at least one localized tumor (solid tumor) in an amount effective to treat the cancer.

Evidence suggests that cancer progression (e.g., tumor proliferation/growth, invasion, angiogenesis and metastasis) may be at least in part driven by tumor-stroma interaction. In particular, CAFs may contribute to this process by secretion of various cytokines and growth factors and ECM remodeling. Factors involved in the process include but are not limited to stromal-cell-derived factor 1 (SCD-1), MMP2, MMP9, MMP3, MMP-13, TNF-α, TGFβ1, VEGF, IL-6, M-CSF. In addition, CAFs may recruit TAMs by secreting factors such as CCL2/MCP-1 and SDF-1/CXCL12 to a tumor site; subsequently, a pro-TAM niche (e.g., hyaluronan-enriched stromal areas) is created where TAMs preferentially attach. Since TGFβ1 has been suggested to promote activation of normal fibroblasts into myofibroblast-like CAFs, administration of an isoform-specific, context-permissive or context-independent TGFβ1 inhibitor such as those described herein may be effective to counter cancer-promoting activities of CAFs. Indeed, data presented herein suggest that an isoform-specific context-independent antibody that blocks activation of TGFβ1 can inhibit UUO-induced upregulation of maker genes such as CCL2/MCP-1, α-SMA. FN1 and Col1, which are also implicated in many cancers.

In certain embodiments, the antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex, as described herein, are administered to a subject having cancer or a tumor, either alone or in combination with an additional agent, e.g., an anti-PD-1 antibody (e.g, an anti-PD-1 antagonist). Other combination therapies which are included in the invention are the administration of an antibody, or antigen binding portion thereof, described herein, with radiation, or a chemotherapeutic agent. Exemplary additional agents include, but are not limited to, a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an anti-CD20 antibody, an oncolytic virus, and a PARP inhibitor.

In some embodiments, determination or selection of therapeutic approach for combination therapy that suits particular cancer types or patient population may involve the following: a) considerations regarding cancer types for which a standard-of-care therapy is available (e.g., immunotherapy-approved indications); b) considerations regarding treatment-resistant subpopylations; and c) considerations regarding cancers/tumors that are “TGFβ1 pathway-active” or otherwise at least in part TGFβ1-dependent (e.g., TGFβ1 inhibition-sensitive). For example, many cancer samples show that TGFβ1 is the predominant isoform by, for instance, TCGA RNAseq analysis. In some embodiments, over 50% (e.g., over 50%, 60%, 70%, 80% and 90%) of samples from each tumor type are positive for TGFβ1 isoform expression. In some embodiments, the cancers/tumors that are “TGFβ1 pathway-active” or otherwise at least in part TGFβ1-dependent (e.g., TGFβ1 inhibition-sensitive) contain at least one Ras mutation, such as mutations in K-ras, N-ras and/or H-ras. In some embodiments, the cancer/tumor comprises at least one K-ras mutation.

In some embodiments, the isoform-specific, context-permissive TGFβ1 inhibitor is administered in conjunction with checkpoint inhibitory therapy to patients diagnosed with cancer for which one or more checkpoint inhibitor therapies are approved. These include, but are not limited to: bladder urothelial carcinoma, squamous cell carcinoma (such as head & neck), kidney clear cell carcinoma, kidney papillary cell carcinoma, liver hepatocellular carcinoma, lung adenocarcinoma, skin cutaneous melanoma, and stomack adenocarcinoma. In preferred embodiments, such patients are poorly responsive or non-responsive to the checkpoint inhibitor therapy.

### Role of TGFβ in Musculoskeletal Conditions:

In musculoskeletal system, which is comprised of the bones of the skeleton, muscles, cartilage, tendons, ligaments, joints, and other connective tissue that supports and binds tissues and organs together, TGFβ plays a variety of roles including inhibition of proliferation and differentiation, induction of atrophy, and development of fibrosis. TGFβ reduces satellite cell proliferation and prevents differentiation (via inhibition of MyoD and myogenin) (Allen, R. E. and L. K. J Cell Physiol, 1987. 133(3): p. 567-72; Brennan, T. J., et al., Proc Natl Acad Sci USA, 1991. 88(9): p. 3822-6; Massague, J., et al., Proc Natl Acad Sci USA, 1986. 83(21): p. 8206-10; Olson, E. N., et al., J Cell Biol, 1986. 103(5): p. 1799-805). The isoform of TGFβ (i.e., TGFβ1, 2, or 3) is not specified in these early papers, but is presumed to be TGFβ1. TGFβ also contributes to muscle fibrosis; direct injection of recombinant TGFβ1 results in skeletal muscle fibrosis, and pan-TGFβ inhibition decreases fibrosis in acute and chronically injured muscle (Li, Y., et al., Am J Pathol, 2004. 164(3): p. 1007-19; Mendias, C. L., et al., Muscle Nerve, 2012. 45(1): p. 55-9; Nelson, C. A., et al., Am J Pathol, 2011. 178(6): p. 2611-21). TGFβ1 is expressed by myofibers, macrophages, regulatory T cells, fibroblasts, and fibrocytes within the skeletal muscle (Li, Y., et al., Am J Pathol, 2004. 164(3): p. 1007-19; Lemos, D. R., et al., Nat Med, 2015. 21(7): p. 786-94; Villalta, S. A., et al., Sci Transl Med, 2014. 6(258): p. 258ra142; Wang, X., et al., J Immunol, 2016. 197(12): p. 4750-4761); and expression is increased upon injury and in disease (Li, Y., et al., Am J Pathol, 2004. 164(3): p. 1007-19; Nelson, C. A., et al., Am J Pathol, 2011. 178(6): p. 2611-21; Bernasconi, P., et al., J Clin Invest, 1995. 96(2): p. 1137-44; Ishitobi, M., et al., Neuroreport, 2000. 11(18): p. 4033-5). TGFβ2 and TGFβ3 are also upregulated (at the mRNA level) in mdx muscle, although to a lesser extent than TGFβ1 (Nelson, C. A., et al., Am J Pathol, 2011. 178(6): p. 2611-21; Zhou, L., et al., Neuromuscul Disord, 2006. 16(1): p. 32-8). Pessina, et al., recently used lineage tracing experiments to show that cells of multiple origins within dystrophic muscle adopt a fibrogenic fate via a TGFβ-dependent pathway (Pessina, P., et al., Stem Cell Reports, 2015. 4(6): p. 1046-60).

The bone is the largest storehouse of TGFβ in the body. Indeed, the TGFβ pathway is thought to play an important role in bone homeostasis and remodeling at least in part by regulating osteoblast differentiation and/or osteoclastic bone resorption. This process is involved in both normal and abnormal situations, which, when dysregulated, may cause or exacerbate disease, such as bone-related conditions and cancer. Thus, TGFβ1-selective inhibitors such as those described herein may be used to treat such conditions. In some embodiments, administration of such inhibitors is effective to restore or normalize bone formation-resorption balance. In some embodiments, the TGFβ1 inhibitor is administered to subjects in conjunction with another therapy, such as a myostatin inhibitor and/or bone-enhancing agents, as combination therapy.

Bone conditions (e.g., skeletal diseases) include osteoporosis, dysplasia and bone cancer. In addition to primary bone cancer that originates in the bone, many malignancies are known to metastasize to bone; these include, but are not limited to. breast cancer, lung cancer (e.g., squamous cell carcinoma), thyroid cancer, testicular cancer, renal cell carcinoma, prostate cancer, and multiple myeloma.

In some embodiments, such conditions are associated with muscle weakness.

TGFβ1 may play a role in fibrotic conditions that accompany chronic inflammation of the affected tissue, such as human muscular dystrophies. Duchenne muscular dystrophy (DMD) is a severe, progressive, and ultimately fatal disease caused by the absence of dystrophin (Bushby, K., et al., Lancet Neurol, 2010. 9(1): p. 77-93). Lack of dystrophin results in increased susceptibility to contraction-induced injury, leading to continual muscle degeneration (Petrof, B. J., et al., Proc Natl Acad Sci USA, 1993. 90(8): p. 3710-4; Dellorusso, C., et al., J Muscle Res Cell Motil, 2001. 22(5): p. 467-75; Pratt, S. J., et al., Cell Mol Life Sci, 2015. 72(1): p. 153-64). Repeated rounds of repair contribute to chronic inflammation, fibrosis, exhaustion of the satellite cell pool, eventual loss of mobility and death (Bushby, K., et al., Lancet Neurol, 2010. 9(1): p. 77-93; McDonald, C. M., et al., Muscle Nerve, 2013. 48(3): p. 343-56). Expression of TGFβ1 is significantly increased in patients with DMD and correlates with the extent of fibrosis observed in these patients (Bernasconi, P., et al., J Clin Invest, 1995. 96(2): p. 1137-44; Chen, Y. W., et al., Neurology, 2005. 65(6): p. 826-34). Excessive ECM deposition has detrimental effects on the contractile properties of the muscle and can limit access to nutrition as the myofibers are isolated from their blood supply (Klingler, W., et al., Acta Myol, 2012. 31(3): p. 184-95). Recently, additional data has further implicated TGFβ1 in muscular dystrophies. Variants in LTBP4 have been found to modify disease severity in mouse and human. In mouse, a variant of LTBP4 is protective in mice lacking dystrophin or γ-sarcoglycan (Coley, W. D., et al., Hum Mol Genet, 2016. 25(1): p. 130-45; Heydemann, A., et al., J Clin Invest, 2009. 119(12): p. 3703-12). In humans, two groups independently identified a variant of LTBP4 as protective in DMD, delaying loss of ambulation by several years (Flanigan, K. M., et al., Ann Neurol, 2013. 73(4): p. 481-8; van den Bergen, J. C., et al., J Neurol Neurosurg Psychiatry, 2015. 86(10): p. 1060-5). Although the nature of the genetic variants in mouse and human differs, in both species the protective variant results in decreased TGFβ signaling (Heydemann, A., et al., J Clin Invest, 2009. 119(12): p. 3703-12); Ceco, E., et al., Sci Transl Med, 2014. 6(259): p. 259ra144). Many of the functions of TGFβ1 in skeletal muscle biology have been inferred from experiments in which purified active growth factor is injected into animals or added to cells in culture (Massague, J., et al., Proc Natl Acad Sci USA, 1986. 83(21): p. 8206-10; Li, Y., et al., Am J Pathol, 2004. 164(3): p. 1007-19; Mendias, C. L., et al., Muscle Nerve, 2012. 45(1): p. 55-9). Given the importance of cellular context for specific functions of TGFβ1 (see, for example, Hinck et al., Cold Spring Harb. Perspect. Biol, 2016. 8(12)) it is possible that some of the effects observed in these experiments do not reflect the endogenous role(s) of the cytokine in vivo. For example, treatment of human dermal fibroblasts with recombinant TGFβ1, myostatin, or GDF11 results in nearly identical changes in gene expression in these cells, although in vivo the roles of these proteins are quite different (Tanner, J. W., Khalil, A., Hill, J., Franti, M., MacDonnell, S. M., Growth Differentiation Factor 11 Potentiates Myofibroblast Activation, in Fibrosis: From Basic Mechanisms to Targeted therapies. 2016: Keystone, Colo.).

Multiple investigators have used inhibitors of TGFβ to clarify the role of the growth factor in vivo. Treatment of mdx mice with the pan-TGFβ neutralizing antibody 1D11 clearly results in reduced fibrosis (by histology and hydroxyproline content), reduced muscle damage (reduced serum creatine kinase and greater myofiber density), and improved muscle function (by plethysmography, force generation of isolated EDL muscles, and increased forelimb grip strength) (Nelson, C. A., et al., Am J Pathol, 2011. 178(6): p. 2611-21; Andreetta, F., et al., J Neuroimmunol, 2006. 175(1-2): p. 77-86; Gumucio, J. P., et al., J Appl Physiol (1985), 2013. 115(4): p. 539-45). In addition, myofiber-specific expression of a dominant negative TGFβ type II receptor protects against muscle damage after cardiotoxin injury and in δ-sarcoglycan−/− mice (Accornero, F., et al., Hum Mol Genet, 2014. 23(25): p. 6903-15). The proteoglycan decorin, which is abundant in skeletal muscle and inhibits TGFβ activity, decreases muscle fibrosis in mdx mice and following laceration injury (Li, Y., et al., Mol Ther, 2007. 15(9): p. 1616-22; Gosselin, L. E., et al., Muscle Nerve, 2004. 30(5): p. 645-53). Other molecules with TGFβ inhibitory activity, such as suramin (an anti-neoplastic agent) and losartan (an angiotensin receptor blocker) have been effective in improving muscle pathology and reducing fibrosis in mouse models of injury, Marfan's syndrome, and muscular dystrophy (Spurney, C. F., et al., J Cardiovasc Pharmacol Ther, 2011. 16(1): p. 87-95; Taniguti, A. P., et al., Muscle Nerve, 2011. 43(1): p. 82-7; Bedair, H. S., et al., Am J Sports Med, 2008. 36(8): p. 1548-54; Cohn, R. D., et al., Nat Med, 2007. 13(2): p. 204-10). While all of the therapeutic agents described above do inhibit TGFβ1 or its signaling, none of them is specific for the TGFβ1 isoform. For example, 1D11 binds to and inhibits the TGFβ1, 2, and 3 isoforms (Dasch, J. R., et al., J Immunol, 1989. 142(5): p. 1536-41). Suramin inhibits the ability of multiple growth factors to bind to their receptors, including PDGF, FGF, and EGF, in addition to TGFβ1 (Hosang, M., J Cell Biochem, 1985. 29(3): p. 265-73; Olivier, S., et al., Eur J Cancer, 1990. 26(8): p. 867-71; Scher, H. I. and W. D. Heston, Cancer Treat Res, 1992. 59: p. 131-51). Decorin also inhibits myostatin activity, both by direct binding and through upregulation of follistatin, a myostatin inhibitor (Miura, T., et al., Biochem Biophys Res Commun, 2006. 340(2): p. 675-80; Brandan, E., C. Cabello-Verrugio, and C. Vial, Matrix Biol, 2008. 27(8): p. 700-8; Zhu, J., et al., J Biol Chem, 2007. 282(35): p. 25852-63). Losartan affects additional signaling pathways through its effects on the renin-angiotensin-aldosterone system, including the IGF-1/AKT/mTOR pathway (Burks, T. N., et al., Sci Transl Med, 2011. 3(82): p. 82ra37; Sabharwal, R. and M. W. Chapleau, Exp Physiol, 2014. 99(4): p. 627-31; McIntyre, M., et al., Pharmacol Ther, 1997. 74(2): p. 181-94). Therefore, all of these therapies inhibit additional molecules which may contribute to their therapeutic effects, as well as toxicities.

Considering the postulated role of TGFβ in muscle homeostasis, repair, and regeneration, agents, such as monoclonal antibodies described herein, that selectively modulate TGFβ1 signaling may be effective for treating damaged muscle fibers, such as in chronic/genetic muscular dystrophies and acute muscle injuries, without the toxicities associated with more broadly-acting TGFβ inhibitors developed to date.

Accordingly, the present invention provides methods for treating damaged muscle fibers using an agent that preferentially modulates a subset, but not all, of TGFβ effects in vivo. Such agents can selectively modulate TGFβ1 signaling (“isoform-specific modulation”).

### Muscle Fiber Repair in Chronic Muscular Diseases:

The invention encompasses methods to improve muscle quality and function in DMD patients, by limiting fibrosis and contributing to a normalization of muscle morphology and function. As TGFβ1 also inhibits myogenesis, TGFβ1 blockade may promote regeneration in dystrophic muscle, adding further therapeutic benefit. TGFβ1 inhibitors may be used in combination with dystrophin upregulating therapies, such as Exondys 51 (Eteplirsen). Given the potential therapeutic benefits of TGFβ1 inhibition in muscular dystrophy, it is critical to (1) differentiate the role(s) of TGFβ1 from those of TGFβ2 and TGFβ3, and (2) clarify in which molecular context(s) TGFβ1 inhibition would be most beneficial. As mentioned above, pan-TGFβ inhibitors have been associated with significant toxicities, limiting the clinical use of these compounds (Anderton, M. J., et al., Toxicol Pathol, 2011. 39(6): p. 916-24; Stauber, A., et al., Clinical Toxicology, 2014. 4(3): p. 1-10). It is unclear which of the TGFβ isoform(s) causes these toxicities. Some of the described toxicities may be due to TGFβ1 inhibition in the immune system. For example, while 1D11 significantly reduced levels of fibrosis in the diaphragm, treatment also increased numbers of CD4+ and CD8+ T cells in the muscle, suggesting an increased inflammatory response upon pan-TGFβ inhibition which could be detrimental with long-term treatment (Andreetta, F., et al., J Neuroimmunol, 2006. 175(1-2): p. 77-86). Indeed, depletion of T cells from muscle improves the muscle pathology of mdx mice, suggesting T-cell mediated inflammatory responses are detrimental to dystrophic muscle (Spencer, M. J., et al., Clin Immunol, 2001. 98(2): p. 235-43). Increases in T cell numbers upon 1D11 administration are likely due to the effects of TGFβ1 on regulatory T (Treg) cells. Tregs present TGFβ1 on their cell surface via GARP, and release of TGFβ1 from this complex enhances Treg suppressive activity, thus limiting T cell mediated inflammation (Wang, R., et al., Mol Biol Cell, 2012. 23(6): p. 1129-39; Edwards, J. P., A. M. Thornton, and E. M. Shevach, J Immunol, 2014. 193(6): p. 2843-9; Nakamura, K., et al., J Immunol, 2004. 172(2): p. 834-42; Nakamura, K., A. Kitani, and W. Strober, J Exp Med, 2001. 194(5): p. 629-44). Indeed, depletion of Tregs using the PC61 antibody resulted in increased inflammation and muscle damage in the diaphragm of mdx mice, while augmentation of Treg numbers and activity reduced muscle damage (Villalta, S. A., et al., Sci Transl Med, 2014. 6(258): p. 258ra142). Interestingly, an additional population of immunosuppressive T cells, Tr1 cells, has recently been identified. These cells produce large amounts of TGFβ3, which is required for their suppressive activity (Gagliani, N., et al., Nat Med, 2013. 19(6): p. 739-46; Okamura, T., et al., Proc Natl Acad Sci USA, 2009. 106(33): p. 13974-9; Okamura, T., et al., Nat Commun, 2015. 6: p. 6329). While the role of Tr1 cells in skeletal muscle is unknown, the possibility exists that inhibition of both TGFβ1 and TGFβ3 by 1D11 could have additive pro-inflammatory effects by inhibiting both Tregs and Tr1 cells.

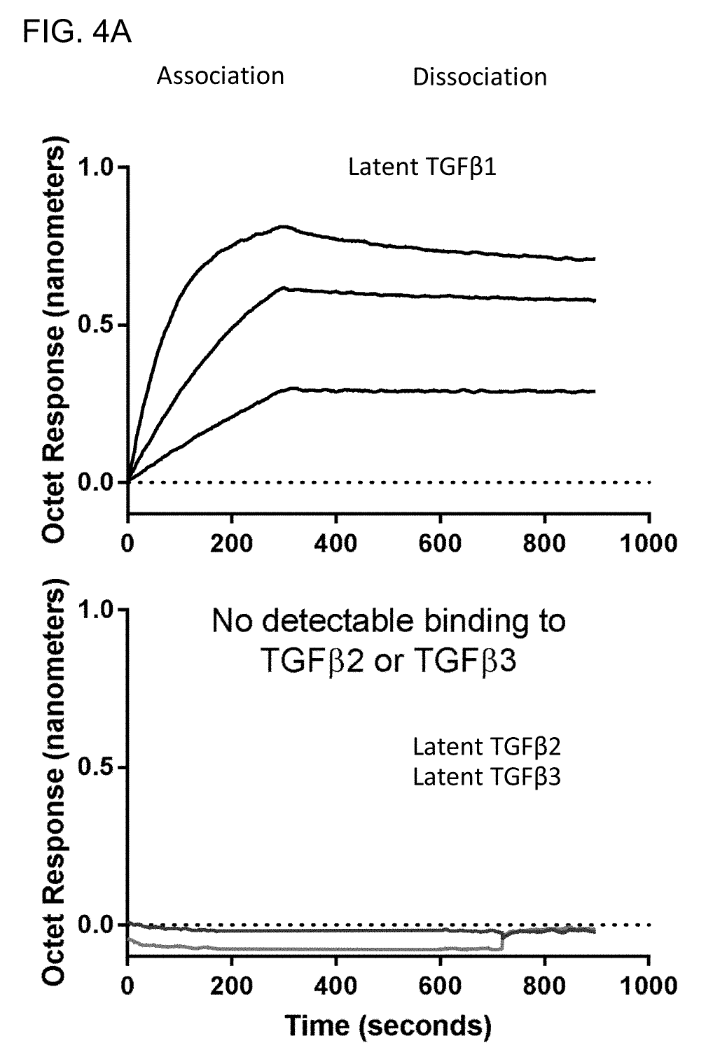

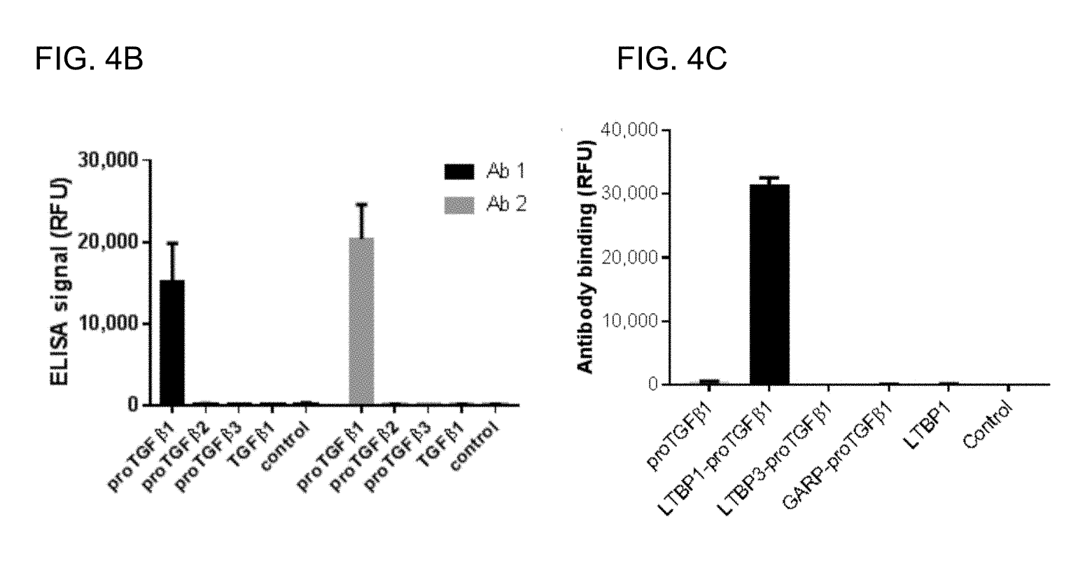

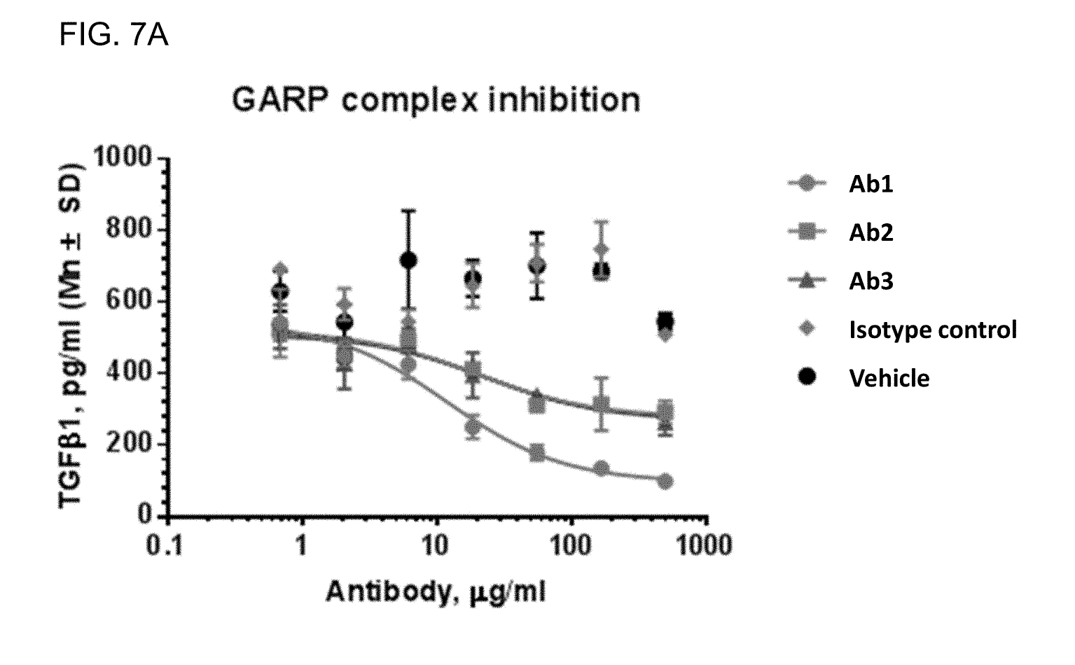

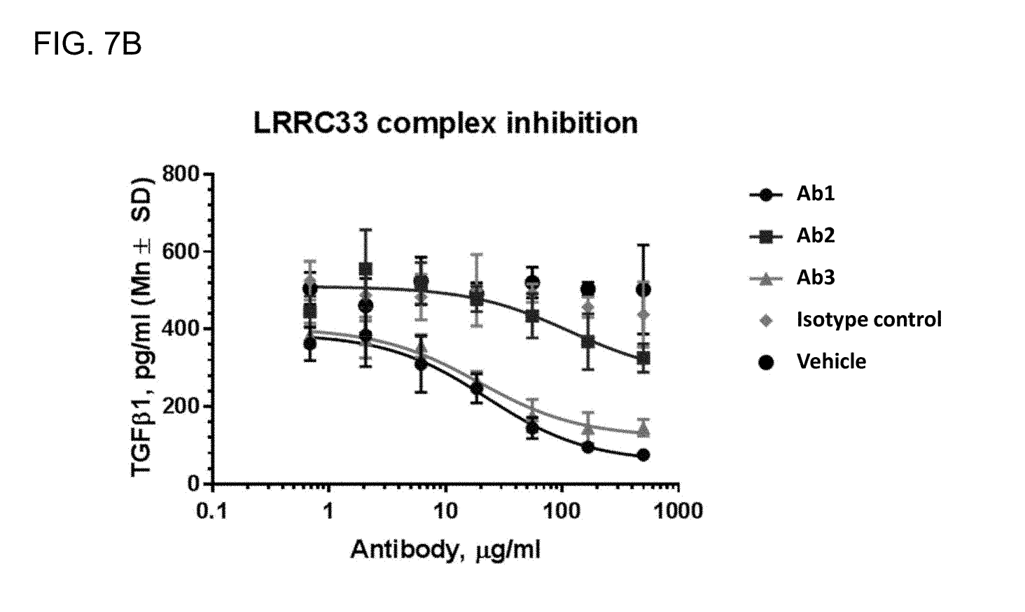

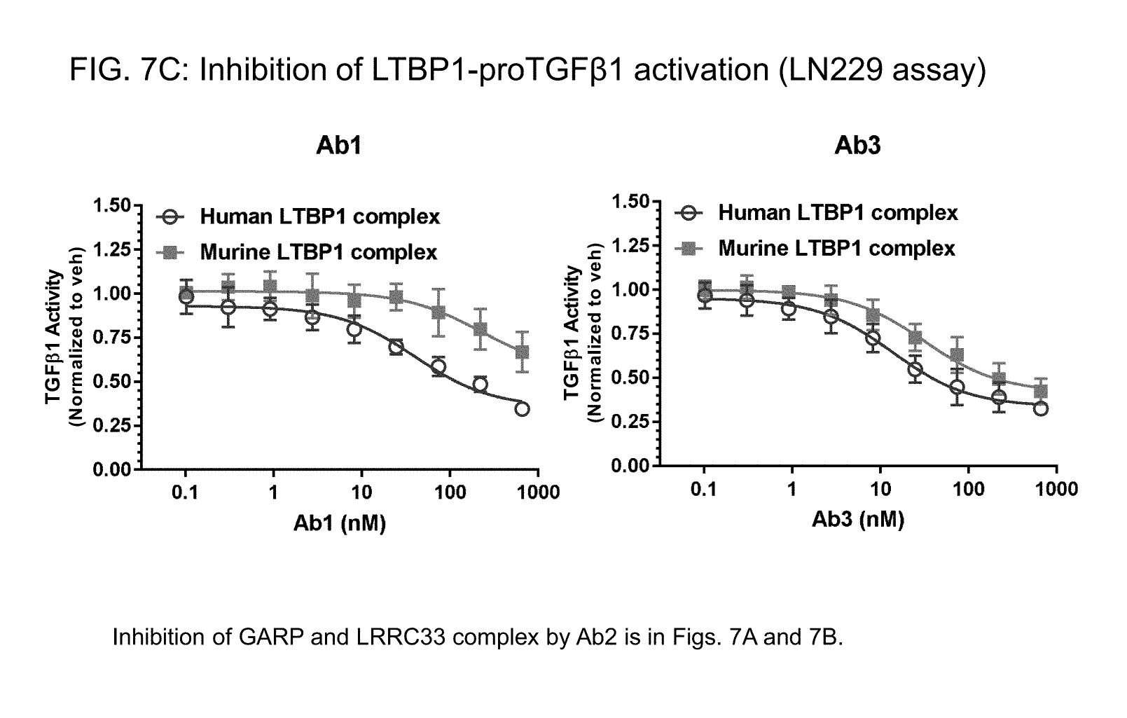

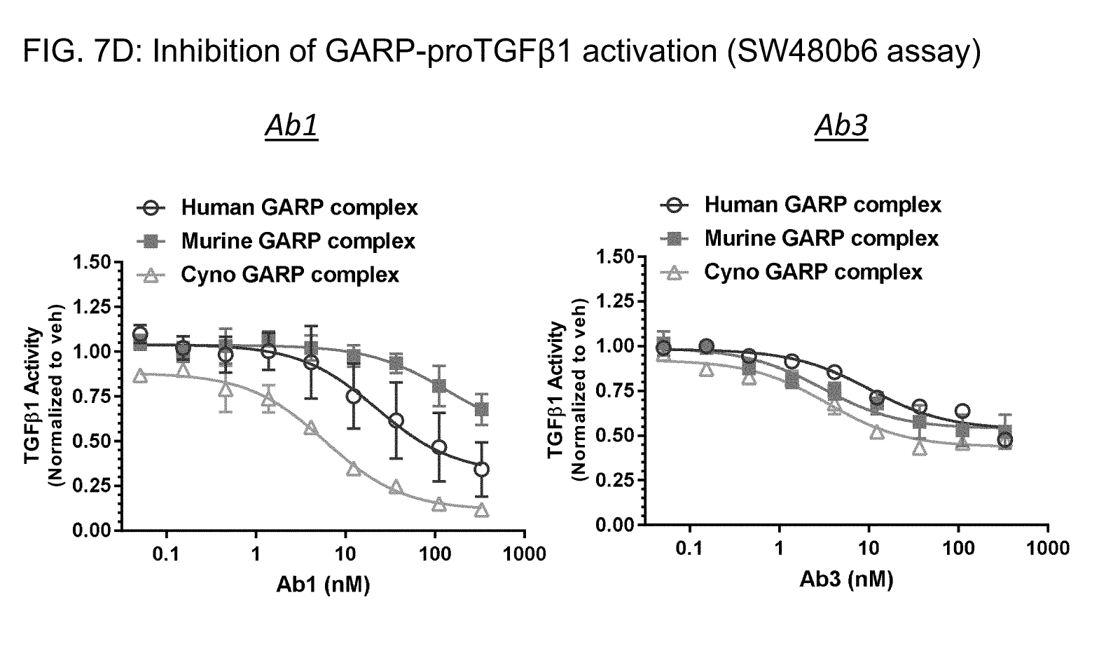

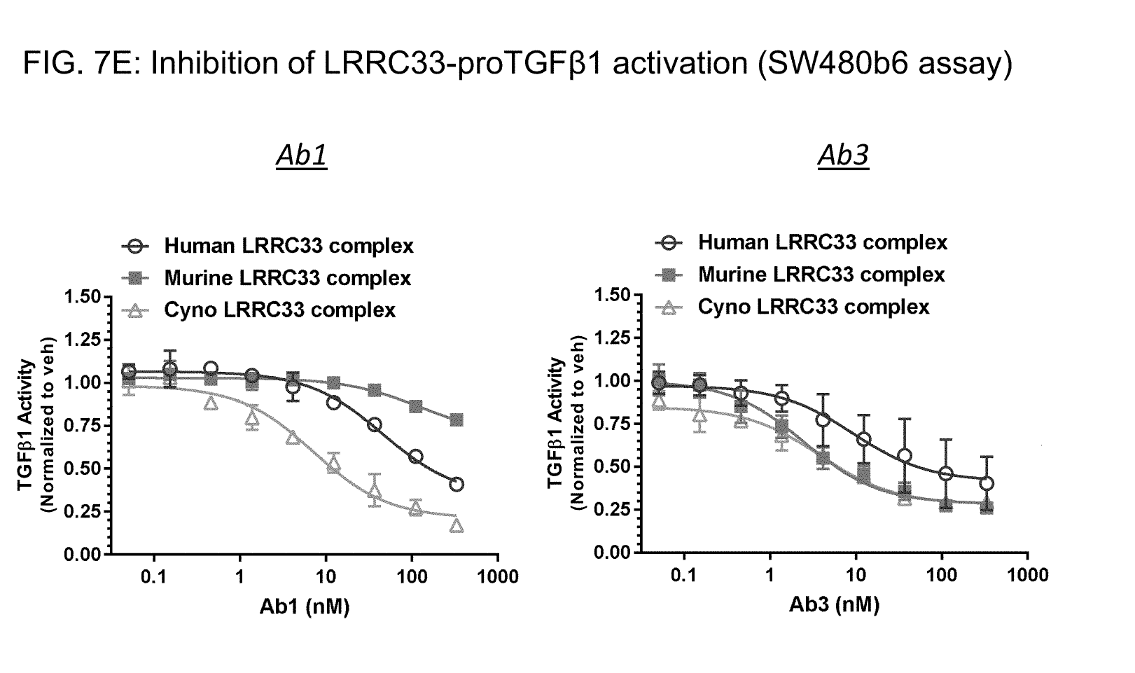

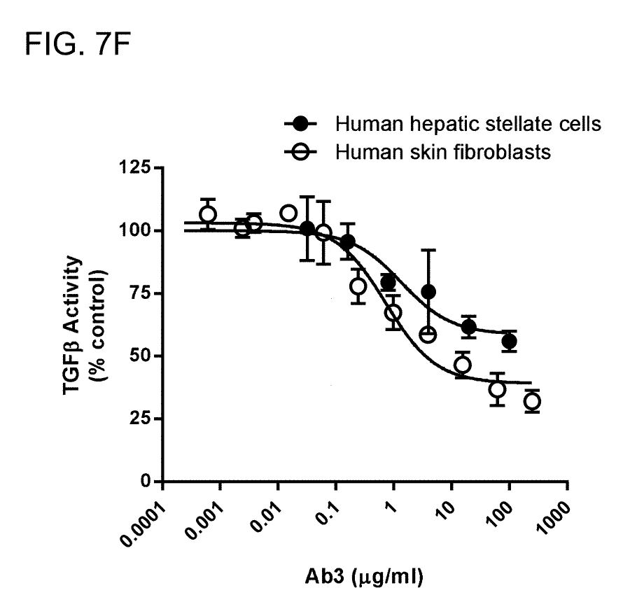

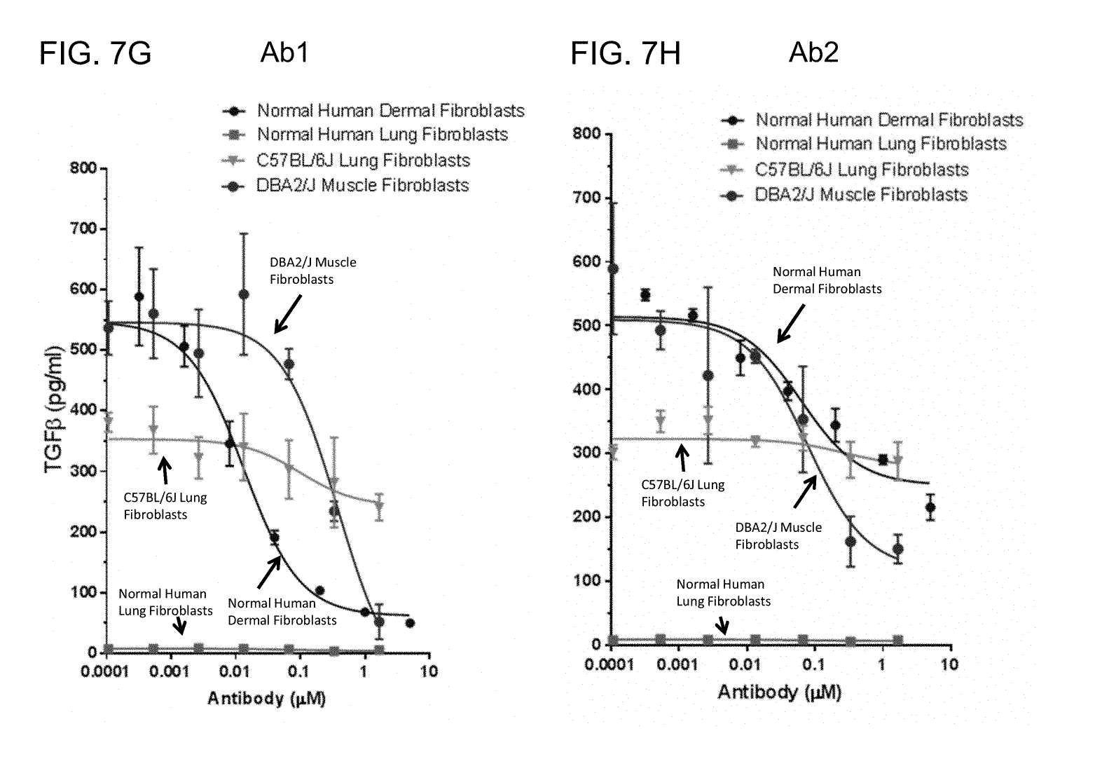

The structural insights described above regarding TGFβ1 latency and activation allow for novel approaches to drugs discovery that specifically target activation of TGFβ1 (Shi, M., et al., Nature, 2011. 474(7351): p. 343-9). The high degree of sequence identity shared between the three mature TGFβ growth factors is not shared by the latent complexes, allowing for the discovery of antibodies that are exquisitely specific to proTGFβ1. Using proprietary approaches to antibody discovery, the instant inventors have identified antibodies (Ab1, Ab2 and Ab3) which specifically bind to proTGFβ1 (see for example FIG. 4B). Using an in vitro co-culture system these antibodies were demonstrated to inhibit integrin-mediated release of TGFβ1. In this system, fibroblasts derived from human skin or mouse skeletal muscles are the source of latent TGFβ1, a cell line expressing αVβ6 allows for release of active TGFβ1, which is then measured using a third cell line expressing a SMAD2/3 responsive luciferase reporter (FIGS. 7G-7H). One of these antibodies, Ab1, has been tested in vivo and shown efficacy in the UUO (unilateral ureteral obstruction) mouse model of kidney fibrosis. In this model, treatment of mice (n=10) with 9 mg/kg/week Ab1 prevented upregulation of TGFβ1-responsive genes (FIGS. 12A-12J) and reduced the extent of fibrosis following injury (by picrosirius red staining) (FIG. 12K). TGFβ1 specific therapies may have improved efficacy and safety profiles compared to pan-TGFβ inhibitors, a critical aspect for a therapeutic which would be used long term as in the DMD population. TGFβ1 inhibitory antibodies can be used to determine if specific TGFβ1 inhibition has potential as a therapeutic for DMD or other muscle diseases, and to clarify the role of TGFβ1 in skeletal muscle regeneration.

Chronic Vs. Acute Myofiber Injuries and Selection of Optimal Therapeutics:

In normal, but regenerating muscle following an acute injury (such as traumatic injury to otherwise healthy muscles or motor neurons), it is believed that the initial infiltration of inflammatory macrophages is required to clear out the damaged tissue and to secrete factors (e.g., cytokines) necessary for satellite cell activation. Subsequently, these cells switch to the M2 phenotype to drive wound resolution.

By contrast, in chronic conditions, such as diseases including DMD, the pro-inflammatory macrophages predominated at all time, and that switch to M2 does not happen (or at least not efficiently enough), and the pro-inflammatory macrophages continue to drive inflammation and muscle damage. In DMD, the NFkB pathway is perpetually active, resulting in constitutive inflammation. In some embodiments, therefore, an NFkB inhibitor may be administered to DMD patients in order to reduce the chronic inflammation.

Thus, in chronic conditions such as DMD, therapeutic focus may be on muscle repair as opposed to muscle regeneration. This is because DMD muscle fibers are defective but not destroyed—they are damaged by tears in the membrane, dysregulation of calcium transients, and ROS damage from the macrophages. In comparison, in cases of injuries to healthy muscles, therapeutic focus may be on regeneration. For example, in cardiotoxin models, muscle fibers are killed and have to be regenerated. This simulates the process of recovery after a traumatic injury, such as crush injury.

Evidence suggests that LRRC33 is expressed in thioglycollate-induced peritoneal macrophages, which have an M2-like phenotype (characterized in that they express high levels of Arginase, no iNOS, and high levels of CD206).

In situations where LRRC33 is expressed primarily on the M2 cells and where its presentation of TGFβ1 (“context”) is important for the pro-wound healing effects of these cells, it may be beneficial to activate LRRC33-mediated TGFβ1 to promote repair and/or myogenesis. On the other hand, in situations where LRRC33 is also expressed on the pro-inflammatory M1 cells, then it may be beneficial to inhibit LRRC33-mediated TGFβ1, given that inflammation drives the fibrosis, especially in the dystrophic setting, such as DMD. Thus, identifying the source/context of disease-associated TGFβ1 can be an important step in selecting the right modulator of the TGFβ signaling, which will inform what level of selectivity should be considered (e.g., isoform-specific, context-permissive TGFβ1 modulators, or, context-specific TGFβ1 modulators; TGFβ1 inhibitors or activators, etc.).

Apart from chronic inflammation, the hallmark of DMD is excessive, and progressive, fibrosis. In advanced disease the fibrosis is so severe that it can actually isolate individual muscle fibers from their blood supply. It also alters the contractile properties of the muscle. In human patients, there is a strong correlation between the extent of TGFβ1 upregulation and fibrosis, and a strong link between the extent of fibrosis and negative mobility outcomes. Therefore, in some embodiments, LTBP-proTGFβ1 inhibitors may be administered to dystrophic patients for the prevention and/or reduction of fibrosis to selectively target the ECM-associated TGFβ1 effects in the disease. In some embodiments, various isoform- and/or context-selective agents described herein can be employed to achieve inhibition of TGFβ1 signaling to prevent fibrosis and promote myogenesis, but without having unwanted effects on the immune system (e.g., through GARP or LRRC33).

### Treatments, Administration

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, antibodies, or antigen binding portions thereof, that specifically bind a GARP-TGFβ1 complex, a LTBP1-TGFβ1 complex, a LTBP3-TGFβ1 complex, and/or a LRRC33-TGFβ1 complex can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a TGFβ-related indication, such as those noted above. A subject having a TGFβ-related indication can be identified by routine medical examination, e.g., laboratory tests, organ functional tests, CT scans, or ultrasounds. A subject suspected of having any of such indication might show one or more symptoms of the indication. A subject at risk for the indication can be a subject having one or more of the risk factors for that indication.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the intended purpose may be to inhibit TGFβ-1 activation in vivo, to achieve clinically meaningful outcome associated with the TGFβ-1 inhibition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a TGFβ-related indication. Alternatively, sustained continuous release formulations of an antibody that specifically binds a GARP-TGFβ1 complex, a LTBP1-TGFβ1 complex, a LTBP3-TGFβ1 complex, and/or a LRRC33-TGFβ1 complex may be appropriate. Various formulations and devices for achieving sustained release would be apparent to the skilled artisan and are within the scope of this disclosure.

In one example, dosages for an antibody that specifically binds a GARP-TGFβ1 complex, a LTBP1-TGFβ1 complex, a LTBP3-TGFβ1 complex, and/or a LRRC33-TGFβ1 complex as described herein may be determined empirically in individuals who have been given one or more administration(s) of the antibody. Individuals are given incremental dosages of the antagonist. To assess efficacy, an indicator of the TGFβ-related indication can be followed. For example, methods for measuring for myofiber damage, myofiber repair, inflammation levels in muscle, and/or fibrosis levels in muscle are well known to one of ordinary skill in the art.

The present invention encompasses the recognition that agents capable of modulating the activation step of TGFβs in an isoform-specific manner may provide improved safety profiles when used as a medicament. Accordingly, the invention includes antibodies and antigen-binding fragments thereof that specifically bind and inhibit activation of TGFβ1, but not TGFβ2 or TGFβ3, thereby conferring specific inhibition of the TGFβ1 signaling in vivo while minimizing unwanted side effects from affecting TGFβ2 and/or TGFβ3 signaling.

In some embodiments, the antibodies, or antigen binding portions thereof, as described herein, are not toxic when administered to a subject. In some embodiments, the antibodies, or antigen binding portions thereof, as described herein, exhibit reduced toxicity when administered to a subject as compared to an antibody that specifically binds to both TGFβ1 and TGFβ2. In some embodiments, the antibodies, or antigen binding portions thereof, as described herein, exhibit reduced toxicity when administered to a subject as compared to an antibody that specifically binds to both TGFβ1 and TGFβ3. In some embodiments, the antibodies, or antigen binding portions thereof, as described herein, exhibit reduced toxicity when administered to a subject as compared to an antibody that specifically binds to TGFβ1, TGFβ2 and TGFβ3.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a TGFβ-related indication, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. Pharmacokinetics experiments have shown that the serum concentration of an antibody disclosed herein (e.g., Ab2) remains stable for at least 7 days after administration to a preclinical animal model (e.g., a mouse model). Without wishing to be bound by any particular theory, this stability post-administration may be advantageous since the antibody may be administered less frequently while maintaining a clinically effective serum concentration in the subject to whom the antibody is administered (e.g., a human subject). In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. The particular dosage regimen, e.g., dose, timing and repetition, will depend on the particular individual and that individual's medical history, as well as the properties of the individual agents (such as the half-life of the agent, and other relevant considerations).

For the purpose of the present disclosure, the appropriate dosage of an antibody that specifically binds a GARP-TGFβ1 complex, a LTBP1-TGFβ1 complex, a LTBP3-TGFβ1 complex, and/or a LRRC33-TGFβ1 complex will depend on the specific antibody (or compositions thereof) employed, the type and severity of the indication, whether the antibody is administered for preventive or therapeutic purposes, previous therapy, the patient's clinical history and response to the antagonist, and the discretion of the attending physician. In some embodiments, a clinician will administer an antibody that specifically binds a GARP-TGFβ1 complex, a LTBP1-TGFβ1 complex, a LTBP3-TGFβ1 complex, and/or a LRRC33-TGFβ1 complex, until a dosage is reached that achieves the desired result. Administration of an antibody that specifically binds a GARP-TGFβ1 complex, a LTBP1-TGFβ1 complex, a LTBP3-TGFβ1 complex, and/or a LRRC33-TGFβ1 complex can be continuous or intermittent, depending, for example, upon the recipient's physiological condition, whether the purpose of the administration is therapeutic or prophylactic, and other factors known to skilled practitioners. The administration of antibody that specifically binds a GARP-TGFβ1 complex, a LTBP1-TGFβ1 complex, a LTBP3-TGFβ1 complex, and/or a LRRC33-TGFβ1 complex may be essentially continuous over a preselected period of time or may be in a series of spaced dose, e.g., either before, during, or after developing a TGFβ-related indication.

As used herein, the term “treating” refers to the application or administration of a composition including one or more active agents to a subject, who has a TGFβ-related indication, a symptom of the indication, or a predisposition toward the indication, with the purpose to cure, heal, alleviate, relieve, alter, remedy, ameliorate, improve, or affect the indication, the symptom of the indication, or the predisposition toward the indication.

Alleviating a TGFβ-related indication with an antibody that specifically binds a GARP-TGFβ1 complex, a LTBP1-TGFβ1 complex, a LTBP3-TGFβ1 complex, and/or a LRRC33-TGFβ1 complex includes delaying the development or progression of the indication, or reducing indication's severity. Alleviating the indication does not necessarily require curative results. As used therein, “delaying” the development of an indication associated with a TGFβ-related indication means to defer, hinder, slow, retard, stabilize, and/or postpone progression of the indication. This delay can be of varying lengths of time, depending on the history of the indication and/or individuals being treated. A method that “delays” or alleviates the development of an indication, or delays the onset of the indication, is a method that reduces probability of developing one or more symptoms of the indication in a given time frame and/or reduces extent of the symptoms in a given time frame, when compared to not using the method. Such comparisons are typically based on clinical studies, using a number of subjects sufficient to give a statistically significant result.

DBA2/J mice have a 40 bp deletion in the LTBP4 allele. Dysregulation of the ECM to which latent TGFb1 is associated may expose the epitope to which Ab1 binds. There may be diseases in which the epitope to which Ab1 binds gets exposed, and those diseases may be therapeutic opportunities for Ab1 if TGFb1 inhibition is indicated.

### Combination Therapies

The disclosure further encompasses pharmaceutical compositions and related methods used as combination therapies for treating subjects who may benefit from TGFβ inhibition in vivo. In any of these embodiments, such subjects may receive combination therapies that include a first composition comprising at least one TGFβ inhibitor, e.g., antibody or antigen-binding portion thereof, described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. To give but one example, the first composition may treat a disease or condition associated with TGFβ signaling, while the second composition may treat inflammation or fibrosis associated with the same disease, etc. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

Accordingly, the invention provides pharmaceutical compositions and methods for use in combination therapies for the reduction of TGFβ1 protein activation and the treatment or prevention of diseases or conditions associated with TGFβ1 signaling, as described herein. Accordingly, the methods or the pharmaceutical compositions further comprise a second therapy. In some embodiments, the second therapy may be useful in treating or preventing diseases or conditions associated with TGFβ1 signaling. The second therapy may diminish or treat at least one symptom(s) associated with the targeted disease. The first and second therapies may exert their biological effects by similar or unrelated mechanisms of action; or either one or both of the first and second therapies may exert their biological effects by a multiplicity of mechanisms of action.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

The one or more anti-TGFβ antibodies, or antigen binding portions thereof, of the invention may be used in combination with one or more of additional therapeutic agents. Examples of the additional therapeutic agents which can be used with an anti-TGFβ antibody of the invention include, but are not limited to: a modulator of a member of the TGFβ superfamily, such as a myostatin inhibitor and a GDF11 inhibitor; a VEGF agonist; an IGF1 agonist; an FXR agonist; a CCR2 inhibitor; a CCR5 inhibitor; a dual CCR2/CCR5 inhibitor; a lysyl oxidase-like-2 inhibitor; an ASK1 inhibitor; an Acetyl-CoA Carboxylase (ACC) inhibitor; a p38 kinase inhibitor; Pirfenidone; Nintedanib; an M-CSF inhibitor (e.g., M-CSF receptor antagonist and M-CSF neutralizing agents); a MAPK inhibitor (e.g., Erk inhibitor), an immune checkpoint agonist or antagonist; an IL-11 antagonist; and IL-6 antagonist, and the like. Other examples of the additional therapeutic agents which can be used with the TGFβ inhibitors include, but are not limited to, an indoleamine 2,3-dioxygenase (IDO) inhibitor, a tyrosine kinase inhibitor, Ser/Thr kinase inhibitor, a dual-specific kinase inhibitor. In some embodiments, such an agent may be a PI3K inhibitor, a PKC inhibitor, or a JAK inhibitor.

In some embodiments, the additional agent is a checkpoint inhibitor. In some embodiments, the additional agent is selected from the group consisting of a PD-1 antagonist, a PDL1 antagonist, a PD-L1 or PDL2 fusion protein, a CTLA4 antagonist, a GITR agonist, an anti-ICOS antibody, an anti-ICOSL antibody, an anti-B7H3 antibody, an anti-B7H4 antibody, an anti-TIM3 antibody, an anti-LAG3 antibody, an anti-OX40 antibody, an anti-CD27 antibody, an anti-CD70 antibody, an anti-CD47 antibody, an anti-41 BB antibody, an anti-PD-1 antibody, an oncolytic virus, and a PARP inhibitor.

In some embodiments, the additional agent binds a T-cell costimulation molecule, such as inhibitory costimulation molecules and activating costimulation molecules. In some embodiments, the additional agent is selected from the group consisting of an anti-CD40 antibody, an anti-CD38 antibody, an anti-KIR antibody, an anti-CD33 antibody, an anti-CD137 antibody, and an anti-CD74 antibody.

In some embodiments, the additional therapy is radiation. In some embodiments, the additional agent is a chemotherapeutic agent. In some embodiments, the chemotherapeutic agent is Taxol. In some embodiments, the additional agent is an anti-inflammatory agent. In some embodiments, the additional agent inhibits the process of monocyte/macrophage recruitment and/or tissue infiltration. In some embodiments, the additional agent is an inhibitor of hepatic stellate cell activation. In some embodiments, the additional agent is a chemokine receptor antagonist, e.g., CCR2 antagonists and CCR5 antagonists. In some embodiments, such chemokine receptor antagonist is a dual specific antagonist, such as a CCR2/CCR5 antagonist. In some embodiments, the additional agent to be administered as combination therapy is or comprises a member of the TGFβ superfamily of growth factors or regulators thereof. In some embodiments, such agent is selected from modulators (e.g., inhibitors and activators) of GDF8/myostatin and GDF11. In some embodiments, such agent is an inhibitor of GDF8/myostatin signaling. In some embodiments, such agent is a monoclonal antibody that specifically binds a pro/latent myostatin complex and blocks activation of myostatin. In some embodiments, the monoclonal antibody that specifically binds a pro/latent myostatin complex and blocks activation of myostatin does not bind free, mature myostatin.

In some embodiments, an additional therapy comprises CAR-T therapy.

Such combination therapies may advantageously utilize lower dosages of the administered therapeutic agents, thus avoiding possible toxicities or complications associated with the various monotherapies. In some embodiments, use of an isoform-specific inhibitor of TGFβ1 described herein may render those who are poorly responsive or not responsive to a therapy (e.g., standard of care) more responsive. In some embodiments, use of an isoform-specific inhibitor of TGFβ1 described herein may allow reduced dosage of the therapy (e.g., standard of care) which still produces equivalent clinical efficacy in patients but fewer or lesser degrees of drug-related toxicities or adverse events.

### Inhibition of TGFβ1 Activity

Methods of the present disclosure include methods of inhibiting TGFβ1 growth factor activity in one or more biological system. Such methods may include contacting one or more biological system with an antibody and/or composition of the disclosure. In some cases, these methods include modifying the level of free growth factor in a biological system (e.g. in a cell niche or subject). Antibodies and/or compositions according to such methods may include, but are not limited to biomolecules, including, but not limited to recombinant proteins, protein complexes and/or antibodies, or antigen portions thereof, described herein.

In some embodiments, methods of the present disclosure may be used to reduce or eliminate growth factor activity, termed “inhibiting methods” herein. Some such methods may comprise mature growth factor retention in a TGFβ complex (e.g., a TGFβ1 complexed with GARP, LTBP1, LTBP3 and/or LRRC33) and/or promotion of reassociation of growth factor into a TGFβ complex. In some cases, inhibiting methods may comprise the use of an antibody that specifically binds a GARP-TGFβ1 complex, a LTBP1-TGFβ1 complex, a LTBP3-TGFβ1 complex, and/or a LRRC33-TGFβ1 complex. According to some inhibiting methods, one or more inhibiting antibody is provided.

In some embodiments, antibodies, antigen binding portions thereof, and compositions of the disclosure may be used for inhibiting TGFβ1 activation. In some embodiments, provided herein is a method for inhibiting TGFβ1 activation comprising exposing a GARP-TGFβ1 complex, a LTBP1-TGFβ1 complex, a LTBP3-TGFβ1 complex, and/or a LRRC33-TGFβ1 complex to an antibody, an antigen binding portion thereof, or a pharmaceutical composition described herein. In some embodiments, the antibody, antigen binding portion thereof, or pharmaceutical composition, inhibits the release of mature TGFβ1 from the GARP-TGFβ1 complex, the LTBP1-TGFβ1 complex, a LTBP3-TGFβ1 complex, and/or the LRRC33-TGFβ1 complex. In some embodiments, the method is performed in vitro. In some embodiments, the method is performed in vivo. In some embodiments, the method is performed ex vivo.

In some embodiments, the GARP-TGFβ1 complex or the LRRC33-TGFβ1 complex is present at the outer surface of a cell.

In some embodiments, the cell expressing the GARP-TGFβ1 complex or the LRRC33-TGFβ1 complex is a T-cell, a fibroblast, a myofibroblast, a macrophage, a monocyte, a dendritic cell, an antigen presenting cell, a neutrophil, a myeloid-derived suppressor cell (MDSC), a lymphocyte, a mast cell, or a microglia. The T-cell may be a regulatory T cell (e.g., immunosuppressive T cell). The neuprophil may be an activated neutrophil. The macrophage may be an activated (e.g., polarized) macrophage, including profibrotic and/or tumor-associated macrophages (TAM), e.g., M2c subtype and M2d subtype macrophages. In some embodiments, macrophages are exposed to tumor-derived factors (e.g., cytokines, growth factors, etc.) which may further induce pro-cancer phenotypes in macrophages. In some embodiments, such tumor-derived factor is CSF-1/M-CSF.

In some embodiments, the cell expressing the GARP-TGFβ1 complex or the LRRC33-TGFβ1 complex is a cancer cell, e.g., circulating cancer cells and tumor cells.

In some embodiments, the LTBP1-TGFβ1 complex or the LTBP3-TGFβ1 complex is bound to an extracellular matrix (i.e., components of the ECM). In some embodiments, the extracellular matrix comprises fibrillin and/or fibronectin. In some embodiments, the extracellular matrix comprises a protein comprising an RGD motif.

LRRC33 is expressed in selective cell types, in particular those of myeloid lineage, including monocytes and macrophages. Monocytes originated from progenitors in the bone marrow and circulate in the bloodstream and reach peripheral tissues. Circulating monocytes can then migrate into tissues where they become exposed to the local environment (e.g., tissue-specific, disease-associated, etc.) that includes a panel of various factors, such as cytokines and chemokines, triggering differentiation of monocytes into macrophages, dendritic cells, etc. These include, for example, alveolar macrophages in the lung, osteoclasts in bone marrow, microglia in the CNS, histiocytes in connective tissues, Kupffer cells in the liver, and brown adipose tissue macrophages in brown adipose tissues. In a solid tumor, infiltrated macrophages may be tumor-associated macrophages (TAMs), tumor-associated neutrophils (TANs), and myeloid-derived suppressor cells (MDSCs), etc. Such macrophages may activate and/or be associated with activated fibroblasts, such as carcinoma-associated (or cancer-associated) fibroblasts (CAFs) and/or the stroma. Thus, inhibitors of TGFβ1 activation described herein which inhibits release of mature TGFβ1 from LRRC33-containing complexes can target any of these cells expressing LRRC33-proTGFβ1 on cell surface.

In some embodiments, the LRRC33-TGFβ1 complex is present at the outer surface of profibrotic (M2-like) macrophages. In some embodiments, the profibrotic (M2-like) macrophages are present in the fibrotic microenvironment. In some embodiments, targeting of the LRRC33-TGFβ1 complex at the outer surface of profibrotic (M2-like) macrophages provides a superior effect as compared to solely targeting LTBP1-TGFβ1 and/or LTBP1-TGFβ1 complexes. In some embodiments, M2-like macrophages, are further polarized into multiple subtypes with differential phenotypes, such as M2c and M2d TAM-like macrophages. In some embodiments, macrophages may become activated by various factors (e.g., growth factors, chemokines, cytokines and ECM-remodeling molecules) present in the tumor microenvironment, including but are not limited to TGFβ1, CCL2 (MCP-1), CCL22, SDF-1/CXCL12, M-CSF (CSF-1), IL-6, IL-8, IL-10, IL-11, CXCR4, VEGF, PDGF, prostaglandin-regulating agents such as arachidonic acid and cyclooxygenase-2 (COX-2), parathyroid hormone-related protein (PTHrP), RUNX2, HIF1α, and metalloproteinases. Exposures to one or more of such factors may further drive monocytes/macrophages into pro-tumor phenotypes. In turn, these activated tumor-associated cells may also facilitate recruitment and/or differentiation of other cells into pro-tumor cells, e.g., CAFs, TANs, MDSCs, and the like. Stromal cells may also respond to macrophage activation and affect ECM remodeling, and ultimately vascularization, invasion, and metastasis.

In some embodiments, the GARP-TGFβ1 complex, the LTBP1-TGFβ1 complex, the LTBP3-TGFβ1 complex, and/or the LRRC33-TGFβ1 complex is bound to an extracellular matrix. In some embodiments, the extracellular matrix comprises fibrillin. In some embodiments, the extracellular matrix comprises a protein comprising an RGD motif.

In some embodiments, provided herein is a method for reducing TGFβ1 protein activation in a subject comprising administering an antibody, an antigen binding portion thereof, or a pharmaceutical composition described herein to the subject, thereby reducing TGFβ1 protein activation in the subject. In some embodiments, the subject has or is at risk of having fibrosis. In some embodiments, the subject has or is at risk of having cancer. In some embodiments, the subject has or is at risk of having dementia.

In some embodiments, the antibodies, or the antigen binding portions thereof, as described herein, reduce the suppressive activity of regulatory T cells (Tregs).

Kits for Use in Alleviating Diseases/Disorders Associated with a TGFβ-Related Indication

The present disclosure also provides kits for use in alleviating diseases/disorders associated with a TGFβ-related indication. Such kits can include one or more containers comprising an antibody, or antigen binding portion thereof, that specifically binds to a GARP-TGFβ1 complex, a LTBP1-TGFβ1 complex, a LTBP3-TGFβ1 complex, and/or a LRRC33-TGFβ1 complex, e.g., any of those described herein.

In some embodiments, the kit can comprise instructions for use in accordance with any of the methods described herein. The included instructions can comprise a description of administration of the antibody, or antigen binding portion thereof, that specifically binds a GARP-TGFβ1 complex, a LTBP1-TGFβ1 complex, a LTBP3-TGFβ1 complex, and/or a LRRC33-TGFβ1 complex to treat, delay the onset, or alleviate a target disease as those described herein. The kit may further comprise a description of selecting an individual suitable for treatment based on identifying whether that individual has the target disease. In still other embodiments, the instructions comprise a description of administering an antibody, or antigen binding portion thereof, to an individual at risk of the target disease.

The instructions relating to the use of antibodies, or antigen binding portions thereof, that specifically binds a GARP-TGFβ1 complex, a LTBP1-TGFβ1 complex, a LTBP3-TGFβ1 complex, and/or a LRRC33-TGFβ1 complex generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with a TGFβ-related indication. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody, or antigen binding portion thereof, that specifically binds a GARP-TGFβ1 complex, a LTBP1-TGFβ1 complex, a LTBP3-TGFβ1 complex, and/or a LRRC33-TGFβ1 complex as those described herein.

Kits may optionally provide additional components such as buffers and interpretive information. Normally, the kit comprises a container and a label or package insert(s) on or associated with the container. In some embodiments, the disclosure provides articles of manufacture comprising contents of the kits described above.

Assays for Detecting a GARP-TGFβ1 Complex, a LTBP1-TGFβ1 Complex, a LTBP3-TGFβ1 Complex, and/or a LRRC33-TGFβ1 Complex

In some embodiments, methods and compositions provided herein relate to a method for detecting a GARP-TGFβ1 complex, a LTBP1-TGFβ1 complex, a LTBP3-TGFβ1 complex, and/or a LRRC33-TGFβ1 complex in a sample obtained from a subject. As used herein, a “subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, poultry, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a patient or a healthy volunteer.

In some embodiments, a method for detecting a GARP-TGFβ1 complex, a LTBP1-TGFβ1 complex, a LTBP3-TGFβ1 complex, and/or a LRRC33-TGFβ1 complex in a sample obtained from a subject involves (a) contacting the sample with an antibody that specifically binds a GARP-TGFβ1 complex, a LTBP1-TGFβ1 complex, a LTBP3-TGFβ1 complex, and/or a LRRC33-TGFβ1 complex under conditions suitable for binding of the antibody to the antigen, if the antigen is present in the sample, thereby forming binding complexes; and (b) determining the level of the antibody bound to the antigen (e.g., determining the level of the binding complexes).

In one embodiment, a screening assay that utilizes biotinylated latent TGFβ1 complexes immobilized onto a surface, which allows for the activation of latent TGFβ by integrins by providing tether. Other, non-integrin activators could also be tested in that system. Readout can be through reporter cells or other TGFβ-dependent cellular responses.

### Cell-Based Assays for Measuring TGFβ Activation

Activation of TGFβ (and inhibition thereof by a TGFβ test inhibitor, such as an antibody) may be measured by any suitable method known in the art. For example, integrin-mediated activation of TGFβ can be utilized in a cell-based assay, such as the “CAGA12” luciferase assay, described in more detail herein. As shown, such an assay system may comprise the following components: i) a source of TGFβ (recombinant, endogenous or transfected); ii) a source of activator such as integrin (recombinant, endogenous, or transfected); and iii) a reporter system that responds to TGFβ activation, such as cells expressing TGFβ receptors capable of responding to TGFβ and translating the signal into a readable output (e.g., luciferase activity in CAGA12 cells or other reporter cell lines). In some embodiments, the reporter cell line comprises a reporter gene (e.g., a luciferase gene) under the control of a TGFβ-responsive promoter (e.g., a PAI-1 promoter). In some embodiments, certain promoter elements that confer sensitivity may be incorporated into the reporter system. In some embodiments, such promoter element is the CAGA12 element. Reporter cell lines that may be used in the assay have been described, for example, in Abe et al. (1994) Anal Biochem. 216(2): 276-84, incorporated herein by reference. In some embodiments, each of the aforementioned assay components are provided from the same source (e.g., the same cell). In some embodiments, two of the aforementioned assay components are provided from the same source, and a third assay component is provided from a different source. In some embodiments, all three assay components are provided from different sources. For example, in some embodiments, the integrin and the latent TGFβ complex (proTGFβ and a presenting molecule) are provided for the assay from the same source (e.g., the same transfected cell line). In some embodiments, the integrin and the TGF are provided for the assay from separate sources (e.g., two different cell lines, a combination of purified integrin and a transfected cell). When cells are used as the source of one or more of the assay components, such components of the assay may be endogenous to the cell, stably expressed in the cell, transiently transfected, or any combination thereof. The results from a non-limiting exemplary embodiment of a cell-based assay for measuring TGFβ activation demonstrating the inhibition of either GARP-proTGFβ1 complex or LRRC33-proTGFβ1 complex using antibodies Ab1 and Ab2 is disclosed herein. In this exemplary assay, the IC50 (μg/mL) of Ab1 for the GARP-TGFβ1 complex was 0.445, and the IC50 (μg/mL) of Ab1 for the LRRC33-TGFβ1 complex was 1.325.

A skilled artisan could readily adapt such assays to various suitable configurations. For instance, a variety of sources of TGFβ may be considered. In some embodiments, the source of TGFβ is a cell that expresses and deposits TGFβ (e.g., a primary cell, a propagated cell, an immortalized cell or cell line, etc.). In some embodiments, the source of TGFβ is purified and/or recombinant TGFβ immobilized in the assay system using suitable means. In some embodiments, TGFβ immobilized in the assay system is presented within an extracellular matrix (ECM) composition on the assay plate, with or without de-cellularization, which mimics fibroblast-originated TGFβ. In some embodiments, TGFβ is presented on the cell surface of a cell used in the assay. Additionally, a presenting molecule of choice may be included in the assay system to provide suitable latent-TGFβ complex. One of ordinary skill in the art can readily determine which presenting molecule(s) may be present or expressed in certain cells or cell types. Using such assay systems, relative changes in TGFβ activation in the presence or absence of a test agent (such as an antibody) may be readily measured to evaluate the effects of the test agent on TGFβ activation in vitro. Data from exemplary cell-based assays are provided in the Example section below.

Such cell-based assays may be modified or tailored in a number of ways depending on the TGFβ isoform being studied, the type of latent complex (e.g., presenting molecule), and the like. In some embodiments, a cell known to express integrin capable of activating TGFβ may be used as the source of integrin in the assay. Such cells include SW480/136 cells (e.g., clone 1E7). In some embodiments, integrin-expressing cells may be co-transfected with a plasmid encoding a presenting molecule of interest (such as GARP, LRRC33, LTBP (e.g., LTBP1 or LTBP3), etc.) and a plasmid encoding a pro-form of the TGFβ isoform of interest (such as proTGFβ1). After transfection, the cells are incubated for sufficient time to allow for the expression of the transfected genes (e.g., about 24 hours), cells are washed, and incubated with serial dilutions of a test agent (e.g., an antibody). Then, a reporter cell line (e.g., CAGA12 cells) is added to the assay system, followed by appropriate incubation time to allow TGFβ signaling. After an incubation period (e.g., about 18-20 hours) following the addition of the test agent, signal/read-out (e.g., luciferase activity) is detected using suitable means (e.g., for luciferase-expressing reporter cell lines, the Bright-Glo reagent (Promega) can be used). In some embodiments, Luciferase fluorescence may be detected using a BioTek (Synergy H1) plate reader, with autogain settings.

Representative results of cell-based TGFβ assays are provided in FIG. 7 herein. Data demonstrate that exemplary antibodies of the invention which are capable of selectively inhibiting the activation of TGFβ1 in a context-independent manner.

### Nucleic Acids

In some embodiments, antibodies, antigen binding portions thereof, and/or compositions of the present disclosure may be encoded by nucleic acid molecules. Such nucleic acid molecules include, without limitation, DNA molecules, RNA molecules, polynucleotides, oligonucleotides, mRNA molecules, vectors, plasmids and the like. In some embodiments, the present disclosure may comprise cells programmed or generated to express nucleic acid molecules encoding compounds and/or compositions of the present disclosure. In some cases, nucleic acids of the disclosure include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety.

The present invention is further illustrated by the following examples, which are not intended to be limiting in any way. The entire contents of all references, patents and published patent applications cited throughout this application, as well as the Figures, are hereby incorporated herein by reference.

This invention is further illustrated by the following examples which should not be construed as limiting.

### EXAMPLES

### Example 1: Inhibition of TGFβ1

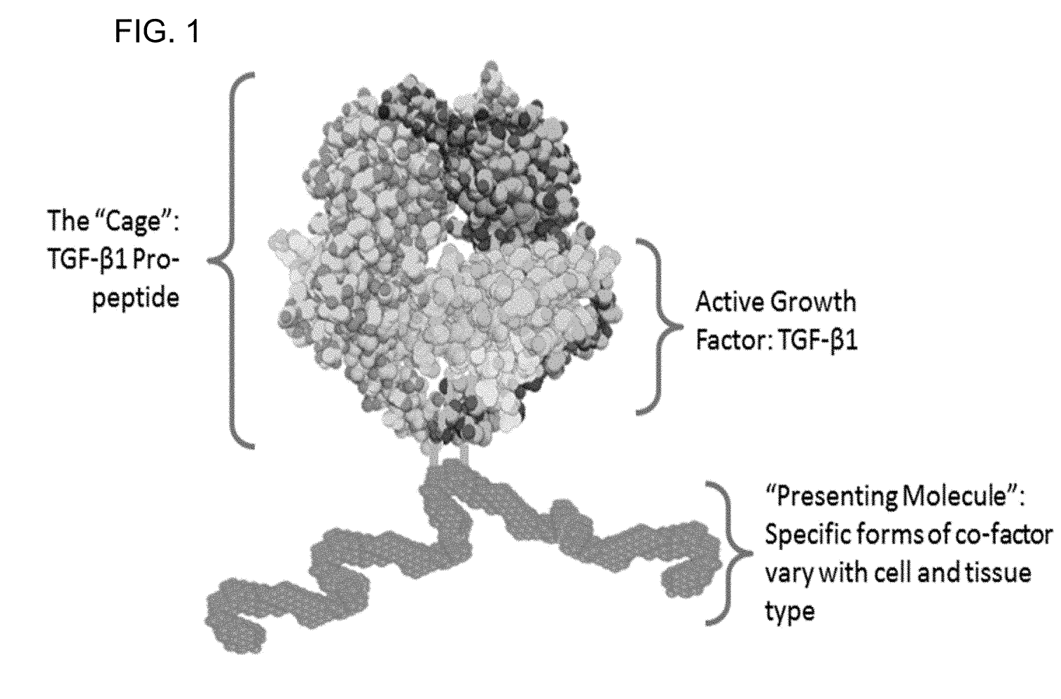

The TGFβ superfamily includes propeptides complexed with active growth factors (FIG. 1). Selection strategies to obtain antibodies that stabilize the complex, resulting in more selective and potent inhibition, were developed.

Using a HEK293-based expression system, NiNTA affinity and gel filtration were performed to obtain multimilligram quantities of purified protein, which were used to generate TGFβ1 complexed to LTBP (LTBP-TGFβ1 complex) and TGFβ1 complexed to GARP (GARP-TGFβ1 complex) (FIG. 3). The diversity of proteins manufactured enabled the testing of species cross-reactivity and epitope mapping.

The candidate antibodies were tested using an in vitro luminescence assays. In the screen, antibodies that inhibited growth factor release turned reporter cells “off” when faced with a stimulus for normal activation. Ab1 and Ab2 were shown to be inhibitors of activation of latent TGFβ1 complexes and were cross-reactive to mouse.

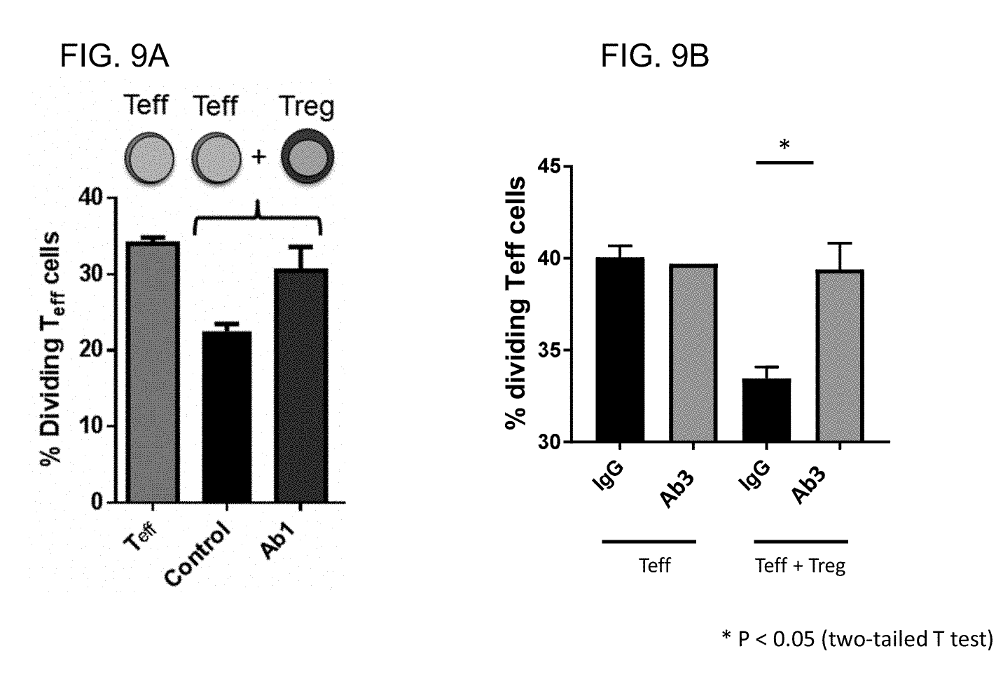

Initial dose-response analysis curves of Ab1 in cells expressing human TGFβ1 showed TGFβ1 activity inhibition. Using a more sensitive CAGA12 reporter cell line, Ab1 showed similar inhibition of human proTGFβ1 activity. Furthermore, the inhibition of a GARP complex was shown to block the suppressive activity of T regulatory cells (Tregs) as measured by the percent of dividing T effector cells (Teff) in T cells isolated from healthy donor blood (FIG. 9A). Similar results were observed for Ab3. Dose-response analysis curves of Ab3 in human hepatic stellate cells and human skin fibroblasts showed TGFβ1 activity inhibition (FIG. 7F) and Ab3 was also shown to inhibit suppressive Treg activity (FIG. 9B).

The affinity of GARP-proTGFβ1 inhibitors was measured by Octet assay on human GARP-proTGFβ1 cells, while activity was measured by CAGA12 reporter cells testing human GARP-proTGFβ1 inhibition. The protocol used to measure the affinity of antibodies Ab1 and Ab2 to the complexes provided herein is summarized in Table 6. The results are shown in Table 7.

TABLE 6
Protocol for performing Octet binding assay

Materials:
96 well black polypropylene plates
Streptavidin-coated tips for Octet
10x kinetics buffer (diluted 1:10 in PBS)
1. Soak required amount of streptavidin tips in 1X kinetics
buffer; place in machine to equilibrate
2. Load sample plate:
200 μl of buffer or antibody dilution to each well
a. Column 1-baseline (buffer)
b. Column 2-biotinylated protein (e.g., sGARP-proTGFβ1 or
LTBP1-proTGFβ1); diluted to 5 μg/mL
c. Column 3-baseline 2 (buffer)
d. Column 4-antibody association for Ab1
e. Column 5-antibody association for Ab2
f. Column 6-dissociation Ab 1 (buffer)
g. Column 7-dissociation Ab2 (buffer)
3. Make dilutions in the 96 well plate:
a. Dilute both antibodies to 50 μg/mL in 300 μl of 1x buffer in row A.
b. Add 200 μl of buffer to the rest of each column
c. Transfer 100 μl down the column to make 3-fold dilutions
4. Place the sample plate in the machine next to the tips plate
5. Set up the software
a. Indicate buffer, load, sample (one assay per antibody tested)
b. Indicate steps of the protocol (baseline, load, association,
dissociation) for set amounts of time:
Baseline: 60 seconds
Loading: 300 seconds
Baseline 2: 60 seconds
Association: 300 seconds
Dissociation: 600 seconds
6. Analyze data
a. Subtract baseline from reference well
b. Set normalization to last five seconds of baseline
c. Align to dissociation
d. Analyze to association and dissociation (1:1 binding model, fit curves)
e. Determine the best R² values; include concentrations with best R² values
f. Select global fit
g. Set colors of samples by sensor type
h. Analyze
i. Save table and export

TABLE 7
Affinity and Activity of GARP-proTGFβ1 Inhibitors

| | Affinity for GARP- | Inhibition (IC50) of | |
|---|---|---|---|
| | proTGFβ1 | GARP-proTGFβ1 | Max effect |
| Clone | (nM ± SEM) | (nM; 95% CI) | (% inhibition) |
| Ab1 | 0.046 ± 0.043 | 3.4 (2.1-5.4) | 75% |
| Ab2 | 0.561 ± 0.014 | 3.9 (1.5-10.3) | 50% |

The clones were further screened for binding selectivity (Table 8) and species cross-reactivity (Table 9). Ab1 and Ab2 did not bind to TGFβ1, TGFβ2, or TGFβ3, but did bind the proTGFβ1 complexes and showed species cross-reactivity.

TABLE 8
Selectivity of GARP-proTGFβ1 Inhibitors

| Clone | GARP-proTGFβ1 | LTBP1-proTGFβ1 | LTBP3-proTGFβ1 |
|---|---|---|---|
| Ab1 | +++ | +++ | +++ |
| Ab2 | +++ | +++ | +++ |

TABLE 9
Species Cross-Reactivity of GARP-proTGFβ1 Inhibitors

| Clone | huGARP-proTGFβ1 | muGARP-proTGFβ1 | cyGARP-proTGFβ1 |
|---|---|---|---|
| Ab1 | +++ | ++ | +++ |
| Ab2 | +++ | +++ | +++ |

+++ KD < 1 nM,
++ KD 1-10 nM
+ KD 10-100 nM
− No binding

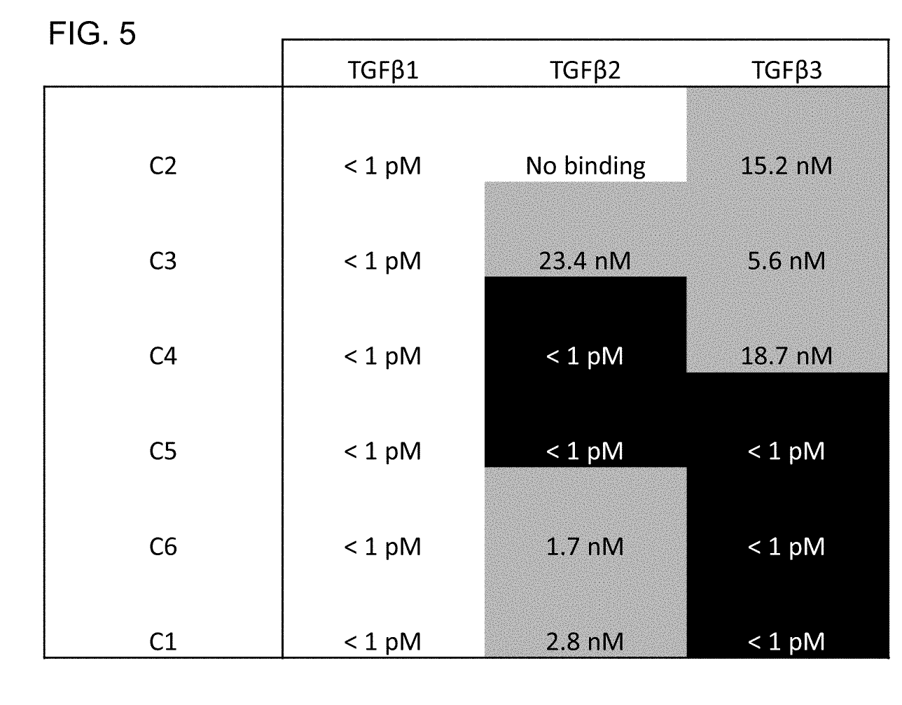

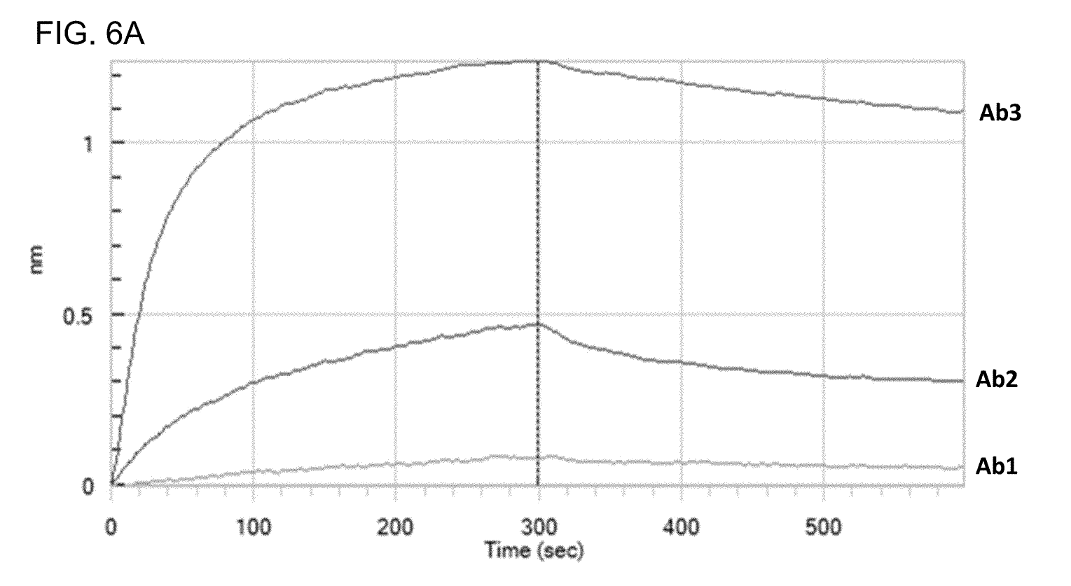

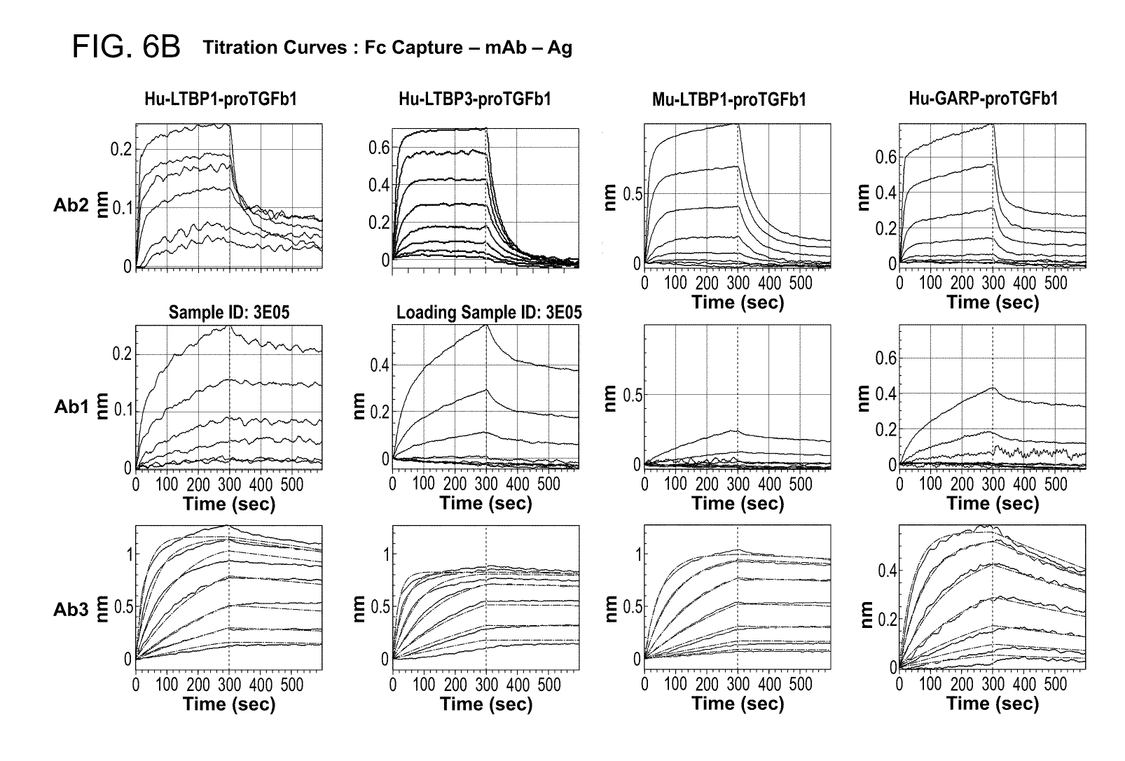

Binding specificity for Ab3 was further tested by Octet binding assay. As demonstrated in FIG. 4A, Ab3 bound specifically to latent TGFβ1, but not to latent TGFβ2 or latent TGFβ3, whereas pan-TGFbeta antibodies are not isoform specific (FIG. 5). These data demonstrate that Ab3 binds to TGFβ in an isoform specific manner.

### Example 2: Ab1, Ab2 and Ab3 Specifically Bind to proTGFβ1 Complexes from Multiple Species

To determine if Ab1, Ab2 and Ab3 are capable of specifically binding to proTGFβ1 complexes from multiple species, Octet binding assays were performed as described in Table 6. As shown in Table 10 (below), all three antibodies (i.e., Ab1, Ab2 and Ab3) specifically bound to human and murine LTBP1-proTGFβ1 complexes, human LTBP3-proTGFβ1 complexes, and human GARP-proTGFβ1 complexes. However, only Ab2 and Ab3 specifically bound to rat LTBP1-proTGFβ1 complexes.

TABLE 10
Affinity of Ab1, Ab2 and
Ab3 for proTGFβ1 Complexes from Multiple Species

| | Ab1 (K_{D}) | Ab2 (K_{D}) | Ab3 (K_{D}) |
|---|---|---|---|
| human LTBP1-proTGFβ1 | 16 ± 1.3 | 5.8 ± 0.6 | 1.1 ± 0.07 |
| human LTBP3-proTGFβ1 | 85 ± 5.0 | 122 ± 3.9 | 0.12 ± 0.04 |
| mouse LTBP1-proTGFβ1 | 203 ± 13 | 61 ± 4.0 | 0.68 ± 0.06 |
| rat LTBP1-proTGFβ1 | No binding detected | 38 ± 6.8 | 0.93 ± 0.03 |
| human GARP-proTGFβ1 | 293 ± 22 | 58 ± 6.2 | 4.9 ± 0.11 |

### Example 3: Ab2 and Ab3 Bind to LRRC33-Pro TGFβ1

To determine whether Ab1, Ab2 and Ab3 bind to proTGFβ1 that is complexed with LRRC33, Octet binding assays were performed. As shown in FIG. 12C, Ab1, Ab2 and Ab3 are capable of binding to the LRRC33-proTGFβ1 protein complex. However, Ab1 shows a slow on-rate for binding the LRRC33-proTGFβ1 protein complex. Binding of Ab1, Ab2 and Ab3 to the LRRC33-proTGFβ1 protein complex was further confirmed using ELISA.

### Example 4: Ab1, Ab2 and Ab3 Inhibit the Activity of Both GARP-proTGFβ1 and LRRC33-proTGFβ1

To determine whether Ab1, Ab2 and Ab3 inhibit the activity of GARP-proTGF-31 and/or LRRC33-proTGF-131, an in vitro cell-based assay was performed. In this assay system, an engineered human colon cancer cell line (SW480/136 cells) stably transfected with 136 integrin was co-transfected with a construct to express proTGF-131 and a construct to express a presenting molecule (i.e., GARP or LRRC33). To express the presenting molecules, constructs encoding chimeric LRRC33-GARP (SEQ ID NO: 101) or GARP were employed. The transfected cells were incubated to allow for sufficient expression and deposition of the components (integrins and proTGFβ1 complexed with a respective presenting molecule). Activation of TGF131 in the presence or absence of Ab1 or Ab2 or Ab3 was assayed using reporter cells (CAGA12 cells) expressing TGFβ receptors coupled to its downstream signal transduction pathway, to measure the inhibitory activity of the antibody. As shown in FIGS. 7A and 7B, Ab1, Ab2 and Ab3 inhibited both GARP-proTGF-31 and LRRC33-proTGF-131.

An additional cell-based assay was performed to detect inhibition of either GARP-proTGF11 complex or LRRC33-proTGF11 complex using antibodies Ab1 and Ab2. Ab1 and Ab2 inhibited both GARP-proTGF-31 and LRRC33-proTGF-11. In this assay, the IC50 (μg/mL) of Ab1 for the GARP-TGF131 complex was 0.445, and the IC50 (μg/mL) of Ab1 for the LRRC33-TGF11 complex was 1.325.

### Example 5: Assays for Detecting a LTBP-TGFβ1-Specific Activation

In some embodiments, methods and compositions provided herein relate to a method for detecting a LTBP-TGF11 complex, e.g., a LTBP1- or LTBP3-TGF11 complex, in a sample.

A. Activation of Latent TGFβ1 Deposited in the ECM

In this assay, presenting molecules are co-transfected with proTGF11 in integrin-expressing cells. Transiently transfected cells are seeded in assay plates in the presence of inhibitors. Latent LTBP-proTGF11 complex is embedded in the ECM. TGF1 reporter cells are then added to the system; free growth factor (released by integrin) signals and is detected by luciferase assay.

The following protocol is one example for measuring extracellular matrix (LTBP presented) activation by integrin cells. Materials include: MvLu1-CAGA12 cells (Clone 4A4); SW480/β6 cells (Clone 1E7) (αV subunit is endogenously expressed at high levels; 136 subunit is stably overexpressed); LN229 cell line (high levels of endogenous αVβ8 integrin); Costar white walled TC treated 96 well assay plate #3903; Greiner Bio-One High Binding white uclear 96 well assay plate #655094; Human Fibronectin (Corning #354008); P200 multichannel pipet; P20, P200, and P1000 pipets with sterile filter tips for each; sterile microfuge tubes and rack; sterile reagent reservoirs; 0.4% trypan blue; 2 mL, 5 mL, 10 mL, and 25 mL sterile pipets; tissue culture treated 100 mm or 150 mm plates; 70% ethanol; Opti-MEM reduced serum media (Life Tech #31985-070); Lipofectamine 3000 (Life Tech #L3000015); Bright-Glo luciferase assay reagent (Promega #E2620); 0.25% Tryspin+0.53 mM EDTA; proTGFb1 expression plasmid, human (SR005); LTBP1S expression plasmid, human (SR044); LTBP3 expression plasmid, human (SR117); LRRC32 (GARP) expression plasmid, human (SR116); and LRRC33 expression plasmid, human (SR386). Equipment utilized includes: BioTek Synergy H1 plate reader; TC hood; Bench top centrifuge; CO₂ incubator 37° C. 5% CO₂; 37° C. water/bead bath; platform shaker; microscope; and hemocytometer/countess.

“CAGA12 4A4 cells” are a derivative of MvLu1 cells (Mink Lung Epithelial Cells), stably transfected with CAGA12 synthetic promoter, driving luciferase gene expression. “DMEM-0.1% BSA” is an assay media; base media is DMEM (Gibco Cat#11995-065), media also contains BSA diluted to 0.1% w/v, penicillin/streptinomycin, and 4 mM glutamine. “D10” refers to DMEM 10% FBS, P/S, 4 mM glutamine, 1% NEAA, 1× GlutaMAX (Gibco Cat#35050061). “SW480/(6 Media” refers to D10+1000 ug/mL G-418. “CAGA12 (4A4) media” refers to D10+0.75 ug/mL puromycin.

On Day 0, cells are seeded for transfection. SW480/136 (clone 1E7) cells are detached with trypsin and pelleted (spin 5 min @ 200×g). Cell pellet is re-suspended in D10 media and viable cells per ml are counted. Cells are seeded at 5.0e6 cells/12 ml/100 mm TC dish. For CAGA12 cells, cells are passaged at a density of 1.0 million per T75 flask, to be used for the assay on Day 3. Cultures are incubated at 37° C. and 5% CO₂.

On Day 1, integrin-expressing cells are transfected. Manufacturer's protocol for transfection with Lipofectamine 3000 reagent is followed. Briefly, the following are diluted into OptiMEM I, for 125 ul per well: 7.5 ug DNA (presenting molecule)+7.5 ug DNA (proTGFβ1), 30 ul P3000, and up to 125 ul with OptiMEM I. The well is mixed by pipetting DNA together, then OptiMEM is added. P3000 is added, and everything is mixed well by pipetting. A master mix of Lipofectamine3000 is made, to be added to DNA mixes: for the LTBP1 assay: 15 ul Lipofectamine3000, up to 125 ul in OptiMEM I, per well; for the LTBP3 assay: 45 ul Lipofectamine3000, up to 125 ul in OptiMEM I, per well. Diluted Lipofectamine3000 is added to DNA, mixed well by pipetting, and incubated at room temp for 15 min. After the incubation, the solution is mixed a few times by pipetting, and then 250 ul of DNA:Lipofectamine3000 (2×125 ul) per dish is added dropwise. Each dish is gently swirled to mix and the dish is returned to the tissue culture incubator for 24 hrs.

Equivalent amounts of each plasmid are typically optimal for co-transfection. However, co-transfection may be optimized by changing the ratio of plasmid DNAs for presenting molecule and proTGFβ1.

On Days 1-2, the assay plates are coated with human fibronectin. Specifically, lyophilized fibronectin is diluted to 1 mg/ml in ultra-pure distilled water (sterile). 1 mg/ml stock solution is diluted to 19.2 ug/ml in PBS (sterile). 50 ul/well is added to assay plate (high binding) and incubated O/N in tissue culture incubator (37° C. and 5% CO₂). Final concentration is 3.0 ug/cm².

On Day 2, transfected cells are plated for assay and inhibitor addition. First, the fibronectin coating is washed by adding 200 ul/well PBS to the fibronectin solution already in the assay plate. Wash is removed manually with multichannel pipette. Wash is repeated for two washes total. The plate is allowed to dry at room temperature with lid off prior to cell addition. The cells are then plated by detaching with trypsin and pellet (spin 5 min @ 200×g). The pellet is resuspended in assay media and viable cells were counted per ml. For the LTBP1 assay cells are diluted to 0.10e6 cells/ml and seed 50 ul per well (5,000 cells per well). For the LTBP3 assay, cells are diluted to 0.05e6 cells/ml and seeded 50 ul per well (2,500 cells per well). To prepare functional antibody dilutions, antibodies are pre-diluted to a consistent working concentration in vehicle. Stock antibodies are serially diluted in vehicle (PBS is optimal, avoid sodium citrate buffer). Each point of serial dilution is diluted into assay media for a 4× final concentration of antibody. 25 ul per well of 4× antibody is added and cultures are incubated at 37° C. and 5% CO₂ for 24 hours.

On Day 3, the TGFβ reporter cells are added. CAGA12 (clone 4A4) cells for the assay are detached with trypsin and pelleted (spin 5 min @ 200×g.). The pellet is resuspended in assay media and viable cells per ml are counted. Cells are diluted to 0.4e⁶ cells/ml and seed 50 ul per well (20,000 cells per well). Cells are returned to incubator.

On Day 4, the assay is read (16-20 hours after antibody and/or reporter cell addition). Bright-Glo reagent and test plate are allowed to come to room temperature before reading. Read settings on BioTek Synergy H1 are set using TMLC_std protocol—this method has an auto-gain setting. Positive control wells are selected for autoscale (high). 100 uL of Bright-Glo reagent is added per well. Incubate for 2 min with shaking, at room temperature; protect plate from light. The plate is read on BioTek Synergy H1.

Data generated from this assay reflects LTBP1-TGFβ1 and/or LTBP3-TGFβ1 binding activity in cell supernatants.

B. Activation of Latent TGFβ1 Presented on the Cell Surface

To detect activation of latent TGFβ1 present on the cell surface, presenting molecules are co-transfected with proTGFβ1 in integrin-expressing cells. Latent TGFβ1 is expressed on the cell surface by GARP or LRRC33. TGFβ reporter cells and inhibitors are then added to the system; free growth factor (released by integrin) signals and is detected by luciferase assay. This assay, or “direct-transfection” protocol, is optimal for cell-surface presented TGFβ1 (GARP or LRRC33 presenter) activation by integrin cells.

Materials used included: MvLu1-CAGA12 cells (Clone 4A4); SW480/(6 cells (Clone 1E7) (αV subunit is endogenously expressed at high levels; 136 subunit is stably overexpressed); LN229 cell line (high levels of endogenous αVβ8 integrin); Costar white walled TC treated 96 well assay plate #3903; Greiner Bio-One High Binding white uclear 96 well assay plate #655094; Human Fibronectin (Corning #354008); P200 multichannel pipet; P20, P200, and P1000 pipets with sterile filter tips for each; sterile microfuge tubes and rack; sterile reagent reservoirs; 0.4% trypan blue; 2 mL, 5 mL, 10 mL, and 25 mL sterile pipets; tissue culture treated 100 mm or 150 mm plates; 70% ethanol; Opti-MEM reduced serum media (Life Tech #31985-070); Lipofectamine 3000 (Life Tech #L3000015); Bright-Glo luciferase assay reagent (Promega #E2620); 0.25% Tryspin+0.53 mM EDTA; proTGFb1 expression plasmid, human (SR005); LTBP1S expression plasmid, human (SR044); LTBP3 expression plasmid, human (SR117); LRRC32 (GARP) expression plasmid, human (SR116); and LRRC33 expression plasmid, human (SR386).

Equipment used includes: BioTek Synergy H1 plate reader; TC hood; bench top centrifuge; CO₂ incubator 37° C. 5% CO₂; 37° C. water/bead bath; platform shaker; microscope; hemocytometer/countess.

The term “CAGA12 4A4 cells” refers to a derivative of MvLu1 cells (Mink Lung Epithelial Cells), stably transfected with CAGA12 synthetic promoter, driving luciferase gene expression. “DMEM-0.1% BSA” refers to an assay media; base media is DMEM (Gibco Cat#11995-065), media also contains BSA diluted to 0.1% w/v, penicillin/streptinomycin, and 4 mM glutamine. “D10” refers to DMEM 10% FBS, P/S, 4 mM glutamine, 1% NEAA, 1× GlutaMAX (Gibco Cat#35050061). “SW480/β6 Media” refers to D10+1000 ug/mL G-418. “CAGA12 (4A4) media” refers to D10+0.75 ug/mL puromycin.

On Day 0, integrin expressing cells are seeded for transfection. Cells are detached with trypsin and pelleted (spin 5 min @ 200×g). Cell pellet is resuspended in D10 media and viable cells per ml are counted. Cells are diluted to 0.1e⁶ cells/ml and seeded 100 ul per well (10,000 cells per well) in an assay plate. For CAGA12 cells, passage at a density of 1.5 million per T75 flask, to be used for the assay on Day 2. Cultures are incubated at 37° C. and 5% CO₂.

On Day 1, cells are transfected. The manufacturer's protocol is followed for transfection with Lipofectamine 3000 reagent. Briefly, the following is diluted into OptiMEM I, for 5 ul per well: 0.1 ug DNA (presenting molecule)+0.1 ug DNA (proTGFβ1), 0.4 ul P3000, and up to 5 ul with OptiMEM I. The well is mixed by pipetting DNA together, then OptiMEM is added. P3000 is added, and everything is mixed well by pipetting. A master is was made with Lipofectamine3000, to be added to DNA mixes: 0.2 ul Lipofectamine3000, up to 5 ul in OptiMEM I, per well. Diluted Lipofectamine3000 is added to DNA, mixed well by pipetting, and incubated at room temp for 15 min. After the incubation, the solution is mixed a few times by pipetting, and then 10 ul per well of DNA:Lipofectamine3000 (2×5 ul) was added. The cell plate is returned to the tissue culture incubator for 24 hrs.

On Day 2, the antibody and TGFβ reporter cells are added. In order to prepare functional antibody dilutions, stock antibody in vehicle (PBS is optimal) is serially diluted. Then each point is diluted into assay media for 2× final concentration of antibody. After preparing antibodies, the cell plate is wished twice with assay media, by aspirating (vacuum aspirator) followed by the addition of 100 ul per well assay media. After second wash, the assay media is replaced with 50 ul per well of 2× antibody. The cell plate is returned to the incubator for 15-20 min.

In order to prepare the CAGA12 (clone 4A4) cells for the assay, the cells are detached with trypsin and pelleted (spin 5 min @ 200×g.). The pellet is resuspended in assay media and viable cells per ml are counted. Cells are diluted to 0.3e⁶ cells/ml and seeded 50 ul per well (15,000 cells per well). Cells are returned to incubator.

On Day 3, the assay is read about 16-20 hours after the antibody and/or reporter cell addition. Bright-Glo reagent and test plate are allowed to come to room temperature before reading. The read settings on BioTek Synergy H1 are set to use TMLC_std protocol—this method has an auto-gain setting. Positive control wells are set for autoscale (high). 100 uL of Bright-Glo reagent is added per well. Incubate for 2 min with shaking, at room temperature; protect plate from light. The plate is read on BioTek Synergy H1.

Data generated from this assay reflects TGFβ1 activity in cell supernatants. Raw data units are relative light units (RLU). Samples with high RLU values contain high amounts of free TGFβ1, samples with low RLU values contain low levels of TGFβ1.

### Example 6: Ab1 and Ab2 Inhibit Endogenous TGFβ1 in Human and Murine Fibroblasts

To determine if Ab1 and Ab2 were capable of inhibiting endogenous TGF-β1 secreted by primary cultured fibroblasts of different origin, a quantitative in vitro assay was performed in which the activity of secreted TGF-β1 was determined by measuring luciferase levels produced by mink lung epithelial cells that were stably transfected with a nucleic acid comprising a luciferase reporter gene fused to a CAGA12 synthetic promoter, and co-cultured with fibroblasts treated with either Ab1 or Ab2. As shown in FIGS. 7G and 7H, both Ab1 and Ab2 were inhibited endogenous TGF-β1 secreted by normal human dermal fibroblasts, murine C57BL.6J lung fibroblasts, and DBA2/J muscle fibroblasts. Differences in the maximal inhibition observed with each antibody were cell line-specific.

### Example 7: Role of Matrix Stiffness and Effects of TGFβ1-Specific, Context-Independent Antibodies on Integrin-Induced Activation of TGFβ1 In Vitro

To examine whether substrates with different degrees of stiffness can modulate TGFβ1 activation, silicon-based substrates of controlled stiffness (5 kPa 15 kPa, and 100 kPa) were used to measure integrin-dependent activation of TGFβ1 in primary fibroblasts plated thereon. Briefly, SW480 cells were co-transfected with proTGFβ1 and LTBP1 to allow extracellular presentation of the latent TGFβ1 complex. Cells overexpressing αvβ6 integrin were added to the assay system to trigger activation of TGFβ1. TGFβ1 activation was determined by measuring TGFβ-responsive reporter gene activation. In this setting, αvβ6 integrin caused approximately two-fold increase in LTBP1-mediated TGFβ1 activation in cells plated on silicon substrates of high stiffness (100 kPa) tested, as compared to cells cultured on silicon substrates with lower (5 or 15 kPa) stiffness, under otherwise identical conditions. The present inventors have found that isoform-specific, context-permissive inhibitors of TGFβ1 activation, such as those described herein, can suppress this effect, reducing TGFβ1 activation to approximately half the level, as compared to no antibody control at all stiffness tested.

### Example 8: Effects of TGFβ1-Specific, Context-Independent Antibodies on Protease-Induced Activation of TGFβ1 in Vitro

To test integrin-independent, protease-dependent activation of TGFβ1 in vitro, purified recombinant LTBP3-proTGFβ1 complex was incubated with Kallikrein (KLK), and TGFβ1 activation was measured using a reporter cell system as described. TGFβ1 was released from the latent complex following incubation with KLK but not with vehicle alone, suggesting that ECM-associated TGFβ1 activity may be triggered in a protease-dependent manner.

To further test the ability of an isoform-specific, context-independent inhibitor antibody to inhibit an alternate mode (e.g., integrin-independent) of TGFβ1 activation, an in vitro assay was established to evaluate Kallikrein-activation of TGFβ1.

Briefly, CAGA reporter cells were seeded 24 hours prior to the start of the assay. ProTGFβ-C4S was titered onto CAGA cells. Plasma-KLK protease was added at a fixed concentration of 1 microgram per mL or 500 nanogram per mL. The assay mixture was incubated for approximately 18 hours. TGFβ activation was measured by Luciferase assay. Data are shown in FIG. 8. In the presence of KLK, proTGFβ1 was activated (positive control). This TGFβ activation was effectively inhibited by the addition of Ab3, indicating that, in addition to integrin-dependent activation of TGFβ1, the isoform-specific, context-independent inhibitory antibody can also block KLK-dependent activation of TGFβ1 in vitro. Similarly, inhibition of KLK-activated TGFβ1 was also observed with addition of Ab1 (data not shown).

### Example 9: LRRC33 Expression in Polarized and Activated Macrophages

It was previously described that TGFβ signaling is involved in maturation and differentiation of and eventual phenotypes of macrophages. Monocyte-derived macrophages have been suggested to express LRRC33. Further studies of polarized macrophages have revealed that not all polarized macrophages express LRRC33. We found that so-called classic M1-type macrophages show low expression of LRRC33, while M2 macrophages showed elevated LRRC33 expression. Unexpectedly, among the subtypes of M2 macrophages, we observed LRRC33 expression only in M2c and M2d, TAM-like macrophages. The former is so-called “pro-fibrotic” macrophages, and the latter is “TAM-like” or mimicking tumor-associated phenotype. These results show that LRRC33 expression is restricted to a selective subset of polarized macrophages.

Evidence suggests that tumor cells and/or surrounding tumor stromal cells secrete a number of cytokines, growth factors and chemokines, which may influence the phenotypes (e.g., activation, differentiation) of various cells in the TME. For example, macrophage colony-stimulating factor (M-CSF also referred to as CSF-1) is a known tumor-derived factor, which may regulate TAM activation and phenotype.

Fluorescence-activated cell sorting (FACS) analyses were carried out to examine effects of an M-CSF exposure on LRRC33 expression in macrophages. Briefly, human PBMCs were collected from healthy donors. The primary cells were cultured for one week, in a medium containing 10% human serum, plus GM-CSF or M-CSF. To induce various M2 macrophage phenotypes, cells were cultured for additional 2-3 days in the presence of IL-10 and TGFβ for the M2c subtype, and IL-6 for the M2d subtype. Antibodies against the cell surface markers as indicated in the figure were used in the FACS analyses. CD14+ immunomagnetic selection indicates monocytes.

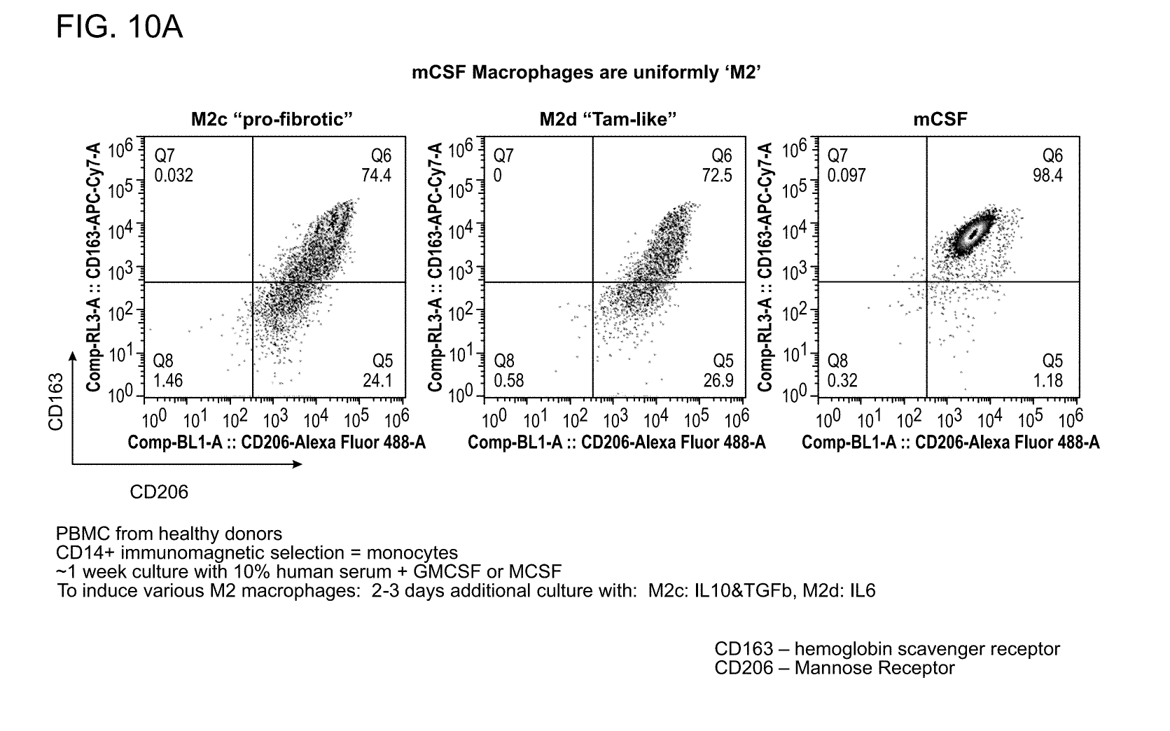

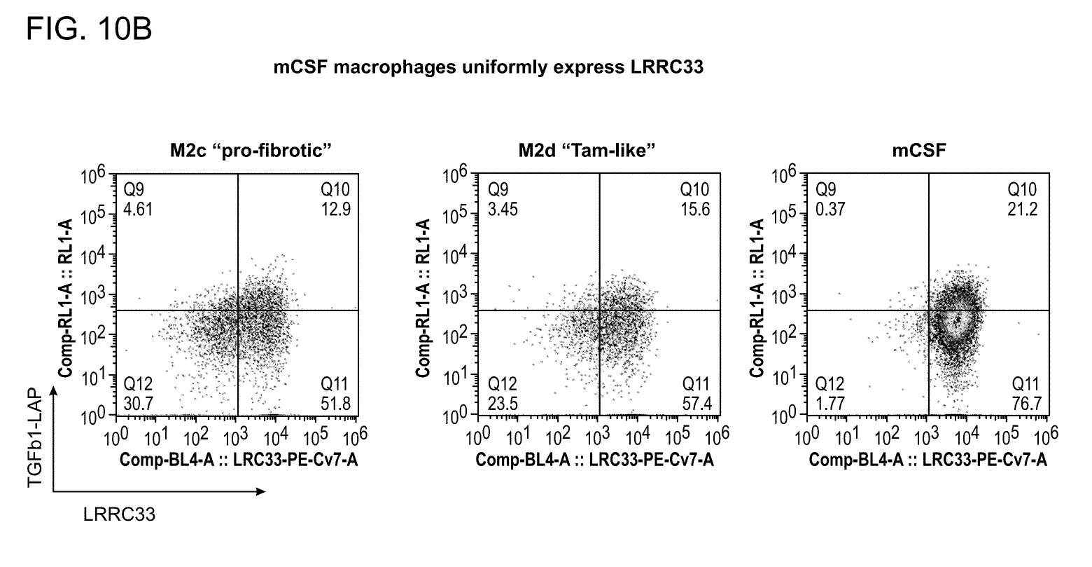

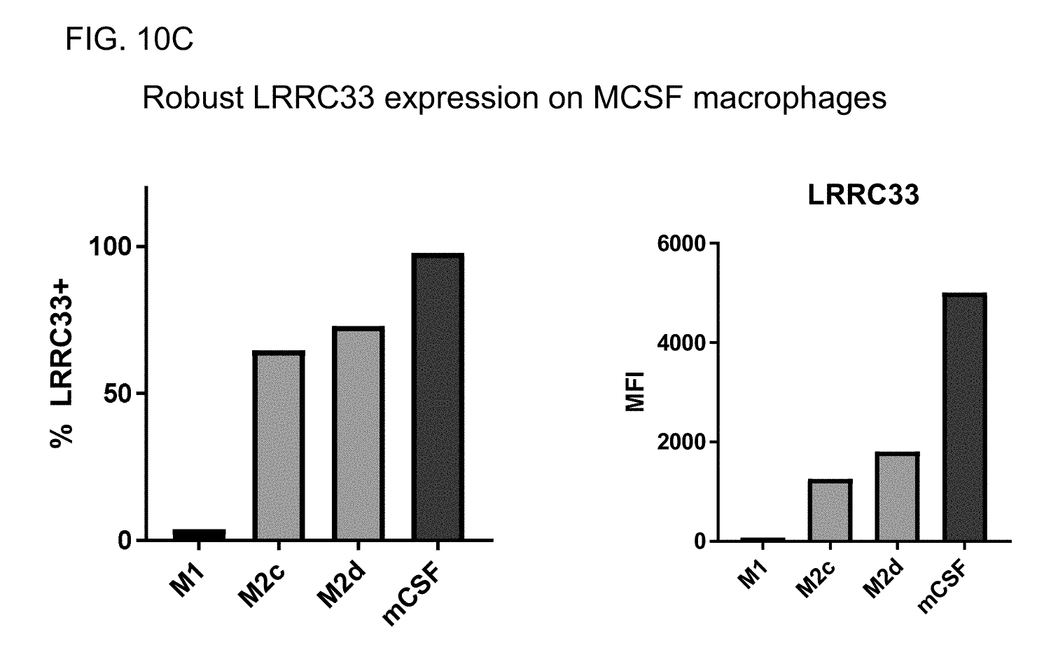

Surprisingly, results showed that upregulation of cell surface LRRC33 on macrophages was significantly augmented upon exposure to M-CSF (also known as CSF-1). FIG. 10A shows that M-CSF-treated macrophages are uniformly that of M2-polarized macrophages. Moreover, M-CSF exposure causes macrophages to uniformly express LRRC33 on the cell surface (see FIG. 10B). As summarized in FIG. 10C, robust LRRC33 expression on M-CSF-activated macrophages was observed. These results suggest that tumor-derived factors such as M-CSF may induce local macrophage activation to support tumor growth.

### Example 10: Effect of Ab3 on Regulatory T (Treg) Cell Activity In Vivo

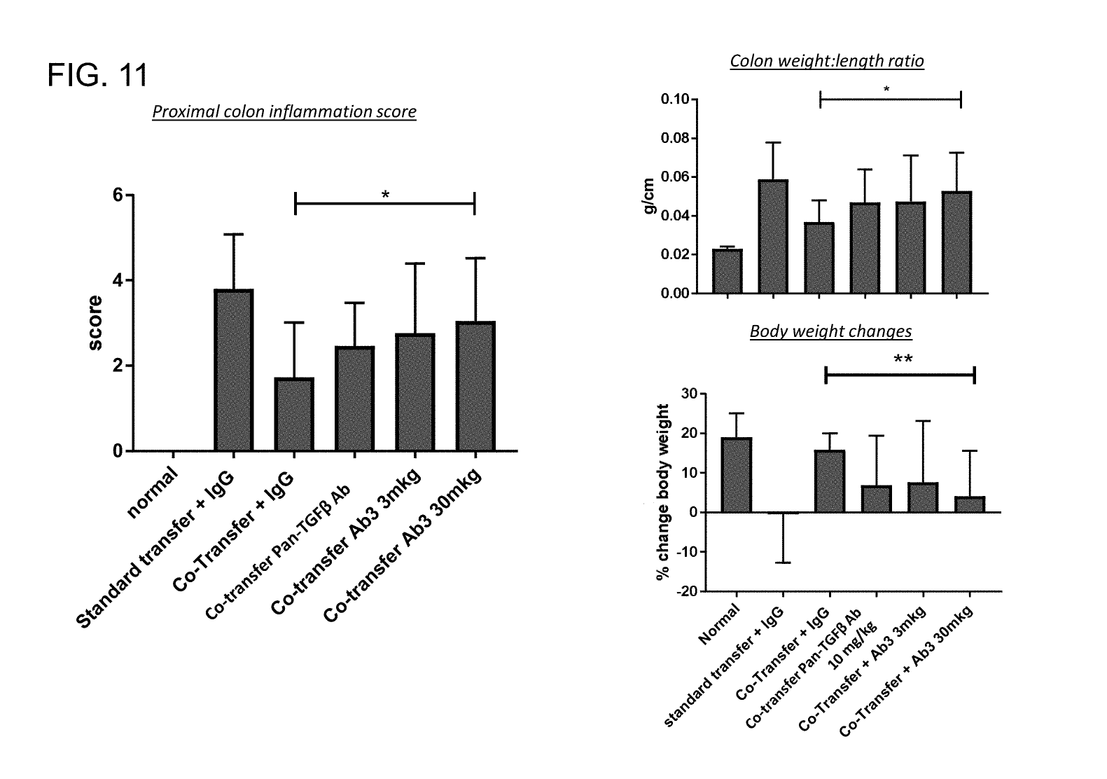

GARP has been shown to be expressed on regulatory T cells. The effect of Ab3 on regulatory T cell activity in vivo was assessed using a T cell transfer colitis model (Powrie et al., 1993 International Immunology, 5(11): 1464-1474; Powrie et al., 1994 Immunity, 1: 553-562; Powrie et al., 1996 J. Exp. Med., 186: 2669-2674). Transfer of CD45Rbhi T cells into severe combined immune deficiency (SCID) mice is known to induce colitis, and co-transfer of CD45Rblo CD25+ regulatory T cells (Treg) inhibits colitis development and exhibits protective effect on mice. As demonstrated in FIG. 11, mice receiving 30 mg/kg Ab3 eliminated the protective effect demonstrated by co-transfer of CD45Rblo CD25+ Treg. Specifically, mice receiving 30 mg/kg Ab3 demonstrated a significant increase in the proximal colon inflammation score and colon weight to length ratio, and a significant reduction in body weight gain as compared to IgG control. These data demonstrate that Ab3 is capable of suppressing regulatory T cell activity in vivo.

### Example 11: Effects of Ab1 and Ab2 Alone or in Combination with Anti-PD-1 Antibody on Tumor Progression in the MC38 Murine Colon Carcinoma Syngeneic Mouse Model

To evaluate the effects of Ab1 and Ab2, alone or in combination with an anti-PD-1 antibody to decrease colon carcinoma tumor progression, the MC38 murine colon carcinoma C57BL/6 mouse syngeneic model was used.

### Tumor Cell Culture

MC38 murine colon carcinoma cells were grown in Dulbecco's Modified Eagle's Medium (DMEM) containing 10% fetal bovine serum, 100 units/mL penicillin G sodium, 100 μg/mL streptomycin sulfate, 25 μg/mL gentamicin, and 2 mM glutamine. Cell cultures were maintained in tissue culture flasks in a humidified incubator at 37° C., in an atmosphere of 5% CO₂ and 95% air.

### In Vivo Implantation and Tumor Growth

The MC38 cells used for implantation were harvested during log phase growth and resuspended in phosphate buffered saline (PBS). On the day of tumor implant, each test mouse was injected subcutaneously in the right flank with 5×10⁵ cells (0.1 mL cell suspension), and tumor growth was monitored as the average size approached the target range of 80 to 120 mm³. Eleven days later, designated as Day 1 of the study, mice were sorted according to calculated tumor size into groups each consisting of twelve animals with individual tumor volumes ranging from 63 to 196 mm³ and group mean tumor volumes of 95 to 98 mm³. Tumors were measured in two dimensions using calipers, and volume was calculated using the formula:

$$Tumor Volume ( {mm}^{3} ) = \frac{{w}^{2} \times l}{2}$$

where w=width and l=length, in mm, of the tumor. Tumor weight may be estimated with the assumption that 1 mg is equivalent to 1 mm³ of tumor volume.

### Treatment

Briefly eight week old female C57BL/6 mice (n=12) bearing subcutaneous MC38 tumors (63-172 mm³) on Day 1 were administered intraperitoneally (i.p.) twice a week for four weeks either Ab1, Ab2, murine IgG1 control antibody (each at 30 mg/kg in a dosing volume of 10 mL/kg). When tumors reached 150 mm³ (Day 6) in the control groups, mice were administered either rat anti-mouse PD-1 antibody (RMP1-14) or rat IgG2A control antibody i.p. twice a week for two weeks (each antibody at 5 mg/kg in a dosing volume of 10 mL/kg).

Group 1 served as tumor growth controls, and received murine IgG1 isotype control antibody in combination with rat IgG2a control antibody. Group 2 received Ab1 in combination with rat IgG2a control antibody. Group 3 received Ab2 in combination with rat IgG2a control antibody. Group 3 received murine IgG1 control antibody in combination with anti-PD-1 antibody. Group 4 received Ab1 in combination with anti-PD-1 antibody. Group 5 received Ab2 in combination with anti-PD-1 antibody. Group 6 (n=16) was not treated and served as a sampling control group.

### Endpoint and Tumor Growth Delay (TGD) Analysis

Tumors were measured using calipers twice per week, and each animal was euthanized when its tumor reached the endpoint volume of 1,000 mm³ or at the end of the study (Day 60), whichever happened earlier. Mice that exited the study for tumor volume endpoint were documented as euthanized for tumor progression (TP), with the date of euthanasia. The time to endpoint (TTE) for analysis was calculated for each mouse according to the methods described in U.S. Provisional Application No. 62/558,311, filed on Sep. 13, 2017.

### MTV and Criteria for Regression Responses

Treatment efficacy may be determined from the tumor volumes of animals remaining in the study on the last day. The MTV (n) was defined as the median tumor volume on the last day of the study in the number of animals remaining (n) whose tumors had not attained the endpoint volume.

Treatment efficacy may also be determined from the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study. An animal with a CR response at the termination of a study was additionally classified as a tumor-free survivor (TFS). Animals were monitored for regression responses.

### Tumor Growth Inhibition

Tumor growth inhibition (TGI) analysis evaluates the difference in median tumor volumes (MTVs) of treated and control mice. For this study, the endpoint for determining TGI was Day 29, which was the day control mice reached the mean tumor volume of 1500 mm³. The MTV (n), the median tumor volume for the number of animals, n, on the day of TGI analysis, was determined for each group. Percent tumor growth inhibition (% TGI) was defined as the difference between the MTV of the designated control group and the MTV of the drug-treated group, expressed as a percentage of the MTV of the control group:

The data set for TGI analysis included all mice in a group, except those that died due to treatment-related (TR) or non-treatment-related (NTR) causes prior to the day of TGI analysis.

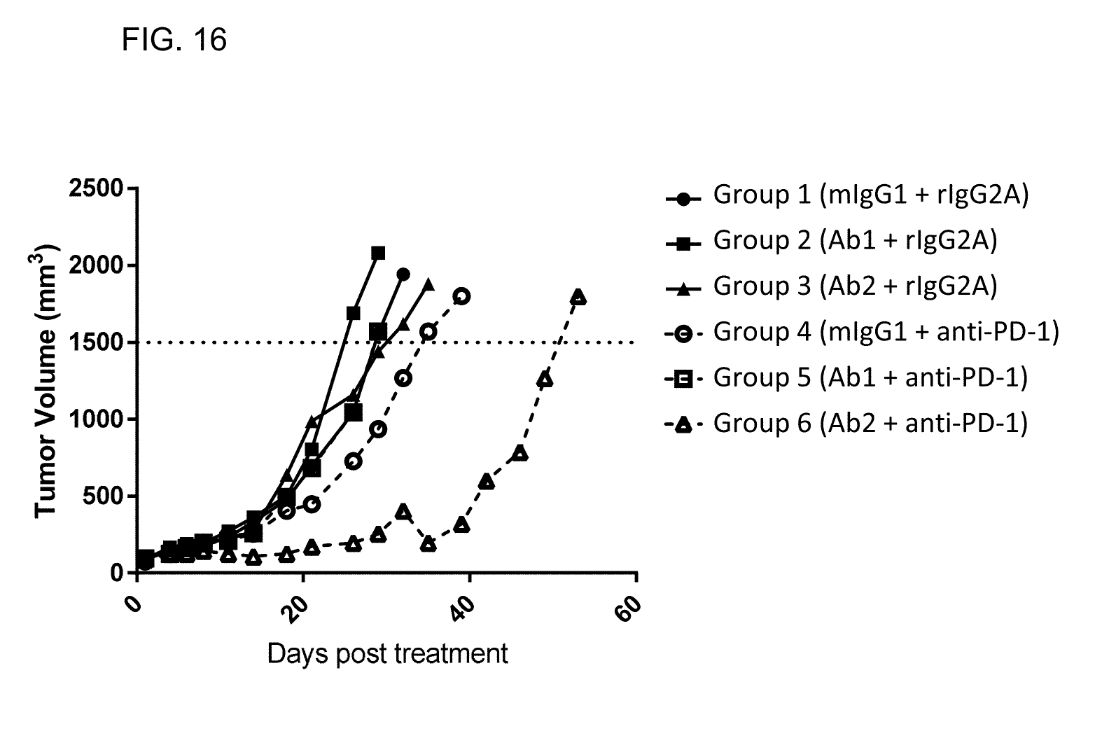

In the present study, Ab1 and Ab2 were evaluated alone and in combination with anti-PD-1 in the MC38 murine colon carcinoma C57BL/6 mouse syngeneic model. Mice that were administered Ab2 in combination with anti-PD-1 resulted in significant Day 29 TGI (P<0.05, Mann-Whitney U test), producing survival benefit that was statistically significantly different from vehicle-treated controls using logrank survival analyses (P<0.05, logrank) (see FIG. 16). Mice receiving Ab1 or Ab2 in combination with rat IgG2a control antibody had regression responses of 1 CR and 1 PR respectively. In combination with anti-PD-1 the regressions responses of Ab1 and Ab2 were 1 PR and 1 CR, and 4 CRs, respectively. Ab2 in combination with anti-PD-1 produced significant short-term efficacy on Day 29 and produced overall survival benefit in this 60-day TGD study in the MC38 murine colon carcinoma C57BL/6 mouse syngeneic model.

### Example 12: In Vivo Effects of Ab3 on Survival in Combination with PD-1 Inhibitor in TGFβ1/3-Model

EMT-6 is an orthotopic mouse tumor model in which immune checkpoint inhibitor treatment alone has shown limited effects on tumor growth and survival. The inventors have recognized that in certain syngeneic tumor models, multiple isoforms of TGFβ are expressed, as assessed by RNAseq. Both TGFβ1 and TGFβ3 are co-dominant in EMT-6 (see FIG. 21) which are expressed in almost equal amounts. The inventors therefore reasoned that in this particular model, a pan-inhibitor of TGFβ isoforms may provide broader in vivo efficacy, as compared to an isoform-selective inhibitor.

### Study Design

To test this hypothesis, 8-12 week old female Balb/c mice were injected with 0.1 mL containing 5×10⁶ EMT6 breast cancer cells in 0% Matrigel subcutaneously in the flank. Animals were monitored throughout the study biweekly for weight and tumor caliper measurement. Upon tumors reaching the volume of 30-80 mm³ animals were randomized into 6 groups and dosing began as follows: Group 1: HuNeg-rIgG1/HuNeg-mIgG1; Group 2: anti-PD1-rIgG1/HuNeg-mIgG1; Group 3: anti-PD1-rIgG1/pan-TGFβ Ab-mIgG1; Group 4: anti-PD1-rIgG1/Ab3-mIgG1; Group 5: HuNeg-rIgG1/pan-TGFβ Ab-mIgG1; and, Group 6: HuNeg-rIgG1/Ab3-mIgG1. The anti-PD1 clone was RMP1-14 (BioXCell) and administered at 5 mg/kg, twice a week. HuNeg-rIgG1 was used as an isotype control and dosed similarly. Ab3-mIgG1 was dosed at 30 mg/kg, once a week and HuNeg-mIgG1 was dosed similarly. Pan-TGFβ Ab-mIgG1 was dosed at 5 mg/kg twice a week. All dosing was done intraperitoneally at 10 ml/kg. When tumors exceeded 2000 mm³ animals were sacrificed, serum was collected, and the tumor was removed and flash frozen for eventual analysis. No animals were sacrificed due to significant body weight loss, and one animal in Group 2 was found dead (not determined to be treatment related).

### Results

EMT6 is a fast progressing syngeneic tumor model. Group 1 and Group 6 animals had a median survival of 18 days, which is typical of no treatment effect in this model. It is known that anti-PD1 has a limited effect in this model, and as such, increased the median survival to 19.5 days when administered alone (Group 2). Group 5 also had a small increase in median survival to 21 days. Group 4 had a modest increase in survival to 25 days with two animals still alive at day 34. Group 3 had only 3 death events by day 34, indicating a significant survival effect of this combination. TGFβ1 inhibition via administration of Ab3-mIgG1 alone had no effect on tumor volume growth, however in conjunction with anti-PD1 5 animals showed slower tumor growth and one animal exhibiting complete response. Pan-TGFβ Ab alone slowed tumor growth in 3 animals, but in combination with anti-PD1 4 animals saw significantly slower tumor growth and 5 animals exhibiting complete response. These findings are consistant with publically available information, e.g., whole tumor RNAseq database (Crown Bioscience MuBase) showing that EMT6 tumors exhibit near equal levels of TGFβ1 and TGFβ3 expression.

### Example 13: Effects of Ab2 and Ab3 on Renal Biomarkers and Fibrosis in a Unilateral Ureteral Occluded (UUO) Mouse Model

The unilateral ureteral occluded mouse model has been widely used to study interstitial fibrosis, a common pathological process which may lead to end-stage renal disease (see Isaka et al. (2008) Contrib. Nephrol. 159: 109-21, and Chevalier (1999) Pediatr. Nephrol. 13: 612-9). UUO mice are characterized by renal myofibroblast activation, tubular atrophy and interstitial fibrosis with minimal glomerular lesions (see Lian et al. (2011) Acta Pharmacol. Sin. 32: 1513-21). Increased expression of TGFβ1 is considered to play a role in the phenotype observed in UUO mice. To evaluate the effect of Ab2 on the presentation of interstitial fibrosis in the UUO mouse model, the following experiment was performed.

Briefly, 7-8 week-old male CD-1 mice (Charles River Laboratories) were 4 groups of mice (n=10) were administered either Ab2 (3 mg/kg or 30 mg/kg; dosing volume of 10 mL/kg), murine IgG1 control antibody (30 mg/kg; dosing volume of 10 mL/kg), or PBS, as vehicle control intraperitoneally (i.p.) prior to surgical intervention. Treatments were administered one day prior to surgery (d−1), one day after surgery (dl), and 3 days after surgery (d3). On day 0 (d0), mice were anesthetized with isoflurane anesthesia on a nosecone, and a laparotomy performed followed by permanent right unilateral UUO surgery. An additional control group of mice (n=8) was administered PBS as described above, but solely underwent sham surgery (i.e., laparotomy with no occlusion of the ureter). Immediately following completion of the surgical procedure, all mice received one subcutaneous injection of 0.001 mg/kg buprenorphine. Mice were sacrificed five days after surgery and tissues were harvested for analysis. After harvest, both kidneys were placed in ice-cold 0.9% NaCl, de-encapsulated and weighed. Hydroxyproline levels to assess collagen content of the kidney tissue were assessed. Kidney hydroxyproline levels, a marker of tissue fibrosis and collagen deposition, were significantly increased in mice that received surgical intervention as compared to mice receiving sham surgery.

A mid traverse section of each right kidney was immersion fixed in 10% neutral buffered formalin for 48 hours, which was then transferred to 70% ethanol for histological processing and analysis. Fixed kidney sections were paraffin embedded, sectioned (three 5 μm serial sections acquired 200-250 μm apart per animal kidney to enable greater sampling and representation of kidney injury), stained with Picrosirius Red, and subjected to quantitative histological analyses using color spectrum segmentation to determine cortical collagen volume fraction (CVF). One composite CVF score was calculated for each animal by determining the average CVF score for each of the three serial sections. Statistical analyses were performed using the unpaired t-test. As shown in FIG. 12K, renal cortical fibrosis, as determined by CVF, was increased in UUO obstructed kidneys as compared to control sham-treated mice. Mice receiving either 3 mg/kg or 30 mg/kg of Ab2 showed a significant attenuation in UUO-induced increases in CVF, as compared to mice receiving either vehicle control (PBS) or IgG control.

Relative mRNA expression levels of plasminogen activator inhibitor-1 (PAI-1), connective tissue growth factor (CTGF), TGFβ1, fibronectin-1, α-smooth muscle actin (α-SMA), monocyte chemotactic protein 1 (MCP-1), collagen type I alpha 1 (Col1a1), and collagen type III alpha 1 chain (Col3a1), in the harvested kidney tissue was determined (FIG. 12A-12H). mRNA levels were normalized using the housekeeping gene hypoxanthine phosphoribosyltransferase 1 (HPRT1) mRNA levels. Moreover, in mice receiving either 3 mg/kg or 30 mg/kg of Ab2 prior to surgical intervention, mRNA levels of PAI-1, CTGF, TGFβ1, fibronectin 1, Col1a1, and Col3a1 were significantly decreased, as compared to mice receiving 30 mg/kg IgG1 control. Mice receiving 3 mg/kg of Ab2 prior to surgical intervention, mRNA levels of α-SMA was significantly decreased, as compared to mice receiving 30 mg/kg IgG1 control. Further, mice receiving 30 mg/kg of Ab2 prior to surgical intervention, mRNA levels of MCP-1 were significantly decreased, as compared to mice receiving 30 mg/kg IgG1 control.

The effect of Ab3 on mRNA expression levels of known fibrosis markers was also evaluated. As shown in FIGS. 121 and 12J, in mice receiving 3 mg/kg or 30 mg/kg of Ab3 prior to surgical intervention, mRNA levels of PAI-1 and Col1a1 were significantly decreased, as compared to mice receiving IgG1 control.

In summary, significant effects were observed in mice treated with Ab2 or Ab3 in the UUO mouse model, with the exception of hydroxyproline levels. As shown in FIGS. 12A-12H and 12K, Ab2 treatment significantly attenuated UUO-induced increases in CVF, and significantly decreased gene expression of known fibrosis markers, such as PAI-1, CTGF, TGFβ1, fibronectin 1, Col1a1, and Col3a1. Similarly, as shown in FIGS. 121-12J, Ab3 treatment significantly decreased gene expression of known fibrosis markers, such as PAI-1 and Col1a1. These data demonstrate that TGFβ1 is the major form of TGFβ playing a role in renal disease and that, surprisingly, TGFβ2 and TGFβ3 are likely not involved in pathogenesis.

### Example 14: Effect of TGFβ1-Specific, Context-Independent Antibody on Murine Alport Model of Renal Fibrosis

The murine Col4a3 −/− model is an established genetic model of autosomal recessive Alport syndrome. Alport mice lack functional collagen 4 A3 (Col4A3−/−) and therefore cannot form type IV collagen, which requires a3, a4, and a5 chains. Col4a3−/− mice develop fibrosis in the kidney consistent with renal fibrosis in human patients, including glomerulosclerosis, interstitial fibrosis, and tubular atrophy, and all Col4a3−/− mice develop end-stage renal disease (ESRD) between 10 and 30 week of age, depending on the genetic background of the mouse. The structural and functional manifestation of renal pathology in Col4a3−/− mice, combined with the progression to ESRD make Col4a3−/− mice an ideal model to understand kidney fibrosis. Previous reports point to the importance of the TGFβ signaling pathway in this process, and treatment with either αvβ6 integrin, a known activator of TGFβ, or with a TGFβ ligand trap has been reported to prevent renal fibrosis and inflammation in Alport mice (Hahm et al. (2007) The American Journal of Pathology, 170(1): 110-125).

Ab3, which is an isoform-specific, context-independent inhibitor of TGFβ1 activation, was tested for its ability to inhibit or mitigate renal fibrosis in Alport mice as follows.

F1 offspring from 129:B16 heterozigous×heterozygous cross (medium progressing model) were employed for the study. Antibody dosing for Ab3 began six weeks after birth, at 5 mg/kg, twice a week (i.e., 10 mg/kg/week) for a test duration of six weeks. A pan-TGFβ neutralizing antibody was used as positive control (dosed at 5 mg/kg, twice a week), while IgG was used as negative control. All antibodies were administered via intraperitoneal injection. Following six weeks of antibody treatment (12 weeks after birth), animals were sacrificed, and the kidneys were collected for analyses.

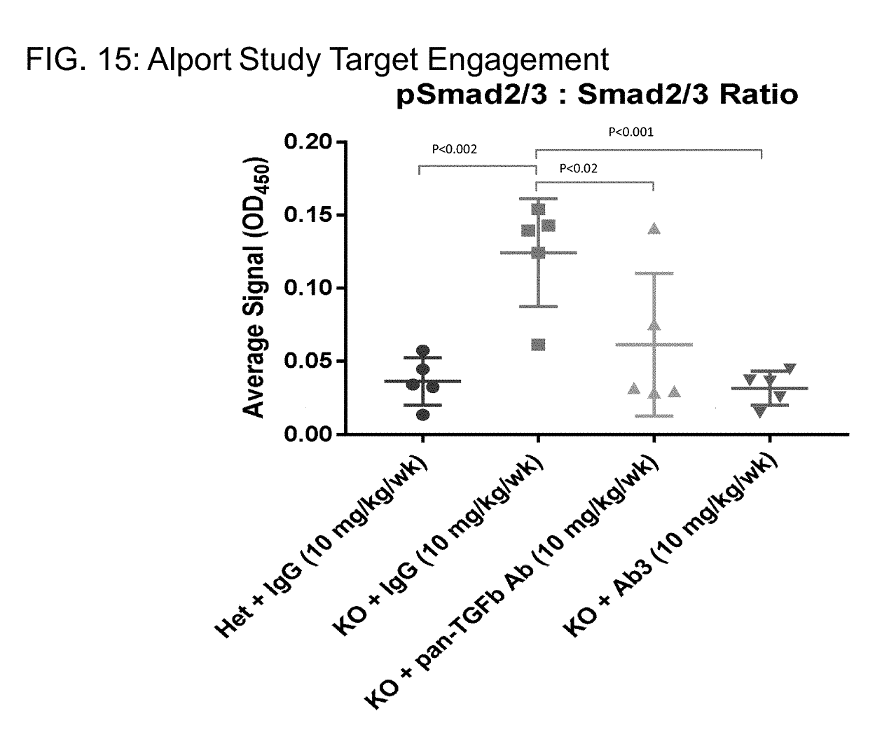

It is well documented that TGFβ receptor activation leads to a downstream signaling cascade of intracellular events, including phosphorylation of Smad2/3. Therefore, effects of the Ab3 antibody treatment were assessed in kidney lysate samples by measuring relative phosphorylation levels of Smad2/3 as assayed by ELISA (Cell Signaling) according to the manufacturer's instructions. FIG. 15 provides a graph showing relative ratios of phosphorylated vs. total (phosphorylated and unphospohrylated) Smad2/3. Whole kidney lysates prepared from samples of animals treated with Ab3 showed a significant reduction in relative phosphorylation of Smad2/3, as compared to negative control. The average ratios were equivalent to those of heterozygous control.

The 12 week-old Alport F1 mice described above exhibited early evidence of kidney fibrosis at the time of the completion of the study, as measured by both collagen deposits (Picrosirius Red quantification) and accumulation of blood urea nitrogen (BUN), each of which is indicative of fibrosis. Consistent with the inhibitory activities of Ab3 observed in downstream TGFβ receptor signaling, Ab3-treated tissues showed reduced signs of fibrosis. For example, the average BUN level for control Alport animals that did not receive Ab3 treatment was over 50 mg/dL, while the average BUN level in Ab3-treated animals was reduced to less than 30 mg/dL, suggesting that Ab3 may be capable of ameliorating fibrosis.

### Example 15: Effect of TGFβ1-Specific, Context-Independent Antibody on Carbon Tetrachloride-Induced Liver Fibrosis

TGFβ activities have been implicated to play a role in the pathology of organ fibrosis, such as liver fibrosis. It was previously reported that a soluble TGFBRII agent prevents liver fibrosis in the carbon tetrachloride (CCl4) model of liver fibrosis (Yata et al., Hepatology, 2002). Similarly, antisense inhibition of TGFβ1 (via adenoviral delivery) ameliorates liver fibrosis due to bile duct ligation (Arias et al., BMC Gastroenterology, 2003). In addition, 1D11, a pan-TGFβ antibody that neutralizes all isoforms of TGFβ, has been shown to reduce liver fibrosis and cholangiocarcinomas in TAA-treated rats (Ling et al., PLoS ONE, 2013).

Here, carbon tetrachloride (CCl4)-induced liver fibrosis model in mice was used to evaluate effects of a context-independent inhibitor of TGFβ1 activation on fibrosis in vivo. Liver fibrosis was induced in male BALB/c mice with CCl4, which was given twice a week for six weeks via i.p. After the first two weeks of CCl4 treatment, animals were treated with therapeutic weekly dosing of Ab3 (30 mg/kg). Therapeutic dosing with antibodies was initiated after two weeks and continued for four weeks.

Animals were randomized based on blood chemistry data. During the four weeks of Ab3 dosing of the study, blood samples were drawn for serum AST/ALT and total bilirubin analysis. Animals were weighed twice a week to monitor body weight during the study. After the six week study, the liver and spleen were collected and weighed to determine liver:spleen weight ratio. Liver pathology was assessed by histology on picrosirius-red stained liver slices. The extent of liver fibrosis was scored according to Masson or Picosirius red stained sections and viewing under 10 or 20× objective lens on entire section with the criteria listed below:

TABLE 14
Fibrosis Scoring Criteria

| | | | | Fibrosis |
|---|---|---|---|---|
| | Central vein | | | Areas of involvement |
| | thickening | Inter-sinusoidal | Portal | (NS) |
| Score | (CLV) | (PS) | (PT) | Layers of fibers (WS) |
| 0 | Normal | None | None | None |
| 1 | Slightly thickened | Focal | Mild amount | ≤6 layers |
| | | | | thin and not connected |
| 2 | Moderately | Moderate amount | Moderate amount | >6 layers |
| | thickened | | | thick and connected |
| 3 | Indistiguishable | Extensive amount | Cirrhosis | Nodule formation |
| | | | | dense fibrotic tissues |
| 4 | — | — | — | >⅔ of the section |

Fibrotic scores were then calculated using the formula SSS═CLV+PS+PT+2×(NS×WS), which takes into account Central vein thickening, Inter-sinusoidal, Portal, and affected areas and layers of the tissue.

As summarized in FIG. 14, four weekly doses of Ab3 treatment significantly reduced CCl4-induced liver fibrosis.

Similarly, the anti-fibrotic effects of Ab2 and Ab3 at multiple doses (3, 10 and 30 mg/kg) were examined by histological quantification (% area) of Picrosirius Red staining in formalin-fixed, paraffin-embedded sections of a single lobe of the liver. The quantification was performed by a pathologist in a blind manner. Consistent with the observation provided above, liver sections from antibody-treated animals showed significantly reduced CCl4-induced fibrosis as measured by Picrosirius Red staining which corresponds to relative amounts of tissue collagen. Results showed that each of Ab2 and Ab3 was effective in reducing liver fibrosis even at the lowest dose tested (3 mg/kg). More specifically, CCl4-treated animals that received Ab2 treatment at 3 mg/kg reduced collagen volume fraction (% area) to 2.03%, as compared to IgG control (3.356%) (p<0.0005). Similarly, CCl4-treated animals that received Ab3 treatment at 3 mg/kg reduced collagen volume fraction (% area) to 1.92%, as compared to IgG control (3.356%) (p<0.0005). Double negative control animals that received no CCl4 treatment showed a background collagen volume fraction of 1.14%.

Furthermore, preliminary data indicate that Ab3 treatment caused significant reduction in levels of phosphorylated SMAD2/3, as measured by ELISA as ratios of phospho-to-total SMAD2/3, indicating that TGFβ downstream signal transduction pathway was suppressed by administration of the context-independent inhibitor of TGFβ1 in vivo.

### Example 16: Role of TGFβ1 in Muscular Dystrophy

TGFβ plays multiple roles in skeletal muscle function, including inhibition of myogenesis, regulation of inflammation and muscle repair, and promotion of fibrosis. While there is considerable interest in TGFβ inhibition as a therapy for a wide range of diseases, including muscular dystrophies, these therapies inhibit TGFβ1, TGFβ2, and TGFβ3 regardless of molecular context. The lack of specificity/selectivity of these inhibitors may result in unwanted side effects leading to clinical doses with insufficient efficacy. While pan-TGFβ inhibitory molecules have been reported to improve muscle function and reduce fibrosis in the mdx mouse, whether those effects are due to inactivation of TGFβ1, 32, or 133 has yet to be addressed.

To that end, antibodies have been generated that specifically block the integrin-mediated activation of latent TGFβ1, while sparing TGFβ2 and 133. D2.mdx mice are treated with proTGFβ1-specific antibodies, so as to ascertain the role of TGFβ1 specifically in muscle repair in dystrophic muscle. The functional effects of TGFβ1 inhibition on protection from contraction-induced injury are assessed, as well as on recovery from the same method of injury. Histological evaluation includes whether treatment affects muscle damage, fibrosis, and inflammation. Additionally, possible toxicities may be assessed to determine whether the observed negative effects reported with pan-TGF inhibition in muscle (e.g., increased inflammation, long-term deficits in muscle function) are due to inhibition of TGFβ1 or TGFβ2/3. To understand whether inhibition of TGFβ1 in specific molecular contexts is more efficacious and/or has fewer negative effects (adverse effects), the efficacy of LTBP-proTGFβ1 inhibitors in this model may be assessed in order to deconvolute the role of immune cell presented TGFβ1 from that presented in the extracellular matrix (ECM), potentially leading to safer and/or more effective anti-fibrotic therapies.

Dystrophic muscle is highly susceptible to contraction-induced injury. Following injury, muscle from mdx mice shows a significant reduction in force generation and increased uptake of Evan's Blue dye, indicative of physical injury/damage to the muscle fiber, compared to WT (Lovering, R. M., et al., Arch Phys Med Rehabil, 2007. 88(5): p. 617-25). Therapeutic agents which reduce the extent of contraction-induced injury, or improve recovery following injury, would be of significant clinical benefit to muscular dystrophy patients (Bushby, K., et al., Lancet Neurol, 2010. 9(1): p. 77-93). Test inhibitors, such as Ab1, Ab2 and Ab3 may be evaluated for their ability to i) prevent contraction-induced injury, as well as to ii) promote recovery from injury. The D2.mdx strain may be used for our experiments, as opposed to the traditional mdx strain on the B10 background. These mice, generated by crossing the mdx onto a DBA2/J background, have the non-protective variant of LTBP4 described above, and therefore exhibit disease pathology that is more severe, progressive, and similar to the human disease than the standard mdx strain (Coley, W. D., et al., Hum Mol Genet, 2016. 25(1): p. 130-45). Since the D2.mdx mice are being used, DBA2/J mice can serve as wild-type controls. Since DMD affects primarily males, the studies may focus on male mice.

To examine the ability of Ab1 and Ab2 to prevent/limit contraction-induced injury, 6 week-old male D2.mdx mice (n=10) are treated with 10 mg/kg/week of either IgG control, Ab1, or Ab2 for 6 weeks. To allow for comparison to published work using a pan-TGFβ inhibitor, a fourth group is dosed with 10 mg/kg/week 1D11. All antibodies are mIgG1 isotype and this dose has previously been shown to be effective in the UUO model (FIGS. 12A-12K). A WT group dosed with the IgG control is also included. 24 hours prior to sacrifice, mice are administered 1% Evan's Blue dye (EBD) in PBS (volume 1% of body weight) to allow assessment of myofiber damage by fluorescence microscopy. At the end of treatment, mice are subjected to an in vivo eccentric contraction protocol. Eccentric injury of the gastrocnemius muscle will be may be performed with a 305B muscle lever system (Aurora Scientific) as described (Khairallah, R. J., et al., Sci Signal, 2012. 5(236): p. ra56). Briefly, 20 eccentric contractions with 1-minute pauses in between are performed, and the decrease in peak isometric force before the eccentric phase may be taken as an indication of muscle damage. The extent of force loss and the percent of EBD positive fibers may be determined. DBA2/J mice subjected to this protocol lose 30-40% of initial force after 20 eccentric contractions. In contrast, D2.mdx mice lose 80% of initial force following the same protocol, as previously described (Pratt, S. J., et al., Cell Mol Life Sci, 2015. 72(1): p. 153-64; Khairallah, R. J., et al., Sci Signal, 2012. 5(236): p. ra56). The ability of Ab1 and Ab2 to reduce force loss following injury may be assessed. Mice are sacrificed at the end of the experiment and both the injured and uninjured gastrocnemius muscles may be collected for histological analyses. EBD uptake may be assessed from both muscles. Myofiber cross-sectional area and the extent of fibrosis may be measured. For cross-sectional area determination, sections from the mid-belly of the muscle may be stained with wheat germ agglutinin conjugated to a fluorophore to visualize cell membranes. Sections may be digitized using fluorescent microscopy, cell boundary traced using predictive software and cross-sectional area determined via unbiased automated measurements. For analysis of fibrosis, sections may be stained with picrosirius red (PSR) and the area of PSR+ per slide computed.

The ability of Ab1, Ab2 or Ab3 to accelerate recovery from contraction-induced injury is assessed. 12 week old DBA2/J and D2.mdx mice may undergo the same eccentric contraction protocol described above. Following injury, mice are divided into treatment groups (n=10) and administered either an IgG control (for WT and D2.mdx mice), 1D11, Ab1, Ab2 or Ab3 (D2.mdx only). Antibodies may be dosed at 10 mg/kg/week for the duration of the experiment. Seven and 14 days post injury, maximal peak isometric force, twitch-to-tetanic ratio, and force-frequency relationship may be measured to evaluate the effect of treatment on recovery from injury. While Ab1, Ab2 and Ab3 inhibit release of TGFβ1 regardless of presenting molecule, selective release of TGFβ1 from the extracellular matrix (i.e., LTBP-presented) could have greater benefit in DMD due to the preservation of TGFβ1 driven Treg activity. To address this question, specific LTBP-proTGFβ1 inhibitory antibodies may also be assessed for both the ability to prevent contraction-induced injury and to accelerate recovery from injury.

### Example 17: Role of TGFβ1 in Skeletal Muscle Following Acute Injury

The role of TGFβ1 specifically in myofiber regeneration following muscle injury may be investigated. TGFβ1-specific antibodies may be employed in the cardiotoxin injury model to determine the role of TGFβ1 specifically during myofiber regeneration. Regeneration may be assessed histologically and functional assessments of muscle strength and quality may be conducted. Given the potential benefits of TGFβ1 inhibition for muscle regeneration, therapies which have beneficial effects without the toxicities observed with pan-TGFβ inhibition would be of great benefit. This allows an investigation of the effects of TGFβ1-specific inhibition on satellite cell function and may provide insights into satellite cell transplant studies.

As described above, TGFβ appears to have multiple effects on muscle biology, including inhibition of myoblast proliferation and differentiation, as well as promotion of atrophy and fibrosis (Allen, R. E. and L. K. Boxhorn, J Cell Physiol, 1987. 133(3): p. 567-72; Brennan, T. J., et al., Proc Natl Acad Sci USA, 1991. 88(9): p. 3822-6; Massague, J., et al., Proc Natl Acad Sci USA, 1986. 83(21): p. 8206-10; Olson, E. N., et al., J Cell Biol, 1986. 103(5): p. 1799-805; Li, Y., et al., Am J Pathol, 2004. 164(3): p. 1007-19; Mendias, C. L., et al., Muscle Nerve, 2012. 45(1): p. 55-9; Nelson, C. A., et al., Am J Pathol, 2011. 178(6): p. 2611-21). However, these studies either used recombinant TGFβ1 in culture or injected into mice which may have non-physiological results as the growth factor is removed from its molecular context. Alternatively, investigators used TGFβ inhibitors which are not selective for TGFβ1.

To evaluate isoform-specific, context-permissive effects of TGFβ1, multiple proTGFβ1 antibodies (e.g., Ab3) may be examined for their ability to affect muscle regeneration following CTX-induced injury. These antibodies are “isoform-specific” and “context-permissive” inhibitors of TGFβ1 activation, such that they specifically inhibit release of TGFβ1 (as opposed to TGFβ2 or TGFβ3) from any presenting molecule and do not bind the mature growth factors (FIG. 4B).

Muscle regeneration may be induced in male DBA2/J mice (n=10) via CTX injection into the right gastrocnemius muscle. One day prior to injury, mice may be administered 10 mg/kg IgG control, 1D11, Ab1, or Ab2. Antibodies are continued to be dosed weekly until end of study. At 7 and 14 days post injury, muscle force measurements may be measured in vivo with a 305C muscle lever system (Aurora Scientific Inc., Aurora, CAN). Briefly, for the plantarflexor muscle group, contractions are elicited by percutaneous electrical stimulation of the sciatic nerve in anaesthetized mice, and a series of stimulations is then performed at increasing frequency of stimulation (0.2 ms pulse, 500 ms train duration): 1, 10, 20, 40, 60, 80, 100, 150 Hz, followed by a final stimulation at 1 Hz. Maximal peak isometric force, twitch-to-tetanic ratio, and force-frequency relationship will be determined. Following force measurements, the injured gastrocnemius and soleus muscles are collected and prepared for histology. Myofiber cross sectional area and % PSR+ area may be determined as described in Example 8 above.

Treatment with Ab3 may result in reduced fibrosis and improved muscle function. However, given the role of TGFβ1 in regulating immune activation, it is possible that we may observe increased inflammation with the antibodies, as has been reported with 1D11 treatment (Andreetta, F., et al., J Neuroimmunol, 2006. 175(1-2): p. 77-86). In the event increased inflammation may limit the therapeutic effects of TGFβ1 inhibition, context-specific antibodies may be subsequently evaluated to provide further degree of specificity, which may limit toxicity. For example, antibodies that inhibit release of TGFβ1 from LTBPs only may be used, using the readouts and methods described above. These antibodies may limit release of TGFβ1 only from the ECM, without affecting release from Tregs or macrophages.

### Example 18: Selection of Suitable TGFβ1 Inhibitory Agents in Muscular Disorders

Expression analysis of proTGFβ1 and its presenting molecules in healthy, regenerating, and diseased muscle may provide useful information to aid the selection of optimal therapeutic approach. Given the potential benefits of TGFβ1 inhibition in muscle regeneration and repair, understanding the context of proTGFβ1 presentation (e.g., in the ECM or on immune cells) in skeletal muscle under different conditions (healthy, acutely injured, and chronically injured) can help inform the therapeutic utility of antibodies, and ultimately provide insight into the degree of specificity/selectivity required to achieve both clinical efficacy and safety. The nature of TGFβ1 presentation may vary depending on the health status of the muscle and over the course of disease, which could have implications for any TGFβ1 targeted therapies. Understanding the expression profiles of these molecules will also aid in selection of appropriate time of dosing for potential therapeutic molecules. Using western blot, immunohistochemistry, and immunoprecipitation, expression of proTGFβ1 and its presenting molecules may be assessed in normal, acutely injured (cardiotoxin injury), and chronically regenerating (D2.mdx mouse) muscle. Expression of these molecules may be investigated specifically in key cell types or subset of cell types (e.g., satellite cells, macrophages, fibro-adipogenic progenitors, etc.) in the different conditions described above.

While expression of TGFβ isoforms has been examined in muscles from mdx mice, previous work focused on expression of the mature growth factors (Nelson, C. A., et al., Am J Pathol, 2011. 178(6): p. 2611-21; Zhou, L., et al., Neuromuscul Disord, 2006. 16(1): p. 32-8). Given the target specificity of the TGFβ1 antibodies described herein, it is essential that the expression patterns be examined not only for mature and proTGFβ1, but those of the presenting molecules as well, which should provide information as to the source and/or context of a pool of TGFβ1 of interest. Ideally, it is desirable to gain understanding of the expression patterns of the latent complexes, not merely of each component.

Antibodies are screened for western blot and IHC for targets of interest. Antibodies against mouse TGFβ1-LAP, LTBP1, LTBP3, and LTBP4 are commercially available. The antibody against TGFβ1-LAP (clone TW7-16B4) has been extensively characterized and is effective in both flow cytometry and western blot (Oida, T. and H. L. Weiner, PLoS One, 2010. 5(11): p. e15523). Antibodies against LTBP1 (ProteinTech #22065-1-AP) and LTBP3 (Millipore #ABT316) have been validated internally using SW480 cells transfected with LTBP1-proTGFβ1 or LTBP3-proTGFβ1 and shown to be specific for their targets. The utility of these antibodies for IHC may be determined. Muscles from healthy and D2.mdx mice are sectioned and the antibodies tested on frozen and FFPE sections. Antibodies may be validated by including conditions with 100× excess of purified target protein or complex (made in house) to ensure that the signal observed is specific.

Previous work has identified antibodies which specifically bind a given latent complex but have no inhibitory activity. Antigen binding by these antibodies has been confirmed by ELISA (FIG. 4C) and may also be evaluated for their utility in IHC (given the three-dimensional structure of these epitopes these antibodies are unlikely to be effective as western blot reagents). The presence of latent TGFβ1 complexes from bulk tissue may also be assessed by western blot or immunoprecipitation. Latent complexes can be identified by western blot by running the same sample under reducing and non-reducing conditions. Under reducing conditions, TGFβ1, LAP and the presenting molecule separate, and the three molecules can be identified on the same blot but using dual-color western blot methods. Under non-reducing conditions, the LAP:presenting molecule complex remains associated while TGFβ1 is released; the complex migrates slower than the empty presenting molecule and migrates together with TGFβ1-LAP. Various antibodies are also evaluated for their ability to immunoprecipitate latent complexes from muscle to demonstrate direct binding of TGFβ1 to specific presenting molecules.

Once appropriate antibodies have been identified, expression in healthy, regenerating, and dystrophic muscle is assessed, by western and/or IHC, depending on the antibodies available. Tibialis anterior (TA) and diaphragm muscles may be collected from DBA2/J and D2.mdx mice at 4, 8, and 12 weeks of age. For regenerating muscle, cardiotoxin may be injected into the TA of 12 week old DBA2/J mice, and muscles collected 3, 7, and 14 days post injury. Tissue from at least 4 mice may be used for each condition/time point. Co-staining experiments may also be conducted to identify cell populations expressing the various molecules (for example: CD11b for macrophages, FoxP3 for Tregs, MyoD for myogenic cells).

### Example 19: Ab2 and Ab3 Exhibit Reduced Toxicity as Compared to the ALK5 Kinase Inhibitor LY2109761 and a Pan-TGFβ Antibody

To evaluate the toxicity of Ab2 and Ab3, as compared to the small molecule TGF-β type I receptor (ALK5) kinase inhibitor LY2109761 and to a pan-TGFβ antibody (hIgG4), toxicity studies were performed in rats. The rat was selected as the species for this safety study based on the previous reports that rats are more sensitive to TGFβ inhibition as compared to mice. Similar toxicities observed in rats have been also observed in other mammalian species, such as dogs, non-human primates, as well as humans.

A. Phase I of the Study

Briefly, female F344/NHsd rats were administered either Ab2 at 3 mg/kg (1 group, n=5), at 30 mg/kg (1 group, n=5), or at 100 mg/kg (1 group, n=5); a pan-TGFβ antibody at 3 mg/kg (1 group, n=5), at 30 mg/kg (1 group, n=5), or at 100 mg/kg (1 group, n=5); LY2109761 at 200 mg/kg (1 group, n=5) or 300 mg/kg (1 group, n=5); or PBS (pH 7.4) vehicle control (1 group, n=5). Animals receiving either Ab2, the pan-TGFβ antibody, or the vehicle control were dosed once intravenously (at day 1), and the rats receiving LY2109761 were dosed by oral gavage once daily during 7 days (7 doses). Animal body weight was determined at days 1, 3, and 7 of the dosing phase. Animals were sacrificed at day 8 and necropsies performed.

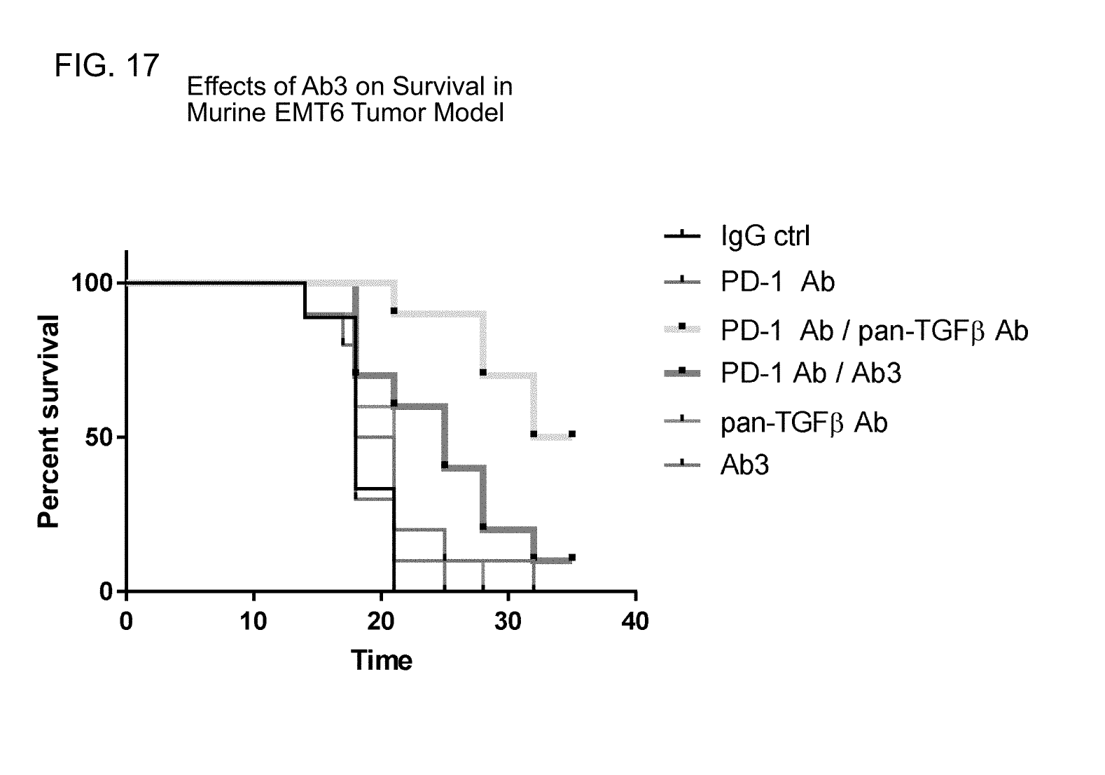

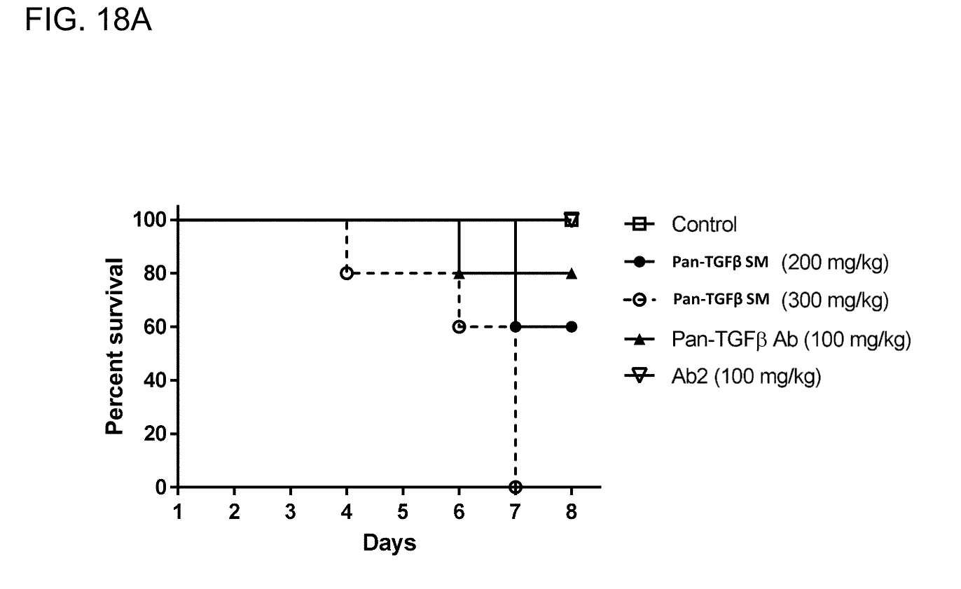

As shown in the survival data shown FIG. 17A, Ab2 exhibited reduced toxicity as compared to the other treatment groups. All animals administered 300 mg/kg of the ALK5 kinase inhibitor LY2109761 were sacrificed in a moribund condition or found dead on days 3, 6, or 7 of the study. Two of the animals administered 200 mg/kg of LY2109761 were found dead at day 7 of the study. One animal administered 100 mg/kg of the pan-TGFβ antibody was found dead at day 6 of the study. All animals administered up to 100 mg/kg of Ab2 survived until terminal sacrifice.

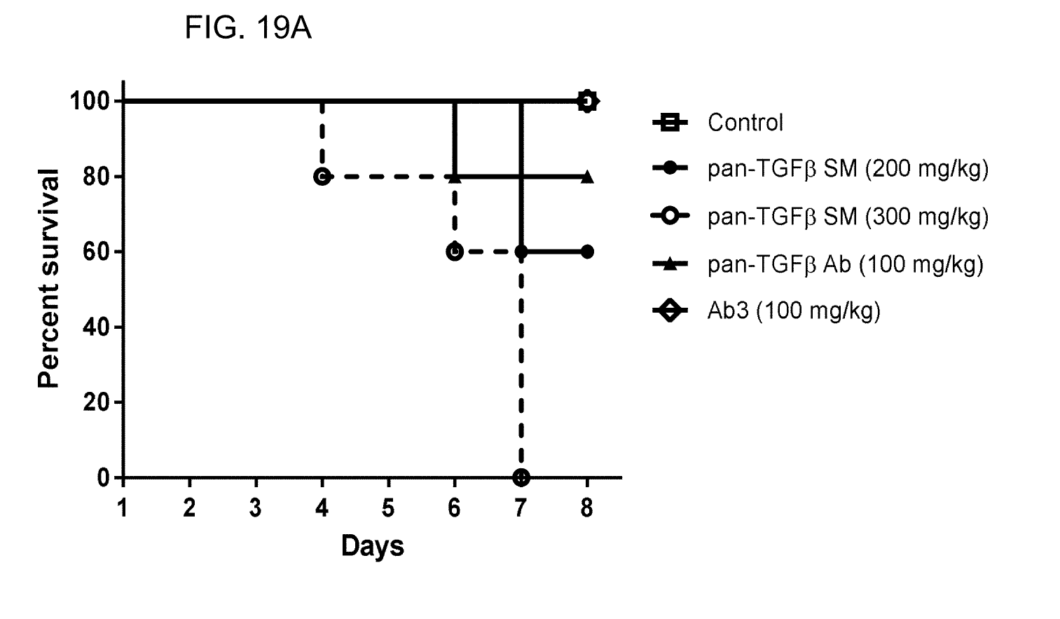

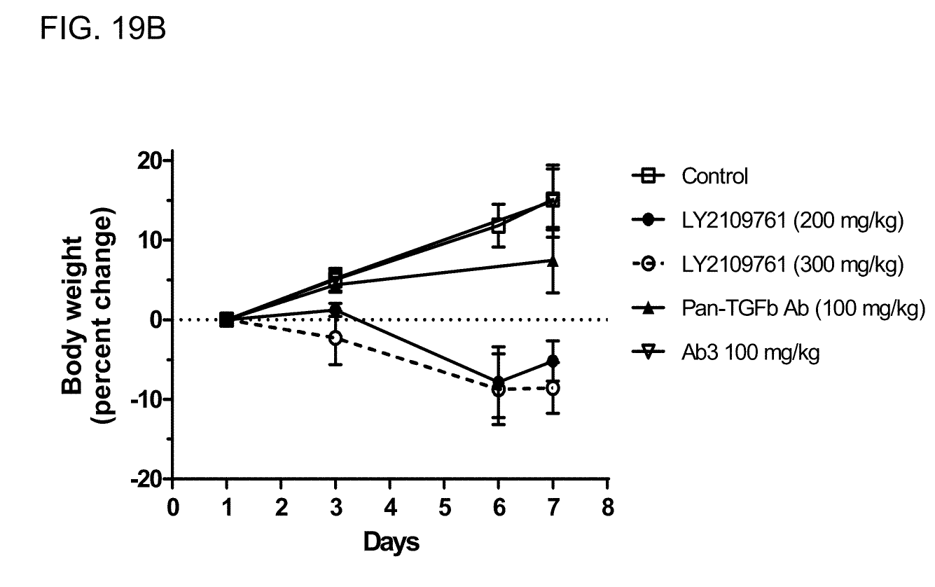

Similarly, as shown in the survival data shown FIG. 19A, rats treated with Ab3 exhibited reduced toxicity as compared to the other treatment groups. An animal administered 100 mg/kg of the pan-TGFβ antibody was found dead at day 6 of the study. All animals administered up to 100 mg/kg of Ab3 survived until terminal sacrifice.

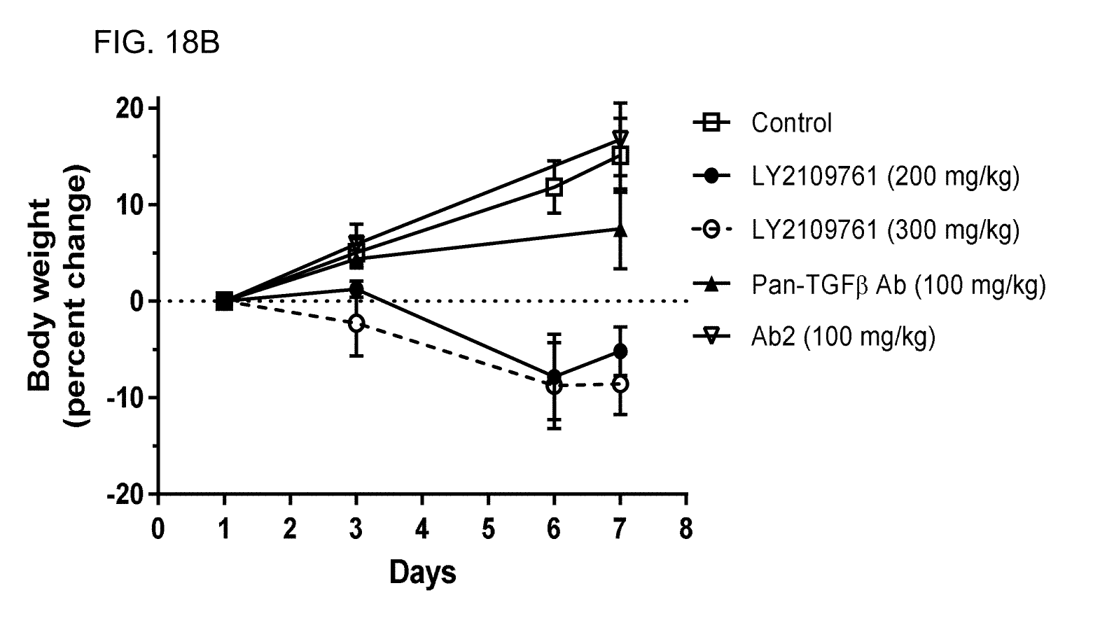

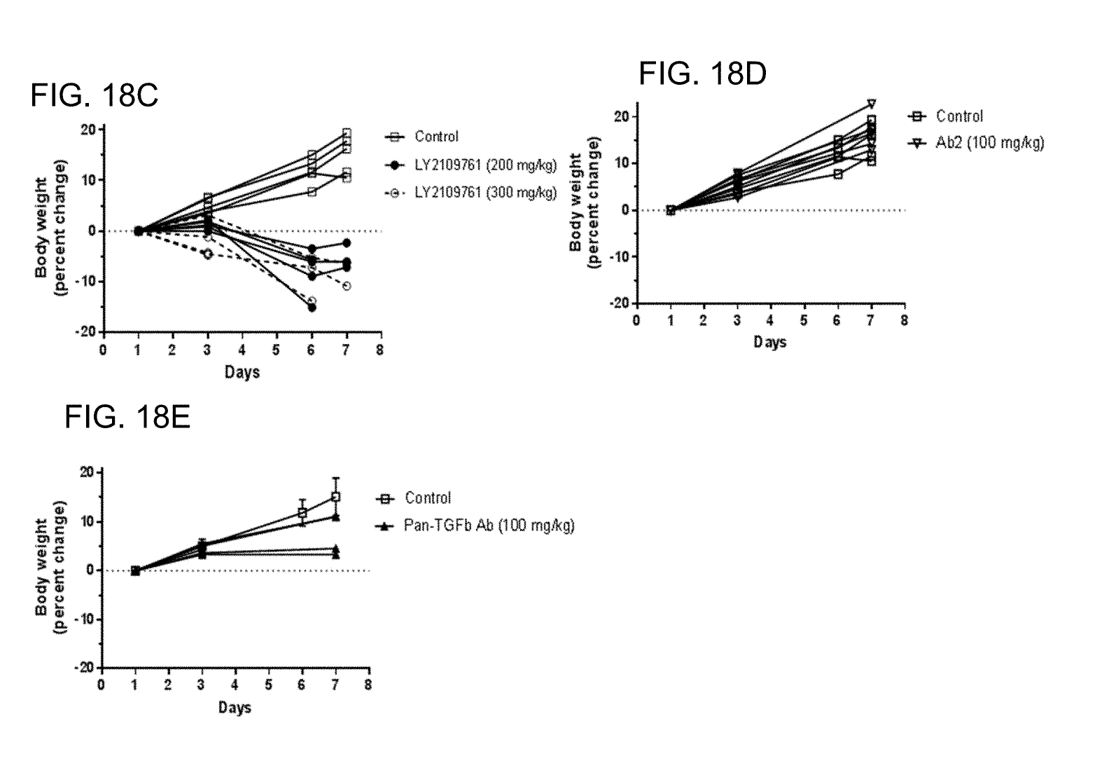

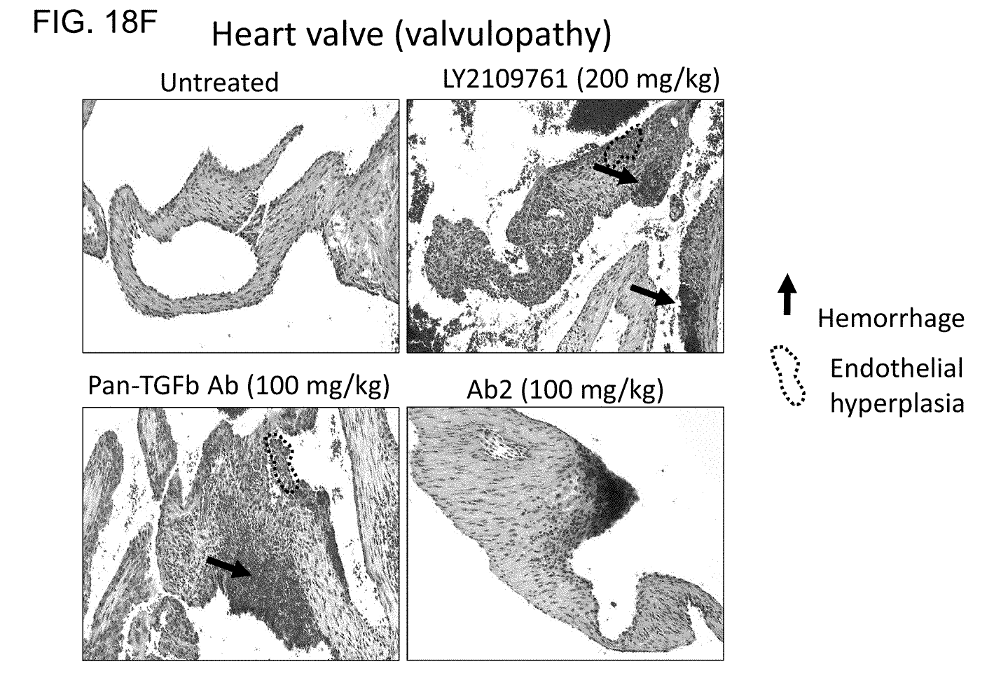

Further, the toxicity of the treatments was assessed by monitoring the body weights of the animals during the dosing phase. As shown in FIGS. 18B-18E, animals receiving LY2109761 at either 200 mg/kg or 300 mg/kg exhibited decreased body weight during the course of the study.

Animal organ weight was also assessed post-mortem. As shown in Table 11, Increased heart weights were observed in animals administered ≥200 mg/kg of LY2109761. Increased heart weights were also observed in animals administered ≥30 mg/kg of the pan-TGFβ antibody. No effects on organ weight were observed in animals administered up to 100 mg/kg of Ab2 or Ab3.

TABLE 11
Organ Weight Changes in Treatment Groups

| | Treatment Group | | | | | |
|---|---|---|---|---|---|---|
| | Vehicle | | | Pan-TGFβ | | |
| Dose Level | Control^{a} | LY2109761 | | Antibody | | |
| (mg/kg/day) | 0 | 200 | 300 | 3 | 30 | 100 |
| Heart | | | | | | |
| Absolute Weight (g) | 0.4084 | 112 | NE | 99 | 123 | 119 |
| Body Weight Ratio (%) | 0.3952 | 132 | NE | 96 | 122 | 122 |
| Brain Weight Ratio (%) | 26.3420 | 113 | NE | 98 | 123 | 116 |

NE = not evaluated due to early mortality.
Note:
Values for absolute weight and ratio of organ weights (relative to body or brain) for each treatment groups expressed as percentage control mean value.
^{a}Vehicle control = phosphate buffered saline (PBS), pH 7.4.

While no macroscopic findings were observed in animals administered up to 100 mg/kg of Ab2 or of the pan-TGFβ antibody, abnormally-shaped sternum was observed in four animals of each treatment group receiving either 200 mg/kg or 300 mg/kg of LY2109761. 2.5 mL of clear fluid in the thoracic cavity and an enlarged thymus due to excess fluid (i.e., edema) was observed in one animal administered 300 mg/kg of LY2109761, which was found dead on Day 3 of the study.

As shown in Table 12, at the microscopic level, animals administered ≥200 mg/kg of LY2109761 exhibited heart valve findings (i.e., valvulopathy). Valvulopathy was characterized by heart valve thickening due to hemorrhage, endothelial hyperplasia, mixed inflammatory cell infiltrates, and/or stromal hyperplasia (see FIG. 18F, upper right panel). Most animals had multiple valves affected. Additionally, atrium findings were observed including minimal to slight mixed inflammatory cell infiltrates, minimal hemorrhage, and/or minimal endothelium (endocardium) hyperplasia resulting in increased basophilic staining of the atrium in hematoxylin and eosin-stained sections. Myocardium findings were also observed mostly in the base of the heart and consisted of minimal to slight degeneration/necrosis, slight hemorrhage, and/or slight mixed inflammatory cell infiltrates. One animal administered 300 mg/kg of LY2109761 had slight necrosis with inflammation of a coronary artery. Further, two animals administered 200 mg/kg of LY2109761 had minimal mixed inflammatory cell infiltrates or hemorrhage in the aortic root.

TABLE 12
Microscopic Heart Findings in Animals Receiving LY2109761

| | LY2109761 | | |
|---|---|---|---|
| Dose Level (mg/kg/day) | 0 | 200 | 300 |
| Heart | | | |
| Heart valves | | | |
| Valvulopathy | | | |
| Minimal | 0 | 1 | 2 |
| Slight | 0 | 3 | 3 |
| Moderate | 0 | 1 | 0 |
| Atrium | | | |
| Infiltrate, mixed cell | | | |
| Minimal | 0 | 2 | 3 |
| Slight | 0 | 0 | 1 |
| Hyperplasia, endothelium | | | |
| Minimal | 0 | 1 | 3 |
| Hemorrhage | | | |
| Minimal | 0 | 1 | 2 |
| Myocardium | | | |
| Degeneration/necrosis | | | |
| Minimal | 0 | 0 | 1 |
| Slight | 0 | 1 | 1 |
| Hemorrhage | | | |
| Slight | 0 | 1 | 0 |
| Infiltrate, mixed cell | | | |
| Slight | 0 | 0 | 1 |
| Coronary artery | | | |
| Necrosis with inflammation | | | |
| Slight | 0 | 0 | 1 |
| Aortic root | | | |
| Hemorrhage | | | |
| Minimal | 0 | 1 | 0 |
| Infiltrate, mixed cell | | | |
| Minimal | 0 | 1 | 0 |

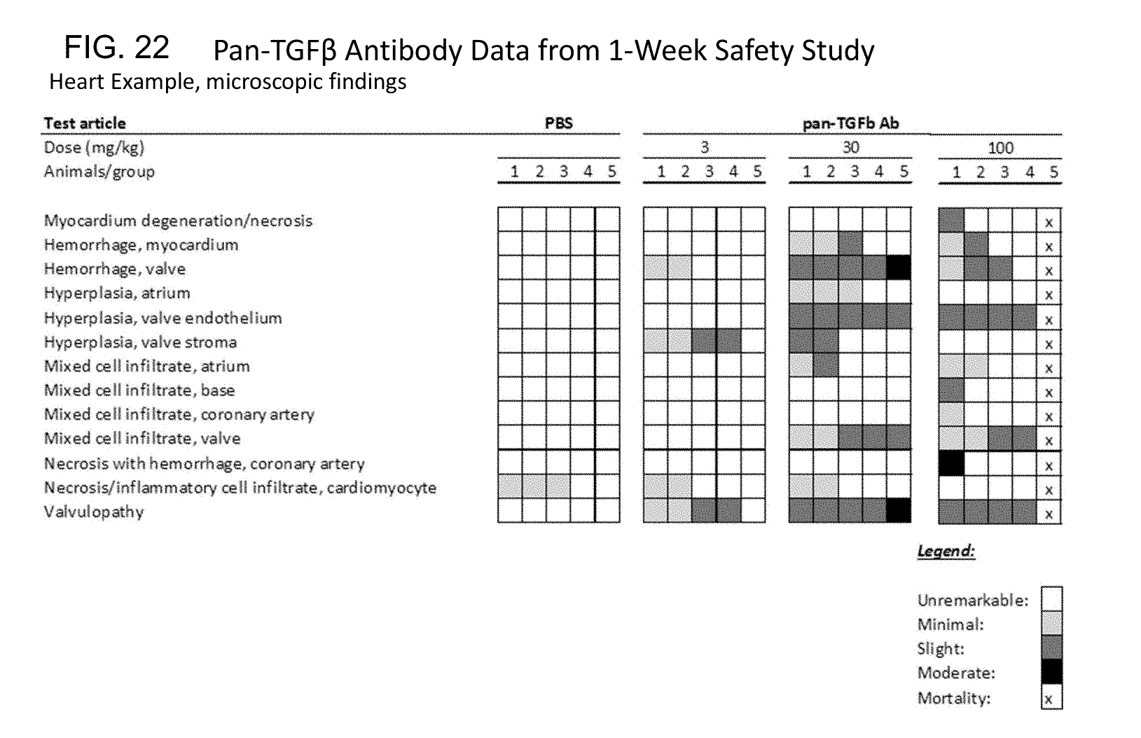

As shown in Table 13 and FIG. 22, animals administered 23 mg/kg of the pan-TGFβ antibody exhibited heart valve findings (i.e., valvulopathy) similar to those described in the animals administered LY2109761, as described above (see also FIG. 17F, lower left panel). Animals administered 230 mg/kg of the pan-TGFβ antibody exhibited atrium findings similar to those described in animals administered LY2109761. Animals administered 100 mg/kg of the pan-TGFβ antibody exhibited myocardium findings similar to those described in animals administered LY2109761, and animals administered 30 mg/kg of pan-TGFβ antibody had hemorrhage in the myocardium. One animal administered 100 mg/kg of the pan-TGFβ antibody had moderate intramural necrosis with hemorrhage in a coronary artery, which was associated with slight perivascular mixed inflammatory cell infiltrates. Bone findings in animals administered the pan-TGFβ antibody and LY2109761 consisted of macroscopic abnormally shaped sternum and microscopic increased thickness of the hypertrophic zone in the endplate of the sternum and physis of the femur and tibia; these findings were of higher incidence and/or severity in animals administered LY2109761 compared with pan-TGFβ antibody.

TABLE 13
Microscopic Heart Findings in
Animals Receiving the Pan-TGFβ Antibody

| | Pan-TGFβ Antibody | | | |
|---|---|---|---|---|
| Dose Level (mg/kg/day) | 0 | 3 | 30 | 100 |
| Heart | | | | |
| Heart valves | | | | |
| Valvulopathy | | | | |
| Minimal | 0 | 2 | 0 | 0 |
| Slight | 0 | 2 | 4 | 5 |
| Moderate | 0 | 0 | 1 | 0 |
| Atrium | | | | |
| Infiltrate, mixed cell | | | | |
| Minimal | 0 | 0 | 1 | 2 |
| Slight | 0 | 0 | 1 | 1 |
| Hyperplasia, endothelium | | | | |
| Minimal | 0 | 0 | 3 | 1 |
| Hemorrhage | | | | |
| Minimal | 0 | 0 | 1 | 0 |
| Myocardium | | | | |
| Degeneration/necrosis | | | | |
| Slight | 0 | 0 | 0 | 2 |
| Hemorrhage | | | | |
| Minimal | 0 | 0 | 2 | 1 |
| Slight | 0 | 0 | 1 | 1 |
| Infiltrate, mixed cell, base | | | | |
| Slight | 0 | 0 | 0 | 1 |
| Coronary artery | | | | |
| Necrosis with hemorrhage | | | | |
| Moderate | 0 | 0 | 0 | 1 |
| Infiltrate, mixed cell, | | | | |
| perivascular | | | | |
| Slight | 0 | 0 | 0 | 1 |

Although minimal or slight heart valve findings occurred in a small number of animals administered Ab2, these findings were considered unlikely test article related due to the low incidence (animal and number of heart valves within an animal), lack of a dose response, and/or lack of concurrent bone findings.

B. Phase II of the Study

In a second phase of the study, female rats were assigned to groups and administered either Ab2 at 3 mg/kg (1 group, n=5), at 30 mg/kg (1 group, n=5), or at 100 mg/kg (1 group, n=5); Ab3 at 3 mg/kg (1 group, n=5), 30 mg/kg (1 group, n=5), 100 mg/kg (1 group, n=5), or 60 mg/kg (1 group, n=5); LY2109761 at 200 mg/kg (1 group, n=5); or PBS (pH 7.4) (1 group, n=5), as discussed above. Animals receiving either Ab2, Ab3, or the vehicle control were dosed intravenously once weekly for 4 weeks at a volume of 10 mL/kg, and the rats receiving LY2109761 were dosed by oral gavage once daily for five days. Animals were sacrificed and necropsies performed.

Similar to the observations in the first phase of the study, the test article-related heart findings occurred for a shorter duration (i.e., 5 days instead of 7 days) in animals administered 200 mg/kg LY2109761. Microscopic heart findings were associated with increased heart weights for animals administered 200 mg/kg LY2109761 or ≥3 mg/kg pan-TGFβ antibody.

Although minimal or slight heart valve findings occurred in a small number of Phase II animals administered Ab2 or Ab3, these findings were considered unlikely test article related due to the low incidence (animal and number of heart valves within an animal), lack of a dose response, and/or lack of concurrent bone findings.

Additional tissues were evaluated in Phase II; no microscopic findings were attributed to Ab2 or Ab3. However, microscopic findings occurred in the bones (sternum, femur, and tibia), liver, pancreas (artery), thymus, thyroid, female reproductive tissues (ovary, uterus, cervix, and vagina), and mammary gland of Phase II animals administered 200 mg/kg LY2109761. Thymus findings consisted of minimally to slightly decreased lymphocytes in the cortex, which correlated with macroscopically small thymus and decreased thymus weights. Decreased thymus lymphocytes were consistent with a primary test article effect or were secondary stress effect (i.e., increased endogenous glucocorticoids). Minimal thyroid follicular cell hypertrophy, which correlated with increased thyroid weights, was consistent with liver enzyme induction, which resulted in increased metabolism of thyroxine. Increased liver weights for animals administered LY2109761 were suggestive of liver enzyme induction, but they lacked a microscopic correlate. Microscopic findings in the female reproductive tissues and mammary gland were consistent with decreased estrus cycling and were correlated with decreased uterus weights. Some animals also had mammary gland findings characterized by lobular hyperplasia/hypertrophy of the alveolar and/or ductal epithelial cells (i.e., masculinization), which was consistent with decreased estrogen.

C. Study Conclusion

In summary, animals treated with Ab2 and Ab3 at all doses tested (3 mg/kg, 30 mg/kg or 100 mg/kg) over a period of 4 weeks exhibited no toxic effects over background in any of the following parameters: myocardium degeneration or necrosis, atrium hemorrhage, myocardium hemorrhage, valve hemorrhage, valve endothelium hyperplasia, valve stroma hyperplasia, mixed inflammatory cell infiltrates in heart valves, mineralization, necrosis with hemorrhage in coronary artery, necrosis with inflammation in aortic root, necrosis or inflammatory cell infiltrate in cardiomyocyte, and valvulopathy. Thus, treatment with isoform-specific inhibitors of TGFβ1 activation surprisingly resulted in significantly improved safety profiles, e.g., reduced mortality and reduced cardiotoxicity as compared to pan-TGFβ inhibitor treatment (e.g., the ALK5 kinase inhibitor LY2109761 or the pan-TGFβ antibody).

### Example 20: Isoform-Selectivity of Ab3 In Vivo

To confirm isoform-selective inhibition of TGFβ1 in vivo, a pharmacodynamics study was conducted in which effects of Ab3 on tonic phospho-SMAD2/3 levels were assessed in bronchoalveolar lavage (BAL) cells collected from healthy rats. It is reported in the literature that under homeostatic conditions, BAL cells predominantly express TGFβ2/3, but little TGFβ1, while the latter becomes preferentially elevated in pathologic conditions.

Healthy Sprague Dawley rats (approximately 6-8 weeks old, weighing 200-250 g at the beginning of the study; Charles River) were randomized by bodyweight into study groups and dosed as described below.

Animals received test antibodies (huNEG-mIgG1, anti-integrin 136 antibody, or Ab3) on Days 1, 8, and 15 by intraperitoneal injection. Animals are euthanized on Day 16 for BAL and serum collections. One group of control animals was dosed with a single oral gavage (PO) dose of LY2109761 (small molecule ALK5 inhibitor) at 100 mg/kg and was euthanized at 2 hours (+/−20 min) post-dosing for BAL collections.

To collect BAL samples, the whole lung was lavaged three times with 5.0 mL of ice-cold Dulbecco's phosphate buffered saline. Lavagates were pooled and immediately placed on wet ice until processed as follows. A small portion (100-150 μL) from each sample was set aside on ice for cell counts. Remaining samples were centrifuged at 1,300 g (2-8° C.) for ≥10 minutes. Cell pellets were immediately placed on ice. 250 μL of the freshly prepared, ice-cold pSMAD lysis buffer was used to lyse the pellets. Lysed samples were centrifuged at 14,000 g for 10 minutes (2-8° C.). The resulting supernatant was aliquoted and immediately flash frozen in liquid nitrogen or on dry ice.

Serum samples were processed by centrifuging at 2,500 g, 2-8° C., for 10 minutes. Serum samples were frozen at −70 to −90° C.

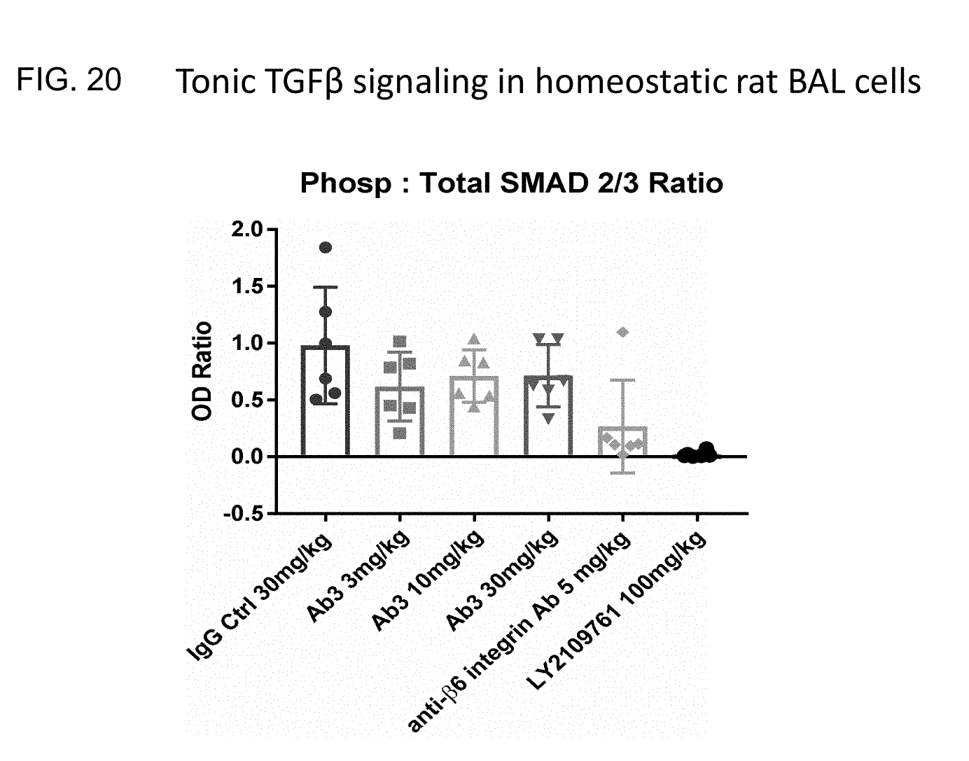

Phospho-SMAD2/3 assays were performed by ELISA (Cell Signaling Technologies) according to the manufacturer's instructions. Results were assessed by phosphorylated-to-total SMAD2/3 ratios. As shown in FIG. 20, tonic SMAD2/3 signaling was significantly suppressed in animals treated either with the small molecule pan-TGFβ inhibitor, LY2109761, or a monoclonal antibody against the 36 chain of integrin, which blocks integrin-mediated activation of TGFβ1/3. By comparison, animals treated with the TGFβ1 isoform-specific antibody, Ab3, maintained the tonic phosphorylation levels in BAL cells, supporting the notion that Ab3 is capable of selectively inhibiting TGFβ1 activation without perturbing the homeostatic function of TGFβ2 or TGFβ3 in vivo.

### Example 21: Ab3: A Novel and Highly Specific TGFβ1 Inhibiting Antibody with Antifibrotic Activity

Transforming growth factor-β1 (TGFβ1) has diverse biological functions, including regulation of immune responses and tissue homeostasis. Dysregulated TGFβ1 activation has been associated with a number of diseases, including kidney fibrosis, where chronic activation is a key disease driver. However, because of high homology between the TGFβ1 growth factor and its close relatives TGFβ2 and TGFβ3, truly TGFβ1-specific inhibitors have remained elusive. Pan-TGFβ inhibition, on the other hand, can cause dose-limiting heart valvulopathies, leading to toxicity concerns with long-term dosing. TGFβs are expressed as pro-proteins that are proteolytically cleaved into an N-terminal prodomain and a C-terminal growth factor. The prodomain remains noncovalently associated with the growth factor, preventing receptor binding. This latent TGFβ complex resides on cells or in the extracellular matrix until the complex is activated by integrins, freeing the growth factor and allowing receptor binding. To identify TGFβ1-specific antibodies, the prodomain, which shares much lower homology to TGFβ2 and TGFβ3 than the growth factor, was targeted. A monoclonal antibody Ab3 that specifically binds to latent TGFβ1, with no detectable binding to latent TGFβ2 or TGFβ3, was identified. Ab3 was shown to block latent TGFβ1 activation by αVβ6 or αVβ8 integrins, providing specificity unachieved by biologics that target the TGFβ1 growth factor/receptor interaction. Ab3 binds and inhibits latent TGFβ1 in complex with all four known TGFβ-presenting molecules, allowing targeting of latent TGFβ1 in multiple tissues. Ab3 blocks the activation of endogenous TGFβ1 in a number of primary cells, including dermal myofibroblasts and hepatic stellate cells. Finally, the in vivo efficacy of TGFβ1 inhibition via this novel mechanism was tested in the UUO model of kidney fibrosis, showing that Ab3 suppresses fibrosis markers to levels similar to those achieved in pan-TGFβ antibody-treated animals. Taken together, these data demonstrate that inhibition of latent TGFβ1 activation is efficacious in a preclinical fibrosis model and has a superior safety profile compared to pan-TGFβ inhibition.

### Example 22: Highly Specific Inhibition of TGFβ1 Activation by Ab1, an Antibody Having Antifibrotic Activity

Transforming growth factor-β1 (TGFβ1) is a cytokine with crucial and diverse biological functions, including regulation of immune responses and tissue homeostasis. TGFβs are expressed as pro-proteins that are proteolytically cleaved into an N-terminal prodomain and a C-terminal growth factor. The secreted growth factor remains noncovalently associated with the prodomain, preventing receptor binding and signaling. Latent TGFβ1 is covalently associated with presenting molecules through disulfide bonds that link latent TGFβ1 to the extracellular matrix or to the cell surface. To date, four TGFβ-presenting molecules (LTBP1, LTBP3, GARP, and LRRC33) have been identified. These presenting molecules play a critical role in the activation of the latent complex, as they provide an anchor for integrins to exert traction force on latent TGFβ1, thus releasing the active growth factor. Dysregulated TGFβ1 activation has been associated with a number of pathologies, including fibrotic diseases, where chronic TGFβ1 activation drives myofibroblast transdifferentiation and overexpression of extracellular matrix proteins. The role of TGFβ1 in driving fibrosis has led to the development of multiple therapeutics to inhibit its activity. However, inhibition with potent anti-pan-TGFβ antibodies was found to cause dose-limiting heart valvulopathies, leading to concerns about toxicity of this therapeutic approach. The alternative strategy of specifically targeting TGFβ1 is complicated by high homology between the TGFβ1 growth factor and its close relatives TGFβ2 and TGFβ3. The TGFβ1 prodomain, which has much lower homology to the prodomains of TGFβ2 and TGFβ3, was targeted and Ab3, a fully human monoclonal antibody that specifically binds to and inhibits activation of latent TGFβ1 with no detectable binding to latent TGFβ2 or TGFβ3, was identified. This novel mechanism allows isoform specificity unachieved by biologics that bind and block the TGFβ1 growth factor/receptor interaction and prevents latent TGFβ1 activation by both αVβ6 and αVβ8 integrins. Ab3 binds and inhibits latent TGFβ1 in complex with all four known TGFβ-presenting molecules, allowing targeting of latent TGFβ1 in multiple tissues. Ab3 inhibits endogenous TGFβ1 in a number of primary cells in vitro, including dermal myofibroblasts and hepatic stellate cells. In addition, the in vivo efficacy of TGFβ1 inhibition via this novel mechanism was tested in the unilateral ureteral obstruction model of kidney fibrosis. Ab3 was found to suppress the induction of profibrotic genes to levels similar to those achieved in pan-TGFβ antibody-treated animals. Taken together, these data demonstrate that inhibition of latent TGFβ1 activation is efficacious in a preclinical fibrosis model and has a potentially superior safety profile as compared to pan-TGFβ inhibition.

### Example 23: Bioinformatic Analysis of Relative Expressions of TGFβ1, TGFβ2 and TGFβ3

To evaluate the expression of TGFβ isoforms in cancerous tumors, gene expression (RNAseq) data from publically available datasets was examined. Using a publically available online interface tool (Firebrowse) to examine expression of TGFβ isoforms in The Cancer Genome Atlas (TCGA), the differential expression of RNA encoding TGFβ isoforms in both normal and cancerous tissue were first examined. All tumor RNAseq datasets in the TCGA database for which there were normal tissue comparators were selected, and expression of the TGFB1, TGFB2, and TGFB3 genes was examined (FIG. 21A). Data from the Firebrowse interface are represented as log 2 of reads per kilobase million (RPKM).

These data suggest that in most tumor types (gray), TGFB1 is the most abundantly expressed transcript of the TGFβ isoforms, with log 2(RPKM) values generally in the range of 4-6, vs. 0-2 for TGFB2 and 2-4 for TGFB3. We also note that in several tumor types, the average level of both TGFB1 and TGFB3 expression are elevated relative to normal comparator samples (black), suggesting that increased expression of these TGFβ isoforms may be associated with cancerous cells. Because of the potential role of TGFβ signaling in suppressing the host immune system in the cancer microenvironment, we were interested to note that TGFB1 transcripts were elevated in cancer types for which anti-PD1 or anti-PDL1 therapies are approved—these indications are labeled in gray on FIG. 21A.

Note that while RPKM >1 is generally considered to be the minimum value associated with biologically relevant gene expression (Hebenstreit et al., 2011; Wagner et al., 2013), however for subsequent analyses, more stringent cutoffs of RPKM (or of the related measure FPKM (see Conesa et al, 2016)) >10 or >30 to avoid false positives were used. For comparison, all three of those thresholds are indicated on FIG. 21A.

The large interquartile ranges in FIG. 21A indicate significant variability in TGFβ isoform expression among individual patients. To identify cancers where at least a subset of the patient population have tumors that differentially express the TGFB1 isoform, RNAseq data from individual tumor samples in the TCGA dataset was analyzed, calculating the number of fragments per kilobase million (FPKM). RPKM and FPKM are roughly equivalent, though FPKM corrects for double-counting reads at opposite ends of the same transcript (Conesa et al., 2016). Tumor samples were scored as positive for TGFB1, TGFB2, or TGFB3 expression if the FPKM value the transcript was >30 and the fraction of patients (expressed as %) of each cancer type that expressed each TGFβ isoform were calculated (FIG. 21B).

As shown in FIG. 21B, a majority of tumor types in the TGCA dataset show a significant percentage of individual samples that are TGFB1 positive, with some cancer types, including acute myeloid leukemia, diffuse large B-cell lymphoma, and head and neck squamous cell carcinoma, expressing TGFB1 in more than 80% of all tumor samples. Consistent with the data in FIG. 21A, fewer cancer types are positive for TGFB2 or TGFB3, though several cancers show an equal or greater percentage of tumor samples that are TGFB3 positive, including breast invasive carcinoma, mesothelioma, and sarcoma. These data suggest that cancer types may be stratified for TGFβ isoform expression, and that such stratification may be useful in identifying patients who are candidates for treatment with TGFβ isoform-specific inhibitors.

To further investigate this hypothesis, the log 2(FPKM) RNAseq data from a subset of individual tumor samples was plotted in a heat map (FIG. 21C), setting the color threshold to reflect FPKM >30 as a minimum transcript level to be scored TGFB isoform-positive.

Each sample is represented as a single row in the heat map, and samples are arranged by level of TGFB1 expression (highest expression levels at top). Consistent with the analysis in FIG. 21B, a significant number of samples in each cancer type are positive for TGFB1 expression. However, this representation also highlights the fact that many tumors express solely TGFB1 transcripts, particularly in the esophageal carcinoma, bladder urothelial, lung adenocarcinoma, and cutaneous melanoma cancer types. Interestingly, such TGFB1 skewing is not a feature of all cancers, as samples from breast invasive carcinoma show a much larger number of samples that are TGFB3-positive than are TGFB1 positive. Nonetheless, this analysis indicates that the 31 isoform is the predominant, and in most cases, the only, TGFβ family member present in tumors from a large number of cancer patients. Taken together with data suggesting that TGFβ signaling plays a significant role in immunosuppression in the cancer microenvironment, these findings also point to the utility of TGFβ1-specific inhibition in treatment of these tumors.

To identify mouse models in which to test the efficacy of TGFβ1-specific inhibition as a cancer therapeutic, TGFβ isoform expression in RNAseq data from a variety of cell lines used in mouse syngeneic tumor models was analyzed. For this analysis, two representations of the data were generated. First, similar to the data in FIG. 3, we generated a heat map of the log 2(FPKM) values for tumors derived from each cell line (FIG. 21D, left). Because this analysis was used to identify syngeneic models expressing high TGFB1 that are TGFB2 and TGFB3 negative, we were primarily concerned with avoiding false negatives, and we set our “positive” threshold at FPKM>1, well below that in the representations in FIGS. 21B and 21C.

As the data representation in FIG. 21D (left) makes clear, a number of syngeneic tumors commonly, including MC-38, 4T-1, and EMT6, express significant levels of both TGFβ1 and TGFβ. In contrast, the A20 and EL4 models express TGFβ1 almost exclusively, and the S91 and P815 tumors show a strong bias for TGFB1 expression.

To further evaluate the differential expression of TGFB1 vs TGFB2 and/or TGFB3, the minΔTGFB1 was calculated, defined as the smaller value of log 2(FPKM_{TGFB1})−log 2(FPKM_{TGFB2}) or log 2(FPKM_{TGFB1})−log 2(FPKM_{TGFB3}). The minΔTGFB1 for each model is shown as a heat map in FIG. 21D (right), and underscores the conclusion from FIG. 21D (left) that syngeneic tumors from the A20, EL4, S91, and/or P815 cell lines may represent excellent models in which to test the efficacy of TGFβ1-specific inhibitors.

The various features and embodiments of the present invention, referred to in individual sections above apply, as appropriate, to other sections, mutatis mutandis. Consequently, features specified in one section may be combined with features specified in other sections, as appropriate.

Those skilled in the art will recognize, or be able to ascertain using no more than routine experimentation, many equivalents to the specific embodiments of the invention described herein. Such equivalents are intended to be encompassed by the following claims.

1. A method for identifying a TGFβ1 inhibitor for therapeutic use, the method comprising the step of:
selecting an antibody, or an antigen-binding fragment thereof, that binds at least one type of extracellular matrix (ECM)-associated proTGFβ1 complex and at least one type of cell-associated proTGFβ1 complex, wherein the antibody, or the antigen-binding fragment thereof, inhibits activation of TGFβ1;
and, wherein the antibody, or the antigen-binding fragment thereof, binds a hLTBP1-proTGFβ1 complex and/or a hLTBP3-proTGFβ1 complex with a K_{D} of ≤0.5 nM as measured by bio-layer interferometry.

2. The method of claim 1, wherein the ECM-associated proTGFβ1 complex comprises hLTBP1 or hLTBP3.

3. The method of claim 1, wherein the cell-associated proTGFβ1 complex comprises hGARP or hLRRC33.

4. The method of claim 1, wherein the antibody, or the antigen-binding fragment thereof, is capable of specifically binding each of the following complexes:
a hLTBP1-proTGFβ1 complex, a hLTBP3-proTGFβ1 complex, a hGARP-proTGFβ1 complex, and a hLRRC33-proTGFβ1 complex.

5. The method of claim 1, wherein the antibody, or the antigen-binding fragment thereof, preferentially binds the hLTBP1-proTGFβ1 complex and/or the hLTBP3-proTGFβ1 complex over a hGARP-proTGFβ1 complex.

6. The method of claim 5, wherein the antibody, or the antigen-binding fragment thereof, binds the hGARP-proTGFβ1 complex with a KD of ≥4 nM, as measured by bio-layer interferometry.

7. The method of claim 1, further comprising the steps of:
carrying out a preclinical study that comprises administration of a therapeutic dose of the antibody, or the antigen-binding fragment thereof, to evaluate in vivo efficacy; and
carrying out a toxicology/tolerability study in an animal model to evaluate in vivo safety;
wherein the therapeutic dose is shown to be both safe and efficacious in vivo.

8. The method of claim 7, wherein the administration of the therapeutic dose is sufficient to reduce expression of one or more genes selected from the group consisting of:
Serpine 1, MCP-1/CCL2, Col1a1, Col3a1, FN1, TGFB1, CTGF, and ACTA2.

9. The method of claim 7, wherein the administration of the therapeutic dose is sufficient to reduce phosphorylation of SMAD2/3.

10. The method of claim 7, wherein the toxicology/tolerability study evaluates cardiovascular toxicity, gastrointestinal toxicity, immunotoxicity, bone toxicity, cartilage toxicity, reproductive system toxicity, and/or renal toxicity.

11. The method of claim 7, wherein the toxicology/tolerability of the antibody, or the antigen-binding fragment thereof, is evaluated at a dosage of at least up to 100 mg/kg/week.

12. The method of claim 7, wherein no test article-related toxicities are observed when the antibody, or the antigen-binding fragment thereof, is administered at 100 mg/kg/week for 4 weeks.

13. The method of claim 1, wherein the antibody, or the antigen-binding fragment thereof, does not bind TGFβ2 or proTGFβ2.

14. The method of claim 13, wherein the antibody, or the antigen-binding fragment thereof, does not bind TGFβ3 or proTGFβ3.

15. A method for making a pharmaceutical composition comprising a TGFβ1 inhibitor, the method comprising the step of:
formulating the antibody or the antigen-binding fragment identified in the method of claim 1 into a pharmaceutical composition with one or more pharmaceutically acceptable excipients.


SEQUENCE LISTING (101 sequences)

SEQ ID NO: 1 - protein, 5 aa, Artificial Sequence
  Synthetic Ab1 CDRH1
SYGMH

SEQ ID NO: 2 - protein, 5 aa, Artificial Sequence
  Synthetic Ab2 CDRH1
SDWIG

SEQ ID NO: 3 - protein, 17 aa, Artificial Sequence
  Synthetic Ab1 CDRH2
VISYDGSNKYYADSVKG

SEQ ID NO: 4 - protein, 17 aa, Artificial Sequence
  Synthetic Ab2 CDRH2
VIYPGDSDTRYSASFQG

SEQ ID NO: 5 - protein, 14 aa, Artificial Sequence
  Synthetic Ab1 CDRH3
DIRPYGDYSAAFDI

SEQ ID NO: 6 - protein, 15 aa, Artificial Sequence
  Synthetic Ab2 CDRH3
AAGIAAAGHVTAFDI

SEQ ID NO: 7 - protein, 13 aa, Artificial Sequence
  Synthetic Ab1 CDRL1
TGSSGSIASNYVQ

SEQ ID NO: 8 - protein, 17 aa, Artificial Sequence
  Synthetic Ab2 CDRL1
KSSQSVLYSSNNKNYLA

SEQ ID NO: 9 - protein, 7 aa, Artificial Sequence
  Synthetic Ab1 CDRL2
EDNQRPS

SEQ ID NO: 10 - protein, 7 aa, Artificial Sequence
  Synthetic Ab2 CDRL2
WASTRES

SEQ ID NO: 11 - protein, 11 aa, Artificial Sequence
  Synthetic Ab1 CDRL3
QSYDSSNHGGV

SEQ ID NO: 12 - protein, 9 aa, Artificial Sequence
  Synthetic Ab2 CDRL3
QQYYSTPVT

SEQ ID NO: 13 - protein, 123 aa, Artificial Sequence
  Synthetic Ab1  Heavy chain variable region amino acid sequence
EVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCARDIRPYGDYSAAFDIWGQGTLVTVSS

SEQ ID NO: 14 - protein, 112 aa, Artificial Sequence
  Synthetic Ab1  Light chain variable region amino acid sequence
NFMLTQPHSVSESPGKTVTISCTGSSGSIASNYVQWYQQRPGSAPSIVIFEDNQRPSGAPDRFSGSIDSSSNSASLTISGLKTEDEADYYCQSYDSSNHGGVFGGGTQLTVL

SEQ ID NO: 15 - protein, 449 aa, Artificial Sequence
  Synthetic Ab1  Heavy chain amino acid sequence
EVQLVESGGGLVQPGRSLRLSCAASGFTFSSYGMHWVRQAPGKGLEWVAVISYDGSNKYYADSVKGRFTISRDNSKNTLYLQMNSLRAEDTAVYYCARDIRPYGDYSAAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 218 aa, Artificial Sequence
  Synthetic Ab1  Light chain amino acid sequence
NFMLTQPHSVSESPGKTVTISCTGSSGSIASNYVQWYQQRPGSAPSIVIFEDNQRPSGAPDRFSGSIDSSSNSASLTISGLKTEDEADYYCQSYDSSNHGGVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 124 aa, Artificial Sequence
  Synthetic Ab2  Heavy chain variable region amino acid sequence
EVQLVQSGAEMKKPGESLKISCKGSGYNFASDWIGWVRQTPGKGLEWMGVIYPGDSDTRYSASFQGQVTISADKSINTAYLQWSSLKASDTAMYYCASAAGIAAAGHVTAFDIWGQGTMVTVSS

SEQ ID NO: 18 - protein, 113 aa, Artificial Sequence
  Synthetic Ab2  Light chain variable region amino acid sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPVTFGQGTKLEIK

SEQ ID NO: 19 - protein, 450 aa, Artificial Sequence
  Synthetic Ab2  Heavy chain amino acid sequence
EVQLVQSGAEMKKPGESLKISCKGSGYNFASDWIGWVRQTPGKGLEWMGVIYPGDSDTRYSASFQGQVTISADKSINTAYLQWSSLKASDTAMYYCASAAGIAAAGHVTAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 220 aa, Artificial Sequence
  Synthetic Ab2  Light chain amino acid sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 361 aa, Artificial Sequence
  Synthetic TGF-beta-1
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 22 - protein, 395 aa, Artificial Sequence
  Synthetic TGF-beta-2
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 23 - protein, 392 aa, Artificial Sequence
  Synthetic TGF-beta-3
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 24 - protein, 361 aa, Artificial Sequence; identical to SEQ ID NO: 21
  Synthetic proTGF-beta-1

SEQ ID NO: 25 - protein, 361 aa, Artificial Sequence
  Synthetic proTGF-beta-1 C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 26 - protein, 360 aa, Artificial Sequence
  Synthetic proTGF-beta-1 D2G
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 27 - protein, 360 aa, Artificial Sequence
  Synthetic proTGF-beta-1 C4S D2G
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 28 - protein, 395 aa, Artificial Sequence; identical to SEQ ID NO: 22
  Synthetic proTGF-beta-2

SEQ ID NO: 29 - protein, 395 aa, Artificial Sequence
  Synthetic proTGF-beta-2 C5S
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 30 - protein, 394 aa, Artificial Sequence
  Synthetic proTGF-beta-2 C5S D2G
SLSTSSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 31 - protein, 394 aa, Artificial Sequence
  Synthetic proTGF-beta-2 D2G
SLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKGALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 32 - protein, 392 aa, Artificial Sequence; identical to SEQ ID NO: 23
  Synthetic proTGF-beta-3

SEQ ID NO: 33 - protein, 392 aa, Artificial Sequence
  Synthetic proTGF-beta-3 C7S
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 34 - protein, 391 aa, Artificial Sequence
  Synthetic proTGF-beta-3 C7S D2G
SLSLSTSTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 35 - protein, 391 aa, Artificial Sequence
  Synthetic proTGF-beta-3 D2G
SLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKGALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 36 - protein, 361 aa, Artificial Sequence
  Synthetic proTGF-beta-1
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 37 - protein, 361 aa, Artificial Sequence
  Synthetic proTGF-beta-1
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 38 - protein, 249 aa, Artificial Sequence
  Synthetic TGF-beta-1 LAP C4S
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRR

SEQ ID NO: 39 - protein, 249 aa, Artificial Sequence
  Synthetic TGF-beta-1 LAP C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRR

SEQ ID NO: 40 - protein, 360 aa, Artificial Sequence
  Synthetic proTGF-beta-1 C4S D2G
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHGALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 41 - protein, 361 aa, Artificial Sequence
  Synthetic proTGF-beta-1 C4S
LSTSKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 42 - protein, 361 aa, Artificial Sequence
  Synthetic proTGF-beta-1 C4S
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 43 - protein, 360 aa, Artificial Sequence
  Synthetic proTGF-beta-1 C4S D2G
LSTSKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHGALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 44 - protein, 1260 aa, Artificial Sequence
  Synthetic LTBP3
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRAGALSTGALPPLAPEGDSVASKHAIYAVQVIADPPGPGEGPPAQHAAFLVPLGPGQISAEVQAPPPVVNVRVHHPPEASVQVHRIESSNAEGAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPADGTAAFKEICPAGKGYHILTSHQTLTIQGESDFSLFLHPDGPPKPQQLPESPSQAPPPEDTEEERGVTTDSPVSEERSVQQSHPTATTSPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCVDVDECEAGDVCDNGICTNTPGSFQCQCLSGYHLSRDRSHCEDIDECDFPAACIGGDCINTNGSYRCLCPQGHRLVGGRKCQDIDECTQDPGLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGAEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPQAGPALTFDDCCCRQGRGWGAQCRPCPPRGAGSQCPTSQSESNSFWDTSPLLLGKPRRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 45 - protein, 1228 aa, Artificial Sequence
  Synthetic LTBP3
GPAGERGTGGGGALARERFKVVFAPVICKRTCLKGQCRDSCQQGSNMTLIGENGHSTDTLTGSAFRVVVCPLPCMNGGQCSSRNQCLCPPDFTGRFCQVPAAGTGAGTGSSGPGLARTGAMSTGPLPPLAPEGESVASKHAIYAVQVIADPPGPGEGPPAQHAAFLVPLGPGQISAEVQAPPPVVNVRVHHPPEASVQVHRIEGPNAEGPASSQHLLPHPKPPHPRPPTQKPLGRCFQDTLPKQPCGSNPLPGLTKQEDCCGSIGTAWGQSKCHKCPQLQYTGVQKPVPVRGEVGADCPQGYKRLNSTHCQDINECAMPGNVCHGDCLNNPGSYRCVCPPGHSLGPLAAQCIADKPEEKSLCFRLVSTEHQCQHPLTTRLTRQLCCCSVGKAWGARCQRCPADGTAAFKEICPGKGYHILTSHQTLTIQGESDFSLFLHPDGPPKPQQLPESPSRAPPLEDTEEERGVTMDPPVSEERSVQQSHPTTTTSPPRPYPELISRPSPPTFHRFLPDLPPSRSAVEIAPTQVTETDECRLNQNICGHGQCVPGPSDYSCHCNAGYRSHPQHRYCVDVNECEAEPCGPGKGICMNTGGSYNCHCNRGYRLHVGAGGRSCVDLNECAKPHLCGDGGFCINFPGHYKCNCYPGYRLKASRPPICEDIDECRDPSTCPDGKCENKPGSFKCIACQPGYRSQGGGACRDVNECSEGTPCSPGWCENLPGSYRCTCAQYEPAQDGLSCIDVDECEAGKVCQDGICTNTPGSFQCQCLSGYHLSRDRSRCEDIDECDFPAACIGGDCINTNGSYRCLCPLGHRLVGGRKCKKDIDECSQDPGLCLPHACENLQGSYVCVCDEGFTLTQDQHGCEEVEQPHHKKECYLNFDDTVFCDSVLATNVTQQECCCSLGAGWGDHCEIYPCPVYSSAEFHSLVPDGKRLHSGQQHCELCIPAHRDIDECILFGAEICKEGKCVNTQPGYECYCKQGFYYDGNLLECVDVDECLDESNCRNGVCENTRGGYRCACTPPAEYSPAQAQCLIPERWSTPQRDVKCAGASEERTACVWGPWAGPALTFDDCCCRQPRLGTQCRPCPPRGTGSQCPTSQSESNSFWDTSPLLLGKSPRDEDSSEEDSDECRCVSGRCVPRPGGAVCECPGGFQLDASRARCVDIDECRELNQRGLLCKSERCVNTSGSFRCVCKAGFTRSRPHGPACLSAAADDAAIAHTSVIDHRGYFH

SEQ ID NO: 46 - protein, 1373 aa, Artificial Sequence
  Synthetic LTBP1S
NHTGRIKVVFTPSICKVTCTKGSCQNSCEKGNTTTLISENGHAADTLTATNFRVVLCHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYKFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCTNGDCSNLEGSYMCSCHKGYTRTPDHKHCKDIDECQQGNLCVNGQCKNTEGSFRCTCGQGYQLSAAKDQCEDIDECQHHHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVEQPKEEKKECYYNLNDASLCDNVLAPNVTKQECCCTSGAGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPMKDSDDYAQLCNIPVTGRRQPYGRDALVDFSEQYAPEADPYFIQDRFLNSFEELQAEECGILNGCENGRCVRVQEGYTCDCFDGYHLDTAKMTCVDVNECDELNNRMSLCKNAKCINTEGSYKCLCLPGYVPSDKPNYCTPLNTALNLEKDSDLE

SEQ ID NO: 47 - protein, 1374 aa, Artificial Sequence
  Synthetic LTBP1S
NHTGRIKVVFTPSICKVTCTKGNCQNSCQKGNTTTLISENGHAADTLTATNFRVVICHLPCMNGGQCSSRDKCQCPPNFTGKLCQIPVLGASMPKLYQHAQQQGKALGSHVIHSTHTLPLTMTSQQGVKVKFPPNIVNIHVKHPPEASVQIHQVSRIDSPGGQKVKEAQPGQSQVSYQGLPVQKTQTVHSTYSHQQLIPHVYPVAAKTQLGRCFQETIGSQCGKALPGLSKQEDCCGTVGTSWGFNKCQKCPKKQSYHGYTQMMECLQGYKRVNNTFCQDINECQLQGVCPNGECLNTMGSYRCSCKMGFGPDPTFSSCVPDPPVISEEKGPCYRLVSPGRHCMHPLSVHLTKQICCCSVGKAWGPHCEKCPLPGTAAFKEICPGGMGYTVSGVHRRRPIHQHIGKEAVYVKPKNTQPVAKSTHPPPLPAKEEPVEALTSSWEHGPRGAEPEVVTAPPEKEIPSLDQEKTRLEPGQPQLSPGVSTIHLHPQFPVVVEKTSPPVPVEVAPEASTSSASQVIAPTQVTEINECTVNPDICGAGHCINLPVRYTCICYEGYKFSEQLRKCVDIDECAQVRHLCSQGRCENTEGSFLCVCPAGFMASEEGTNCIDVDECLRPDMCRDGRCINTAGAFRCEYCDSGYRMSRRGYCEDIDECLKPSTCPEEQCVNTPGSYQCVPCTEGFRGWNGQCLDVDECLQPKVCTNGSCTNLEGSYMCSCHRGYSPTPDHRHCQDIDECQQGNLCMNGQCRNTDGSFRCTCGQGYQLSAAKDQCEDIDECEHHHLCSHGQCRNTEGSFQCVCNQGYRASVLGDHCEDINECLEDSSVCQGGDCINTAGSYDCTCPDGFQLNDNKGCQDINECAQPGLCGSHGECLNTQGSFHCVCEQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRVTEDSGVDRQPREEKKECYYNLNDASLCDNVLAPNVTKQECCCTSGAGWGDNCEIFPCPVQGTAEFTEMCPRGKGLVPAGESSYDTGGENYKDADECLLFGEEICKNGYCLNTQPGYECYCKQGTYYDPVKLQCFDMDECQDPNSCIDGQCVNTEGSYNCFCTHPMVLDASEKRCVQPTESNEQIEETDVYQDLCWEHLSEEYVCSRPLVGKQTTYTECCCLYGEAWGMQCALCPMKDSDDYAQLCNIPVTGRRRPYGRDALVDFSEQYGPETDPYFIQDRFLNSFEELQAEECGILNGCENGRCVRVQEGYTCDCFDGYHLDMAKMTCVDVNECSELNNRMSLCKNAKCINTEGSYKCLCLPGYIPSDKPNYCTPLNSALNLDKESDLE

SEQ ID NO: 48 - protein, 646 aa, Artificial Sequence
  Synthetic GARP
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVNLILLLSFTLVSAIVLTTLATICFLRRQKLSQQYKA

SEQ ID NO: 49 - protein, 611 aa, Artificial Sequence
  Synthetic sGARP
ISQRREQVPCRTVNKEALCHGLGLLQVPSVLSLDIQALYLSGNQLQSILVSPLGFYTALRHLDLSDNQISFLQAGVFQALPYLEHLNLAHNRLATGMALNSGGLGRLPLLVSLDLSGNSLHGNLVERLLGETPRLRTLSLAENSLTRLARHTFWGMPAVEQLDLHSNVLMDIEDGAFEALPHLTHLNLSRNSLTCISDFSLQQLQVLDLSCNSIEAFQTAPEPQAQFQLAWLDLRENKLLHFPDLAVFPRLIYLNVSNNLIQLPAGLPRGSEDLHAPSEGWSASPLSNPSRNASTHPLSQLLNLDLSYNEIELVPASFLEHLTSLRFLNLSRNCLRSFEARQVDSLPCLVLLDLSHNVLEALELGTKVLGSLQTLLLQDNALQELPPYTFASLASLQRLNLQGNQVSPCGGPAEPGPPGCVDFSGIPTLHVLNMAGNSMGMLRAGSFLHTPLTELDLSTNPGLDVATGALVGLEASLEVLELQGNGLTVLRVDLPCFLRLKRLNLAENQLSHLPAWTRAVSLEVLDLRNNSFSLLPGNAMGGLETSLRRLYLQGNPLSCCGNGWLAAQLHQGRVDVDATQDLICRFGSQEELSLSLVRPEDCEKGGLKNVN

SEQ ID NO: 50 - protein, 1375 aa, Artificial Sequence
  Synthetic LTBP1S
NHTGRIKVVFTPSICKVTCTKGSCQNSCEKGNTTTLISENGHAADTLTATNFRVVICHLPCMNGGQCSSRDKCQCPPNFTGKLCQIPVHGASVPKLYQHSQQPGKALGTHVIHSTHTLPLTVTSQQGVKVKFPPNIVNIHVKHPPEASVQIHQVSRIDGPTGQKTKEAQPGQSQVSYQGLPVQKTQTIHSTYSHQQVIPHVYPVAAKTQLGRCFQETIGSQCGKALPGLSKQEDCCGTVGTSWGFNKCQKCPKKPSYHGYNQMMECLPGYKRVNNTFCQDINECQLQGVCPNGECLNTMGSYRCTCKIGFGPDPTFSSCVPDPPVISEEKGPCYRLVSSGRQCMHPLSVHLTKQLCCCSVGKAWGPHCEKCPLPGTAAFKEICPGGMGYTVSGVHRRRPIHHHVGKGPVFVKPKNTQPVAKSTHPPPLPAKEEPVEALTFSREHGPGVAEPEVATAPPEKEIPSLDQEKTKLEPGQPQLSPGISTIHLHPQFPVVIEKTSPPVPVEVAPEASTSSASQVIAPTQVTEINECTVNPDICGAGHCINLPVRYTCICYEGYRFSEQQRKCVDIDECTQVQHLCSQGRCENTEGSFLCICPAGFMASEEGTNCIDVDECLRPDVCGEGHCVNTVGAFRCEYCDSGYRMTQRGRCEDIDECLNPSTCPDEQCVNSPGSYQCVPCTEGFRGWNGQCLDVDECLEPNVCANGDCSNLEGSYMCSCHKGYTRTPDHKHCRDIDECQQGNLCVNGQCKNTEGSFRCTCGQGYQLSAAKDQCEDIDECQHRHLCAHGQCRNTEGSFQCVCDQGYRASGLGDHCEDINECLEDKSVCQRGDCINTAGSYDCTCPDGFQLDDNKTCQDINECEHPGLCGPQGECLNTEGSFHCVCQQGFSISADGRTCEDIDECVNNTVCDSHGFCDNTAGSFRCLCYQGFQAPQDGQGCVDVNECELLSGVCGEAFCENVEGSFLCVCADENQEYSPMTGQCRSRTSTDLDVDVDQPKEEKKECYYNLNDASLCDNVLAPNVTKQECCCTSGVGWGDNCEIFPCPVLGTAEFTEMCPKGKGFVPAGESSSEAGGENYKDADECLLFGQEICKNGFCLNTRPGYECYCKQGTYYDPVKLQCFDMDECQDPSSCIDGQCVNTEGSYNCFCTHPMVLDASEKRCIRPAESNEQIEETDVYQDLCWEHLSDEYVCSRPLVGKQTTYTECCCLYGEAWGMQCALCPLKDSDDYAQLCNIPVTGRRQPYGRDALVDFSEQYTPEADPYFIQDRFLNSFEELQAEECGILNGCENGRCVRVQEGYTCDCFDGYHLDTAKMTCVDVNECDELNNRMSLCKNAKCINTDGSYKCLCLPGYVPSDKPNYCTPLNTALNLEKDSDLE

SEQ ID NO: 51 - protein, 1260 aa, Artificial Sequence
  Synthetic LTBP3
GPAGERGAGGGGALARERFKVVFAPVICKRTCLKGQCRDSCQQGSNMTLIGENGHSTDTLTGSGFRVVVCPLPCMNGGQCSSRNQCLCPPDFTGRFCQVPAGGAGGGTGGSGPGLSRTGALSTGALPPLAPEGDSVASKHAIYAVQVIADPPGPGEGPPAQHAAFLVPLGPGQISAEVQAPPPVVNVRVHHPPEASVQVHRIESSNAESAAPSQHLLPHPKPSHPRPPTQKPLGRCFQDTLPKQPCGSNPLPGLTKQEDCCGSIGTAWGQSKCHKCPQLQYTGVQKPGPVRGEVGADCPQGYKRLNSTHCQDINECAMPGVCRHGDCLNNPGSYRCVCPPGHSLGPSRTQCIADKPEEKSLCFRLVSPEHQCQHPLTTRLTRQLCCCSVGKAWGARCQRCPTDGTAAFKEICPAGKGYHILTSHQTLTIQGESDFSLFLHPDGPPKPQQLPESPSQAPPPEDTEEERGVTTDSPVSEERSVQQSHPTATTTPARPYPELISRPSPPTMRWFLPDLPPSRSAVEIAPTQVTETDECRLNQNICGHGECVPGPPDYSCHCNPGYRSHPQHRYCVDVNECEAEPCGPGRGICMNTGGSYNCHCNRGYRLHVGAGGRSCVDLNECAKPHLCGDGGFCINFPGHYKCNCYPGYRLKASRPPVCEDIDECRDPSSCPDGKCENKPGSFKCIACQPGYRSQGGGACRDVNECAEGSPCSPGWCENLPGSFRCTCAQGYAPAPDGRSCLDVDECEAGDVCDNGICSNTPGSFQCQCLSGYHLSRDRSHCEDIDECDFPAACIGGDCINTNGSYRCLCPQGHRLVGGRKCQDIDECSQDPSLCLPHGACKNLQGSYVCVCDEGFTPTQDQHGCEEVEQPHHKKECYLNFDDTVFCDSVLATNVTQQECCCSLGAGWGDHCEIYPCPVYSSAEFHSLCPDGKGYTQDNNIVNYGIPAHRDIDECMLFGSEICKEGKCVNTQPGYECYCKQGFYYDGNLLECVDVDECLDESNCRNGVCENTRGGYRCACTPPAEYSPAQRQCLSPEEMDVDECQDPAACRPGRCVNLPGSYRCECRPPWVPGPSGRDCQLPESPAERAPERRDVCWSQRGEDGMCAGPLAGPALTFDDCCCRQGRGWGAQCRPCPPRGAGSHCPTSQSESNSFWDTSPLLLGKPPRDEDSSEEDSDECRCVSGRCVPRPGGAVCECPGGFQLDASRARCVDIDECRELNQRGLLCKSERCVNTSGSFRCVCKAGFARSRPHGACVPQRRR

SEQ ID NO: 52 - protein, 645 aa, Artificial Sequence
  Synthetic GARP
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 53 - protein, 610 aa, Artificial Sequence
  Synthetic sGARP
AQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNIN

SEQ ID NO: 54 - protein, 5 aa, Artificial Sequence
  Synthetic peptide
CPPCP

SEQ ID NO: 55 - protein, 13 aa, Artificial Sequence
  Synthetic Linker
ASTKGPSVFPLAP

SEQ ID NO: 56 - protein, 6 aa, Artificial Sequence
  Synthetic Linker
ASTKGP

SEQ ID NO: 57 - protein, 12 aa, Artificial Sequence
  Synthetic Linker
TVAAPSVFIFPP

SEQ ID NO: 58 - protein, 5 aa, Artificial Sequence
  Synthetic Linker
TVAAP

SEQ ID NO: 59 - protein, 16 aa, Artificial Sequence
  Synthetic Linker
AKTTPKLEEGEFSEAR

SEQ ID NO: 60 - protein, 17 aa, Artificial Sequence
  Synthetic Linker
AKTTPKLEEGEFSEARV

SEQ ID NO: 61 - protein, 9 aa, Artificial Sequence
  Synthetic Linker
AKTTPKLGG

SEQ ID NO: 62 - protein, 10 aa, Artificial Sequence
  Synthetic Linker
SAKTTPKLGG

SEQ ID NO: 63 - protein, 6 aa, Artificial Sequence
  Synthetic Linker
SAKTTP

SEQ ID NO: 64 - protein, 6 aa, Artificial Sequence
  Synthetic Linker
RADAAP

SEQ ID NO: 65 - protein, 9 aa, Artificial Sequence
  Synthetic Linker
RADAAPTVS

SEQ ID NO: 66 - protein, 12 aa, Artificial Sequence
  Synthetic Linker
RADAAAAGGPGS

SEQ ID NO: 67 - protein, 27 aa, Artificial Sequence
  Synthetic Linker
RADAAAAGGGGSGGGGSGGGGSGGGGS

SEQ ID NO: 68 - protein, 18 aa, Artificial Sequence
  Synthetic Linker
SAKTTPKLEEGEFSEARV

SEQ ID NO: 69 - protein, 5 aa, Artificial Sequence
  Synthetic Linker
ADAAP

SEQ ID NO: 70 - protein, 12 aa, Artificial Sequence
  Synthetic Linker
ADAAPTVSIFPP

SEQ ID NO: 71 - protein, 6 aa, Artificial Sequence
  Synthetic Linker
QPKAAP

SEQ ID NO: 72 - protein, 13 aa, Artificial Sequence
  Synthetic Linker
QPKAAPSVTLFPP

SEQ ID NO: 73 - protein, 6 aa, Artificial Sequence
  Synthetic Linker
AKTTPP

SEQ ID NO: 74 - protein, 13 aa, Artificial Sequence
  Synthetic Linker
AKTTPPSVTPLAP

SEQ ID NO: 75 - protein, 6 aa, Artificial Sequence
  Synthetic Linker
AKTTAP

SEQ ID NO: 76 - protein, 13 aa, Artificial Sequence
  Synthetic Linker
AKTTAPSVYPLAP

SEQ ID NO: 77 - protein, 15 aa, Artificial Sequence
  Synthetic Linker
GGGGSGGGGSGGGGS

SEQ ID NO: 78 - protein, 15 aa, Artificial Sequence
  Synthetic Linker
GENKVEYAPALMALS

SEQ ID NO: 79 - protein, 15 aa, Artificial Sequence
  Synthetic Linker
GPAKELTPLKEAKVS

SEQ ID NO: 80 - protein, 15 aa, Artificial Sequence
  Synthetic Linker
GHEAAAVMQVQYPAS

SEQ ID NO: 81 - protein, 24 aa, Artificial Sequence
  Synthetic Linker
TVAAPSVFIFPPTVAAPSVFIFPP

SEQ ID NO: 82 - protein, 26 aa, Artificial Sequence
  Synthetic Linker
ASTKGPSVFPLAPASTKGPSVFPLAP

SEQ ID NO: 83 - protein, 692 aa, Homo sapiens
  misc_feature: LRRC33 (also known as NRROS; Uniprot Accession No. Q86YC3)
MELLPLWLCLGFHFLTVGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLYLVLILPSCLTLLVACTVIVLTFKKPLLQVIKSRCHWSSVY

SEQ ID NO: 84 - protein, 660 aa, Homo sapiens
  misc_feature: soluble LRRC33 (sLRRC33)
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLHHHHHH

SEQ ID NO: 85 - protein, 5 aa, Artificial Sequence
  Synthetic Ab3 CDRH1
NYAMS

SEQ ID NO: 86 - protein, 17 aa, Artificial Sequence
  Synthetic Ab3 CDRH2
SISGSGGATYYADSVKG

SEQ ID NO: 87 - protein, 12 aa, Artificial Sequence
  Synthetic Ab3 CDRH3
ARVSSGHWDFDY

SEQ ID NO: 88 - protein, 11 aa, Artificial Sequence
  Synthetic Ab3 CDRL1
RASQSISSYLN

SEQ ID NO: 89 - protein, 5 aa, Artificial Sequence
  Synthetic Ab3 CDRL2
SSLQS

SEQ ID NO: 90 - protein, 9 aa, Artificial Sequence
  Synthetic Ab3 CDRL3
QQSYSAPFT

SEQ ID NO: 91 - DNA, 369 bp, Artificial Sequence
  Synthetic Ab1  Heavy chain variable region nucleic acid sequence
gaggtgcaactcgtggagtcaggcggtggacttgttcagcctgggcgaagtctgagactctcatgtgcagcaagtggattcactttctccagttacggcatgcactgggtgagacaggcgcctggaaagggtttggaatgggtcgctgtgatctcttacgacgggtcaaacaaatattacgcggattcagtgaaagggcggttcactatttcacgggataactccaagaacaccctgtatctgcagatgaatagcctgagggcagaggacaccgctgtgtactattgtgcccgggacataaggccttacggcgattacagcgccgcatttgatatttggggacaaggcacccttgtgacagtatcttct

SEQ ID NO: 92 - DNA, 338 bp, Artificial Sequence
  Synthetic Ab1  Light chain variable region nucleic acid sequence
aattttatgcttacccaaccacatagtgtgagtgagtctcccggcaagactgtaacaatttcatgtaccggcagcagtggctccatcgctagcaattatgtgcaatggtaccaacagcgccccgggagcgcaccttcaatagtgatattcgaggataaccaacggcctagtggggctcccgatagatttagtgggagtatagatagctcctccaactctgcctctctcaccattagcgggctgaaaacagaggatgaagccgactattactgccaaagctatgattctagcaaccacggcggagtgtttggcggaggaacacagctgacagtcctagg

SEQ ID NO: 93 - DNA, 372 bp, Artificial Sequence
  Synthetic Ab2  Heavy chain variable region nucleic acid sequence
gaggtgcaactggtgcaatccggagccgagatgaaaaagccaggggagagcctgaagatctcttgtaagggctctggctataacttcgctagtgattggatcggatgggtgaggcaaacccccggaaagggcctcgagtggatgggcgtgatctaccccggcgactccgacacacgctatagcgcctcattccagggccaggtcaccataagtgctgataaatcaataaatacagcctacttgcaatggtcaagtctgaaagcctcagatactgccatgtactattgtgcctctgccgccggcattgccgcggccggtcacgtcaccgccttcgacatttggggtcagggcactatggtcactgtaagctcc

SEQ ID NO: 94 - DNA, 339 bp, Artificial Sequence
  Synthetic Ab2  Light chain variable region nucleic acid sequence
gacatagtcatgacccagtcacctgactctttggccgtgtctctgggggagagagccacaataaattgcaagtcatcacagagcgtcctgtactcctccaataataaaaattacctggcctggtaccagcaaaagcccgggcaaccccccaaattgttgatttactgggctagtacaagggaatctggagtgccagaccggttttctggttctggatctggtactgacttcaccctgacaatcagctccctgcaggccgaagacgtggctgtgtactattgtcagcagtactatagtacaccagttactttcggccaaggcactaaactcgaaatcaag

SEQ ID NO: 95 - protein, 119 aa, Artificial Sequence
  Synthetic Ab3  Heavy chain variable region amino acid sequence
EVQLLESGGGLVQPGGSLRLSCAASGFTFRNYAMSWVRQAPGKGLEWVSSISGSGGATYYADSVKGRFTISRDNSKNTLYLQMNSLRAEDTAVYYCARVSSGHWDFDYWGQGTLVTVSS

SEQ ID NO: 96 - DNA, 357 bp, Artificial Sequence
  Synthetic Ab3  Heavy chain variable region nucleic acid sequence
gaggttcagcttctggagagcggcggtggtcttgtacaacctggaggatcactcaggttgtcatgtgccgcaagcgggtttacattcaggaactatgcaatgagctgggtcagacaggctcccggcaagggacttgagtgggtatcttccatcagcggatctggaggagcaacatattatgcagatagtgtcaaaggcaggttcacaataagccgcgacaattctaaaaatactctttatcttcaaatgaatagccttagggctgaggatacggcggtgtattattgtgcccgcgtctcaagcgggcattgggacttcgattattgggggcagggtactctggttactgtttcctcc

SEQ ID NO: 97 - protein, 107 aa, Artificial Sequence
  Synthetic Ab3  Light chain variable region amino acid sequence
DIQMTQSPSSLSASVGDRVTITCRASQSISSYLNWYQQKPGKAPKLLIYDASSLQSGVPSRFSGSGSGTDFTLTISSLQPEDFATYYCQQSYSAPFTFGQGTKVEIK

SEQ ID NO: 98 - DNA, 322 bp, Artificial Sequence
  Synthetic Ab3  Light chain variable region nucleic acid sequence
gacatccaaatgacacagagcccgtcttccctctcagcttcagtcggtgatcgagtgacgattacgtgccgcgccagccaaagcatctcctcctatcttaactggtatcagcagaaacccggaaaggccccaaagttgcttatttacgacgcatcctcccttcaatctggtgtgcccagcaggttctcaggcagcggttcaggaacggattttactcttaccatttctagtcttcaacctgaggattttgcgacgtattactgtcaacagagctacagtgcgccgttcacctttgggcagggtactaaggttgagataaagc

SEQ ID NO: 99 - protein, 445 aa, Artificial Sequence
  Synthetic Ab3  Heavy chain amino acid sequence
EVQLLESGGGLVQPGGSLRLSCAASGFTFRNYAMSWVRQAPGKGLEWVSSISGSGGATYYADSVKGRFTISRDNSKNTLYLQMNSLRAEDTAVYYCARVSSGHWDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 214 aa, Artificial Sequence
  Synthetic Ab3  Light chain amino acid sequence
DIQMTQSPSSLSASVGDRVTITCRASQSISSYLNWYQQKPGKAPKLLIYDASSLQSGVPSRFSGSGSGTDFTLTISSLQPEDFATYYCQQSYSAPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 689 aa, Artificial Sequence
  Synthetic Human LRRC33-GARP chimera
MDMRVPAQLLGLLLLWFSGVLGWRNRSGTATAASQGVCKLVGGAADCRGQSLASVPSSLPPHARMLTLDANPLKTLWNHSLQPYPLLESLSLHSCHLERISRGAFQEQGHLRSLVLGDNCLSENYEETAAALHALPGLRRLDLSGNALTEDMAALMLQNLSSLRSVSLAGNTIMRLDDSVFEGLERLRELDLQRNYIFEIEGGAFDGLAELRHLNLAFNNLPCIVDFGLTRLRVLNVSYNVLEWFLATGGEAAFELETLDLSHNQLLFFPLLPQYSKLRTLLLRDNNMGFYRDLYNTSSPREMVAQFLLVDGNVTNITTVSLWEEFSSSDLADLRFLDMSQNQFQYLPDGFLRKMPSLSHLNLHQNCLMTLHIREHEPPGALTELDLSHNQLSELHLAPGLASCLGSLRLFNLSSNQLLGVPPGLFANARNITTLDMSHNQISLCPLPAASDRVGPPSCVDFRNMASLRSLSLEGCGLGALPDCPFQGTSLTYLDLSSNWGVLNGSLAPLQDVAPMLQVLSLRNMGLHSSFMALDFSGFGNLRDLDLSGNCLTTFPRFGGSLALETLDLRRNSLTALPQKAVSEQLSRGLRTIYLSQNPYDCCGVDGWGALQHGQTVADWAMVTCNLSSKIIRVTELPGGVPRDCKWERLDLGLLIIILTFILVSAILLTTLAACCCVRRQKFNQQYKA